SEQUENCE LISTING (388 sequences)

SEQ ID NO: 1 - protein, 21 aa, synthetic construct
VLTTGLPALISWIRRRHRRHC

SEQ ID NO: 2 - protein, 21 aa, synthetic construct
VLTTGLPALISWIRRRHRRHG

SEQ ID NO: 3 - protein, 25 aa, synthetic construct
VLTTGLPALISWIKRKRQHRWRRRR

SEQ ID NO: 4 - protein, 189 aa, Homo sapiens [sequence omitted]

SEQ ID NO: 5 - RNA, 19 nt, synthetic construct
  source 1..19: mol_type = other RNA
cggctcggccagtactccc

SEQ ID NO: 6 - RNA, 19 nt, synthetic construct
  source 1..19: mol_type = other RNA
ttgacgatacagctaattc

SEQ ID NO: 7 - RNA, 19 nt, synthetic construct
  source 1..19: mol_type = other RNA
tgacgatacagctaattca

SEQ ID NO: 8 - RNA, 19 nt, synthetic construct
  source 1..19: mol_type = other RNA
gacgatacagctaattcag

SEQ ID NO: 9 - RNA, 19 nt, synthetic construct
  source 1..19: mol_type = other RNA
acgatacagctaattcaga

SEQ ID NO: 10 - RNA, 19 nt, synthetic construct
  source 1..19: mol_type = other RNA
gatacagctaattcagaat

SEQ ID NO: 11 - RNA, 19 nt, synthetic construct
  source 1..19: mol_type = other RNA
ggacgaatatgatccaaca

SEQ ID NO: 12 - RNA, 19 nt, synthetic construct
  source 1..19: mol_type = other RNA
gacgaatatgatccaacaa

SEQ ID NO: 13 - RNA, 19 nt, synthetic construct
  source 1..19: mol_type = other RNA
acgaatatgatccaacaat

SEQ ID NO: 14 - RNA, 19 nt, synthetic construct
  source 1..19: mol_type = other RNA
cgaatatgatccaacaata

SEQ ID NO: 15 - RNA, 19 nt, synthetic construct
  source 1..19: mol_type = other RNA
gtctcttggatattctcga

SEQ ID NO: 16 - RNA, 19 nt, synthetic construct
  source 1..19: mol_type = other RNA
ggatattctcgacacagca

SEQ ID NO: 17 - RNA, 19 nt, synthetic construct
  source 1..19: mol_type = other RNA
gcaatgagggaccagtaca

SEQ ID NO: 18 - RNA, 19 nt, synthetic construct
  source 1..19: mol_type = other RNA
caatgagggaccagtacat

SEQ ID NO: 19 - RNA, 19 nt, synthetic construct
  source 1..19: mol_type = other RNA
agatattcaccattataga

SEQ ID NO: 20 - RNA, 19 nt, synthetic construct
  source 1..19: mol_type = other RNA
gatattcaccattatagag

SEQ ID NO: 21 - RNA, 19 nt, synthetic construct
  source 1..19: mol_type = other RNA
atattcaccattatagaga

SEQ ID NO: 22 - RNA, 19 nt, synthetic construct
  source 1..19: mol_type = other RNA
tattcaccattatagagaa

SEQ ID NO: 23 - RNA, 19 nt, synthetic construct
  source 1..19: mol_type = other RNA
acctatggtcctagtagga

SEQ ID NO: 24 - RNA, 19 nt, synthetic construct
  source 1..19: mol_type = other RNA
ggacttagcaagaagttat

SEQ ID NO: 25 - RNA, 19 nt, synthetic construct
  source 1..19: mol_type = other RNA
ttctatacattagttcgag

SEQ ID NO: 26 - RNA, 19 nt, synthetic construct
  source 1..19: mol_type = other RNA
tatacattagttcgagaaa

SEQ ID NO: 27 - RNA, 19 nt, synthetic construct
  source 1..19: mol_type = other RNA
cattagttcgagaaattcg

SEQ ID NO: 28 - RNA, 19 nt, synthetic construct
  source 1..19: mol_type = other RNA
attagttcgagaaattcga

SEQ ID NO: 29 - RNA, 19 nt, synthetic construct
  source 1..19: mol_type = other RNA
ttagttcgagaaattcgaa

SEQ ID NO: 30 - RNA, 19 nt, synthetic construct
  source 1..19: mol_type = other RNA
agttcgagaaattcgaaaa

SEQ ID NO: 31 - RNA, 19 nt, synthetic construct
  source 1..19: mol_type = other RNA
agaaattcgaaaacataaa

SEQ ID NO: 32 - RNA, 19 nt, synthetic construct
  source 1..19: mol_type = other RNA
aattcgaaaacataaagaa

SEQ ID NO: 33 - RNA, 19 nt, synthetic construct
  source 1..19: mol_type = other RNA
tgtacattacactaaatta

SEQ ID NO: 34 - RNA, 19 nt, synthetic construct
  source 1..19: mol_type = other RNA
gcagttgattacttcttat

SEQ ID NO: 35 - RNA, 19 nt, synthetic construct
  source 1..19: mol_type = other RNA
gaatcatccctattctgtg

SEQ ID NO: 36 - RNA, 19 nt, synthetic construct
  source 1..19: mol_type = other RNA
gcatcatgtcctatagttt

SEQ ID NO: 37 - RNA, 19 nt, synthetic construct
  source 1..19: mol_type = other RNA
cactgctattagtcatggt

SEQ ID NO: 38 - RNA, 19 nt, synthetic construct
  source 1..19: mol_type = other RNA
tgctattagtcatggtcac

SEQ ID NO: 39 - RNA, 19 nt, synthetic construct
  source 1..19: mol_type = other RNA
acattagataaattactat

SEQ ID NO: 40 - RNA, 19 nt, synthetic construct
  source 1..19: mol_type = other RNA
agtatgaaatggggattat

SEQ ID NO: 41 - RNA, 19 nt, synthetic construct
  source 1..19: mol_type = other RNA
accattttggggctatatt

SEQ ID NO: 42 - RNA, 19 nt, synthetic construct
  source 1..19: mol_type = other RNA
tttgggccagttatagctt

SEQ ID NO: 43 - RNA, 19 nt, synthetic construct
  source 1..19: mol_type = other RNA
ggtcctgctgacaaatcaa

SEQ ID NO: 44 - RNA, 19 nt, synthetic construct
  source 1..19: mol_type = other RNA
acaatctctaggtttggct

SEQ ID NO: 45 - RNA, 19 nt, synthetic construct
  source 1..19: mol_type = other RNA
tctaggtttggctagttct

SEQ ID NO: 46 - RNA, 19 nt, synthetic construct
  source 1..19: mol_type = other RNA
gtttggctagttctcttaa

SEQ ID NO: 47 - RNA, 19 nt, synthetic construct
  source 1..19: mol_type = other RNA
ttggctagttctcttaaca

SEQ ID NO: 48 - RNA, 19 nt, synthetic construct
  source 1..19: mol_type = other RNA
gcatggtgaggtgaaagta

SEQ ID NO: 49 - RNA, 19 nt, synthetic construct
  source 1..19: mol_type = other RNA
catggtgaggtgaaagtat

SEQ ID NO: 50 - RNA, 19 nt, synthetic construct
  source 1..19: mol_type = other RNA
gtgacttaggttctagata

SEQ ID NO: 51 - RNA, 19 nt, synthetic construct
  source 1..19: mol_type = other RNA
tgacttaggttctagatag

SEQ ID NO: 52 - RNA, 19 nt, synthetic construct
  source 1..19: mol_type = other RNA
aggacatcacttactatcc

SEQ ID NO: 53 - RNA, 19 nt, synthetic construct
  source 1..19: mol_type = other RNA
ggatacacttatttgtcaa

SEQ ID NO: 54 - RNA, 19 nt, synthetic construct
  source 1..19: mol_type = other RNA
tttaacctatgttacacca

SEQ ID NO: 55 - RNA, 19 nt, synthetic construct
  source 1..19: mol_type = other RNA
ttaacctatgttacaccat

SEQ ID NO: 56 - RNA, 19 nt, synthetic construct
  source 1..19: mol_type = other RNA
tgggcaaaattgtgcaaga

SEQ ID NO: 57 - RNA, 19 nt, synthetic construct
  source 1..19: mol_type = other RNA
tttgaatatccattctcgt

SEQ ID NO: 58 - RNA, 19 nt, synthetic construct
  source 1..19: mol_type = other RNA
tcccctaaccataagattt

SEQ ID NO: 59 - RNA, 19 nt, synthetic construct
  source 1..19: mol_type = other RNA
cccctaaccataagattta

SEQ ID NO: 60 - RNA, 19 nt, synthetic construct
  source 1..19: mol_type = other RNA
ccctaaccataagatttac

SEQ ID NO: 61 - RNA, 19 nt, synthetic construct
  source 1..19: mol_type = other RNA
cctaaccataagatttact

SEQ ID NO: 62 - RNA, 19 nt, synthetic construct
  source 1..19: mol_type = other RNA
agggatttgacctaatcac

SEQ ID NO: 63 - RNA, 19 nt, synthetic construct
  source 1..19: mol_type = other RNA
tgacctaatcactaatttt

SEQ ID NO: 64 - RNA, 19 nt, synthetic construct
  source 1..19: mol_type = other RNA
ctgcccaatccattagcga

SEQ ID NO: 65 - RNA, 19 nt, synthetic construct
  source 1..19: mol_type = other RNA
tgcccaatccattagcgac

SEQ ID NO: 66 - RNA, 19 nt, synthetic construct
  source 1..19: mol_type = other RNA
gcccaatccattagcgaca

SEQ ID NO: 67 - RNA, 19 nt, synthetic construct
  source 1..19: mol_type = other RNA
cccaatccattagcgacag

SEQ ID NO: 68 - RNA, 19 nt, synthetic construct
  source 1..19: mol_type = other RNA
ccaatccattagcgacagt

SEQ ID NO: 69 - RNA, 19 nt, synthetic construct
  source 1..19: mol_type = other RNA
caatccattagcgacagta

SEQ ID NO: 70 - RNA, 19 nt, synthetic construct
  source 1..19: mol_type = other RNA
atccattagcgacagtagg

SEQ ID NO: 71 - RNA, 19 nt, synthetic construct
  source 1..19: mol_type = other RNA
cattagcgacagtaggatt

SEQ ID NO: 72 - RNA, 19 nt, synthetic construct
  source 1..19: mol_type = other RNA
ggtatgaatagacagaacc

SEQ ID NO: 73 - RNA, 19 nt, synthetic construct
  source 1..19: mol_type = other RNA
aatagacagaaccctatcc

SEQ ID NO: 74 - RNA, 19 nt, synthetic construct
  source 1..19: mol_type = other RNA
tcctggtaacagtaataca

SEQ ID NO: 75 - RNA, 19 nt, synthetic construct
  source 1..19: mol_type = other RNA
agttttatctgggtacaaa

SEQ ID NO: 76 - RNA, 19 nt, synthetic construct
  source 1..19: mol_type = other RNA
gtagggtgttaagacttac

SEQ ID NO: 77 - RNA, 19 nt, synthetic construct
  source 1..19: mol_type = other RNA
ggggatatttaggcctctt

SEQ ID NO: 78 - RNA, 19 nt, synthetic construct
  source 1..19: mol_type = other RNA
cttaggcattaacatgttt

SEQ ID NO: 79 - RNA, 19 nt, synthetic construct
  source 1..19: mol_type = other RNA
cctaacttttataggttat

SEQ ID NO: 80 - RNA, 19 nt, synthetic construct
  source 1..19: mol_type = other RNA
gaatagtcataactagatt

SEQ ID NO: 81 - RNA, 19 nt, synthetic construct
  source 1..19: mol_type = other RNA
cataactagattaagatct

SEQ ID NO: 82 - RNA, 19 nt, synthetic construct
  source 1..19: mol_type = other RNA
gggataatgataggtaatt

SEQ ID NO: 83 - RNA, 19 nt, synthetic construct
  source 1..19: mol_type = other RNA
ggataatgataggtaattt

SEQ ID NO: 84 - RNA, 19 nt, synthetic construct
  source 1..19: mol_type = other RNA
gtgttttatccgaaagttt

SEQ ID NO: 85 - RNA, 19 nt, synthetic construct
  source 1..19: mol_type = other RNA
tgttttatccgaaagtttc

SEQ ID NO: 86 - RNA, 19 nt, synthetic construct
  source 1..19: mol_type = other RNA
tatccgaaagtttccaatt

SEQ ID NO: 87 - RNA, 19 nt, synthetic construct
  source 1..19: mol_type = other RNA
ccgaaagtttccaattcca

SEQ ID NO: 88 - RNA, 19 nt, synthetic construct
  source 1..19: mol_type = other RNA
gccaatttcttactagtac

SEQ ID NO: 89 - RNA, 19 nt, synthetic construct
  source 1..19: mol_type = other RNA
ccaatttcttactagtact

SEQ ID NO: 90 - RNA, 19 nt, synthetic construct
  source 1..19: mol_type = other RNA
ccatcatttggttgcgctg

SEQ ID NO: 91 - RNA, 19 nt, synthetic construct
  source 1..19: mol_type = other RNA
tcatttggttgcgctgacc

SEQ ID NO: 92 - RNA, 19 nt, synthetic construct
  source 1..19: mol_type = other RNA
acctaggaatgttggtcat

SEQ ID NO: 93 - RNA, 19 nt, synthetic construct
  source 1..19: mol_type = other RNA
cctaggaatgttggtcata

SEQ ID NO: 94 - RNA, 19 nt, synthetic construct
  source 1..19: mol_type = other RNA
ctaggaatgttggtcatat

SEQ ID NO: 95 - RNA, 19 nt, synthetic construct
  source 1..19: mol_type = other RNA
taggaatgttggtcatatc

SEQ ID NO: 96 - RNA, 19 nt, synthetic construct
  source 1..19: mol_type = other RNA
ggaatgttggtcatatcaa

SEQ ID NO: 97 - RNA, 19 nt, synthetic construct
  source 1..19: mol_type = other RNA
actgtactactcctaatta

SEQ ID NO: 98 - RNA, 19 nt, synthetic construct
  source 1..19: mol_type = other RNA
ctgtactactcctaattat

SEQ ID NO: 99 - RNA, 19 nt, synthetic construct
  source 1..19: mol_type = other RNA
tgtactactcctaattatt

SEQ ID NO: 100 - RNA, 19 nt, synthetic construct
  source 1..19: mol_type = other RNA
gtactactcctaattattg

SEQ ID NO: 101 - RNA, 19 nt, synthetic construct
  source 1..19: mol_type = other RNA
gggagtactggccgagccg

SEQ ID NO: 102 - RNA, 19 nt, synthetic construct
  source 1..19: mol_type = other RNA
gaattagctgtatcgtcaa

SEQ ID NO: 103 - RNA, 19 nt, synthetic construct
  source 1..19: mol_type = other RNA
tgaattagctgtatcgtca

SEQ ID NO: 104 - RNA, 19 nt, synthetic construct
  source 1..19: mol_type = other RNA
ctgaattagctgtatcgtc

SEQ ID NO: 105 - RNA, 19 nt, synthetic construct
  source 1..19: mol_type = other RNA
tctgaattagctgtatcgt

SEQ ID NO: 106 - RNA, 19 nt, synthetic construct
  source 1..19: mol_type = other RNA
attctgaattagctgtatc

SEQ ID NO: 107 - RNA, 19 nt, synthetic construct
  source 1..19: mol_type = other RNA
tgttggatcatattcgtcc

SEQ ID NO: 108 - RNA, 19 nt, synthetic construct
  source 1..19: mol_type = other RNA
ttgttggatcatattcgtc

SEQ ID NO: 109 - RNA, 19 nt, synthetic construct
  source 1..19: mol_type = other RNA
attgttggatcatattcgt

SEQ ID NO: 110 - RNA, 19 nt, synthetic construct
  source 1..19: mol_type = other RNA
tattgttggatcatattcg

SEQ ID NO: 111 - RNA, 19 nt, synthetic construct
  source 1..19: mol_type = other RNA
tcgagaatatccaagagac

SEQ ID NO: 112 - RNA, 19 nt, synthetic construct
  source 1..19: mol_type = other RNA
tgctgtgtcgagaatatcc

SEQ ID NO: 113 - RNA, 19 nt, synthetic construct
  source 1..19: mol_type = other RNA
tgtactggtccctcattgc

SEQ ID NO: 114 - RNA, 19 nt, synthetic construct
  source 1..19: mol_type = other RNA
atgtactggtccctcattg

SEQ ID NO: 115 - RNA, 19 nt, synthetic construct
  source 1..19: mol_type = other RNA
tctataatggtgaatatct

SEQ ID NO: 116 - RNA, 19 nt, synthetic construct
  source 1..19: mol_type = other RNA
ctctataatggtgaatatc

SEQ ID NO: 117 - RNA, 19 nt, synthetic construct
  source 1..19: mol_type = other RNA
tctctataatggtgaatat

SEQ ID NO: 118 - RNA, 19 nt, synthetic construct
  source 1..19: mol_type = other RNA
ttctctataatggtgaata

SEQ ID NO: 119 - RNA, 19 nt, synthetic construct
  source 1..19: mol_type = other RNA
tcctactaggaccataggt

SEQ ID NO: 120 - RNA, 19 nt, synthetic construct
  source 1..19: mol_type = other RNA
ataacttcttgctaagtcc

SEQ ID NO: 121 - RNA, 19 nt, synthetic construct
  source 1..19: mol_type = other RNA
ctcgaactaatgtatagaa

SEQ ID NO: 122 - RNA, 19 nt, synthetic construct
  source 1..19: mol_type = other RNA
tttctcgaactaatgtata

SEQ ID NO: 123 - RNA, 19 nt, synthetic construct
  source 1..19: mol_type = other RNA
cgaatttctcgaactaatg

SEQ ID NO: 124 - RNA, 19 nt, synthetic construct
  source 1..19: mol_type = other RNA
tcgaatttctcgaactaat

SEQ ID NO: 125 - RNA, 19 nt, synthetic construct
  source 1..19: mol_type = other RNA
ttcgaatttctcgaactaa

SEQ ID NO: 126 - RNA, 19 nt, synthetic construct
  source 1..19: mol_type = other RNA
ttttcgaatttctcgaact

SEQ ID NO: 127 - RNA, 19 nt, synthetic construct
  source 1..19: mol_type = other RNA
tttatgttttcgaatttct

SEQ ID NO: 128 - RNA, 19 nt, synthetic construct
  source 1..19: mol_type = other RNA
ttctttatgttttcgaatt

SEQ ID NO: 129 - RNA, 19 nt, synthetic construct
  source 1..19: mol_type = other RNA
taatttagtgtaatgtaca

SEQ ID NO: 130 - RNA, 19 nt, synthetic construct
  source 1..19: mol_type = other RNA
ataagaagtaatcaactgc

SEQ ID NO: 131 - RNA, 19 nt, synthetic construct
  source 1..19: mol_type = other RNA
cacagaatagggatgattc

SEQ ID NO: 132 - RNA, 19 nt, synthetic construct
  source 1..19: mol_type = other RNA
aaactataggacatgatgc

SEQ ID NO: 133 - RNA, 19 nt, synthetic construct
  source 1..19: mol_type = other RNA
accatgactaatagcagtg

SEQ ID NO: 134 - RNA, 19 nt, synthetic construct
  source 1..19: mol_type = other RNA
gtgaccatgactaatagca

SEQ ID NO: 135 - RNA, 19 nt, synthetic construct
  source 1..19: mol_type = other RNA
atagtaatttatctaatgt

SEQ ID NO: 136 - RNA, 19 nt, synthetic construct
  source 1..19: mol_type = other RNA
ataatccccatttcatact

SEQ ID NO: 137 - RNA, 19 nt, synthetic construct
  source 1..19: mol_type = other RNA
aatatagccccaaaatggt

SEQ ID NO: 138 - RNA, 19 nt, synthetic construct
  source 1..19: mol_type = other RNA
aagctataactggcccaaa

SEQ ID NO: 139 - RNA, 19 nt, synthetic construct
  source 1..19: mol_type = other RNA
ttgatttgtcagcaggacc

SEQ ID NO: 140 - RNA, 19 nt, synthetic construct
  source 1..19: mol_type = other RNA
agccaaacctagagattgt

SEQ ID NO: 141 - RNA, 19 nt, synthetic construct
  source 1..19: mol_type = other RNA
agaactagccaaacctaga

SEQ ID NO: 142 - RNA, 19 nt, synthetic construct
  source 1..19: mol_type = other RNA
ttaagagaactagccaaac

SEQ ID NO: 143 - RNA, 19 nt, synthetic construct
  source 1..19: mol_type = other RNA
tgttaagagaactagccaa

SEQ ID NO: 144 - RNA, 19 nt, synthetic construct
  source 1..19: mol_type = other RNA
tactttcacctcaccatgc

SEQ ID NO: 145 - RNA, 19 nt, synthetic construct
  source 1..19: mol_type = other RNA
atactttcacctcaccatg

SEQ ID NO: 146 - RNA, 19 nt, synthetic construct
  source 1..19: mol_type = other RNA
tatctagaacctaagtcac

SEQ ID NO: 147 - RNA, 19 nt, synthetic construct
  source 1..19: mol_type = other RNA
ctatctagaacctaagtca

SEQ ID NO: 148 - RNA, 19 nt, synthetic construct
  source 1..19: mol_type = other RNA
ggatagtaagtgatgtcct

SEQ ID NO: 149 - RNA, 19 nt, synthetic construct
  source 1..19: mol_type = other RNA
ttgacaaataagtgtatcc

SEQ ID NO: 150 - RNA, 19 nt, synthetic construct
  source 1..19: mol_type = other RNA
tggtgtaacataggttaaa

SEQ ID NO: 151 - RNA, 19 nt, synthetic construct
  source 1..19: mol_type = other RNA
atggtgtaacataggttaa

SEQ ID NO: 152 - RNA, 19 nt, synthetic construct
  source 1..19: mol_type = other RNA
tcttgcacaattttgccca

SEQ ID NO: 153 - RNA, 19 nt, synthetic construct
  source 1..19: mol_type = other RNA
acgagaatggatattcaaa

SEQ ID NO: 154 - RNA, 19 nt, synthetic construct
  source 1..19: mol_type = other RNA
aaatcttatggttagggga

SEQ ID NO: 155 - RNA, 19 nt, synthetic construct
  source 1..19: mol_type = other RNA
taaatcttatggttagggg

SEQ ID NO: 156 - RNA, 19 nt, synthetic construct
  source 1..19: mol_type = other RNA
gtaaatcttatggttaggg

SEQ ID NO: 157 - RNA, 19 nt, synthetic construct
  source 1..19: mol_type = other RNA
agtaaatcttatggttagg

SEQ ID NO: 158 - RNA, 19 nt, synthetic construct
  source 1..19: mol_type = other RNA
gtgattaggtcaaatccct

SEQ ID NO: 159 - RNA, 19 nt, synthetic construct
  source 1..19: mol_type = other RNA
aaaattagtgattaggtca

SEQ ID NO: 160 - RNA, 19 nt, synthetic construct
  source 1..19: mol_type = other RNA
tcgctaatggattgggcag

SEQ ID NO: 161 - RNA, 19 nt, synthetic construct
  source 1..19: mol_type = other RNA
gtcgctaatggattgggca

SEQ ID NO: 162 - RNA, 19 nt, synthetic construct
  source 1..19: mol_type = other RNA
tgtcgctaatggattgggc

SEQ ID NO: 163 - RNA, 19 nt, synthetic construct
  source 1..19: mol_type = other RNA
ctgtcgctaatggattggg

SEQ ID NO: 164 - RNA, 19 nt, synthetic construct
  source 1..19: mol_type = other RNA
actgtcgctaatggattgg

SEQ ID NO: 165 - RNA, 19 nt, synthetic construct
  source 1..19: mol_type = other RNA
tactgtcgctaatggattg

SEQ ID NO: 166 - RNA, 19 nt, synthetic construct
  source 1..19: mol_type = other RNA
cctactgtcgctaatggat

SEQ ID NO: 167 - RNA, 19 nt, synthetic construct
  source 1..19: mol_type = other RNA
aatcctactgtcgctaatg

SEQ ID NO: 168 - RNA, 19 nt, synthetic construct
  source 1..19: mol_type = other RNA
ggttctgtctattcatacc

SEQ ID NO: 169 - RNA, 19 nt, synthetic construct
  source 1..19: mol_type = other RNA
ggatagggttctgtctatt

SEQ ID NO: 170 - RNA, 19 nt, synthetic construct
  source 1..19: mol_type = other RNA
tgtattactgttaccagga

SEQ ID NO: 171 - RNA, 19 nt, synthetic construct
  source 1..19: mol_type = other RNA
tttgtacccagataaaact

SEQ ID NO: 172 - RNA, 19 nt, synthetic construct
  source 1..19: mol_type = other RNA
gtaagtcttaacaccctac

SEQ ID NO: 173 - RNA, 19 nt, synthetic construct
  source 1..19: mol_type = other RNA
aagaggcctaaatatcccc

SEQ ID NO: 174 - RNA, 19 nt, synthetic construct
  source 1..19: mol_type = other RNA
aaacatgttaatgcctaag

SEQ ID NO: 175 - RNA, 19 nt, synthetic construct
  source 1..19: mol_type = other RNA
ataacctataaaagttagg

SEQ ID NO: 176 - RNA, 19 nt, synthetic construct
  source 1..19: mol_type = other RNA
aatctagttatgactattc

SEQ ID NO: 177 - RNA, 19 nt, synthetic construct
  source 1..19: mol_type = other RNA
agatcttaatctagttatg

SEQ ID NO: 178 - RNA, 19 nt, synthetic construct
  source 1..19: mol_type = other RNA
aattacctatcattatccc

SEQ ID NO: 179 - RNA, 19 nt, synthetic construct
  source 1..19: mol_type = other RNA
aaattacctatcattatcc

SEQ ID NO: 180 - RNA, 19 nt, synthetic construct
  source 1..19: mol_type = other RNA
aaactttcggataaaacac

SEQ ID NO: 181 - RNA, 19 nt, synthetic construct
  source 1..19: mol_type = other RNA
gaaactttcggataaaaca

SEQ ID NO: 182 - RNA, 19 nt, synthetic construct
  source 1..19: mol_type = other RNA
aattggaaactttcggata

SEQ ID NO: 183 - RNA, 19 nt, synthetic construct
  source 1..19: mol_type = other RNA
tggaattggaaactttcgg

SEQ ID NO: 184 - RNA, 19 nt, synthetic construct
  source 1..19: mol_type = other RNA
gtactagtaagaaattggc

SEQ ID NO: 185 - RNA, 19 nt, synthetic construct
  source 1..19: mol_type = other RNA
agtactagtaagaaattgg

SEQ ID NO: 186 - RNA, 19 nt, synthetic construct
  source 1..19: mol_type = other RNA
cagcgcaaccaaatgatgg

SEQ ID NO: 187 - RNA, 19 nt, synthetic construct
  source 1..19: mol_type = other RNA
ggtcagcgcaaccaaatga

SEQ ID NO: 188 - RNA, 19 nt, synthetic construct
  source 1..19: mol_type = other RNA
atgaccaacattcctaggt

SEQ ID NO: 189 - RNA, 19 nt, synthetic construct
  source 1..19: mol_type = other RNA
tatgaccaacattcctagg

SEQ ID NO: 190 - RNA, 19 nt, synthetic construct
  source 1..19: mol_type = other RNA
atatgaccaacattcctag

SEQ ID NO: 191 - RNA, 19 nt, synthetic construct
  source 1..19: mol_type = other RNA
gatatgaccaacattccta

SEQ ID NO: 192 - RNA, 19 nt, synthetic construct
  source 1..19: mol_type = other RNA
ttgatatgaccaacattcc

SEQ ID NO: 193 - RNA, 19 nt, synthetic construct
  source 1..19: mol_type = other RNA
taattaggagtagtacagt

SEQ ID NO: 194 - RNA, 19 nt, synthetic construct
  source 1..19: mol_type = other RNA
ataattaggagtagtacag

SEQ ID NO: 195 - RNA, 19 nt, synthetic construct
  source 1..19: mol_type = other RNA
aataattaggagtagtaca

SEQ ID NO: 196 - RNA, 19 nt, synthetic construct
  source 1..19: mol_type = other RNA
caataattaggagtagtac

SEQ ID NO: 197 - RNA, 19 nt, synthetic construct
  modified_base 1..3: mod_base = OTHER; note = linked via phosphorothioate
  modified_base 1: mod_base = cm
  modified_base 2: mod_base = gm
  modified_base 3: mod_base = OTHER; note = 2'-Fluoro-guanosine
  modified_base 4: mod_base = cm
  modified_base 5: mod_base = um
  modified_base 6: mod_base = cm
  modified_base 7: mod_base = OTHER; note = 2'-Fluoro-guanosine
  modified_base 8: mod_base = OTHER; note = 2'-Fluoro-guanosine
  modified_base 9: mod_base = OTHER; note = 2 -Fluoro-cytidine
  modified_base 10: mod_base = cm
  modified_base 11: mod_base = OTHER; note = 2 -O-methyl-adenosine
  modified_base 12: mod_base = OTHER; note = 2'-Fluoro-guanosine
  modified_base 13: mod_base = um
  modified_base 14: mod_base = OTHER; note = 2 -O-methyl-adenosine
  modified_base 15: mod_base = cm
  modified_base 16: mod_base = um
  modified_base 17: mod_base = OTHER; note = 2 -Fluoro-cytidine
  modified_base 18: mod_base = cm
  modified_base 19: mod_base = OTHER; note = 2 -O-methyl-adenosine
  source 1..19: mol_type = other RNA
cggctcggccagtactcca

SEQ ID NO: 198 - RNA, 19 nt, synthetic construct
  modified_base 1..3: mod_base = OTHER; note = linked via phosphorothioate
  modified_base 1: mod_base = um
  modified_base 2: mod_base = um
  modified_base 3: mod_base = OTHER; note = 2'-Fluoro-guanosine
  modified_base 4: mod_base = OTHER; note = 2 -O-methyl-adenosine
  modified_base 5: mod_base = cm
  modified_base 6: mod_base = gm
  modified_base 7: mod_base = OTHER; note = 2'-Fluoro-adenosine
  modified_base 8: mod_base = OTHER; note = 2'-Fluoro-uridine
  modified_base 9: mod_base = OTHER; note = 2'-Fluoro-adenosine
  modified_base 10: mod_base = cm
  modified_base 11: mod_base = OTHER; note = 2 -O-methyl-adenosine
  modified_base 12: mod_base = OTHER; note = 2'-Fluoro-guanosine
  modified_base 13: mod_base = cm
  modified_base 14: mod_base = um
  modified_base 15: mod_base = OTHER; note = 2 -O-methyl-adenosine
  modified_base 16: mod_base = OTHER; note = 2 -O-methyl-adenosine
  modified_base 17: mod_base = OTHER; note = 2'-Fluoro-uridine
  modified_base 18: mod_base = um
  modified_base 19: mod_base = OTHER; note = 2 -O-methyl-adenosine
  source 1..19: mol_type = other RNA
ttgacgatacagctaatta

SEQ ID NO: 199 - RNA, 19 nt, synthetic construct
  modified_base 1..3: mod_base = OTHER; note = linked via phosphorothioate
  modified_base 1: mod_base = um
  modified_base 2: mod_base = gm
  modified_base 3: mod_base = OTHER; note = 2'-Fluoro-adenosine
  modified_base 4: mod_base = cm
  modified_base 5: mod_base = gm
  modified_base 6: mod_base = OTHER; note = 2 -O-methyl-adenosine
  modified_base 7: mod_base = OTHER; note = 2'-Fluoro-uridine
  modified_base 8: mod_base = OTHER; note = 2'-Fluoro-adenosine
  modified_base 9: mod_base = OTHER; note = 2 -Fluoro-cytidine
  modified_base 10: mod_base = OTHER; note = 2 -O-methyl-adenosine
  modified_base 11: mod_base = gm
  modified_base 12: mod_base = OTHER; note = 2 -Fluoro-cytidine
  modified_base 13: mod_base = um
  modified_base 14: mod_base = OTHER; note = 2 -O-methyl-adenosine
  modified_base 15: mod_base = OTHER; note = 2 -O-methyl-adenosine
  modified_base 16: mod_base = um
  modified_base 17: mod_base = OTHER; note = 2'-Fluoro-uridine
  modified_base 18: mod_base = cm
  modified_base 19: mod_base = OTHER; note = 2 -O-methyl-adenosine
  source 1..19: mol_type = other RNA
tgacgatacagctaattca

SEQ ID NO: 200 - RNA, 19 nt, synthetic construct
  modified_base 1..3: mod_base = OTHER; note = linked via phosphorothioate
  modified_base 1: mod_base = gm
  modified_base 2: mod_base = OTHER; note = 2 -O-methyl-adenosine
  modified_base 3: mod_base = OTHER; note = 2 -Fluoro-cytidine
  modified_base 4: mod_base = gm
  modified_base 5: mod_base = OTHER; note = 2 -O-methyl-adenosine
  modified_base 6: mod_base = um
  modified_base 7: mod_base = OTHER; note = 2'-Fluoro-adenosine
  modified_base 8: mod_base = OTHER; note = 2 -Fluoro-cytidine
  modified_base 9: mod_base = OTHER; note = 2'-Fluoro-adenosine
  modified_base 10: mod_base = gm
  modified_base 11: mod_base = cm
  modified_base 12: mod_base = OTHER; note = 2'-Fluoro-uridine
  modified_base 13: mod_base = OTHER; note = 2 -O-methyl-adenosine
  modified_base 14: mod_base = OTHER; note = 2 -O-methyl-adenosine
  modified_base 15: mod_base = um
  modified_base 16: mod_base = um
  modified_base 17: mod_base = OTHER; note = 2 -Fluoro-cytidine
  modified_base 18: mod_base = OTHER; note = 2 -O-methyl-adenosine
  modified_base 19: mod_base = OTHER; note = 2 -O-methyl-adenosine
  source 1..19: mol_type = other RNA
gacgatacagctaattcaa

SEQ ID NO: 201 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = cm
  modified_base 3: mod_base = OTHER; note = 2'-Fluoro-guanosine
  modified_base 4: mod_base = OTHER; note = 2 -O-methyl-adenosine
  modified_base 5: mod_base = um
  modified_base 6: mod_base = OTHER; note = 2 -O-methyl-adenosine
  modified_base 7: mod_base = OTHER; note = 2 -Fluoro-cytidine
  modified_base 8: mod_base = OTHER; note = 2'-Fluoro-adenosine
  modified_base 9: mod_base = OTHER; note = 2'-Fluoro-guanosine
  modified_base 10: mod_base = cm
  modified_base 11: mod_base = um
  modified_base 12: mod_base = OTHER; note = 2'-Fluoro-adenosine
  modified_base 13: mod_base = OTHER; note = 2 -O-methyl-adenosine
  modified_base 14: mod_base = um
  modified_base 15: mod_base = um
  modified_base 16: mod_base = cm
  modified_base 17: mod_base = OTHER; note = 2'-Fluoro-adenosine
  modified_base 18: mod_base = gm
  modified_base 19: mod_base = OTHER; note = 2 -O-methyl-adenosine
  source 1..19: mol_type = other RNA
acgatacagctaattcaga

SEQ ID NO: 202 - RNA, 19 nt, synthetic construct
  modified_base 1..3: mod_base = OTHER; note = linked via phosphorothioate
  modified_base 1: mod_base = gm
  modified_base 2: mod_base = OTHER; note = 2 -O-methyl-adenosine
  modified_base 3: mod_base = OTHER; note = 2'-Fluoro-uridine
  modified_base 4: mod_base = OTHER; note = 2 -O-methyl-adenosine
  modified_base 5: mod_base = cm
  modified_base 6: mod_base = OTHER; note = 2 -O-methyl-adenosine
  modified_base 7: mod_base = OTHER; note = 2'-Fluoro-guanosine
  modified_base 8: mod_base = OTHER; note = 2 -Fluoro-cytidine
  modified_base 9: mod_base = OTHER; note = 2'-Fluoro-uridine
  modified_base 10: mod_base = OTHER; note = 2 -O-methyl-adenosine
  modified_base 11: mod_base = OTHER; note = 2 -O-methyl-adenosine
  modified_base 12: mod_base = OTHER; note = 2'-Fluoro-uridine
  modified_base 13: mod_base = um
  modified_base 14: mod_base = cm
  modified_base 15: mod_base = OTHER; note = 2 -O-methyl-adenosine
  modified_base 16: mod_base = gm
  modified_base 17: mod_base = OTHER; note = 2'-Fluoro-adenosine
  modified_base 18: mod_base = OTHER; note = 2 -O-methyl-adenosine
  modified_base 19: mod_base = OTHER; note = 2 -O-methyl-adenosine
  source 1..19: mol_type = other RNA
gatacagctaattcagaaa

SEQ ID NO: 203 - RNA, 19 nt, synthetic construct
  modified_base 1..3: mod_base = OTHER; note = linked via phosphorothioate
  modified_base 1: mod_base = gm
  modified_base 2: mod_base = gm
  modified_base 3: mod_base = OTHER; note = 2'-Fluoro-adenosine
  modified_base 4: mod_base = cm
  modified_base 5: mod_base = gm
  modified_base 6: mod_base = OTHER; note = 2 -O-methyl-adenosine
  modified_base 7: mod_base = OTHER; note = 2'-Fluoro-adenosine
  modified_base 8: mod_base = OTHER; note = 2'-Fluoro-uridine
  modified_base 9: mod_base = OTHER; note = 2'-Fluoro-adenosine
  modified_base 10: mod_base = um
  modified_base 11: mod_base = gm
  modified_base 12: mod_base = OTHER; note = 2'-Fluoro-adenosine
  modified_base 13: mod_base = um
  modified_base 14: mod_base = cm
  modified_base 15: mod_base = cm
  modified_base 16: mod_base = OTHER; note = 2 -O-methyl-adenosine
  modified_base 17: mod_base = OTHER; note = 2'-Fluoro-adenosine
  modified_base 18: mod_base = cm
  modified_base 19: mod_base = OTHER; note = 2 -O-methyl-adenosine
  source 1..19: mol_type = other RNA
ggacgaatatgatccaaca

SEQ ID NO: 204 - RNA, 19 nt, synthetic construct
  modified_base 1..3: mod_base = OTHER; note = linked via phosphorothioate
  modified_base 1: mod_base = gm
  modified_base 2: mod_base = OTHER; note = 2 -O-methyl-adenosine
  modified_base 3: mod_base = OTHER; note = 2 -Fluoro-cytidine
  modified_base 4: mod_base = gm
  modified_base 5: mod_base = OTHER; note = 2 -O-methyl-adenosine
  modified_base 6: mod_base = OTHER; note = 2 -O-methyl-adenosine
  modified_base 7: mod_base = OTHER; note = 2'-Fluoro-uridine
  modified_base 8: mod_base = OTHER; note = 2'-Fluoro-adenosine
  modified_base 9: mod_base = OTHER; note = 2'-Fluoro-uridine
  modified_base 10: mod_base = gm
  modified_base 11: mod_base = OTHER; note = 2 -O-methyl-adenosine
  modified_base 12: mod_base = OTHER; note = 2'-Fluoro-uridine
  modified_base 13: mod_base = cm
  modified_base 14: mod_base = cm
  modified_base 15: mod_base = OTHER; note = 2 -O-methyl-adenosine
  modified_base 16: mod_base = OTHER; note = 2 -O-methyl-adenosine
  modified_base 17: mod_base = OTHER; note = 2 -Fluoro-cytidine
  modified_base 18: mod_base = OTHER; note = 2 -O-methyl-adenosine
  modified_base 19: mod_base = OTHER; note = 2 -O-methyl-adenosine
  source 1..19: mol_type = other RNA
gacgaatatgatccaacaa

SEQ ID NO: 205 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = cm
  modified_base 3: mod_base = OTHER; note = 2'-Fluoro-guanosine
  modified_base 4: mod_base = OTHER; note = 2 -O-methyl-adenosine
  modified_base 5: mod_base = OTHER; note = 2 -O-methyl-adenosine
  modified_base 6: mod_base = um
  modified_base 7: mod_base = OTHER; note = 2'-Fluoro-adenosine
  modified_base 8: mod_base = OTHER; note = 2'-Fluoro-uridine
  modified_base 9: mod_base = OTHER; note = 2'-Fluoro-guanosine
  modified_base 10: mod_base = OTHER; note = 2 -O-methyl-adenosine
  modified_base 11: mod_base = um
  modified_base 12: mod_base = OTHER; note = 2 -Fluoro-cytidine
  modified_base 13: mod_base = cm
  modified_base 14: mod_base = OTHER; note = 2 -O-methyl-adenosine
  modified_base 15: mod_base = OTHER; note = 2 -O-methyl-adenosine
  modified_base 16: mod_base = cm
  modified_base 17: mod_base = OTHER; note = 2'-Fluoro-adenosine
  modified_base 18: mod_base = OTHER; note = 2 -O-methyl-adenosine
  modified_base 19: mod_base = OTHER; note = 2 -O-methyl-adenosine
  source 1..19: mol_type = other RNA
acgaatatgatccaacaaa

SEQ ID NO: 206 - RNA, 19 nt, synthetic construct
  modified_base 1..3: mod_base = OTHER; note = linked via phosphorothioate
  modified_base 1: mod_base = cm
  modified_base 2: mod_base = gm
  modified_base 3: mod_base = OTHER; note = 2'-Fluoro-adenosine
  modified_base 4: mod_base = OTHER; note = 2 -O-methyl-adenosine
  modified_base 5: mod_base = um
  modified_base 6: mod_base = OTHER; note = 2 -O-methyl-adenosine
  modified_base 7: mod_base = OTHER; note = 2'-Fluoro-uridine
  modified_base 8: mod_base = OTHER; note = 2'-Fluoro-guanosine
  modified_base 9: mod_base = OTHER; note = 2'-Fluoro-adenosine
  modified_base 10: mod_base = um
  modified_base 11: mod_base = cm
  modified_base 12: mod_base = OTHER; note = 2 -Fluoro-cytidine
  modified_base 13: mod_base = OTHER; note = 2 -O-methyl-adenosine
  modified_base 14: mod_base = OTHER; note = 2 -O-methyl-adenosine
  modified_base 15: mod_base = cm
  modified_base 16: mod_base = OTHER; note = 2 -O-methyl-adenosine
  modified_base 17: mod_base = OTHER; note = 2'-Fluoro-adenosine
  modified_base 18: mod_base = um
  modified_base 19: mod_base = OTHER; note = 2 -O-methyl-adenosine
  source 1..19: mol_type = other RNA
cgaatatgatccaacaata

SEQ ID NO: 207 - RNA, 19 nt, synthetic construct
  modified_base 1..3: mod_base = OTHER; note = linked via phosphorothioate
  modified_base 1: mod_base = gm
  modified_base 2: mod_base = um
  modified_base 3: mod_base = OTHER; note = 2 -Fluoro-cytidine
  modified_base 4: mod_base = um
  modified_base 5: mod_base = cm
  modified_base 6: mod_base = um
  modified_base 7: mod_base = OTHER; note = 2'-Fluoro-uridine
  modified_base 8: mod_base = OTHER; note = 2'-Fluoro-guanosine
  modified_base 9: mod_base = OTHER; note = 2'-Fluoro-guanosine
  modified_base 10: mod_base = OTHER; note = 2 -O-methyl-adenosine
  modified_base 11: mod_base = um
  modified_base 12: mod_base = OTHER; note = 2'-Fluoro-adenosine
  modified_base 13: mod_base = um
  modified_base 14: mod_base = um
  modified_base 15: mod_base = cm
  modified_base 16: mod_base = um
  modified_base 17: mod_base = OTHER; note = 2 -Fluoro-cytidine
  modified_base 18: mod_base = gm
  modified_base 19: mod_base = OTHER; note = 2 -O-methyl-adenosine
  source 1..19: mol_type = other RNA
gtctcttggatattctcga

SEQ ID NO: 208 - RNA, 19 nt, synthetic construct
  modified_base 1..3: mod_base = OTHER; note = linked via phosphorothioate
  modified_base 1: mod_base = gm
  modified_base 2: mod_base = gm
  modified_base 3: mod_base = OTHER; note = 2'-Fluoro-adenosine
  modified_base 4: mod_base = um
  modified_base 5: mod_base = OTHER; note = 2 -O-methyl-adenosine
  modified_base 6: mod_base = um
  modified_base 7: mod_base = OTHER; note = 2'-Fluoro-uridine
  modified_base 8: mod_base = OTHER; note = 2 -Fluoro-cytidine
  modified_base 9: mod_base = OTHER; note = 2'-Fluoro-uridine
  modified_base 10: mod_base = cm
  modified_base 11: mod_base = gm
  modified_base 12: mod_base = OTHER; note = 2'-Fluoro-adenosine
  modified_base 13: mod_base = cm
  modified_base 14: mod_base = OTHER; note = 2 -O-methyl-adenosine
  modified_base 15: mod_base = cm
  modified_base 16: mod_base = OTHER; note = 2 -O-methyl-adenosine
  modified_base 17: mod_base = OTHER; note = 2'-Fluoro-guanosine
  modified_base 18: mod_base = cm
  modified_base 19: mod_base = OTHER; note = 2 -O-methyl-adenosine
  source 1..19: mol_type = other RNA
ggatattctcgacacagca

SEQ ID NO: 209 - RNA, 19 nt, synthetic construct
  modified_base 1..3: mod_base = OTHER; note = linked via phosphorothioate
  modified_base 1: mod_base = gm
  modified_base 2: mod_base = cm
  modified_base 3: mod_base = OTHER; note = 2'-Fluoro-adenosine
  modified_base 4: mod_base = OTHER; note = 2 -O-methyl-adenosine
  modified_base 5: mod_base = um
  modified_base 6: mod_base = gm
  modified_base 7: mod_base = OTHER; note = 2'-Fluoro-adenosine
  modified_base 8: mod_base = OTHER; note = 2'-Fluoro-guanosine
  modified_base 9: mod_base = OTHER; note = 2'-Fluoro-guanosine
  modified_base 10: mod_base = gm
  modified_base 11: mod_base = OTHER; note = 2 -O-methyl-adenosine
  modified_base 12: mod_base = OTHER; note = 2 -Fluoro-cytidine
  modified_base 13: mod_base = cm
  modified_base 14: mod_base = OTHER; note = 2 -O-methyl-adenosine
  modified_base 15: mod_base = gm
  modified_base 16: mod_base = um
  modified_base 17: mod_base = OTHER; note = 2'-Fluoro-adenosine
  modified_base 18: mod_base = cm
  modified_base 19: mod_base = OTHER; note = 2 -O-methyl-adenosine
  source 1..19: mol_type = other RNA
gcaatgagggaccagtaca

SEQ ID NO: 210 - RNA, 19 nt, synthetic construct
  modified_base 1..3: mod_base = OTHER; note = linked via phosphorothioate
  modified_base 1: mod_base = cm
  modified_base 2: mod_base = OTHER; note = 2 -O-methyl-adenosine
  modified_base 3: mod_base = OTHER; note = 2'-Fluoro-adenosine
  modified_base 4: mod_base = um
  modified_base 5: mod_base = gm
  modified_base 6: mod_base = OTHER; note = 2 -O-methyl-adenosine
  modified_base 7: mod_base = OTHER; note = 2'-Fluoro-guanosine
  modified_base 8: mod_base = OTHER; note = 2'-Fluoro-guanosine
  modified_base 9: mod_base = OTHER; note = 2'-Fluoro-guanosine
  modified_base 10: mod_base = OTHER; note = 2 -O-methyl-adenosine
  modified_base 11: mod_base = cm
  modified_base 12: mod_base = OTHER; note = 2 -Fluoro-cytidine
  modified_base 13: mod_base = OTHER; note = 2 -O-methyl-adenosine
  modified_base 14: mod_base = gm
  modified_base 15: mod_base = um
  modified_base 16: mod_base = OTHER; note = 2 -O-methyl-adenosine
  modified_base 17: mod_base = OTHER; note = 2 -Fluoro-cytidine
  modified_base 18: mod_base = OTHER; note = 2 -O-methyl-adenosine
  modified_base 19: mod_base = OTHER; note = 2 -O-methyl-adenosine
  source 1..19: mol_type = other RNA
caatgagggaccagtacaa

SEQ ID NO: 211 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = gm
  modified_base 3: mod_base = OTHER; note = 2'-Fluoro-adenosine
  modified_base 4: mod_base = um
  modified_base 5: mod_base = OTHER; note = 2 -O-methyl-adenosine
  modified_base 6: mod_base = um
  modified_base 7: mod_base = OTHER; note = 2'-Fluoro-uridine
  modified_base 8: mod_base = OTHER; note = 2 -Fluoro-cytidine
  modified_base 9: mod_base = OTHER; note = 2'-Fluoro-adenosine
  modified_base 10: mod_base = cm
  modified_base 11: mod_base = cm
  modified_base 12: mod_base = OTHER; note = 2'-Fluoro-adenosine
  modified_base 13: mod_base = um
  modified_base 14: mod_base = um
  modified_base 15: mod_base = OTHER; note = 2 -O-methyl-adenosine
  modified_base 16: mod_base = um
  modified_base 17: mod_base = OTHER; note = 2'-Fluoro-adenosine
  modified_base 18: mod_base = gm
  modified_base 19: mod_base = OTHER; note = 2 -O-methyl-adenosine
  source 1..19: mol_type = other RNA
agatattcaccattataga

SEQ ID NO: 212 - RNA, 19 nt, synthetic construct
  modified_base 1..3: mod_base = OTHER; note = linked via phosphorothioate
  modified_base 1: mod_base = gm
  modified_base 2: mod_base = OTHER; note = 2 -O-methyl-adenosine
  modified_base 3: mod_base = OTHER; note = 2'-Fluoro-uridine
  modified_base 4: mod_base = OTHER; note = 2 -O-methyl-adenosine
  modified_base 5: mod_base = um
  modified_base 6: mod_base = um
  modified_base 7: mod_base = OTHER; note = 2 -Fluoro-cytidine
  modified_base 8: mod_base = OTHER; note = 2'-Fluoro-adenosine
  modified_base 9: mod_base = OTHER; note = 2 -Fluoro-cytidine
  modified_base 10: mod_base = cm
  modified_base 11: mod_base = OTHER; note = 2 -O-methyl-adenosine
  modified_base 12: mod_base = OTHER; note = 2'-Fluoro-uridine
  modified_base 13: mod_base = um
  modified_base 14: mod_base = OTHER; note = 2 -O-methyl-adenosine
  modified_base 15: mod_base = um
  modified_base 16: mod_base = OTHER; note = 2 -O-methyl-adenosine
  modified_base 17: mod_base = OTHER; note = 2'-Fluoro-guanosine
  modified_base 18: mod_base = OTHER; note = 2 -O-methyl-adenosine
  modified_base 19: mod_base = OTHER; note = 2 -O-methyl-adenosine
  source 1..19: mol_type = other RNA
gatattcaccattatagaa

SEQ ID NO: 213 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = um
  modified_base 3: mod_base = OTHER; note = 2'-Fluoro-adenosine
  modified_base 4: mod_base = um
  modified_base 5: mod_base = um
  modified_base 6: mod_base = cm
  modified_base 7: mod_base = OTHER; note = 2'-Fluoro-adenosine
  modified_base 8: mod_base = OTHER; note = 2 -Fluoro-cytidine
  modified_base 9: mod_base = OTHER; note = 2 -Fluoro-cytidine
  modified_base 10: mod_base = OTHER; note = 2 -O-methyl-adenosine
  modified_base 11: mod_base = um
  modified_base 12: mod_base = OTHER; note = 2'-Fluoro-uridine
  modified_base 13: mod_base = OTHER; note = 2 -O-methyl-adenosine
  modified_base 14: mod_base = um
  modified_base 15: mod_base = OTHER; note = 2 -O-methyl-adenosine
  modified_base 16: mod_base = gm
  modified_base 17: mod_base = OTHER; note = 2'-Fluoro-adenosine
  modified_base 18: mod_base = gm
  modified_base 19: mod_base = OTHER; note = 2 -O-methyl-adenosine
  source 1..19: mol_type = other RNA
atattcaccattatagaga

SEQ ID NO: 214 - RNA, 19 nt, synthetic construct
  modified_base 1..3: mod_base = OTHER; note = linked via phosphorothioate
  modified_base 1: mod_base = um
  modified_base 2: mod_base = OTHER; note = 2 -O-methyl-adenosine
  modified_base 3: mod_base = OTHER; note = 2'-Fluoro-uridine
  modified_base 4: mod_base = um
  modified_base 5: mod_base = cm
  modified_base 6: mod_base = OTHER; note = 2 -O-methyl-adenosine
  modified_base 7: mod_base = OTHER; note = 2 -Fluoro-cytidine
  modified_base 8: mod_base = OTHER; note = 2 -Fluoro-cytidine
  modified_base 9: mod_base = OTHER; note = 2'-Fluoro-adenosine
  modified_base 10: mod_base = um
  modified_base 11: mod_base = um
  modified_base 12: mod_base = OTHER; note = 2'-Fluoro-adenosine
  modified_base 13: mod_base = um
  modified_base 14: mod_base = OTHER; note = 2 -O-methyl-adenosine
  modified_base 15: mod_base = gm
  modified_base 16: mod_base = OTHER; note = 2 -O-methyl-adenosine
  modified_base 17: mod_base = OTHER; note = 2'-Fluoro-guanosine
  modified_base 18: mod_base = OTHER; note = 2 -O-methyl-adenosine
  modified_base 19: mod_base = OTHER; note = 2 -O-methyl-adenosine
  source 1..19: mol_type = other RNA
tattcaccattatagagaa

SEQ ID NO: 215 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = cm
  modified_base 3: mod_base = OTHER; note = 2 -Fluoro-cytidine
  modified_base 4: mod_base = um
  modified_base 5: mod_base = OTHER; note = 2 -O-methyl-adenosine
  modified_base 6: mod_base = um
  modified_base 7: mod_base = OTHER; note = 2'-Fluoro-guanosine
  modified_base 8: mod_base = OTHER; note = 2'-Fluoro-guanosine
  modified_base 9: mod_base = OTHER; note = 2'-Fluoro-uridine
  modified_base 10: mod_base = cm
  modified_base 11: mod_base = cm
  modified_base 12: mod_base = OTHER; note = 2'-Fluoro-uridine
  modified_base 13: mod_base = OTHER; note = 2 -O-methyl-adenosine
  modified_base 14: mod_base = gm
  modified_base 15: mod_base = um
  modified_base 16: mod_base = OTHER; note = 2 -O-methyl-adenosine
  modified_base 17: mod_base = OTHER; note = 2'-Fluoro-guanosine
  modified_base 18: mod_base = gm
  modified_base 19: mod_base = OTHER; note = 2 -O-methyl-adenosine
  source 1..19: mol_type = other RNA
acctatggtcctagtagga

SEQ ID NO: 216 - RNA, 19 nt, synthetic construct
  modified_base 1..3: mod_base = OTHER; note = linked via phosphorothioate
  modified_base 1: mod_base = gm
  modified_base 2: mod_base = gm
  modified_base 3: mod_base = OTHER; note = 2'-Fluoro-adenosine
  modified_base 4: mod_base = cm
  modified_base 5: mod_base = um
  modified_base 6: mod_base = um
  modified_base 7: mod_base = OTHER; note = 2'-Fluoro-adenosine
  modified_base 8: mod_base = OTHER; note = 2'-Fluoro-guanosine
  modified_base 9: mod_base = OTHER; note = 2 -Fluoro-cytidine
  modified_base 10: mod_base = OTHER; note = 2 -O-methyl-adenosine
  modified_base 11: mod_base = OTHER; note = 2 -O-methyl-adenosine
  modified_base 12: mod_base = OTHER; note = 2'-Fluoro-guanosine
  modified_base 13: mod_base = OTHER; note = 2 -O-methyl-adenosine
  modified_base 14: mod_base = OTHER; note = 2 -O-methyl-adenosine
  modified_base 15: mod_base = gm
  modified_base 16: mod_base = um
  modified_base 17: mod_base = OTHER; note = 2'-Fluoro-uridine
  modified_base 18: mod_base = OTHER; note = 2 -O-methyl-adenosine
  modified_base 19: mod_base = OTHER; note = 2 -O-methyl-adenosine
  source 1..19: mol_type = other RNA
ggacttagcaagaagttaa

SEQ ID NO: 217 - RNA, 19 nt, synthetic construct
  modified_base 1..3: mod_base = OTHER; note = linked via phosphorothioate
  modified_base 1: mod_base = um
  modified_base 2: mod_base = um
  modified_base 3: mod_base = OTHER; note = 2 -Fluoro-cytidine
  modified_base 4: mod_base = um
  modified_base 5: mod_base = OTHER; note = 2 -O-methyl-adenosine
  modified_base 6: mod_base = um
  modified_base 7: mod_base = OTHER; note = 2'-Fluoro-adenosine
  modified_base 8: mod_base = OTHER; note = 2 -Fluoro-cytidine
  modified_base 9: mod_base = OTHER; note = 2'-Fluoro-adenosine
  modified_base 10: mod_base = um
  modified_base 11: mod_base = um
  modified_base 12: mod_base = OTHER; note = 2'-Fluoro-adenosine
  modified_base 13: mod_base = gm
  modified_base 14: mod_base = um
  modified_base 15: mod_base = um
  modified_base 16: mod_base = cm
  modified_base 17: mod_base = OTHER; note = 2'-Fluoro-guanosine
  modified_base 18: mod_base = OTHER; note = 2 -O-methyl-adenosine
  modified_base 19: mod_base = OTHER; note = 2 -O-methyl-adenosine
  source 1..19: mol_type = other RNA
ttctatacattagttcgaa

SEQ ID NO: 218 - RNA, 19 nt, synthetic construct
  modified_base 1..3: mod_base = OTHER; note = linked via phosphorothioate
  modified_base 1: mod_base = um
  modified_base 2: mod_base = OTHER; note = 2 -O-methyl-adenosine
  modified_base 3: mod_base = OTHER; note = 2'-Fluoro-uridine
  modified_base 4: mod_base = OTHER; note = 2 -O-methyl-adenosine
  modified_base 5: mod_base = cm
  modified_base 6: mod_base = OTHER; note = 2 -O-methyl-adenosine
  modified_base 7: mod_base = OTHER; note = 2'-Fluoro-uridine
  modified_base 8: mod_base = OTHER; note = 2'-Fluoro-uridine
  modified_base 9: mod_base = OTHER; note = 2'-Fluoro-adenosine
  modified_base 10: mod_base = gm
  modified_base 11: mod_base = um
  modified_base 12: mod_base = OTHER; note = 2'-Fluoro-uridine
  modified_base 13: mod_base = cm
  modified_base 14: mod_base = gm
  modified_base 15: mod_base = OTHER; note = 2 -O-methyl-adenosine
  modified_base 16: mod_base = gm
  modified_base 17: mod_base = OTHER; note = 2'-Fluoro-adenosine
  modified_base 18: mod_base = OTHER; note = 2 -O-methyl-adenosine
  modified_base 19: mod_base = OTHER; note = 2 -O-methyl-adenosine
  source 1..19: mol_type = other RNA
tatacattagttcgagaaa

SEQ ID NO: 219 - RNA, 19 nt, synthetic construct
  modified_base 1..3: mod_base = OTHER; note = linked via phosphorothioate
  modified_base 1: mod_base = cm
  modified_base 2: mod_base = OTHER; note = 2 -O-methyl-adenosine
  modified_base 3: mod_base = OTHER; note = 2'-Fluoro-uridine
  modified_base 4: mod_base = um
  modified_base 5: mod_base = OTHER; note = 2 -O-methyl-adenosine
  modified_base 6: mod_base = gm
  modified_base 7: mod_base = OTHER; note = 2'-Fluoro-uridine
  modified_base 8: mod_base = OTHER; note = 2'-Fluoro-uridine
  modified_base 9: mod_base = OTHER; note = 2 -Fluoro-cytidine
  modified_base 10: mod_base = gm
  modified_base 11: mod_base = OTHER; note = 2 -O-methyl-adenosine
  modified_base 12: mod_base = OTHER; note = 2'-Fluoro-guanosine
  modified_base 13: mod_base = OTHER; note = 2 -O-methyl-adenosine
  modified_base 14: mod_base = OTHER; note = 2 -O-methyl-adenosine
  modified_base 15: mod_base = OTHER; note = 2 -O-methyl-adenosine
  modified_base 16: mod_base = um
  modified_base 17: mod_base = OTHER; note = 2'-Fluoro-uridine
  modified_base 18: mod_base = cm
  modified_base 19: mod_base = OTHER; note = 2 -O-methyl-adenosine
  source 1..19: mol_type = other RNA
cattagttcgagaaattca

SEQ ID NO: 220 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = um
  modified_base 3: mod_base = OTHER; note = 2'-Fluoro-uridine
  modified_base 4: mod_base = OTHER; note = 2 -O-methyl-adenosine
  modified_base 5: mod_base = gm
  modified_base 6: mod_base = um
  modified_base 7: mod_base = OTHER; note = 2'-Fluoro-uridine
  modified_base 8: mod_base = OTHER; note = 2 -Fluoro-cytidine
  modified_base 9: mod_base = OTHER; note = 2'-Fluoro-guanosine
  modified_base 10: mod_base = OTHER; note = 2 -O-methyl-adenosine
  modified_base 11: mod_base = gm
  modified_base 12: mod_base = OTHER; note = 2'-Fluoro-adenosine
  modified_base 13: mod_base = OTHER; note = 2 -O-methyl-adenosine
  modified_base 14: mod_base = OTHER; note = 2 -O-methyl-adenosine
  modified_base 15: mod_base = um
  modified_base 16: mod_base = um
  modified_base 17: mod_base = OTHER; note = 2 -Fluoro-cytidine
  modified_base 18: mod_base = gm
  modified_base 19: mod_base = OTHER; note = 2 -O-methyl-adenosine
  source 1..19: mol_type = other RNA
attagttcgagaaattcga

SEQ ID NO: 221 - RNA, 19 nt, synthetic construct
  modified_base 1..3: mod_base = OTHER; note = linked via phosphorothioate
  modified_base 1: mod_base = um
  modified_base 2: mod_base = um
  modified_base 3: mod_base = OTHER; note = 2'-Fluoro-adenosine
  modified_base 4: mod_base = gm
  modified_base 5: mod_base = um
  modified_base 6: mod_base = um
  modified_base 7: mod_base = OTHER; note = 2 -Fluoro-cytidine
  modified_base 8: mod_base = OTHER; note = 2'-Fluoro-guanosine
  modified_base 9: mod_base = OTHER; note = 2'-Fluoro-adenosine
  modified_base 10: mod_base = gm
  modified_base 11: mod_base = OTHER; note = 2 -O-methyl-adenosine
  modified_base 12: mod_base = OTHER; note = 2'-Fluoro-adenosine
  modified_base 13: mod_base = OTHER; note = 2 -O-methyl-adenosine
  modified_base 14: mod_base = um
  modified_base 15: mod_base = um
  modified_base 16: mod_base = cm
  modified_base 17: mod_base = OTHER; note = 2'-Fluoro-guanosine
  modified_base 18: mod_base = OTHER; note = 2 -O-methyl-adenosine
  modified_base 19: mod_base = OTHER; note = 2 -O-methyl-adenosine
  source 1..19: mol_type = other RNA
ttagttcgagaaattcgaa

SEQ ID NO: 222 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = gm
  modified_base 3: mod_base = OTHER; note = 2'-Fluoro-uridine
  modified_base 4: mod_base = um
  modified_base 5: mod_base = cm
  modified_base 6: mod_base = gm
  modified_base 7: mod_base = OTHER; note = 2'-Fluoro-adenosine
  modified_base 8: mod_base = OTHER; note = 2'-Fluoro-guanosine
  modified_base 9: mod_base = OTHER; note = 2'-Fluoro-adenosine
  modified_base 10: mod_base = OTHER; note = 2 -O-methyl-adenosine
  modified_base 11: mod_base = OTHER; note = 2 -O-methyl-adenosine
  modified_base 12: mod_base = OTHER; note = 2'-Fluoro-uridine
  modified_base 13: mod_base = um
  modified_base 14: mod_base = cm
  modified_base 15: mod_base = gm
  modified_base 16: mod_base = OTHER; note = 2 -O-methyl-adenosine
  modified_base 17: mod_base = OTHER; note = 2'-Fluoro-adenosine
  modified_base 18: mod_base = OTHER; note = 2 -O-methyl-adenosine
  modified_base 19: mod_base = OTHER; note = 2 -O-methyl-adenosine
  source 1..19: mol_type = other RNA
agttcgagaaattcgaaaa

SEQ ID NO: 223 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = gm
  modified_base 3: mod_base = OTHER; note = 2'-Fluoro-adenosine
  modified_base 4: mod_base = OTHER; note = 2 -O-methyl-adenosine
  modified_base 5: mod_base = OTHER; note = 2 -O-methyl-adenosine
  modified_base 6: mod_base = um
  modified_base 7: mod_base = OTHER; note = 2'-Fluoro-uridine
  modified_base 8: mod_base = OTHER; note = 2 -Fluoro-cytidine
  modified_base 9: mod_base = OTHER; note = 2'-Fluoro-guanosine
  modified_base 10: mod_base = OTHER; note = 2 -O-methyl-adenosine
  modified_base 11: mod_base = OTHER; note = 2 -O-methyl-adenosine
  modified_base 12: mod_base = OTHER; note = 2'-Fluoro-adenosine
  modified_base 13: mod_base = OTHER; note = 2 -O-methyl-adenosine
  modified_base 14: mod_base = cm
  modified_base 15: mod_base = OTHER; note = 2 -O-methyl-adenosine
  modified_base 16: mod_base = um
  modified_base 17: mod_base = OTHER; note = 2'-Fluoro-adenosine
  modified_base 18: mod_base = OTHER; note = 2 -O-methyl-adenosine
  modified_base 19: mod_base = OTHER; note = 2 -O-methyl-adenosine
  source 1..19: mol_type = other RNA
agaaattcgaaaacataaa

SEQ ID NO: 224 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = OTHER; note = 2 -O-methyl-adenosine
  modified_base 3: mod_base = OTHER; note = 2'-Fluoro-uridine
  modified_base 4: mod_base = um
  modified_base 5: mod_base = cm
  modified_base 6: mod_base = gm
  modified_base 7: mod_base = OTHER; note = 2'-Fluoro-adenosine
  modified_base 8: mod_base = OTHER; note = 2'-Fluoro-adenosine
  modified_base 9: mod_base = OTHER; note = 2'-Fluoro-adenosine
  modified_base 10: mod_base = OTHER; note = 2 -O-methyl-adenosine
  modified_base 11: mod_base = cm
  modified_base 12: mod_base = OTHER; note = 2'-Fluoro-adenosine
  modified_base 13: mod_base = um
  modified_base 14: mod_base = OTHER; note = 2 -O-methyl-adenosine
  modified_base 15: mod_base = OTHER; note = 2 -O-methyl-adenosine
  modified_base 16: mod_base = OTHER; note = 2 -O-methyl-adenosine
  modified_base 17: mod_base = OTHER; note = 2'-Fluoro-guanosine
  modified_base 18: mod_base = OTHER; note = 2 -O-methyl-adenosine
  modified_base 19: mod_base = OTHER; note = 2 -O-methyl-adenosine
  source 1..19: mol_type = other RNA
aattcgaaaacataaagaa

SEQ ID NO: 225 - RNA, 19 nt, synthetic construct
  modified_base 1..3: mod_base = OTHER; note = linked via phosphorothioate
  modified_base 1: mod_base = um
  modified_base 2: mod_base = gm
  modified_base 3: mod_base = OTHER; note = 2'-Fluoro-uridine
  modified_base 4: mod_base = OTHER; note = 2 -O-methyl-adenosine
  modified_base 5: mod_base = cm
  modified_base 6: mod_base = OTHER; note = 2 -O-methyl-adenosine
  modified_base 7: mod_base = OTHER; note = 2'-Fluoro-uridine
  modified_base 8: mod_base = OTHER; note = 2'-Fluoro-uridine
  modified_base 9: mod_base = OTHER; note = 2'-Fluoro-adenosine
  modified_base 10: mod_base = cm
  modified_base 11: mod_base = OTHER; note = 2 -O-methyl-adenosine
  modified_base 12: mod_base = OTHER; note = 2 -Fluoro-cytidine
  modified_base 13: mod_base = um
  modified_base 14: mod_base = OTHER; note = 2 -O-methyl-adenosine
  modified_base 15: mod_base = OTHER; note = 2 -O-methyl-adenosine
  modified_base 16: mod_base = OTHER; note = 2 -O-methyl-adenosine
  modified_base 17: mod_base = OTHER; note = 2'-Fluoro-uridine
  modified_base 18: mod_base = um
  modified_base 19: mod_base = OTHER; note = 2 -O-methyl-adenosine
  source 1..19: mol_type = other RNA
tgtacattacactaaatta

SEQ ID NO: 226 - RNA, 19 nt, synthetic construct
  modified_base 1..3: mod_base = OTHER; note = linked via phosphorothioate
  modified_base 1: mod_base = gm
  modified_base 2: mod_base = cm
  modified_base 3: mod_base = OTHER; note = 2'-Fluoro-adenosine
  modified_base 4: mod_base = gm
  modified_base 5: mod_base = um
  modified_base 6: mod_base = um
  modified_base 7: mod_base = OTHER; note = 2'-Fluoro-guanosine
  modified_base 8: mod_base = OTHER; note = 2'-Fluoro-adenosine
  modified_base 9: mod_base = OTHER; note = 2'-Fluoro-uridine
  modified_base 10: mod_base = um
  modified_base 11: mod_base = OTHER; note = 2 -O-methyl-adenosine
  modified_base 12: mod_base = OTHER; note = 2 -Fluoro-cytidine
  modified_base 13: mod_base = um
  modified_base 14: mod_base = um
  modified_base 15: mod_base = cm
  modified_base 16: mod_base = um
  modified_base 17: mod_base = OTHER; note = 2'-Fluoro-uridine
  modified_base 18: mod_base = OTHER; note = 2 -O-methyl-adenosine
  modified_base 19: mod_base = OTHER; note = 2 -O-methyl-adenosine
  source 1..19: mol_type = other RNA
gcagttgattacttcttaa

SEQ ID NO: 227 - RNA, 19 nt, synthetic construct
  modified_base 1..3: mod_base = OTHER; note = linked via phosphorothioate
  modified_base 1: mod_base = gm
  modified_base 2: mod_base = OTHER; note = 2 -O-methyl-adenosine
  modified_base 3: mod_base = OTHER; note = 2'-Fluoro-adenosine
  modified_base 4: mod_base = um
  modified_base 5: mod_base = cm
  modified_base 6: mod_base = OTHER; note = 2 -O-methyl-adenosine
  modified_base 7: mod_base = OTHER; note = 2'-Fluoro-uridine
  modified_base 8: mod_base = OTHER; note = 2 -Fluoro-cytidine
  modified_base 9: mod_base = OTHER; note = 2 -Fluoro-cytidine
  modified_base 10: mod_base = cm
  modified_base 11: mod_base = um
  modified_base 12: mod_base = OTHER; note = 2'-Fluoro-adenosine
  modified_base 13: mod_base = um
  modified_base 14: mod_base = um
  modified_base 15: mod_base = cm
  modified_base 16: mod_base = um
  modified_base 17: mod_base = OTHER; note = 2'-Fluoro-guanosine
  modified_base 18: mod_base = um
  modified_base 19: mod_base = OTHER; note = 2 -O-methyl-adenosine
  source 1..19: mol_type = other RNA
gaatcatccctattctgta

SEQ ID NO: 228 - RNA, 19 nt, synthetic construct
  modified_base 1..3: mod_base = OTHER; note = linked via phosphorothioate
  modified_base 1: mod_base = gm
  modified_base 2: mod_base = cm
  modified_base 3: mod_base = OTHER; note = 2'-Fluoro-adenosine
  modified_base 4: mod_base = um
  modified_base 5: mod_base = cm
  modified_base 6: mod_base = OTHER; note = 2 -O-methyl-adenosine
  modified_base 7: mod_base = OTHER; note = 2'-Fluoro-uridine
  modified_base 8: mod_base = OTHER; note = 2'-Fluoro-guanosine
  modified_base 9: mod_base = OTHER; note = 2'-Fluoro-uridine
  modified_base 10: mod_base = cm
  modified_base 11: mod_base = cm
  modified_base 12: mod_base = OTHER; note = 2'-Fluoro-uridine
  modified_base 13: mod_base = OTHER; note = 2 -O-methyl-adenosine
  modified_base 14: mod_base = um
  modified_base 15: mod_base = OTHER; note = 2 -O-methyl-adenosine
  modified_base 16: mod_base = gm
  modified_base 17: mod_base = OTHER; note = 2'-Fluoro-uridine
  modified_base 18: mod_base = um
  modified_base 19: mod_base = OTHER; note = 2 -O-methyl-adenosine
  source 1..19: mol_type = other RNA
gcatcatgtcctatagtta

SEQ ID NO: 229 - RNA, 19 nt, synthetic construct
  modified_base 1..3: mod_base = OTHER; note = linked via phosphorothioate
  modified_base 1: mod_base = cm
  modified_base 2: mod_base = OTHER; note = 2 -O-methyl-adenosine
  modified_base 3: mod_base = OTHER; note = 2 -Fluoro-cytidine
  modified_base 4: mod_base = um
  modified_base 5: mod_base = gm
  modified_base 6: mod_base = cm
  modified_base 7: mod_base = OTHER; note = 2'-Fluoro-uridine
  modified_base 8: mod_base = OTHER; note = 2'-Fluoro-adenosine
  modified_base 9: mod_base = OTHER; note = 2'-Fluoro-uridine
  modified_base 10: mod_base = um
  modified_base 11: mod_base = OTHER; note = 2 -O-methyl-adenosine
  modified_base 12: mod_base = OTHER; note = 2'-Fluoro-guanosine
  modified_base 13: mod_base = um
  modified_base 14: mod_base = cm
  modified_base 15: mod_base = OTHER; note = 2 -O-methyl-adenosine
  modified_base 16: mod_base = um
  modified_base 17: mod_base = OTHER; note = 2'-Fluoro-guanosine
  modified_base 18: mod_base = gm
  modified_base 19: mod_base = OTHER; note = 2 -O-methyl-adenosine
  source 1..19: mol_type = other RNA
cactgctattagtcatgga

SEQ ID NO: 230 - RNA, 19 nt, synthetic construct
  modified_base 1..3: mod_base = OTHER; note = linked via phosphorothioate
  modified_base 1: mod_base = um
  modified_base 2: mod_base = gm
  modified_base 3: mod_base = OTHER; note = 2 -Fluoro-cytidine
  modified_base 4: mod_base = um
  modified_base 5: mod_base = OTHER; note = 2 -O-methyl-adenosine
  modified_base 6: mod_base = um
  modified_base 7: mod_base = OTHER; note = 2'-Fluoro-uridine
  modified_base 8: mod_base = OTHER; note = 2'-Fluoro-adenosine
  modified_base 9: mod_base = OTHER; note = 2'-Fluoro-guanosine
  modified_base 10: mod_base = um
  modified_base 11: mod_base = cm
  modified_base 12: mod_base = OTHER; note = 2'-Fluoro-adenosine
  modified_base 13: mod_base = um
  modified_base 14: mod_base = gm
  modified_base 15: mod_base = gm
  modified_base 16: mod_base = um
  modified_base 17: mod_base = OTHER; note = 2 -Fluoro-cytidine
  modified_base 18: mod_base = OTHER; note = 2 -O-methyl-adenosine
  modified_base 19: mod_base = OTHER; note = 2 -O-methyl-adenosine
  source 1..19: mol_type = other RNA
tgctattagtcatggtcaa

SEQ ID NO: 231 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = cm
  modified_base 3: mod_base = OTHER; note = 2'-Fluoro-adenosine
  modified_base 4: mod_base = um
  modified_base 5: mod_base = um
  modified_base 6: mod_base = OTHER; note = 2 -O-methyl-adenosine
  modified_base 7: mod_base = OTHER; note = 2'-Fluoro-guanosine
  modified_base 8: mod_base = OTHER; note = 2'-Fluoro-adenosine
  modified_base 9: mod_base = OTHER; note = 2'-Fluoro-uridine
  modified_base 10: mod_base = OTHER; note = 2 -O-methyl-adenosine
  modified_base 11: mod_base = OTHER; note = 2 -O-methyl-adenosine
  modified_base 12: mod_base = OTHER; note = 2'-Fluoro-adenosine
  modified_base 13: mod_base = um
  modified_base 14: mod_base = um
  modified_base 15: mod_base = OTHER; note = 2 -O-methyl-adenosine
  modified_base 16: mod_base = cm
  modified_base 17: mod_base = OTHER; note = 2'-Fluoro-uridine
  modified_base 18: mod_base = OTHER; note = 2 -O-methyl-adenosine
  modified_base 19: mod_base = OTHER; note = 2 -O-methyl-adenosine
  source 1..19: mol_type = other RNA
acattagataaattactaa

SEQ ID NO: 232 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = gm
  modified_base 3: mod_base = OTHER; note = 2'-Fluoro-uridine
  modified_base 4: mod_base = OTHER; note = 2 -O-methyl-adenosine
  modified_base 5: mod_base = um
  modified_base 6: mod_base = gm
  modified_base 7: mod_base = OTHER; note = 2'-Fluoro-adenosine
  modified_base 8: mod_base = OTHER; note = 2'-Fluoro-adenosine
  modified_base 9: mod_base = OTHER; note = 2'-Fluoro-adenosine
  modified_base 10: mod_base = um
  modified_base 11: mod_base = gm
  modified_base 12: mod_base = OTHER; note = 2'-Fluoro-guanosine
  modified_base 13: mod_base = gm
  modified_base 14: mod_base = gm
  modified_base 15: mod_base = OTHER; note = 2 -O-methyl-adenosine
  modified_base 16: mod_base = um
  modified_base 17: mod_base = OTHER; note = 2'-Fluoro-uridine
  modified_base 18: mod_base = OTHER; note = 2 -O-methyl-adenosine
  modified_base 19: mod_base = OTHER; note = 2 -O-methyl-adenosine
  source 1..19: mol_type = other RNA
agtatgaaatggggattaa

SEQ ID NO: 233 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = cm
  modified_base 3: mod_base = OTHER; note = 2 -Fluoro-cytidine
  modified_base 4: mod_base = OTHER; note = 2 -O-methyl-adenosine
  modified_base 5: mod_base = um
  modified_base 6: mod_base = um
  modified_base 7: mod_base = OTHER; note = 2'-Fluoro-uridine
  modified_base 8: mod_base = OTHER; note = 2'-Fluoro-uridine
  modified_base 9: mod_base = OTHER; note = 2'-Fluoro-guanosine
  modified_base 10: mod_base = gm
  modified_base 11: mod_base = gm
  modified_base 12: mod_base = OTHER; note = 2'-Fluoro-guanosine
  modified_base 13: mod_base = cm
  modified_base 14: mod_base = um
  modified_base 15: mod_base = OTHER; note = 2 -O-methyl-adenosine
  modified_base 16: mod_base = um
  modified_base 17: mod_base = OTHER; note = 2'-Fluoro-adenosine
  modified_base 18: mod_base = um
  modified_base 19: mod_base = OTHER; note = 2 -O-methyl-adenosine
  source 1..19: mol_type = other RNA
accattttggggctatata

SEQ ID NO: 234 - RNA, 19 nt, synthetic construct
  modified_base 1..3: mod_base = OTHER; note = linked via phosphorothioate
  modified_base 1: mod_base = um
  modified_base 2: mod_base = um
  modified_base 3: mod_base = OTHER; note = 2'-Fluoro-uridine
  modified_base 4: mod_base = gm
  modified_base 5: mod_base = gm
  modified_base 6: mod_base = gm
  modified_base 7: mod_base = OTHER; note = 2 -Fluoro-cytidine
  modified_base 8: mod_base = OTHER; note = 2 -Fluoro-cytidine
  modified_base 9: mod_base = OTHER; note = 2'-Fluoro-adenosine
  modified_base 10: mod_base = gm
  modified_base 11: mod_base = um
  modified_base 12: mod_base = OTHER; note = 2'-Fluoro-uridine
  modified_base 13: mod_base = OTHER; note = 2 -O-methyl-adenosine
  modified_base 14: mod_base = um
  modified_base 15: mod_base = OTHER; note = 2 -O-methyl-adenosine
  modified_base 16: mod_base = gm
  modified_base 17: mod_base = OTHER; note = 2 -Fluoro-cytidine
  modified_base 18: mod_base = um
  modified_base 19: mod_base = OTHER; note = 2 -O-methyl-adenosine
  source 1..19: mol_type = other RNA
tttgggccagttatagcta

SEQ ID NO: 235 - RNA, 19 nt, synthetic construct
  modified_base 1..3: mod_base = OTHER; note = linked via phosphorothioate
  modified_base 1: mod_base = gm
  modified_base 2: mod_base = gm
  modified_base 3: mod_base = OTHER; note = 2'-Fluoro-uridine
  modified_base 4: mod_base = cm
  modified_base 5: mod_base = cm
  modified_base 6: mod_base = um
  modified_base 7: mod_base = OTHER; note = 2'-Fluoro-guanosine
  modified_base 8: mod_base = OTHER; note = 2 -Fluoro-cytidine
  modified_base 9: mod_base = OTHER; note = 2'-Fluoro-uridine
  modified_base 10: mod_base = gm
  modified_base 11: mod_base = OTHER; note = 2 -O-methyl-adenosine
  modified_base 12: mod_base = OTHER; note = 2 -Fluoro-cytidine
  modified_base 13: mod_base = OTHER; note = 2 -O-methyl-adenosine
  modified_base 14: mod_base = OTHER; note = 2 -O-methyl-adenosine
  modified_base 15: mod_base = OTHER; note = 2 -O-methyl-adenosine
  modified_base 16: mod_base = um
  modified_base 17: mod_base = OTHER; note = 2 -Fluoro-cytidine
  modified_base 18: mod_base = OTHER; note = 2 -O-methyl-adenosine
  modified_base 19: mod_base = OTHER; note = 2 -O-methyl-adenosine
  source 1..19: mol_type = other RNA
ggtcctgctgacaaatcaa

SEQ ID NO: 236 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = cm
  modified_base 3: mod_base = OTHER; note = 2'-Fluoro-adenosine
  modified_base 4: mod_base = OTHER; note = 2 -O-methyl-adenosine
  modified_base 5: mod_base = um
  modified_base 6: mod_base = cm
  modified_base 7: mod_base = OTHER; note = 2'-Fluoro-uridine
  modified_base 8: mod_base = OTHER; note = 2 -Fluoro-cytidine
  modified_base 9: mod_base = OTHER; note = 2'-Fluoro-uridine
  modified_base 10: mod_base = OTHER; note = 2 -O-methyl-adenosine
  modified_base 11: mod_base = gm
  modified_base 12: mod_base = OTHER; note = 2'-Fluoro-guanosine
  modified_base 13: mod_base = um
  modified_base 14: mod_base = um
  modified_base 15: mod_base = um
  modified_base 16: mod_base = gm
  modified_base 17: mod_base = OTHER; note = 2'-Fluoro-guanosine
  modified_base 18: mod_base = cm
  modified_base 19: mod_base = OTHER; note = 2 -O-methyl-adenosine
  source 1..19: mol_type = other RNA
acaatctctaggtttggca

SEQ ID NO: 237 - RNA, 19 nt, synthetic construct
  modified_base 1..3: mod_base = OTHER; note = linked via phosphorothioate
  modified_base 1: mod_base = um
  modified_base 2: mod_base = cm
  modified_base 3: mod_base = OTHER; note = 2'-Fluoro-uridine
  modified_base 4: mod_base = OTHER; note = 2 -O-methyl-adenosine
  modified_base 5: mod_base = gm
  modified_base 6: mod_base = gm
  modified_base 7: mod_base = OTHER; note = 2'-Fluoro-uridine
  modified_base 8: mod_base = OTHER; note = 2'-Fluoro-uridine
  modified_base 9: mod_base = OTHER; note = 2'-Fluoro-uridine
  modified_base 10: mod_base = gm
  modified_base 11: mod_base = gm
  modified_base 12: mod_base = OTHER; note = 2 -Fluoro-cytidine
  modified_base 13: mod_base = um
  modified_base 14: mod_base = OTHER; note = 2 -O-methyl-adenosine
  modified_base 15: mod_base = gm
  modified_base 16: mod_base = um
  modified_base 17: mod_base = OTHER; note = 2'-Fluoro-uridine
  modified_base 18: mod_base = cm
  modified_base 19: mod_base = OTHER; note = 2 -O-methyl-adenosine
  source 1..19: mol_type = other RNA
tctaggtttggctagttca

SEQ ID NO: 238 - RNA, 19 nt, synthetic construct
  modified_base 1..3: mod_base = OTHER; note = linked via phosphorothioate
  modified_base 1: mod_base = gm
  modified_base 2: mod_base = um
  modified_base 3: mod_base = OTHER; note = 2'-Fluoro-uridine
  modified_base 4: mod_base = um
  modified_base 5: mod_base = gm
  modified_base 6: mod_base = gm
  modified_base 7: mod_base = OTHER; note = 2 -Fluoro-cytidine
  modified_base 8: mod_base = OTHER; note = 2'-Fluoro-uridine
  modified_base 9: mod_base = OTHER; note = 2'-Fluoro-adenosine
  modified_base 10: mod_base = gm
  modified_base 11: mod_base = um
  modified_base 12: mod_base = OTHER; note = 2'-Fluoro-uridine
  modified_base 13: mod_base = cm
  modified_base 14: mod_base = um
  modified_base 15: mod_base = cm
  modified_base 16: mod_base = um
  modified_base 17: mod_base = OTHER; note = 2'-Fluoro-uridine
  modified_base 18: mod_base = OTHER; note = 2 -O-methyl-adenosine
  modified_base 19: mod_base = OTHER; note = 2 -O-methyl-adenosine
  source 1..19: mol_type = other RNA
gtttggctagttctcttaa

SEQ ID NO: 239 - RNA, 19 nt, synthetic construct
  modified_base 1..3: mod_base = OTHER; note = linked via phosphorothioate
  modified_base 1: mod_base = um
  modified_base 2: mod_base = um
  modified_base 3: mod_base = OTHER; note = 2'-Fluoro-guanosine
  modified_base 4: mod_base = gm
  modified_base 5: mod_base = cm
  modified_base 6: mod_base = um
  modified_base 7: mod_base = OTHER; note = 2'-Fluoro-adenosine
  modified_base 8: mod_base = OTHER; note = 2'-Fluoro-guanosine
  modified_base 9: mod_base = OTHER; note = 2'-Fluoro-uridine
  modified_base 10: mod_base = um
  modified_base 11: mod_base = cm
  modified_base 12: mod_base = OTHER; note = 2'-Fluoro-uridine
  modified_base 13: mod_base = cm
  modified_base 14: mod_base = um
  modified_base 15: mod_base = um
  modified_base 16: mod_base = OTHER; note = 2 -O-methyl-adenosine
  modified_base 17: mod_base = OTHER; note = 2'-Fluoro-adenosine
  modified_base 18: mod_base = cm
  modified_base 19: mod_base = OTHER; note = 2 -O-methyl-adenosine
  source 1..19: mol_type = other RNA
ttggctagttctcttaaca

SEQ ID NO: 240 - RNA, 19 nt, synthetic construct
  modified_base 1..3: mod_base = OTHER; note = linked via phosphorothioate
  modified_base 1: mod_base = gm
  modified_base 2: mod_base = cm
  modified_base 3: mod_base = OTHER; note = 2'-Fluoro-adenosine
  modified_base 4: mod_base = um
  modified_base 5: mod_base = gm
  modified_base 6: mod_base = gm
  modified_base 7: mod_base = OTHER; note = 2'-Fluoro-uridine
  modified_base 8: mod_base = OTHER; note = 2'-Fluoro-guanosine
  modified_base 9: mod_base = OTHER; note = 2'-Fluoro-adenosine
  modified_base 10: mod_base = gm
  modified_base 11: mod_base = gm
  modified_base 12: mod_base = OTHER; note = 2'-Fluoro-uridine
  modified_base 13: mod_base = gm
  modified_base 14: mod_base = OTHER; note = 2 -O-methyl-adenosine
  modified_base 15: mod_base = OTHER; note = 2 -O-methyl-adenosine
  modified_base 16: mod_base = OTHER; note = 2 -O-methyl-adenosine
  modified_base 17: mod_base = OTHER; note = 2'-Fluoro-guanosine
  modified_base 18: mod_base = um
  modified_base 19: mod_base = OTHER; note = 2 -O-methyl-adenosine
  source 1..19: mol_type = other RNA
gcatggtgaggtgaaagta

SEQ ID NO: 241 - RNA, 19 nt, synthetic construct
  modified_base 1..3: mod_base = OTHER; note = linked via phosphorothioate
  modified_base 1: mod_base = cm
  modified_base 2: mod_base = OTHER; note = 2 -O-methyl-adenosine
  modified_base 3: mod_base = OTHER; note = 2'-Fluoro-uridine
  modified_base 4: mod_base = gm
  modified_base 5: mod_base = gm
  modified_base 6: mod_base = um
  modified_base 7: mod_base = OTHER; note = 2'-Fluoro-guanosine
  modified_base 8: mod_base = OTHER; note = 2'-Fluoro-adenosine
  modified_base 9: mod_base = OTHER; note = 2'-Fluoro-guanosine
  modified_base 10: mod_base = gm
  modified_base 11: mod_base = um
  modified_base 12: mod_base = OTHER; note = 2'-Fluoro-guanosine
  modified_base 13: mod_base = OTHER; note = 2 -O-methyl-adenosine
  modified_base 14: mod_base = OTHER; note = 2 -O-methyl-adenosine
  modified_base 15: mod_base = OTHER; note = 2 -O-methyl-adenosine
  modified_base 16: mod_base = gm
  modified_base 17: mod_base = OTHER; note = 2'-Fluoro-uridine
  modified_base 18: mod_base = OTHER; note = 2 -O-methyl-adenosine
  modified_base 19: mod_base = OTHER; note = 2 -O-methyl-adenosine
  source 1..19: mol_type = other RNA
catggtgaggtgaaagtaa

SEQ ID NO: 242 - RNA, 19 nt, synthetic construct
  modified_base 1..3: mod_base = OTHER; note = linked via phosphorothioate
  modified_base 1: mod_base = gm
  modified_base 2: mod_base = um
  modified_base 3: mod_base = OTHER; note = 2'-Fluoro-guanosine
  modified_base 4: mod_base = OTHER; note = 2 -O-methyl-adenosine
  modified_base 5: mod_base = cm
  modified_base 6: mod_base = um
  modified_base 7: mod_base = OTHER; note = 2'-Fluoro-uridine
  modified_base 8: mod_base = OTHER; note = 2'-Fluoro-adenosine
  modified_base 9: mod_base = OTHER; note = 2'-Fluoro-guanosine
  modified_base 10: mod_base = gm
  modified_base 11: mod_base = um
  modified_base 12: mod_base = OTHER; note = 2'-Fluoro-uridine
  modified_base 13: mod_base = cm
  modified_base 14: mod_base = um
  modified_base 15: mod_base = OTHER; note = 2 -O-methyl-adenosine
  modified_base 16: mod_base = gm
  modified_base 17: mod_base = OTHER; note = 2'-Fluoro-adenosine
  modified_base 18: mod_base = um
  modified_base 19: mod_base = OTHER; note = 2 -O-methyl-adenosine
  source 1..19: mol_type = other RNA
gtgacttaggttctagata

SEQ ID NO: 243 - RNA, 19 nt, synthetic construct
  modified_base 1..3: mod_base = OTHER; note = linked via phosphorothioate
  modified_base 1: mod_base = um
  modified_base 2: mod_base = gm
  modified_base 3: mod_base = OTHER; note = 2'-Fluoro-adenosine
  modified_base 4: mod_base = cm
  modified_base 5: mod_base = um
  modified_base 6: mod_base = um
  modified_base 7: mod_base = OTHER; note = 2'-Fluoro-adenosine
  modified_base 8: mod_base = OTHER; note = 2'-Fluoro-guanosine
  modified_base 9: mod_base = OTHER; note = 2'-Fluoro-guanosine
  modified_base 10: mod_base = um
  modified_base 11: mod_base = um
  modified_base 12: mod_base = OTHER; note = 2 -Fluoro-cytidine
  modified_base 13: mod_base = um
  modified_base 14: mod_base = OTHER; note = 2 -O-methyl-adenosine
  modified_base 15: mod_base = gm
  modified_base 16: mod_base = OTHER; note = 2 -O-methyl-adenosine
  modified_base 17: mod_base = OTHER; note = 2'-Fluoro-uridine
  modified_base 18: mod_base = OTHER; note = 2 -O-methyl-adenosine
  modified_base 19: mod_base = OTHER; note = 2 -O-methyl-adenosine
  source 1..19: mol_type = other RNA
tgacttaggttctagataa

SEQ ID NO: 244 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = gm
  modified_base 3: mod_base = OTHER; note = 2'-Fluoro-guanosine
  modified_base 4: mod_base = OTHER; note = 2 -O-methyl-adenosine
  modified_base 5: mod_base = cm
  modified_base 6: mod_base = OTHER; note = 2 -O-methyl-adenosine
  modified_base 7: mod_base = OTHER; note = 2'-Fluoro-uridine
  modified_base 8: mod_base = OTHER; note = 2 -Fluoro-cytidine
  modified_base 9: mod_base = OTHER; note = 2'-Fluoro-adenosine
  modified_base 10: mod_base = cm
  modified_base 11: mod_base = um
  modified_base 12: mod_base = OTHER; note = 2'-Fluoro-uridine
  modified_base 13: mod_base = OTHER; note = 2 -O-methyl-adenosine
  modified_base 14: mod_base = cm
  modified_base 15: mod_base = um
  modified_base 16: mod_base = OTHER; note = 2 -O-methyl-adenosine
  modified_base 17: mod_base = OTHER; note = 2'-Fluoro-uridine
  modified_base 18: mod_base = cm
  modified_base 19: mod_base = OTHER; note = 2 -O-methyl-adenosine
  source 1..19: mol_type = other RNA
aggacatcacttactatca

SEQ ID NO: 245 - RNA, 19 nt, synthetic construct
  modified_base 1..3: mod_base = OTHER; note = linked via phosphorothioate
  modified_base 1: mod_base = gm
  modified_base 2: mod_base = gm
  modified_base 3: mod_base = OTHER; note = 2'-Fluoro-adenosine
  modified_base 4: mod_base = um
  modified_base 5: mod_base = OTHER; note = 2 -O-methyl-adenosine
  modified_base 6: mod_base = cm
  modified_base 7: mod_base = OTHER; note = 2'-Fluoro-adenosine
  modified_base 8: mod_base = OTHER; note = 2 -Fluoro-cytidine
  modified_base 9: mod_base = OTHER; note = 2'-Fluoro-uridine
  modified_base 10: mod_base = um
  modified_base 11: mod_base = OTHER; note = 2 -O-methyl-adenosine
  modified_base 12: mod_base = OTHER; note = 2'-Fluoro-uridine
  modified_base 13: mod_base = um
  modified_base 14: mod_base = um
  modified_base 15: mod_base = gm
  modified_base 16: mod_base = um
  modified_base 17: mod_base = OTHER; note = 2 -Fluoro-cytidine
  modified_base 18: mod_base = OTHER; note = 2 -O-methyl-adenosine
  modified_base 19: mod_base = OTHER; note = 2 -O-methyl-adenosine
  source 1..19: mol_type = other RNA
ggatacacttatttgtcaa

SEQ ID NO: 246 - RNA, 19 nt, synthetic construct
  modified_base 1..3: mod_base = OTHER; note = linked via phosphorothioate
  modified_base 1: mod_base = um
  modified_base 2: mod_base = um
  modified_base 3: mod_base = OTHER; note = 2'-Fluoro-uridine
  modified_base 4: mod_base = OTHER; note = 2 -O-methyl-adenosine
  modified_base 5: mod_base = OTHER; note = 2 -O-methyl-adenosine
  modified_base 6: mod_base = cm
  modified_base 7: mod_base = OTHER; note = 2 -Fluoro-cytidine
  modified_base 8: mod_base = OTHER; note = 2'-Fluoro-uridine
  modified_base 9: mod_base = OTHER; note = 2'-Fluoro-adenosine
  modified_base 10: mod_base = um
  modified_base 11: mod_base = gm
  modified_base 12: mod_base = OTHER; note = 2'-Fluoro-uridine
  modified_base 13: mod_base = um
  modified_base 14: mod_base = OTHER; note = 2 -O-methyl-adenosine
  modified_base 15: mod_base = cm
  modified_base 16: mod_base = OTHER; note = 2 -O-methyl-adenosine
  modified_base 17: mod_base = OTHER; note = 2 -Fluoro-cytidine
  modified_base 18: mod_base = cm
  modified_base 19: mod_base = OTHER; note = 2 -O-methyl-adenosine
  source 1..19: mol_type = other RNA
tttaacctatgttacacca

SEQ ID NO: 247 - RNA, 19 nt, synthetic construct
  modified_base 1..3: mod_base = OTHER; note = linked via phosphorothioate
  modified_base 1: mod_base = um
  modified_base 2: mod_base = um
  modified_base 3: mod_base = OTHER; note = 2'-Fluoro-adenosine
  modified_base 4: mod_base = OTHER; note = 2 -O-methyl-adenosine
  modified_base 5: mod_base = cm
  modified_base 6: mod_base = cm
  modified_base 7: mod_base = OTHER; note = 2'-Fluoro-uridine
  modified_base 8: mod_base = OTHER; note = 2'-Fluoro-adenosine
  modified_base 9: mod_base = OTHER; note = 2'-Fluoro-uridine
  modified_base 10: mod_base = gm
  modified_base 11: mod_base = um
  modified_base 12: mod_base = OTHER; note = 2'-Fluoro-uridine
  modified_base 13: mod_base = OTHER; note = 2 -O-methyl-adenosine
  modified_base 14: mod_base = cm
  modified_base 15: mod_base = OTHER; note = 2 -O-methyl-adenosine
  modified_base 16: mod_base = cm
  modified_base 17: mod_base = OTHER; note = 2 -Fluoro-cytidine
  modified_base 18: mod_base = OTHER; note = 2 -O-methyl-adenosine
  modified_base 19: mod_base = OTHER; note = 2 -O-methyl-adenosine
  source 1..19: mol_type = other RNA
ttaacctatgttacaccaa

SEQ ID NO: 248 - RNA, 19 nt, synthetic construct
  modified_base 1..3: mod_base = OTHER; note = linked via phosphorothioate
  modified_base 1: mod_base = um
  modified_base 2: mod_base = gm
  modified_base 3: mod_base = OTHER; note = 2'-Fluoro-guanosine
  modified_base 4: mod_base = gm
  modified_base 5: mod_base = cm
  modified_base 6: mod_base = OTHER; note = 2 -O-methyl-adenosine
  modified_base 7: mod_base = OTHER; note = 2'-Fluoro-adenosine
  modified_base 8: mod_base = OTHER; note = 2'-Fluoro-adenosine
  modified_base 9: mod_base = OTHER; note = 2'-Fluoro-adenosine
  modified_base 10: mod_base = um
  modified_base 11: mod_base = um
  modified_base 12: mod_base = OTHER; note = 2'-Fluoro-guanosine
  modified_base 13: mod_base = um
  modified_base 14: mod_base = gm
  modified_base 15: mod_base = cm
  modified_base 16: mod_base = OTHER; note = 2 -O-methyl-adenosine
  modified_base 17: mod_base = OTHER; note = 2'-Fluoro-adenosine
  modified_base 18: mod_base = gm
  modified_base 19: mod_base = OTHER; note = 2 -O-methyl-adenosine
  source 1..19: mol_type = other RNA
tgggcaaaattgtgcaaga

SEQ ID NO: 249 - RNA, 19 nt, synthetic construct
  modified_base 1..3: mod_base = OTHER; note = linked via phosphorothioate
  modified_base 1: mod_base = um
  modified_base 2: mod_base = um
  modified_base 3: mod_base = OTHER; note = 2'-Fluoro-uridine
  modified_base 4: mod_base = gm
  modified_base 5: mod_base = OTHER; note = 2 -O-methyl-adenosine
  modified_base 6: mod_base = OTHER; note = 2 -O-methyl-adenosine
  modified_base 7: mod_base = OTHER; note = 2'-Fluoro-uridine
  modified_base 8: mod_base = OTHER; note = 2'-Fluoro-adenosine
  modified_base 9: mod_base = OTHER; note = 2'-Fluoro-uridine
  modified_base 10: mod_base = cm
  modified_base 11: mod_base = cm
  modified_base 12: mod_base = OTHER; note = 2'-Fluoro-adenosine
  modified_base 13: mod_base = um
  modified_base 14: mod_base = um
  modified_base 15: mod_base = cm
  modified_base 16: mod_base = um
  modified_base 17: mod_base = OTHER; note = 2 -Fluoro-cytidine
  modified_base 18: mod_base = gm
  modified_base 19: mod_base = OTHER; note = 2 -O-methyl-adenosine
  source 1..19: mol_type = other RNA
tttgaatatccattctcga

SEQ ID NO: 250 - RNA, 19 nt, synthetic construct
  modified_base 1..3: mod_base = OTHER; note = linked via phosphorothioate
  modified_base 1: mod_base = um
  modified_base 2: mod_base = cm
  modified_base 3: mod_base = OTHER; note = 2 -Fluoro-cytidine
  modified_base 4: mod_base = cm
  modified_base 5: mod_base = cm
  modified_base 6: mod_base = um
  modified_base 7: mod_base = OTHER; note = 2'-Fluoro-adenosine
  modified_base 8: mod_base = OTHER; note = 2'-Fluoro-adenosine
  modified_base 9: mod_base = OTHER; note = 2 -Fluoro-cytidine
  modified_base 10: mod_base = cm
  modified_base 11: mod_base = OTHER; note = 2 -O-methyl-adenosine
  modified_base 12: mod_base = OTHER; note = 2'-Fluoro-uridine
  modified_base 13: mod_base = OTHER; note = 2 -O-methyl-adenosine
  modified_base 14: mod_base = OTHER; note = 2 -O-methyl-adenosine
  modified_base 15: mod_base = gm
  modified_base 16: mod_base = OTHER; note = 2 -O-methyl-adenosine
  modified_base 17: mod_base = OTHER; note = 2'-Fluoro-uridine
  modified_base 18: mod_base = um
  modified_base 19: mod_base = OTHER; note = 2 -O-methyl-adenosine
  source 1..19: mol_type = other RNA
tcccctaaccataagatta

SEQ ID NO: 251 - RNA, 19 nt, synthetic construct
  modified_base 1..3: mod_base = OTHER; note = linked via phosphorothioate
  modified_base 1: mod_base = cm
  modified_base 2: mod_base = cm
  modified_base 3: mod_base = OTHER; note = 2 -Fluoro-cytidine
  modified_base 4: mod_base = cm
  modified_base 5: mod_base = um
  modified_base 6: mod_base = OTHER; note = 2 -O-methyl-adenosine
  modified_base 7: mod_base = OTHER; note = 2'-Fluoro-adenosine
  modified_base 8: mod_base = OTHER; note = 2 -Fluoro-cytidine
  modified_base 9: mod_base = OTHER; note = 2 -Fluoro-cytidine
  modified_base 10: mod_base = OTHER; note = 2 -O-methyl-adenosine
  modified_base 11: mod_base = um
  modified_base 12: mod_base = OTHER; note = 2'-Fluoro-adenosine
  modified_base 13: mod_base = OTHER; note = 2 -O-methyl-adenosine
  modified_base 14: mod_base = gm
  modified_base 15: mod_base = OTHER; note = 2 -O-methyl-adenosine
  modified_base 16: mod_base = um
  modified_base 17: mod_base = OTHER; note = 2'-Fluoro-uridine
  modified_base 18: mod_base = um
  modified_base 19: mod_base = OTHER; note = 2 -O-methyl-adenosine
  source 1..19: mol_type = other RNA
cccctaaccataagattta

SEQ ID NO: 252 - RNA, 19 nt, synthetic construct
  modified_base 1..3: mod_base = OTHER; note = linked via phosphorothioate
  modified_base 1: mod_base = cm
  modified_base 2: mod_base = cm
  modified_base 3: mod_base = OTHER; note = 2 -Fluoro-cytidine
  modified_base 4: mod_base = um
  modified_base 5: mod_base = OTHER; note = 2 -O-methyl-adenosine
  modified_base 6: mod_base = OTHER; note = 2 -O-methyl-adenosine
  modified_base 7: mod_base = OTHER; note = 2 -Fluoro-cytidine
  modified_base 8: mod_base = OTHER; note = 2 -Fluoro-cytidine
  modified_base 9: mod_base = OTHER; note = 2'-Fluoro-adenosine
  modified_base 10: mod_base = um
  modified_base 11: mod_base = OTHER; note = 2 -O-methyl-adenosine
  modified_base 12: mod_base = OTHER; note = 2'-Fluoro-adenosine
  modified_base 13: mod_base = gm
  modified_base 14: mod_base = OTHER; note = 2 -O-methyl-adenosine
  modified_base 15: mod_base = um
  modified_base 16: mod_base = um
  modified_base 17: mod_base = OTHER; note = 2'-Fluoro-uridine
  modified_base 18: mod_base = OTHER; note = 2 -O-methyl-adenosine
  modified_base 19: mod_base = OTHER; note = 2 -O-methyl-adenosine
  source 1..19: mol_type = other RNA
ccctaaccataagatttaa

SEQ ID NO: 253 - RNA, 19 nt, synthetic construct
  modified_base 1..3: mod_base = OTHER; note = linked via phosphorothioate
  modified_base 1: mod_base = cm
  modified_base 2: mod_base = cm
  modified_base 3: mod_base = OTHER; note = 2'-Fluoro-uridine
  modified_base 4: mod_base = OTHER; note = 2 -O-methyl-adenosine
  modified_base 5: mod_base = OTHER; note = 2 -O-methyl-adenosine
  modified_base 6: mod_base = cm
  modified_base 7: mod_base = OTHER; note = 2 -Fluoro-cytidine
  modified_base 8: mod_base = OTHER; note = 2'-Fluoro-adenosine
  modified_base 9: mod_base = OTHER; note = 2'-Fluoro-uridine
  modified_base 10: mod_base = OTHER; note = 2 -O-methyl-adenosine
  modified_base 11: mod_base = OTHER; note = 2 -O-methyl-adenosine
  modified_base 12: mod_base = OTHER; note = 2'-Fluoro-guanosine
  modified_base 13: mod_base = OTHER; note = 2 -O-methyl-adenosine
  modified_base 14: mod_base = um
  modified_base 15: mod_base = um
  modified_base 16: mod_base = um
  modified_base 17: mod_base = OTHER; note = 2'-Fluoro-adenosine
  modified_base 18: mod_base = cm
  modified_base 19: mod_base = OTHER; note = 2 -O-methyl-adenosine
  source 1..19: mol_type = other RNA
cctaaccataagatttaca

SEQ ID NO: 254 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = gm
  modified_base 3: mod_base = OTHER; note = 2'-Fluoro-guanosine
  modified_base 4: mod_base = gm
  modified_base 5: mod_base = OTHER; note = 2 -O-methyl-adenosine
  modified_base 6: mod_base = um
  modified_base 7: mod_base = OTHER; note = 2'-Fluoro-uridine
  modified_base 8: mod_base = OTHER; note = 2'-Fluoro-uridine
  modified_base 9: mod_base = OTHER; note = 2'-Fluoro-guanosine
  modified_base 10: mod_base = OTHER; note = 2 -O-methyl-adenosine
  modified_base 11: mod_base = cm
  modified_base 12: mod_base = OTHER; note = 2 -Fluoro-cytidine
  modified_base 13: mod_base = um
  modified_base 14: mod_base = OTHER; note = 2 -O-methyl-adenosine
  modified_base 15: mod_base = OTHER; note = 2 -O-methyl-adenosine
  modified_base 16: mod_base = um
  modified_base 17: mod_base = OTHER; note = 2 -Fluoro-cytidine
  modified_base 18: mod_base = OTHER; note = 2 -O-methyl-adenosine
  modified_base 19: mod_base = OTHER; note = 2 -O-methyl-adenosine
  source 1..19: mol_type = other RNA
agggatttgacctaatcaa

SEQ ID NO: 255 - RNA, 19 nt, synthetic construct
  modified_base 1..3: mod_base = OTHER; note = linked via phosphorothioate
  modified_base 1: mod_base = um
  modified_base 2: mod_base = gm
  modified_base 3: mod_base = OTHER; note = 2'-Fluoro-adenosine
  modified_base 4: mod_base = cm
  modified_base 5: mod_base = cm
  modified_base 6: mod_base = um
  modified_base 7: mod_base = OTHER; note = 2'-Fluoro-adenosine
  modified_base 8: mod_base = OTHER; note = 2'-Fluoro-adenosine
  modified_base 9: mod_base = OTHER; note = 2'-Fluoro-uridine
  modified_base 10: mod_base = cm
  modified_base 11: mod_base = OTHER; note = 2 -O-methyl-adenosine
  modified_base 12: mod_base = OTHER; note = 2 -Fluoro-cytidine
  modified_base 13: mod_base = um
  modified_base 14: mod_base = OTHER; note = 2 -O-methyl-adenosine
  modified_base 15: mod_base = OTHER; note = 2 -O-methyl-adenosine
  modified_base 16: mod_base = um
  modified_base 17: mod_base = OTHER; note = 2'-Fluoro-uridine
  modified_base 18: mod_base = um
  modified_base 19: mod_base = OTHER; note = 2 -O-methyl-adenosine
  source 1..19: mol_type = other RNA
tgacctaatcactaattta

SEQ ID NO: 256 - RNA, 19 nt, synthetic construct
  modified_base 1..3: mod_base = OTHER; note = linked via phosphorothioate
  modified_base 1: mod_base = cm
  modified_base 2: mod_base = um
  modified_base 3: mod_base = OTHER; note = 2'-Fluoro-guanosine
  modified_base 4: mod_base = cm
  modified_base 5: mod_base = cm
  modified_base 6: mod_base = cm
  modified_base 7: mod_base = OTHER; note = 2'-Fluoro-adenosine
  modified_base 8: mod_base = OTHER; note = 2'-Fluoro-adenosine
  modified_base 9: mod_base = OTHER; note = 2'-Fluoro-uridine
  modified_base 10: mod_base = cm
  modified_base 11: mod_base = cm
  modified_base 12: mod_base = OTHER; note = 2'-Fluoro-adenosine
  modified_base 13: mod_base = um
  modified_base 14: mod_base = um
  modified_base 15: mod_base = OTHER; note = 2 -O-methyl-adenosine
  modified_base 16: mod_base = gm
  modified_base 17: mod_base = OTHER; note = 2 -Fluoro-cytidine
  modified_base 18: mod_base = gm
  modified_base 19: mod_base = OTHER; note = 2 -O-methyl-adenosine
  source 1..19: mol_type = other RNA
ctgcccaatccattagcga

SEQ ID NO: 257 - RNA, 19 nt, synthetic construct
  modified_base 1..3: mod_base = OTHER; note = linked via phosphorothioate
  modified_base 1: mod_base = um
  modified_base 2: mod_base = gm
  modified_base 3: mod_base = OTHER; note = 2 -Fluoro-cytidine
  modified_base 4: mod_base = cm
  modified_base 5: mod_base = cm
  modified_base 6: mod_base = OTHER; note = 2 -O-methyl-adenosine
  modified_base 7: mod_base = OTHER; note = 2'-Fluoro-adenosine
  modified_base 8: mod_base = OTHER; note = 2'-Fluoro-uridine
  modified_base 9: mod_base = OTHER; note = 2 -Fluoro-cytidine
  modified_base 10: mod_base = cm
  modified_base 11: mod_base = OTHER; note = 2 -O-methyl-adenosine
  modified_base 12: mod_base = OTHER; note = 2'-Fluoro-uridine
  modified_base 13: mod_base = um
  modified_base 14: mod_base = OTHER; note = 2 -O-methyl-adenosine
  modified_base 15: mod_base = gm
  modified_base 16: mod_base = cm
  modified_base 17: mod_base = OTHER; note = 2'-Fluoro-guanosine
  modified_base 18: mod_base = OTHER; note = 2 -O-methyl-adenosine
  modified_base 19: mod_base = OTHER; note = 2 -O-methyl-adenosine
  source 1..19: mol_type = other RNA
tgcccaatccattagcgaa

SEQ ID NO: 258 - RNA, 19 nt, synthetic construct
  modified_base 1..3: mod_base = OTHER; note = linked via phosphorothioate
  modified_base 1: mod_base = gm
  modified_base 2: mod_base = cm
  modified_base 3: mod_base = OTHER; note = 2 -Fluoro-cytidine
  modified_base 4: mod_base = cm
  modified_base 5: mod_base = OTHER; note = 2 -O-methyl-adenosine
  modified_base 6: mod_base = OTHER; note = 2 -O-methyl-adenosine
  modified_base 7: mod_base = OTHER; note = 2'-Fluoro-uridine
  modified_base 8: mod_base = OTHER; note = 2 -Fluoro-cytidine
  modified_base 9: mod_base = OTHER; note = 2 -Fluoro-cytidine
  modified_base 10: mod_base = OTHER; note = 2 -O-methyl-adenosine
  modified_base 11: mod_base = um
  modified_base 12: mod_base = OTHER; note = 2'-Fluoro-uridine
  modified_base 13: mod_base = OTHER; note = 2 -O-methyl-adenosine
  modified_base 14: mod_base = gm
  modified_base 15: mod_base = cm
  modified_base 16: mod_base = gm
  modified_base 17: mod_base = OTHER; note = 2'-Fluoro-adenosine
  modified_base 18: mod_base = cm
  modified_base 19: mod_base = OTHER; note = 2 -O-methyl-adenosine
  source 1..19: mol_type = other RNA
gcccaatccattagcgaca

SEQ ID NO: 259 - RNA, 19 nt, synthetic construct
  modified_base 1..3: mod_base = OTHER; note = linked via phosphorothioate
  modified_base 1: mod_base = cm
  modified_base 2: mod_base = cm
  modified_base 3: mod_base = OTHER; note = 2 -Fluoro-cytidine
  modified_base 4: mod_base = OTHER; note = 2 -O-methyl-adenosine
  modified_base 5: mod_base = OTHER; note = 2 -O-methyl-adenosine
  modified_base 6: mod_base = um
  modified_base 7: mod_base = OTHER; note = 2 -Fluoro-cytidine
  modified_base 8: mod_base = OTHER; note = 2 -Fluoro-cytidine
  modified_base 9: mod_base = OTHER; note = 2'-Fluoro-adenosine
  modified_base 10: mod_base = um
  modified_base 11: mod_base = um
  modified_base 12: mod_base = OTHER; note = 2'-Fluoro-adenosine
  modified_base 13: mod_base = gm
  modified_base 14: mod_base = cm
  modified_base 15: mod_base = gm
  modified_base 16: mod_base = OTHER; note = 2 -O-methyl-adenosine
  modified_base 17: mod_base = OTHER; note = 2 -Fluoro-cytidine
  modified_base 18: mod_base = OTHER; note = 2 -O-methyl-adenosine
  modified_base 19: mod_base = OTHER; note = 2 -O-methyl-adenosine
  source 1..19: mol_type = other RNA
cccaatccattagcgacaa

SEQ ID NO: 260 - RNA, 19 nt, synthetic construct
  modified_base 1..3: mod_base = OTHER; note = linked via phosphorothioate
  modified_base 1: mod_base = cm
  modified_base 2: mod_base = cm
  modified_base 3: mod_base = OTHER; note = 2'-Fluoro-adenosine
  modified_base 4: mod_base = OTHER; note = 2 -O-methyl-adenosine
  modified_base 5: mod_base = um
  modified_base 6: mod_base = cm
  modified_base 7: mod_base = OTHER; note = 2 -Fluoro-cytidine
  modified_base 8: mod_base = OTHER; note = 2'-Fluoro-adenosine
  modified_base 9: mod_base = OTHER; note = 2'-Fluoro-uridine
  modified_base 10: mod_base = um
  modified_base 11: mod_base = OTHER; note = 2 -O-methyl-adenosine
  modified_base 12: mod_base = OTHER; note = 2'-Fluoro-guanosine
  modified_base 13: mod_base = cm
  modified_base 14: mod_base = gm
  modified_base 15: mod_base = OTHER; note = 2 -O-methyl-adenosine
  modified_base 16: mod_base = cm
  modified_base 17: mod_base = OTHER; note = 2'-Fluoro-adenosine
  modified_base 18: mod_base = gm
  modified_base 19: mod_base = OTHER; note = 2 -O-methyl-adenosine
  source 1..19: mol_type = other RNA
ccaatccattagcgacaga

SEQ ID NO: 261 - RNA, 19 nt, synthetic construct
  modified_base 1..3: mod_base = OTHER; note = linked via phosphorothioate
  modified_base 1: mod_base = cm
  modified_base 2: mod_base = OTHER; note = 2 -O-methyl-adenosine
  modified_base 3: mod_base = OTHER; note = 2'-Fluoro-adenosine
  modified_base 4: mod_base = um
  modified_base 5: mod_base = cm
  modified_base 6: mod_base = cm
  modified_base 7: mod_base = OTHER; note = 2'-Fluoro-adenosine
  modified_base 8: mod_base = OTHER; note = 2'-Fluoro-uridine
  modified_base 9: mod_base = OTHER; note = 2'-Fluoro-uridine
  modified_base 10: mod_base = OTHER; note = 2 -O-methyl-adenosine
  modified_base 11: mod_base = gm
  modified_base 12: mod_base = OTHER; note = 2 -Fluoro-cytidine
  modified_base 13: mod_base = gm
  modified_base 14: mod_base = OTHER; note = 2 -O-methyl-adenosine
  modified_base 15: mod_base = cm
  modified_base 16: mod_base = OTHER; note = 2 -O-methyl-adenosine
  modified_base 17: mod_base = OTHER; note = 2'-Fluoro-guanosine
  modified_base 18: mod_base = um
  modified_base 19: mod_base = OTHER; note = 2 -O-methyl-adenosine
  source 1..19: mol_type = other RNA
caatccattagcgacagta

SEQ ID NO: 262 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = um
  modified_base 3: mod_base = OTHER; note = 2 -Fluoro-cytidine
  modified_base 4: mod_base = cm
  modified_base 5: mod_base = OTHER; note = 2 -O-methyl-adenosine
  modified_base 6: mod_base = um
  modified_base 7: mod_base = OTHER; note = 2'-Fluoro-uridine
  modified_base 8: mod_base = OTHER; note = 2'-Fluoro-adenosine
  modified_base 9: mod_base = OTHER; note = 2'-Fluoro-guanosine
  modified_base 10: mod_base = cm
  modified_base 11: mod_base = gm
  modified_base 12: mod_base = OTHER; note = 2'-Fluoro-adenosine
  modified_base 13: mod_base = cm
  modified_base 14: mod_base = OTHER; note = 2 -O-methyl-adenosine
  modified_base 15: mod_base = gm
  modified_base 16: mod_base = um
  modified_base 17: mod_base = OTHER; note = 2'-Fluoro-adenosine
  modified_base 18: mod_base = gm
  modified_base 19: mod_base = OTHER; note = 2 -O-methyl-adenosine
  source 1..19: mol_type = other RNA
atccattagcgacagtaga

SEQ ID NO: 263 - RNA, 19 nt, synthetic construct
  modified_base 1..3: mod_base = OTHER; note = linked via phosphorothioate
  modified_base 1: mod_base = cm
  modified_base 2: mod_base = OTHER; note = 2 -O-methyl-adenosine
  modified_base 3: mod_base = OTHER; note = 2'-Fluoro-uridine
  modified_base 4: mod_base = um
  modified_base 5: mod_base = OTHER; note = 2 -O-methyl-adenosine
  modified_base 6: mod_base = gm
  modified_base 7: mod_base = OTHER; note = 2 -Fluoro-cytidine
  modified_base 8: mod_base = OTHER; note = 2'-Fluoro-guanosine
  modified_base 9: mod_base = OTHER; note = 2'-Fluoro-adenosine
  modified_base 10: mod_base = cm
  modified_base 11: mod_base = OTHER; note = 2 -O-methyl-adenosine
  modified_base 12: mod_base = OTHER; note = 2'-Fluoro-guanosine
  modified_base 13: mod_base = um
  modified_base 14: mod_base = OTHER; note = 2 -O-methyl-adenosine
  modified_base 15: mod_base = gm
  modified_base 16: mod_base = gm
  modified_base 17: mod_base = OTHER; note = 2'-Fluoro-adenosine
  modified_base 18: mod_base = um
  modified_base 19: mod_base = OTHER; note = 2 -O-methyl-adenosine
  source 1..19: mol_type = other RNA
cattagcgacagtaggata

SEQ ID NO: 264 - RNA, 19 nt, synthetic construct
  modified_base 1..3: mod_base = OTHER; note = linked via phosphorothioate
  modified_base 1: mod_base = gm
  modified_base 2: mod_base = gm
  modified_base 3: mod_base = OTHER; note = 2'-Fluoro-uridine
  modified_base 4: mod_base = OTHER; note = 2 -O-methyl-adenosine
  modified_base 5: mod_base = um
  modified_base 6: mod_base = gm
  modified_base 7: mod_base = OTHER; note = 2'-Fluoro-adenosine
  modified_base 8: mod_base = OTHER; note = 2'-Fluoro-adenosine
  modified_base 9: mod_base = OTHER; note = 2'-Fluoro-uridine
  modified_base 10: mod_base = OTHER; note = 2 -O-methyl-adenosine
  modified_base 11: mod_base = gm
  modified_base 12: mod_base = OTHER; note = 2'-Fluoro-adenosine
  modified_base 13: mod_base = cm
  modified_base 14: mod_base = OTHER; note = 2 -O-methyl-adenosine
  modified_base 15: mod_base = gm
  modified_base 16: mod_base = OTHER; note = 2 -O-methyl-adenosine
  modified_base 17: mod_base = OTHER; note = 2'-Fluoro-adenosine
  modified_base 18: mod_base = cm
  modified_base 19: mod_base = OTHER; note = 2 -O-methyl-adenosine
  source 1..19: mol_type = other RNA
ggtatgaatagacagaaca

SEQ ID NO: 265 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = OTHER; note = 2 -O-methyl-adenosine
  modified_base 3: mod_base = OTHER; note = 2'-Fluoro-uridine
  modified_base 4: mod_base = OTHER; note = 2 -O-methyl-adenosine
  modified_base 5: mod_base = gm
  modified_base 6: mod_base = OTHER; note = 2 -O-methyl-adenosine
  modified_base 7: mod_base = OTHER; note = 2 -Fluoro-cytidine
  modified_base 8: mod_base = OTHER; note = 2'-Fluoro-adenosine
  modified_base 9: mod_base = OTHER; note = 2'-Fluoro-guanosine
  modified_base 10: mod_base = OTHER; note = 2 -O-methyl-adenosine
  modified_base 11: mod_base = OTHER; note = 2 -O-methyl-adenosine
  modified_base 12: mod_base = OTHER; note = 2 -Fluoro-cytidine
  modified_base 13: mod_base = cm
  modified_base 14: mod_base = cm
  modified_base 15: mod_base = um
  modified_base 16: mod_base = OTHER; note = 2 -O-methyl-adenosine
  modified_base 17: mod_base = OTHER; note = 2'-Fluoro-uridine
  modified_base 18: mod_base = cm
  modified_base 19: mod_base = OTHER; note = 2 -O-methyl-adenosine
  source 1..19: mol_type = other RNA
aatagacagaaccctatca

SEQ ID NO: 266 - RNA, 19 nt, synthetic construct
  modified_base 1..3: mod_base = OTHER; note = linked via phosphorothioate
  modified_base 1: mod_base = um
  modified_base 2: mod_base = cm
  modified_base 3: mod_base = OTHER; note = 2 -Fluoro-cytidine
  modified_base 4: mod_base = um
  modified_base 5: mod_base = gm
  modified_base 6: mod_base = gm
  modified_base 7: mod_base = OTHER; note = 2'-Fluoro-uridine
  modified_base 8: mod_base = OTHER; note = 2'-Fluoro-adenosine
  modified_base 9: mod_base = OTHER; note = 2'-Fluoro-adenosine
  modified_base 10: mod_base = cm
  modified_base 11: mod_base = OTHER; note = 2 -O-methyl-adenosine
  modified_base 12: mod_base = OTHER; note = 2'-Fluoro-guanosine
  modified_base 13: mod_base = um
  modified_base 14: mod_base = OTHER; note = 2 -O-methyl-adenosine
  modified_base 15: mod_base = OTHER; note = 2 -O-methyl-adenosine
  modified_base 16: mod_base = um
  modified_base 17: mod_base = OTHER; note = 2'-Fluoro-adenosine
  modified_base 18: mod_base = cm
  modified_base 19: mod_base = OTHER; note = 2 -O-methyl-adenosine
  source 1..19: mol_type = other RNA
tcctggtaacagtaataca

SEQ ID NO: 267 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = gm
  modified_base 3: mod_base = OTHER; note = 2'-Fluoro-uridine
  modified_base 4: mod_base = um
  modified_base 5: mod_base = um
  modified_base 6: mod_base = um
  modified_base 7: mod_base = OTHER; note = 2'-Fluoro-adenosine
  modified_base 8: mod_base = OTHER; note = 2'-Fluoro-uridine
  modified_base 9: mod_base = OTHER; note = 2 -Fluoro-cytidine
  modified_base 10: mod_base = um
  modified_base 11: mod_base = gm
  modified_base 12: mod_base = OTHER; note = 2'-Fluoro-guanosine
  modified_base 13: mod_base = gm
  modified_base 14: mod_base = um
  modified_base 15: mod_base = OTHER; note = 2 -O-methyl-adenosine
  modified_base 16: mod_base = cm
  modified_base 17: mod_base = OTHER; note = 2'-Fluoro-adenosine
  modified_base 18: mod_base = OTHER; note = 2 -O-methyl-adenosine
  modified_base 19: mod_base = OTHER; note = 2 -O-methyl-adenosine
  source 1..19: mol_type = other RNA
agttttatctgggtacaaa

SEQ ID NO: 268 - RNA, 19 nt, synthetic construct
  modified_base 1..3: mod_base = OTHER; note = linked via phosphorothioate
  modified_base 1: mod_base = gm
  modified_base 2: mod_base = um
  modified_base 3: mod_base = OTHER; note = 2'-Fluoro-adenosine
  modified_base 4: mod_base = gm
  modified_base 5: mod_base = gm
  modified_base 6: mod_base = gm
  modified_base 7: mod_base = OTHER; note = 2'-Fluoro-uridine
  modified_base 8: mod_base = OTHER; note = 2'-Fluoro-guanosine
  modified_base 9: mod_base = OTHER; note = 2'-Fluoro-uridine
  modified_base 10: mod_base = um
  modified_base 11: mod_base = OTHER; note = 2 -O-methyl-adenosine
  modified_base 12: mod_base = OTHER; note = 2'-Fluoro-adenosine
  modified_base 13: mod_base = gm
  modified_base 14: mod_base = OTHER; note = 2 -O-methyl-adenosine
  modified_base 15: mod_base = cm
  modified_base 16: mod_base = um
  modified_base 17: mod_base = OTHER; note = 2'-Fluoro-uridine
  modified_base 18: mod_base = OTHER; note = 2 -O-methyl-adenosine
  modified_base 19: mod_base = OTHER; note = 2 -O-methyl-adenosine
  source 1..19: mol_type = other RNA
gtagggtgttaagacttaa

SEQ ID NO: 269 - RNA, 19 nt, synthetic construct
  modified_base 1..3: mod_base = OTHER; note = linked via phosphorothioate
  modified_base 1: mod_base = gm
  modified_base 2: mod_base = gm
  modified_base 3: mod_base = OTHER; note = 2'-Fluoro-guanosine
  modified_base 4: mod_base = gm
  modified_base 5: mod_base = OTHER; note = 2 -O-methyl-adenosine
  modified_base 6: mod_base = um
  modified_base 7: mod_base = OTHER; note = 2'-Fluoro-adenosine
  modified_base 8: mod_base = OTHER; note = 2'-Fluoro-uridine
  modified_base 9: mod_base = OTHER; note = 2'-Fluoro-uridine
  modified_base 10: mod_base = um
  modified_base 11: mod_base = OTHER; note = 2 -O-methyl-adenosine
  modified_base 12: mod_base = OTHER; note = 2'-Fluoro-guanosine
  modified_base 13: mod_base = gm
  modified_base 14: mod_base = cm
  modified_base 15: mod_base = cm
  modified_base 16: mod_base = um
  modified_base 17: mod_base = OTHER; note = 2 -Fluoro-cytidine
  modified_base 18: mod_base = um
  modified_base 19: mod_base = OTHER; note = 2 -O-methyl-adenosine
  source 1..19: mol_type = other RNA
ggggatatttaggcctcta

SEQ ID NO: 270 - RNA, 19 nt, synthetic construct
  modified_base 1..3: mod_base = OTHER; note = linked via phosphorothioate
  modified_base 1: mod_base = cm
  modified_base 2: mod_base = um
  modified_base 3: mod_base = OTHER; note = 2'-Fluoro-uridine
  modified_base 4: mod_base = OTHER; note = 2 -O-methyl-adenosine
  modified_base 5: mod_base = gm
  modified_base 6: mod_base = gm
  modified_base 7: mod_base = OTHER; note = 2 -Fluoro-cytidine
  modified_base 8: mod_base = OTHER; note = 2'-Fluoro-adenosine
  modified_base 9: mod_base = OTHER; note = 2'-Fluoro-uridine
  modified_base 10: mod_base = um
  modified_base 11: mod_base = OTHER; note = 2 -O-methyl-adenosine
  modified_base 12: mod_base = OTHER; note = 2'-Fluoro-adenosine
  modified_base 13: mod_base = cm
  modified_base 14: mod_base = OTHER; note = 2 -O-methyl-adenosine
  modified_base 15: mod_base = um
  modified_base 16: mod_base = gm
  modified_base 17: mod_base = OTHER; note = 2'-Fluoro-uridine
  modified_base 18: mod_base = um
  modified_base 19: mod_base = OTHER; note = 2 -O-methyl-adenosine
  source 1..19: mol_type = other RNA
cttaggcattaacatgtta

SEQ ID NO: 271 - RNA, 19 nt, synthetic construct
  modified_base 1..3: mod_base = OTHER; note = linked via phosphorothioate
  modified_base 1: mod_base = cm
  modified_base 2: mod_base = cm
  modified_base 3: mod_base = OTHER; note = 2'-Fluoro-uridine
  modified_base 4: mod_base = OTHER; note = 2 -O-methyl-adenosine
  modified_base 5: mod_base = OTHER; note = 2 -O-methyl-adenosine
  modified_base 6: mod_base = cm
  modified_base 7: mod_base = OTHER; note = 2'-Fluoro-uridine
  modified_base 8: mod_base = OTHER; note = 2'-Fluoro-uridine
  modified_base 9: mod_base = OTHER; note = 2'-Fluoro-uridine
  modified_base 10: mod_base = um
  modified_base 11: mod_base = OTHER; note = 2 -O-methyl-adenosine
  modified_base 12: mod_base = OTHER; note = 2'-Fluoro-uridine
  modified_base 13: mod_base = OTHER; note = 2 -O-methyl-adenosine
  modified_base 14: mod_base = gm
  modified_base 15: mod_base = gm
  modified_base 16: mod_base = um
  modified_base 17: mod_base = OTHER; note = 2'-Fluoro-uridine
  modified_base 18: mod_base = OTHER; note = 2 -O-methyl-adenosine
  modified_base 19: mod_base = OTHER; note = 2 -O-methyl-adenosine
  source 1..19: mol_type = other RNA
cctaacttttataggttaa

SEQ ID NO: 272 - RNA, 19 nt, synthetic construct
  modified_base 1..3: mod_base = OTHER; note = linked via phosphorothioate
  modified_base 1: mod_base = gm
  modified_base 2: mod_base = OTHER; note = 2 -O-methyl-adenosine
  modified_base 3: mod_base = OTHER; note = 2'-Fluoro-adenosine
  modified_base 4: mod_base = um
  modified_base 5: mod_base = OTHER; note = 2 -O-methyl-adenosine
  modified_base 6: mod_base = gm
  modified_base 7: mod_base = OTHER; note = 2'-Fluoro-uridine
  modified_base 8: mod_base = OTHER; note = 2 -Fluoro-cytidine
  modified_base 9: mod_base = OTHER; note = 2'-Fluoro-adenosine
  modified_base 10: mod_base = um
  modified_base 11: mod_base = OTHER; note = 2 -O-methyl-adenosine
  modified_base 12: mod_base = OTHER; note = 2'-Fluoro-adenosine
  modified_base 13: mod_base = cm
  modified_base 14: mod_base = um
  modified_base 15: mod_base = OTHER; note = 2 -O-methyl-adenosine
  modified_base 16: mod_base = gm
  modified_base 17: mod_base = OTHER; note = 2'-Fluoro-adenosine
  modified_base 18: mod_base = um
  modified_base 19: mod_base = OTHER; note = 2 -O-methyl-adenosine
  source 1..19: mol_type = other RNA
gaatagtcataactagata

SEQ ID NO: 273 - RNA, 19 nt, synthetic construct
  modified_base 1..3: mod_base = OTHER; note = linked via phosphorothioate
  modified_base 1: mod_base = cm
  modified_base 2: mod_base = OTHER; note = 2 -O-methyl-adenosine
  modified_base 3: mod_base = OTHER; note = 2'-Fluoro-uridine
  modified_base 4: mod_base = OTHER; note = 2 -O-methyl-adenosine
  modified_base 5: mod_base = OTHER; note = 2 -O-methyl-adenosine
  modified_base 6: mod_base = cm
  modified_base 7: mod_base = OTHER; note = 2'-Fluoro-uridine
  modified_base 8: mod_base = OTHER; note = 2'-Fluoro-adenosine
  modified_base 9: mod_base = OTHER; note = 2'-Fluoro-guanosine
  modified_base 10: mod_base = OTHER; note = 2 -O-methyl-adenosine
  modified_base 11: mod_base = um
  modified_base 12: mod_base = OTHER; note = 2'-Fluoro-uridine
  modified_base 13: mod_base = OTHER; note = 2 -O-methyl-adenosine
  modified_base 14: mod_base = OTHER; note = 2 -O-methyl-adenosine
  modified_base 15: mod_base = gm
  modified_base 16: mod_base = OTHER; note = 2 -O-methyl-adenosine
  modified_base 17: mod_base = OTHER; note = 2'-Fluoro-uridine
  modified_base 18: mod_base = cm
  modified_base 19: mod_base = OTHER; note = 2 -O-methyl-adenosine
  source 1..19: mol_type = other RNA
cataactagattaagatca

SEQ ID NO: 274 - RNA, 19 nt, synthetic construct
  modified_base 1..3: mod_base = OTHER; note = linked via phosphorothioate
  modified_base 1: mod_base = gm
  modified_base 2: mod_base = gm
  modified_base 3: mod_base = OTHER; note = 2'-Fluoro-guanosine
  modified_base 4: mod_base = OTHER; note = 2 -O-methyl-adenosine
  modified_base 5: mod_base = um
  modified_base 6: mod_base = OTHER; note = 2 -O-methyl-adenosine
  modified_base 7: mod_base = OTHER; note = 2'-Fluoro-adenosine
  modified_base 8: mod_base = OTHER; note = 2'-Fluoro-uridine
  modified_base 9: mod_base = OTHER; note = 2'-Fluoro-guanosine
  modified_base 10: mod_base = OTHER; note = 2 -O-methyl-adenosine
  modified_base 11: mod_base = um
  modified_base 12: mod_base = OTHER; note = 2'-Fluoro-adenosine
  modified_base 13: mod_base = gm
  modified_base 14: mod_base = gm
  modified_base 15: mod_base = um
  modified_base 16: mod_base = OTHER; note = 2 -O-methyl-adenosine
  modified_base 17: mod_base = OTHER; note = 2'-Fluoro-adenosine
  modified_base 18: mod_base = um
  modified_base 19: mod_base = OTHER; note = 2 -O-methyl-adenosine
  source 1..19: mol_type = other RNA
gggataatgataggtaata

SEQ ID NO: 275 - RNA, 19 nt, synthetic construct
  modified_base 1..3: mod_base = OTHER; note = linked via phosphorothioate
  modified_base 1: mod_base = gm
  modified_base 2: mod_base = gm
  modified_base 3: mod_base = OTHER; note = 2'-Fluoro-adenosine
  modified_base 4: mod_base = um
  modified_base 5: mod_base = OTHER; note = 2 -O-methyl-adenosine
  modified_base 6: mod_base = OTHER; note = 2 -O-methyl-adenosine
  modified_base 7: mod_base = OTHER; note = 2'-Fluoro-uridine
  modified_base 8: mod_base = OTHER; note = 2'-Fluoro-guanosine
  modified_base 9: mod_base = OTHER; note = 2'-Fluoro-adenosine
  modified_base 10: mod_base = um
  modified_base 11: mod_base = OTHER; note = 2 -O-methyl-adenosine
  modified_base 12: mod_base = OTHER; note = 2'-Fluoro-guanosine
  modified_base 13: mod_base = gm
  modified_base 14: mod_base = um
  modified_base 15: mod_base = OTHER; note = 2 -O-methyl-adenosine
  modified_base 16: mod_base = OTHER; note = 2 -O-methyl-adenosine
  modified_base 17: mod_base = OTHER; note = 2'-Fluoro-uridine
  modified_base 18: mod_base = um
  modified_base 19: mod_base = OTHER; note = 2 -O-methyl-adenosine
  source 1..19: mol_type = other RNA
ggataatgataggtaatta

SEQ ID NO: 276 - RNA, 19 nt, synthetic construct
  modified_base 1..3: mod_base = OTHER; note = linked via phosphorothioate
  modified_base 1: mod_base = gm
  modified_base 2: mod_base = um
  modified_base 3: mod_base = OTHER; note = 2'-Fluoro-guanosine
  modified_base 4: mod_base = um
  modified_base 5: mod_base = um
  modified_base 6: mod_base = um
  modified_base 7: mod_base = OTHER; note = 2'-Fluoro-uridine
  modified_base 8: mod_base = OTHER; note = 2'-Fluoro-adenosine
  modified_base 9: mod_base = OTHER; note = 2'-Fluoro-uridine
  modified_base 10: mod_base = cm
  modified_base 11: mod_base = cm
  modified_base 12: mod_base = OTHER; note = 2'-Fluoro-guanosine
  modified_base 13: mod_base = OTHER; note = 2 -O-methyl-adenosine
  modified_base 14: mod_base = OTHER; note = 2 -O-methyl-adenosine
  modified_base 15: mod_base = OTHER; note = 2 -O-methyl-adenosine
  modified_base 16: mod_base = gm
  modified_base 17: mod_base = OTHER; note = 2'-Fluoro-uridine
  modified_base 18: mod_base = um
  modified_base 19: mod_base = OTHER; note = 2 -O-methyl-adenosine
  source 1..19: mol_type = other RNA
gtgttttatccgaaagtta

SEQ ID NO: 277 - RNA, 19 nt, synthetic construct
  modified_base 1..3: mod_base = OTHER; note = linked via phosphorothioate
  modified_base 1: mod_base = um
  modified_base 2: mod_base = gm
  modified_base 3: mod_base = OTHER; note = 2'-Fluoro-uridine
  modified_base 4: mod_base = um
  modified_base 5: mod_base = um
  modified_base 6: mod_base = um
  modified_base 7: mod_base = OTHER; note = 2'-Fluoro-adenosine
  modified_base 8: mod_base = OTHER; note = 2'-Fluoro-uridine
  modified_base 9: mod_base = OTHER; note = 2 -Fluoro-cytidine
  modified_base 10: mod_base = cm
  modified_base 11: mod_base = gm
  modified_base 12: mod_base = OTHER; note = 2'-Fluoro-adenosine
  modified_base 13: mod_base = OTHER; note = 2 -O-methyl-adenosine
  modified_base 14: mod_base = OTHER; note = 2 -O-methyl-adenosine
  modified_base 15: mod_base = gm
  modified_base 16: mod_base = um
  modified_base 17: mod_base = OTHER; note = 2'-Fluoro-uridine
  modified_base 18: mod_base = um
  modified_base 19: mod_base = OTHER; note = 2 -O-methyl-adenosine
  source 1..19: mol_type = other RNA
tgttttatccgaaagttta

SEQ ID NO: 278 - RNA, 19 nt, synthetic construct
  modified_base 1..3: mod_base = OTHER; note = linked via phosphorothioate
  modified_base 1: mod_base = um
  modified_base 2: mod_base = OTHER; note = 2 -O-methyl-adenosine
  modified_base 3: mod_base = OTHER; note = 2'-Fluoro-uridine
  modified_base 4: mod_base = cm
  modified_base 5: mod_base = cm
  modified_base 6: mod_base = gm
  modified_base 7: mod_base = OTHER; note = 2'-Fluoro-adenosine
  modified_base 8: mod_base = OTHER; note = 2'-Fluoro-adenosine
  modified_base 9: mod_base = OTHER; note = 2'-Fluoro-adenosine
  modified_base 10: mod_base = gm
  modified_base 11: mod_base = um
  modified_base 12: mod_base = OTHER; note = 2'-Fluoro-uridine
  modified_base 13: mod_base = um
  modified_base 14: mod_base = cm
  modified_base 15: mod_base = cm
  modified_base 16: mod_base = OTHER; note = 2 -O-methyl-adenosine
  modified_base 17: mod_base = OTHER; note = 2'-Fluoro-adenosine
  modified_base 18: mod_base = um
  modified_base 19: mod_base = OTHER; note = 2 -O-methyl-adenosine
  source 1..19: mol_type = other RNA
tatccgaaagtttccaata

SEQ ID NO: 279 - RNA, 19 nt, synthetic construct
  modified_base 1..3: mod_base = OTHER; note = linked via phosphorothioate
  modified_base 1: mod_base = cm
  modified_base 2: mod_base = cm
  modified_base 3: mod_base = OTHER; note = 2'-Fluoro-guanosine
  modified_base 4: mod_base = OTHER; note = 2 -O-methyl-adenosine
  modified_base 5: mod_base = OTHER; note = 2 -O-methyl-adenosine
  modified_base 6: mod_base = OTHER; note = 2 -O-methyl-adenosine
  modified_base 7: mod_base = OTHER; note = 2'-Fluoro-guanosine
  modified_base 8: mod_base = OTHER; note = 2'-Fluoro-uridine
  modified_base 9: mod_base = OTHER; note = 2'-Fluoro-uridine
  modified_base 10: mod_base = um
  modified_base 11: mod_base = cm
  modified_base 12: mod_base = OTHER; note = 2 -Fluoro-cytidine
  modified_base 13: mod_base = OTHER; note = 2 -O-methyl-adenosine
  modified_base 14: mod_base = OTHER; note = 2 -O-methyl-adenosine
  modified_base 15: mod_base = um
  modified_base 16: mod_base = um
  modified_base 17: mod_base = OTHER; note = 2 -Fluoro-cytidine
  modified_base 18: mod_base = cm
  modified_base 19: mod_base = OTHER; note = 2 -O-methyl-adenosine
  source 1..19: mol_type = other RNA
ccgaaagtttccaattcca

SEQ ID NO: 280 - RNA, 19 nt, synthetic construct
  modified_base 1..3: mod_base = OTHER; note = linked via phosphorothioate
  modified_base 1: mod_base = gm
  modified_base 2: mod_base = cm
  modified_base 3: mod_base = OTHER; note = 2 -Fluoro-cytidine
  modified_base 4: mod_base = OTHER; note = 2 -O-methyl-adenosine
  modified_base 5: mod_base = OTHER; note = 2 -O-methyl-adenosine
  modified_base 6: mod_base = um
  modified_base 7: mod_base = OTHER; note = 2'-Fluoro-uridine
  modified_base 8: mod_base = OTHER; note = 2'-Fluoro-uridine
  modified_base 9: mod_base = OTHER; note = 2 -Fluoro-cytidine
  modified_base 10: mod_base = um
  modified_base 11: mod_base = um
  modified_base 12: mod_base = OTHER; note = 2'-Fluoro-adenosine
  modified_base 13: mod_base = cm
  modified_base 14: mod_base = um
  modified_base 15: mod_base = OTHER; note = 2 -O-methyl-adenosine
  modified_base 16: mod_base = gm
  modified_base 17: mod_base = OTHER; note = 2'-Fluoro-uridine
  modified_base 18: mod_base = OTHER; note = 2 -O-methyl-adenosine
  modified_base 19: mod_base = OTHER; note = 2 -O-methyl-adenosine
  source 1..19: mol_type = other RNA
gccaatttcttactagtaa

SEQ ID NO: 281 - RNA, 19 nt, synthetic construct
  modified_base 1..3: mod_base = OTHER; note = linked via phosphorothioate
  modified_base 1: mod_base = cm
  modified_base 2: mod_base = cm
  modified_base 3: mod_base = OTHER; note = 2'-Fluoro-adenosine
  modified_base 4: mod_base = OTHER; note = 2 -O-methyl-adenosine
  modified_base 5: mod_base = um
  modified_base 6: mod_base = um
  modified_base 7: mod_base = OTHER; note = 2'-Fluoro-uridine
  modified_base 8: mod_base = OTHER; note = 2 -Fluoro-cytidine
  modified_base 9: mod_base = OTHER; note = 2'-Fluoro-uridine
  modified_base 10: mod_base = um
  modified_base 11: mod_base = OTHER; note = 2 -O-methyl-adenosine
  modified_base 12: mod_base = OTHER; note = 2 -Fluoro-cytidine
  modified_base 13: mod_base = um
  modified_base 14: mod_base = OTHER; note = 2 -O-methyl-adenosine
  modified_base 15: mod_base = gm
  modified_base 16: mod_base = um
  modified_base 17: mod_base = OTHER; note = 2'-Fluoro-adenosine
  modified_base 18: mod_base = cm
  modified_base 19: mod_base = OTHER; note = 2 -O-methyl-adenosine
  source 1..19: mol_type = other RNA
ccaatttcttactagtaca

SEQ ID NO: 282 - RNA, 19 nt, synthetic construct
  modified_base 1..3: mod_base = OTHER; note = linked via phosphorothioate
  modified_base 1: mod_base = cm
  modified_base 2: mod_base = cm
  modified_base 3: mod_base = OTHER; note = 2'-Fluoro-adenosine
  modified_base 4: mod_base = um
  modified_base 5: mod_base = cm
  modified_base 6: mod_base = OTHER; note = 2 -O-methyl-adenosine
  modified_base 7: mod_base = OTHER; note = 2'-Fluoro-uridine
  modified_base 8: mod_base = OTHER; note = 2'-Fluoro-uridine
  modified_base 9: mod_base = OTHER; note = 2'-Fluoro-uridine
  modified_base 10: mod_base = gm
  modified_base 11: mod_base = gm
  modified_base 12: mod_base = OTHER; note = 2'-Fluoro-uridine
  modified_base 13: mod_base = um
  modified_base 14: mod_base = gm
  modified_base 15: mod_base = cm
  modified_base 16: mod_base = gm
  modified_base 17: mod_base = OTHER; note = 2 -Fluoro-cytidine
  modified_base 18: mod_base = um
  modified_base 19: mod_base = OTHER; note = 2 -O-methyl-adenosine
  source 1..19: mol_type = other RNA
ccatcatttggttgcgcta

SEQ ID NO: 283 - RNA, 19 nt, synthetic construct
  modified_base 1..3: mod_base = OTHER; note = linked via phosphorothioate
  modified_base 1: mod_base = um
  modified_base 2: mod_base = cm
  modified_base 3: mod_base = OTHER; note = 2'-Fluoro-adenosine
  modified_base 4: mod_base = um
  modified_base 5: mod_base = um
  modified_base 6: mod_base = um
  modified_base 7: mod_base = OTHER; note = 2'-Fluoro-guanosine
  modified_base 8: mod_base = OTHER; note = 2'-Fluoro-guanosine
  modified_base 9: mod_base = OTHER; note = 2'-Fluoro-uridine
  modified_base 10: mod_base = um
  modified_base 11: mod_base = gm
  modified_base 12: mod_base = OTHER; note = 2 -Fluoro-cytidine
  modified_base 13: mod_base = gm
  modified_base 14: mod_base = cm
  modified_base 15: mod_base = um
  modified_base 16: mod_base = gm
  modified_base 17: mod_base = OTHER; note = 2'-Fluoro-adenosine
  modified_base 18: mod_base = cm
  modified_base 19: mod_base = OTHER; note = 2 -O-methyl-adenosine
  source 1..19: mol_type = other RNA
tcatttggttgcgctgaca

SEQ ID NO: 284 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = cm
  modified_base 3: mod_base = OTHER; note = 2 -Fluoro-cytidine
  modified_base 4: mod_base = um
  modified_base 5: mod_base = OTHER; note = 2 -O-methyl-adenosine
  modified_base 6: mod_base = gm
  modified_base 7: mod_base = OTHER; note = 2'-Fluoro-guanosine
  modified_base 8: mod_base = OTHER; note = 2'-Fluoro-adenosine
  modified_base 9: mod_base = OTHER; note = 2'-Fluoro-adenosine
  modified_base 10: mod_base = um
  modified_base 11: mod_base = gm
  modified_base 12: mod_base = OTHER; note = 2'-Fluoro-uridine
  modified_base 13: mod_base = um
  modified_base 14: mod_base = gm
  modified_base 15: mod_base = gm
  modified_base 16: mod_base = um
  modified_base 17: mod_base = OTHER; note = 2 -Fluoro-cytidine
  modified_base 18: mod_base = OTHER; note = 2 -O-methyl-adenosine
  modified_base 19: mod_base = OTHER; note = 2 -O-methyl-adenosine
  source 1..19: mol_type = other RNA
acctaggaatgttggtcaa

SEQ ID NO: 285 - RNA, 19 nt, synthetic construct
  modified_base 1..3: mod_base = OTHER; note = linked via phosphorothioate
  modified_base 1: mod_base = cm
  modified_base 2: mod_base = cm
  modified_base 3: mod_base = OTHER; note = 2'-Fluoro-uridine
  modified_base 4: mod_base = OTHER; note = 2 -O-methyl-adenosine
  modified_base 5: mod_base = gm
  modified_base 6: mod_base = gm
  modified_base 7: mod_base = OTHER; note = 2'-Fluoro-adenosine
  modified_base 8: mod_base = OTHER; note = 2'-Fluoro-adenosine
  modified_base 9: mod_base = OTHER; note = 2'-Fluoro-uridine
  modified_base 10: mod_base = gm
  modified_base 11: mod_base = um
  modified_base 12: mod_base = OTHER; note = 2'-Fluoro-uridine
  modified_base 13: mod_base = gm
  modified_base 14: mod_base = gm
  modified_base 15: mod_base = um
  modified_base 16: mod_base = cm
  modified_base 17: mod_base = OTHER; note = 2'-Fluoro-adenosine
  modified_base 18: mod_base = um
  modified_base 19: mod_base = OTHER; note = 2 -O-methyl-adenosine
  source 1..19: mol_type = other RNA
cctaggaatgttggtcata

SEQ ID NO: 286 - RNA, 19 nt, synthetic construct
  modified_base 1..3: mod_base = OTHER; note = linked via phosphorothioate
  modified_base 1: mod_base = cm
  modified_base 2: mod_base = um
  modified_base 3: mod_base = OTHER; note = 2'-Fluoro-adenosine
  modified_base 4: mod_base = gm
  modified_base 5: mod_base = gm
  modified_base 6: mod_base = OTHER; note = 2 -O-methyl-adenosine
  modified_base 7: mod_base = OTHER; note = 2'-Fluoro-adenosine
  modified_base 8: mod_base = OTHER; note = 2'-Fluoro-uridine
  modified_base 9: mod_base = OTHER; note = 2'-Fluoro-guanosine
  modified_base 10: mod_base = um
  modified_base 11: mod_base = um
  modified_base 12: mod_base = OTHER; note = 2'-Fluoro-guanosine
  modified_base 13: mod_base = gm
  modified_base 14: mod_base = um
  modified_base 15: mod_base = cm
  modified_base 16: mod_base = OTHER; note = 2 -O-methyl-adenosine
  modified_base 17: mod_base = OTHER; note = 2'-Fluoro-uridine
  modified_base 18: mod_base = OTHER; note = 2 -O-methyl-adenosine
  modified_base 19: mod_base = OTHER; note = 2 -O-methyl-adenosine
  source 1..19: mol_type = other RNA
ctaggaatgttggtcataa

SEQ ID NO: 287 - RNA, 19 nt, synthetic construct
  modified_base 1..3: mod_base = OTHER; note = linked via phosphorothioate
  modified_base 1: mod_base = um
  modified_base 2: mod_base = OTHER; note = 2 -O-methyl-adenosine
  modified_base 3: mod_base = OTHER; note = 2'-Fluoro-guanosine
  modified_base 4: mod_base = gm
  modified_base 5: mod_base = OTHER; note = 2 -O-methyl-adenosine
  modified_base 6: mod_base = OTHER; note = 2 -O-methyl-adenosine
  modified_base 7: mod_base = OTHER; note = 2'-Fluoro-uridine
  modified_base 8: mod_base = OTHER; note = 2'-Fluoro-guanosine
  modified_base 9: mod_base = OTHER; note = 2'-Fluoro-uridine
  modified_base 10: mod_base = um
  modified_base 11: mod_base = gm
  modified_base 12: mod_base = OTHER; note = 2'-Fluoro-guanosine
  modified_base 13: mod_base = um
  modified_base 14: mod_base = cm
  modified_base 15: mod_base = OTHER; note = 2 -O-methyl-adenosine
  modified_base 16: mod_base = um
  modified_base 17: mod_base = OTHER; note = 2'-Fluoro-adenosine
  modified_base 18: mod_base = um
  modified_base 19: mod_base = OTHER; note = 2 -O-methyl-adenosine
  source 1..19: mol_type = other RNA
taggaatgttggtcatata

SEQ ID NO: 288 - RNA, 19 nt, synthetic construct
  modified_base 1..3: mod_base = OTHER; note = linked via phosphorothioate
  modified_base 1: mod_base = gm
  modified_base 2: mod_base = gm
  modified_base 3: mod_base = OTHER; note = 2'-Fluoro-adenosine
  modified_base 4: mod_base = OTHER; note = 2 -O-methyl-adenosine
  modified_base 5: mod_base = um
  modified_base 6: mod_base = gm
  modified_base 7: mod_base = OTHER; note = 2'-Fluoro-uridine
  modified_base 8: mod_base = OTHER; note = 2'-Fluoro-uridine
  modified_base 9: mod_base = OTHER; note = 2'-Fluoro-guanosine
  modified_base 10: mod_base = gm
  modified_base 11: mod_base = um
  modified_base 12: mod_base = OTHER; note = 2 -Fluoro-cytidine
  modified_base 13: mod_base = OTHER; note = 2 -O-methyl-adenosine
  modified_base 14: mod_base = um
  modified_base 15: mod_base = OTHER; note = 2 -O-methyl-adenosine
  modified_base 16: mod_base = um
  modified_base 17: mod_base = OTHER; note = 2 -Fluoro-cytidine
  modified_base 18: mod_base = OTHER; note = 2 -O-methyl-adenosine
  modified_base 19: mod_base = OTHER; note = 2 -O-methyl-adenosine
  source 1..19: mol_type = other RNA
ggaatgttggtcatatcaa

SEQ ID NO: 289 - RNA, 19 nt, synthetic construct
  modified_base 1..3: mod_base = OTHER; note = linked via phosphorothioate
  modified_base 1: mod_base = OTHER; note = 2 -O-methyl-adenosine
  modified_base 2: mod_base = cm
  modified_base 3: mod_base = OTHER; note = 2'-Fluoro-uridine
  modified_base 4: mod_base = gm
  modified_base 5: mod_base = um
  modified_base 6: mod_base = OTHER; note = 2 -O-methyl-adenosine
  modified_base 7: mod_base = OTHER; note = 2 -Fluoro-cytidine
  modified_base 8: mod_base = OTHER; note = 2'-Fluoro-uridine
  modified_base 9: mod_base = OTHER; note = 2'-Fluoro-adenosine
  modified_base 10: mod_base = cm
  modified_base 11: mod_base = um
  modified_base 12: mod_base = OTHER; note = 2 -Fluoro-cytidine
  modified_base 13: mod_base = cm
  modified_base 14: mod_base = um
  modified_base 15: mod_base = OTHER; note = 2 -O-methyl-adenosine
  modified_base 16: mod_base = OTHER; note = 2 -O-methyl-adenosine
  modified_base 17: mod_base = OTHER; note = 2'-Fluoro-uridine
  modified_base 18: mod_base = um
  modified_base 19: mod_base = OTHER; note = 2 -O-methyl-adenosine
  source 1..19: mol_type = other RNA
actgtactactcctaatta

SEQ ID NO: 290 - RNA, 19 nt, synthetic construct
  modified_base 1..3: mod_base = OTHER; note = linked via phosphorothioate
  modified_base 1: mod_base = cm
  modified_base 2: mod_base = um
  modified_base 3: mod_base = OTHER; note = 2'-Fluoro-guanosine
  modified_base 4: mod_base = um
  modified_base 5: mod_base = OTHER; note = 2 -O-methyl-adenosine
  modified_base 6: mod_base = cm
  modified_base 7: mod_base = OTHER; note = 2'-Fluoro-uridine
  modified_base 8: mod_base = OTHER; note = 2'-Fluoro-adenosine
  modified_base 9: mod_base = OTHER; note = 2 -Fluoro-cytidine
  modified_base 10: mod_base = um
  modified_base 11: mod_base = cm
  modified_base 12: mod_base = OTHER; note = 2 -Fluoro-cytidine
  modified_base 13: mod_base = um
  modified_base 14: mod_base = OTHER; note = 2 -O-methyl-adenosine
  modified_base 15: mod_base = OTHER; note = 2 -O-methyl-adenosine
  modified_base 16: mod_base = um
  modified_base 17: mod_base = OTHER; note = 2'-Fluoro-uridine
  modified_base 18: mod_base = OTHER; note = 2 -O-methyl-adenosine
  modified_base 19: mod_base = OTHER; note = 2 -O-methyl-adenosine
  source 1..19: mol_type = other RNA
ctgtactactcctaattaa

SEQ ID NO: 291 - RNA, 19 nt, synthetic construct
  modified_base 1..3: mod_base = OTHER; note = linked via phosphorothioate
  modified_base 1: mod_base = um
  modified_base 2: mod_base = gm
  modified_base 3: mod_base = OTHER; note = 2'-Fluoro-uridine
  modified_base 4: mod_base = OTHER; note = 2 -O-methyl-adenosine
  modified_base 5: mod_base = cm
  modified_base 6: mod_base = um
  modified_base 7: mod_base = OTHER; note = 2'-Fluoro-adenosine
  modified_base 8: mod_base = OTHER; note = 2 -Fluoro-cytidine
  modified_base 9: mod_base = OTHER; note = 2'-Fluoro-uridine
  modified_base 10: mod_base = cm
  modified_base 11: mod_base = cm
  modified_base 12: mod_base = OTHER; note = 2'-Fluoro-uridine
  modified_base 13: mod_base = OTHER; note = 2 -O-methyl-adenosine
  modified_base 14: mod_base = OTHER; note = 2 -O-methyl-adenosine
  modified_base 15: mod_base = um
  modified_base 16: mod_base = um
  modified_base 17: mod_base = OTHER; note = 2'-Fluoro-adenosine
  modified_base 18: mod_base = um
  modified_base 19: mod_base = OTHER; note = 2 -O-methyl-adenosine
  source 1..19: mol_type = other RNA
tgtactactcctaattata

SEQ ID NO: 292 - RNA, 19 nt, synthetic construct
  modified_base 1..3: mod_base = OTHER; note = linked via phosphorothioate
  modified_base 1: mod_base = gm
  modified_base 2: mod_base = um
  modified_base 3: mod_base = OTHER; note = 2'-Fluoro-adenosine
  modified_base 4: mod_base = cm
  modified_base 5: mod_base = um
  modified_base 6: mod_base = OTHER; note = 2 -O-methyl-adenosine
  modified_base 7: mod_base = OTHER; note = 2 -Fluoro-cytidine
  modified_base 8: mod_base = OTHER; note = 2'-Fluoro-uridine
  modified_base 9: mod_base = OTHER; note = 2 -Fluoro-cytidine
  modified_base 10: mod_base = cm
  modified_base 11: mod_base = um
  modified_base 12: mod_base = OTHER; note = 2'-Fluoro-adenosine
  modified_base 13: mod_base = OTHER; note = 2 -O-methyl-adenosine
  modified_base 14: mod_base = um
  modified_base 15: mod_base = um
  modified_base 16: mod_base = OTHER; note = 2 -O-methyl-adenosine
  modified_base 17: mod_base = OTHER; note = 2'-Fluoro-uridine
  modified_base 18: mod_base = um
  modified_base 19: mod_base = OTHER; note = 2 -O-methyl-adenosine
  source 1..19: mol_type = other RNA
gtactactcctaattatta

SEQ ID NO: 293 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = gm
  modified_base 4: mod_base = OTHER; note = 2 -O-methyl-adenosine
  modified_base 5: mod_base = gm
  modified_base 6: mod_base = um
  modified_base 7: mod_base = OTHER; note = 2 -O-methyl-adenosine
  modified_base 8: mod_base = cm
  modified_base 9: mod_base = um
  modified_base 10: mod_base = gm
  modified_base 11: mod_base = gm
  modified_base 12: mod_base = cm
  modified_base 13: mod_base = cm
  modified_base 14: mod_base = OTHER; note = 2'-Fluoro-guanosine
  modified_base 15: mod_base = OTHER; note = 2 -O-methyl-adenosine
  modified_base 16: mod_base = gm
  modified_base 17: mod_base = cm
  modified_base 18: mod_base = cm
  modified_base 19..21: mod_base = OTHER; note = linked via phosphorothioate
  modified_base 19: mod_base = gm
  modified_base 20: mod_base = cm
  modified_base 21: mod_base = cm
  source 1..21: mol_type = other RNA
tggagtactggccgagccgcc

SEQ ID NO: 294 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = OTHER; note = 2 -O-methyl-adenosine
  modified_base 4: mod_base = um
  modified_base 5: mod_base = um
  modified_base 6: mod_base = OTHER; note = 2 -O-methyl-adenosine
  modified_base 7: mod_base = gm
  modified_base 8: mod_base = cm
  modified_base 9: mod_base = um
  modified_base 10: mod_base = gm
  modified_base 11: mod_base = um
  modified_base 12: mod_base = OTHER; note = 2 -O-methyl-adenosine
  modified_base 13: mod_base = um
  modified_base 14: mod_base = OTHER; note = 2 -Fluoro-cytidine
  modified_base 15: mod_base = gm
  modified_base 16: mod_base = um
  modified_base 17: mod_base = cm
  modified_base 18: mod_base = OTHER; note = 2 -O-methyl-adenosine
  modified_base 19..21: mod_base = OTHER; note = linked via phosphorothioate
  modified_base 19: mod_base = OTHER; note = 2 -O-methyl-adenosine
  modified_base 20: mod_base = gm
  modified_base 21: mod_base = gm
  source 1..21: mol_type = other RNA
taattagctgtatcgtcaagg

SEQ ID NO: 295 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = OTHER; note = 2 -O-methyl-adenosine
  modified_base 4: mod_base = OTHER; note = 2 -O-methyl-adenosine
  modified_base 5: mod_base = um
  modified_base 6: mod_base = um
  modified_base 7: mod_base = OTHER; note = 2 -O-methyl-adenosine
  modified_base 8: mod_base = gm
  modified_base 9: mod_base = cm
  modified_base 10: mod_base = um
  modified_base 11: mod_base = gm
  modified_base 12: mod_base = um
  modified_base 13: mod_base = OTHER; note = 2 -O-methyl-adenosine
  modified_base 14: mod_base = OTHER; note = 2'-Fluoro-uridine
  modified_base 15: mod_base = cm
  modified_base 16: mod_base = gm
  modified_base 17: mod_base = um
  modified_base 18: mod_base = cm
  modified_base 19..21: mod_base = OTHER; note = linked via phosphorothioate
  modified_base 19: mod_base = OTHER; note = 2 -O-methyl-adenosine
  modified_base 20: mod_base = OTHER; note = 2 -O-methyl-adenosine
  modified_base 21: mod_base = gm
  source 1..21: mol_type = other RNA
tgaattagctgtatcgtcaag

SEQ ID NO: 296 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = gm
  modified_base 4: mod_base = OTHER; note = 2 -O-methyl-adenosine
  modified_base 5: mod_base = OTHER; note = 2 -O-methyl-adenosine
  modified_base 6: mod_base = um
  modified_base 7: mod_base = um
  modified_base 8: mod_base = OTHER; note = 2 -O-methyl-adenosine
  modified_base 9: mod_base = gm
  modified_base 10: mod_base = cm
  modified_base 11: mod_base = um
  modified_base 12: mod_base = gm
  modified_base 13: mod_base = um
  modified_base 14: mod_base = OTHER; note = 2'-Fluoro-adenosine
  modified_base 15: mod_base = um
  modified_base 16: mod_base = cm
  modified_base 17: mod_base = gm
  modified_base 18: mod_base = um
  modified_base 19..21: mod_base = OTHER; note = linked via phosphorothioate
  modified_base 19: mod_base = cm
  modified_base 20: mod_base = OTHER; note = 2 -O-methyl-adenosine
  modified_base 21: mod_base = OTHER; note = 2 -O-methyl-adenosine
  source 1..21: mol_type = other RNA
ttgaattagctgtatcgtcaa

SEQ ID NO: 297 - RNA, 21 nt, synthetic construct
  modified_base 1..3: mod_base = OTHER; note = linked via phosphorothioate
  modified_base 1: mod_base = um
  modified_base 2: mod_base = OTHER; note = 2 -Fluoro-cytidine
  modified_base 3: mod_base = um
  modified_base 4: mod_base = gm
  modified_base 5: mod_base = OTHER; note = 2 -O-methyl-adenosine
  modified_base 6: mod_base = OTHER; note = 2 -O-methyl-adenosine
  modified_base 7: mod_base = um
  modified_base 8: mod_base = um
  modified_base 9: mod_base = OTHER; note = 2 -O-methyl-adenosine
  modified_base 10: mod_base = gm
  modified_base 11: mod_base = cm
  modified_base 12: mod_base = um
  modified_base 13: mod_base = gm
  modified_base 14: mod_base = OTHER; note = 2'-Fluoro-uridine
  modified_base 15: mod_base = OTHER; note = 2 -O-methyl-adenosine
  modified_base 16: mod_base = um
  modified_base 17: mod_base = cm
  modified_base 18: mod_base = gm
  modified_base 19..21: mod_base = OTHER; note = linked via phosphorothioate
  modified_base 19: mod_base = um
  modified_base 20: mod_base = cm
  modified_base 21: mod_base = OTHER; note = 2 -O-methyl-adenosine
  source 1..21: mol_type = other RNA
tctgaattagctgtatcgtca

SEQ ID NO: 298 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = um
  modified_base 4: mod_base = cm
  modified_base 5: mod_base = um
  modified_base 6: mod_base = gm
  modified_base 7: mod_base = OTHER; note = 2 -O-methyl-adenosine
  modified_base 8: mod_base = OTHER; note = 2 -O-methyl-adenosine
  modified_base 9: mod_base = um
  modified_base 10: mod_base = um
  modified_base 11: mod_base = OTHER; note = 2 -O-methyl-adenosine
  modified_base 12: mod_base = gm
  modified_base 13: mod_base = cm
  modified_base 14: mod_base = OTHER; note = 2'-Fluoro-uridine
  modified_base 15: mod_base = gm
  modified_base 16: mod_base = um
  modified_base 17: mod_base = OTHER; note = 2 -O-methyl-adenosine
  modified_base 18: mod_base = um
  modified_base 19..21: mod_base = OTHER; note = linked via phosphorothioate
  modified_base 19: mod_base = cm
  modified_base 20: mod_base = gm
  modified_base 21: mod_base = um
  source 1..21: mol_type = other RNA
tttctgaattagctgtatcgt

SEQ ID NO: 299 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = um
  modified_base 4: mod_base = um
  modified_base 5: mod_base = gm
  modified_base 6: mod_base = gm
  modified_base 7: mod_base = OTHER; note = 2 -O-methyl-adenosine
  modified_base 8: mod_base = um
  modified_base 9: mod_base = cm
  modified_base 10: mod_base = OTHER; note = 2 -O-methyl-adenosine
  modified_base 11: mod_base = um
  modified_base 12: mod_base = OTHER; note = 2 -O-methyl-adenosine
  modified_base 13: mod_base = um
  modified_base 14: mod_base = OTHER; note = 2'-Fluoro-uridine
  modified_base 15: mod_base = cm
  modified_base 16: mod_base = gm
  modified_base 17: mod_base = um
  modified_base 18: mod_base = cm
  modified_base 19..21: mod_base = OTHER; note = linked via phosphorothioate
  modified_base 19: mod_base = cm
  modified_base 20: mod_base = OTHER; note = 2 -O-methyl-adenosine
  modified_base 21: mod_base = cm
  source 1..21: mol_type = other RNA
tgttggatcatattcgtccac

SEQ ID NO: 300 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = gm
  modified_base 4: mod_base = um
  modified_base 5: mod_base = um
  modified_base 6: mod_base = gm
  modified_base 7: mod_base = gm
  modified_base 8: mod_base = OTHER; note = 2 -O-methyl-adenosine
  modified_base 9: mod_base = um
  modified_base 10: mod_base = cm
  modified_base 11: mod_base = OTHER; note = 2 -O-methyl-adenosine
  modified_base 12: mod_base = um
  modified_base 13: mod_base = OTHER; note = 2 -O-methyl-adenosine
  modified_base 14: mod_base = OTHER; note = 2'-Fluoro-uridine
  modified_base 15: mod_base = um
  modified_base 16: mod_base = cm
  modified_base 17: mod_base = gm
  modified_base 18: mod_base = um
  modified_base 19..21: mod_base = OTHER; note = linked via phosphorothioate
  modified_base 19: mod_base = cm
  modified_base 20: mod_base = cm
  modified_base 21: mod_base = OTHER; note = 2 -O-methyl-adenosine
  source 1..21: mol_type = other RNA
ttgttggatcatattcgtcca

SEQ ID NO: 301 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = um
  modified_base 4: mod_base = gm
  modified_base 5: mod_base = um
  modified_base 6: mod_base = um
  modified_base 7: mod_base = gm
  modified_base 8: mod_base = gm
  modified_base 9: mod_base = OTHER; note = 2 -O-methyl-adenosine
  modified_base 10: mod_base = um
  modified_base 11: mod_base = cm
  modified_base 12: mod_base = OTHER; note = 2 -O-methyl-adenosine
  modified_base 13: mod_base = um
  modified_base 14: mod_base = OTHER; note = 2'-Fluoro-adenosine
  modified_base 15: mod_base = um
  modified_base 16: mod_base = um
  modified_base 17: mod_base = cm
  modified_base 18: mod_base = gm
  modified_base 19..21: mod_base = OTHER; note = linked via phosphorothioate
  modified_base 19: mod_base = um
  modified_base 20: mod_base = cm
  modified_base 21: mod_base = cm
  source 1..21: mol_type = other RNA
tttgttggatcatattcgtcc

SEQ ID NO: 302 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = um
  modified_base 4: mod_base = um
  modified_base 5: mod_base = gm
  modified_base 6: mod_base = um
  modified_base 7: mod_base = um
  modified_base 8: mod_base = gm
  modified_base 9: mod_base = gm
  modified_base 10: mod_base = OTHER; note = 2 -O-methyl-adenosine
  modified_base 11: mod_base = um
  modified_base 12: mod_base = cm
  modified_base 13: mod_base = OTHER; note = 2 -O-methyl-adenosine
  modified_base 14: mod_base = OTHER; note = 2'-Fluoro-uridine
  modified_base 15: mod_base = OTHER; note = 2 -O-methyl-adenosine
  modified_base 16: mod_base = um
  modified_base 17: mod_base = um
  modified_base 18: mod_base = cm
  modified_base 19..21: mod_base = OTHER; note = linked via phosphorothioate
  modified_base 19: mod_base = gm
  modified_base 20: mod_base = um
  modified_base 21: mod_base = cm
  source 1..21: mol_type = other RNA
tattgttggatcatattcgtc

SEQ ID NO: 303 - RNA, 21 nt, synthetic construct
  modified_base 1..3: mod_base = OTHER; note = linked via phosphorothioate
  modified_base 1: mod_base = um
  modified_base 2: mod_base = OTHER; note = 2 -Fluoro-cytidine
  modified_base 3: mod_base = gm
  modified_base 4: mod_base = OTHER; note = 2 -O-methyl-adenosine
  modified_base 5: mod_base = gm
  modified_base 6: mod_base = OTHER; note = 2 -O-methyl-adenosine
  modified_base 7: mod_base = OTHER; note = 2 -O-methyl-adenosine
  modified_base 8: mod_base = um
  modified_base 9: mod_base = OTHER; note = 2 -O-methyl-adenosine
  modified_base 10: mod_base = um
  modified_base 11: mod_base = cm
  modified_base 12: mod_base = cm
  modified_base 13: mod_base = OTHER; note = 2 -O-methyl-adenosine
  modified_base 14: mod_base = OTHER; note = 2'-Fluoro-adenosine
  modified_base 15: mod_base = gm
  modified_base 16: mod_base = OTHER; note = 2 -O-methyl-adenosine
  modified_base 17: mod_base = gm
  modified_base 18: mod_base = OTHER; note = 2 -O-methyl-adenosine
  modified_base 19..21: mod_base = OTHER; note = linked via phosphorothioate
  modified_base 19: mod_base = cm
  modified_base 20: mod_base = OTHER; note = 2 -O-methyl-adenosine
  modified_base 21: mod_base = gm
  source 1..21: mol_type = other RNA
tcgagaatatccaagagacag

SEQ ID NO: 304 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = cm
  modified_base 4: mod_base = um
  modified_base 5: mod_base = gm
  modified_base 6: mod_base = um
  modified_base 7: mod_base = gm
  modified_base 8: mod_base = um
  modified_base 9: mod_base = cm
  modified_base 10: mod_base = gm
  modified_base 11: mod_base = OTHER; note = 2 -O-methyl-adenosine
  modified_base 12: mod_base = gm
  modified_base 13: mod_base = OTHER; note = 2 -O-methyl-adenosine
  modified_base 14: mod_base = OTHER; note = 2'-Fluoro-adenosine
  modified_base 15: mod_base = um
  modified_base 16: mod_base = OTHER; note = 2 -O-methyl-adenosine
  modified_base 17: mod_base = um
  modified_base 18: mod_base = cm
  modified_base 19..21: mod_base = OTHER; note = linked via phosphorothioate
  modified_base 19: mod_base = cm
  modified_base 20: mod_base = OTHER; note = 2 -O-methyl-adenosine
  modified_base 21: mod_base = OTHER; note = 2 -O-methyl-adenosine
  source 1..21: mol_type = other RNA
tgctgtgtcgagaatatccaa

SEQ ID NO: 305 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = um
  modified_base 4: mod_base = OTHER; note = 2 -O-methyl-adenosine
  modified_base 5: mod_base = cm
  modified_base 6: mod_base = um
  modified_base 7: mod_base = gm
  modified_base 8: mod_base = gm
  modified_base 9: mod_base = um
  modified_base 10: mod_base = cm
  modified_base 11: mod_base = cm
  modified_base 12: mod_base = cm
  modified_base 13: mod_base = um
  modified_base 14: mod_base = OTHER; note = 2 -Fluoro-cytidine
  modified_base 15: mod_base = OTHER; note = 2 -O-methyl-adenosine
  modified_base 16: mod_base = um
  modified_base 17: mod_base = um
  modified_base 18: mod_base = gm
  modified_base 19..21: mod_base = OTHER; note = linked via phosphorothioate
  modified_base 19: mod_base = cm
  modified_base 20: mod_base = OTHER; note = 2 -O-methyl-adenosine
  modified_base 21: mod_base = cm
  source 1..21: mol_type = other RNA
tgtactggtccctcattgcac

SEQ ID NO: 306 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = gm
  modified_base 4: mod_base = um
  modified_base 5: mod_base = OTHER; note = 2 -O-methyl-adenosine
  modified_base 6: mod_base = cm
  modified_base 7: mod_base = um
  modified_base 8: mod_base = gm
  modified_base 9: mod_base = gm
  modified_base 10: mod_base = um
  modified_base 11: mod_base = cm
  modified_base 12: mod_base = cm
  modified_base 13: mod_base = cm
  modified_base 14: mod_base = OTHER; note = 2'-Fluoro-uridine
  modified_base 15: mod_base = cm
  modified_base 16: mod_base = OTHER; note = 2 -O-methyl-adenosine
  modified_base 17: mod_base = um
  modified_base 18: mod_base = um
  modified_base 19..21: mod_base = OTHER; note = linked via phosphorothioate
  modified_base 19: mod_base = gm
  modified_base 20: mod_base = cm
  modified_base 21: mod_base = OTHER; note = 2 -O-methyl-adenosine
  source 1..21: mol_type = other RNA
ttgtactggtccctcattgca

SEQ ID NO: 307 - RNA, 21 nt, synthetic construct
  modified_base 1..3: mod_base = OTHER; note = linked via phosphorothioate
  modified_base 1: mod_base = um
  modified_base 2: mod_base = OTHER; note = 2 -Fluoro-cytidine
  modified_base 3: mod_base = um
  modified_base 4: mod_base = OTHER; note = 2 -O-methyl-adenosine
  modified_base 5: mod_base = um
  modified_base 6: mod_base = OTHER; note = 2 -O-methyl-adenosine
  modified_base 7: mod_base = OTHER; note = 2 -O-methyl-adenosine
  modified_base 8: mod_base = um
  modified_base 9: mod_base = gm
  modified_base 10: mod_base = gm
  modified_base 11: mod_base = um
  modified_base 12: mod_base = gm
  modified_base 13: mod_base = OTHER; note = 2 -O-methyl-adenosine
  modified_base 14: mod_base = OTHER; note = 2'-Fluoro-adenosine
  modified_base 15: mod_base = um
  modified_base 16: mod_base = OTHER; note = 2 -O-methyl-adenosine
  modified_base 17: mod_base = um
  modified_base 18: mod_base = cm
  modified_base 19..21: mod_base = OTHER; note = linked via phosphorothioate
  modified_base 19: mod_base = um
  modified_base 20: mod_base = um
  modified_base 21: mod_base = cm
  source 1..21: mol_type = other RNA
tctataatggtgaatatcttc

SEQ ID NO: 308 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = cm
  modified_base 4: mod_base = um
  modified_base 5: mod_base = OTHER; note = 2 -O-methyl-adenosine
  modified_base 6: mod_base = um
  modified_base 7: mod_base = OTHER; note = 2 -O-methyl-adenosine
  modified_base 8: mod_base = OTHER; note = 2 -O-methyl-adenosine
  modified_base 9: mod_base = um
  modified_base 10: mod_base = gm
  modified_base 11: mod_base = gm
  modified_base 12: mod_base = um
  modified_base 13: mod_base = gm
  modified_base 14: mod_base = OTHER; note = 2'-Fluoro-adenosine
  modified_base 15: mod_base = OTHER; note = 2 -O-methyl-adenosine
  modified_base 16: mod_base = um
  modified_base 17: mod_base = OTHER; note = 2 -O-methyl-adenosine
  modified_base 18: mod_base = um
  modified_base 19..21: mod_base = OTHER; note = linked via phosphorothioate
  modified_base 19: mod_base = cm
  modified_base 20: mod_base = um
  modified_base 21: mod_base = um
  source 1..21: mol_type = other RNA
ttctataatggtgaatatctt

SEQ ID NO: 309 - RNA, 21 nt, synthetic construct
  modified_base 1..3: mod_base = OTHER; note = linked via phosphorothioate
  modified_base 1: mod_base = um
  modified_base 2: mod_base = OTHER; note = 2 -Fluoro-cytidine
  modified_base 3: mod_base = um
  modified_base 4: mod_base = cm
  modified_base 5: mod_base = um
  modified_base 6: mod_base = OTHER; note = 2 -O-methyl-adenosine
  modified_base 7: mod_base = um
  modified_base 8: mod_base = OTHER; note = 2 -O-methyl-adenosine
  modified_base 9: mod_base = OTHER; note = 2 -O-methyl-adenosine
  modified_base 10: mod_base = um
  modified_base 11: mod_base = gm
  modified_base 12: mod_base = gm
  modified_base 13: mod_base = um
  modified_base 14: mod_base = OTHER; note = 2'-Fluoro-guanosine
  modified_base 15: mod_base = OTHER; note = 2 -O-methyl-adenosine
  modified_base 16: mod_base = OTHER; note = 2 -O-methyl-adenosine
  modified_base 17: mod_base = um
  modified_base 18: mod_base = OTHER; note = 2 -O-methyl-adenosine
  modified_base 19..21: mod_base = OTHER; note = linked via phosphorothioate
  modified_base 19: mod_base = um
  modified_base 20: mod_base = cm
  modified_base 21: mod_base = um
  source 1..21: mol_type = other RNA
tctctataatggtgaatatct

SEQ ID NO: 310 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = cm
  modified_base 4: mod_base = um
  modified_base 5: mod_base = cm
  modified_base 6: mod_base = um
  modified_base 7: mod_base = OTHER; note = 2 -O-methyl-adenosine
  modified_base 8: mod_base = um
  modified_base 9: mod_base = OTHER; note = 2 -O-methyl-adenosine
  modified_base 10: mod_base = OTHER; note = 2 -O-methyl-adenosine
  modified_base 11: mod_base = um
  modified_base 12: mod_base = gm
  modified_base 13: mod_base = gm
  modified_base 14: mod_base = OTHER; note = 2'-Fluoro-uridine
  modified_base 15: mod_base = gm
  modified_base 16: mod_base = OTHER; note = 2 -O-methyl-adenosine
  modified_base 17: mod_base = OTHER; note = 2 -O-methyl-adenosine
  modified_base 18: mod_base = um
  modified_base 19..21: mod_base = OTHER; note = linked via phosphorothioate
  modified_base 19: mod_base = OTHER; note = 2 -O-methyl-adenosine
  modified_base 20: mod_base = um
  modified_base 21: mod_base = cm
  source 1..21: mol_type = other RNA
ttctctataatggtgaatatc

SEQ ID NO: 311 - RNA, 21 nt, synthetic construct
  modified_base 1..3: mod_base = OTHER; note = linked via phosphorothioate
  modified_base 1: mod_base = um
  modified_base 2: mod_base = OTHER; note = 2 -Fluoro-cytidine
  modified_base 3: mod_base = cm
  modified_base 4: mod_base = um
  modified_base 5: mod_base = OTHER; note = 2 -O-methyl-adenosine
  modified_base 6: mod_base = cm
  modified_base 7: mod_base = um
  modified_base 8: mod_base = OTHER; note = 2 -O-methyl-adenosine
  modified_base 9: mod_base = gm
  modified_base 10: mod_base = gm
  modified_base 11: mod_base = OTHER; note = 2 -O-methyl-adenosine
  modified_base 12: mod_base = cm
  modified_base 13: mod_base = cm
  modified_base 14: mod_base = OTHER; note = 2'-Fluoro-adenosine
  modified_base 15: mod_base = um
  modified_base 16: mod_base = OTHER; note = 2 -O-methyl-adenosine
  modified_base 17: mod_base = gm
  modified_base 18: mod_base = gm
  modified_base 19..21: mod_base = OTHER; note = linked via phosphorothioate
  modified_base 19: mod_base = um
  modified_base 20: mod_base = OTHER; note = 2 -O-methyl-adenosine
  modified_base 21: mod_base = cm
  source 1..21: mol_type = other RNA
tcctactaggaccataggtac

SEQ ID NO: 312 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = OTHER; note = 2 -O-methyl-adenosine
  modified_base 4: mod_base = OTHER; note = 2 -O-methyl-adenosine
  modified_base 5: mod_base = cm
  modified_base 6: mod_base = um
  modified_base 7: mod_base = um
  modified_base 8: mod_base = cm
  modified_base 9: mod_base = um
  modified_base 10: mod_base = um
  modified_base 11: mod_base = gm
  modified_base 12: mod_base = cm
  modified_base 13: mod_base = um
  modified_base 14: mod_base = OTHER; note = 2'-Fluoro-adenosine
  modified_base 15: mod_base = OTHER; note = 2 -O-methyl-adenosine
  modified_base 16: mod_base = gm
  modified_base 17: mod_base = um
  modified_base 18: mod_base = cm
  modified_base 19..21: mod_base = OTHER; note = linked via phosphorothioate
  modified_base 19: mod_base = cm
  modified_base 20: mod_base = um
  modified_base 21: mod_base = gm
  source 1..21: mol_type = other RNA
ttaacttcttgctaagtcctg

SEQ ID NO: 313 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = cm
  modified_base 4: mod_base = gm
  modified_base 5: mod_base = OTHER; note = 2 -O-methyl-adenosine
  modified_base 6: mod_base = OTHER; note = 2 -O-methyl-adenosine
  modified_base 7: mod_base = cm
  modified_base 8: mod_base = um
  modified_base 9: mod_base = OTHER; note = 2 -O-methyl-adenosine
  modified_base 10: mod_base = OTHER; note = 2 -O-methyl-adenosine
  modified_base 11: mod_base = um
  modified_base 12: mod_base = gm
  modified_base 13: mod_base = um
  modified_base 14: mod_base = OTHER; note = 2'-Fluoro-adenosine
  modified_base 15: mod_base = um
  modified_base 16: mod_base = OTHER; note = 2 -O-methyl-adenosine
  modified_base 17: mod_base = gm
  modified_base 18: mod_base = OTHER; note = 2 -O-methyl-adenosine
  modified_base 19..21: mod_base = OTHER; note = linked via phosphorothioate
  modified_base 19: mod_base = OTHER; note = 2 -O-methyl-adenosine
  modified_base 20: mod_base = gm
  modified_base 21: mod_base = gm
  source 1..21: mol_type = other RNA
ttcgaactaatgtatagaagg

SEQ ID NO: 314 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = um
  modified_base 4: mod_base = cm
  modified_base 5: mod_base = um
  modified_base 6: mod_base = cm
  modified_base 7: mod_base = gm
  modified_base 8: mod_base = OTHER; note = 2 -O-methyl-adenosine
  modified_base 9: mod_base = OTHER; note = 2 -O-methyl-adenosine
  modified_base 10: mod_base = cm
  modified_base 11: mod_base = um
  modified_base 12: mod_base = OTHER; note = 2 -O-methyl-adenosine
  modified_base 13: mod_base = OTHER; note = 2 -O-methyl-adenosine
  modified_base 14: mod_base = OTHER; note = 2'-Fluoro-uridine
  modified_base 15: mod_base = gm
  modified_base 16: mod_base = um
  modified_base 17: mod_base = OTHER; note = 2 -O-methyl-adenosine
  modified_base 18: mod_base = um
  modified_base 19..21: mod_base = OTHER; note = linked via phosphorothioate
  modified_base 19: mod_base = OTHER; note = 2 -O-methyl-adenosine
  modified_base 20: mod_base = gm
  modified_base 21: mod_base = OTHER; note = 2 -O-methyl-adenosine
  source 1..21: mol_type = other RNA
tttctcgaactaatgtataga

SEQ ID NO: 315 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = OTHER; note = 2 -O-methyl-adenosine
  modified_base 4: mod_base = OTHER; note = 2 -O-methyl-adenosine
  modified_base 5: mod_base = um
  modified_base 6: mod_base = um
  modified_base 7: mod_base = um
  modified_base 8: mod_base = cm
  modified_base 9: mod_base = um
  modified_base 10: mod_base = cm
  modified_base 11: mod_base = gm
  modified_base 12: mod_base = OTHER; note = 2 -O-methyl-adenosine
  modified_base 13: mod_base = OTHER; note = 2 -O-methyl-adenosine
  modified_base 14: mod_base = OTHER; note = 2 -Fluoro-cytidine
  modified_base 15: mod_base = um
  modified_base 16: mod_base = OTHER; note = 2 -O-methyl-adenosine
  modified_base 17: mod_base = OTHER; note = 2 -O-methyl-adenosine
  modified_base 18: mod_base = um
  modified_base 19..21: mod_base = OTHER; note = linked via phosphorothioate
  modified_base 19: mod_base = gm
  modified_base 20: mod_base = um
  modified_base 21: mod_base = OTHER; note = 2 -O-methyl-adenosine
  source 1..21: mol_type = other RNA
tgaatttctcgaactaatgta

SEQ ID NO: 316 - RNA, 21 nt, synthetic construct
  modified_base 1..3: mod_base = OTHER; note = linked via phosphorothioate
  modified_base 1: mod_base = um
  modified_base 2: mod_base = OTHER; note = 2 -Fluoro-cytidine
  modified_base 3: mod_base = gm
  modified_base 4: mod_base = OTHER; note = 2 -O-methyl-adenosine
  modified_base 5: mod_base = OTHER; note = 2 -O-methyl-adenosine
  modified_base 6: mod_base = um
  modified_base 7: mod_base = um
  modified_base 8: mod_base = um
  modified_base 9: mod_base = cm
  modified_base 10: mod_base = um
  modified_base 11: mod_base = cm
  modified_base 12: mod_base = gm
  modified_base 13: mod_base = OTHER; note = 2 -O-methyl-adenosine
  modified_base 14: mod_base = OTHER; note = 2'-Fluoro-adenosine
  modified_base 15: mod_base = cm
  modified_base 16: mod_base = um
  modified_base 17: mod_base = OTHER; note = 2 -O-methyl-adenosine
  modified_base 18: mod_base = OTHER; note = 2 -O-methyl-adenosine
  modified_base 19..21: mod_base = OTHER; note = linked via phosphorothioate
  modified_base 19: mod_base = um
  modified_base 20: mod_base = gm
  modified_base 21: mod_base = um
  source 1..21: mol_type = other RNA
tcgaatttctcgaactaatgt

SEQ ID NO: 317 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = cm
  modified_base 4: mod_base = gm
  modified_base 5: mod_base = OTHER; note = 2 -O-methyl-adenosine
  modified_base 6: mod_base = OTHER; note = 2 -O-methyl-adenosine
  modified_base 7: mod_base = um
  modified_base 8: mod_base = um
  modified_base 9: mod_base = um
  modified_base 10: mod_base = cm
  modified_base 11: mod_base = um
  modified_base 12: mod_base = cm
  modified_base 13: mod_base = gm
  modified_base 14: mod_base = OTHER; note = 2'-Fluoro-adenosine
  modified_base 15: mod_base = OTHER; note = 2 -O-methyl-adenosine
  modified_base 16: mod_base = cm
  modified_base 17: mod_base = um
  modified_base 18: mod_base = OTHER; note = 2 -O-methyl-adenosine
  modified_base 19..21: mod_base = OTHER; note = linked via phosphorothioate
  modified_base 19: mod_base = OTHER; note = 2 -O-methyl-adenosine
  modified_base 20: mod_base = um
  modified_base 21: mod_base = gm
  source 1..21: mol_type = other RNA
ttcgaatttctcgaactaatg

SEQ ID NO: 318 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = um
  modified_base 4: mod_base = um
  modified_base 5: mod_base = cm
  modified_base 6: mod_base = gm
  modified_base 7: mod_base = OTHER; note = 2 -O-methyl-adenosine
  modified_base 8: mod_base = OTHER; note = 2 -O-methyl-adenosine
  modified_base 9: mod_base = um
  modified_base 10: mod_base = um
  modified_base 11: mod_base = um
  modified_base 12: mod_base = cm
  modified_base 13: mod_base = um
  modified_base 14: mod_base = OTHER; note = 2 -Fluoro-cytidine
  modified_base 15: mod_base = gm
  modified_base 16: mod_base = OTHER; note = 2 -O-methyl-adenosine
  modified_base 17: mod_base = OTHER; note = 2 -O-methyl-adenosine
  modified_base 18: mod_base = cm
  modified_base 19..21: mod_base = OTHER; note = linked via phosphorothioate
  modified_base 19: mod_base = um
  modified_base 20: mod_base = OTHER; note = 2 -O-methyl-adenosine
  modified_base 21: mod_base = OTHER; note = 2 -O-methyl-adenosine
  source 1..21: mol_type = other RNA
ttttcgaatttctcgaactaa

SEQ ID NO: 319 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = um
  modified_base 4: mod_base = OTHER; note = 2 -O-methyl-adenosine
  modified_base 5: mod_base = um
  modified_base 6: mod_base = gm
  modified_base 7: mod_base = um
  modified_base 8: mod_base = um
  modified_base 9: mod_base = um
  modified_base 10: mod_base = um
  modified_base 11: mod_base = cm
  modified_base 12: mod_base = gm
  modified_base 13: mod_base = OTHER; note = 2 -O-methyl-adenosine
  modified_base 14: mod_base = OTHER; note = 2'-Fluoro-adenosine
  modified_base 15: mod_base = um
  modified_base 16: mod_base = um
  modified_base 17: mod_base = um
  modified_base 18: mod_base = cm
  modified_base 19..21: mod_base = OTHER; note = linked via phosphorothioate
  modified_base 19: mod_base = um
  modified_base 20: mod_base = cm
  modified_base 21: mod_base = gm
  source 1..21: mol_type = other RNA
tttatgttttcgaatttctcg

SEQ ID NO: 320 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = cm
  modified_base 4: mod_base = um
  modified_base 5: mod_base = um
  modified_base 6: mod_base = um
  modified_base 7: mod_base = OTHER; note = 2 -O-methyl-adenosine
  modified_base 8: mod_base = um
  modified_base 9: mod_base = gm
  modified_base 10: mod_base = um
  modified_base 11: mod_base = um
  modified_base 12: mod_base = um
  modified_base 13: mod_base = um
  modified_base 14: mod_base = OTHER; note = 2 -Fluoro-cytidine
  modified_base 15: mod_base = gm
  modified_base 16: mod_base = OTHER; note = 2 -O-methyl-adenosine
  modified_base 17: mod_base = OTHER; note = 2 -O-methyl-adenosine
  modified_base 18: mod_base = um
  modified_base 19..21: mod_base = OTHER; note = linked via phosphorothioate
  modified_base 19: mod_base = um
  modified_base 20: mod_base = um
  modified_base 21: mod_base = cm
  source 1..21: mol_type = other RNA
ttctttatgttttcgaatttc

SEQ ID NO: 321 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = OTHER; note = 2 -O-methyl-adenosine
  modified_base 4: mod_base = um
  modified_base 5: mod_base = um
  modified_base 6: mod_base = um
  modified_base 7: mod_base = OTHER; note = 2 -O-methyl-adenosine
  modified_base 8: mod_base = gm
  modified_base 9: mod_base = um
  modified_base 10: mod_base = gm
  modified_base 11: mod_base = um
  modified_base 12: mod_base = OTHER; note = 2 -O-methyl-adenosine
  modified_base 13: mod_base = OTHER; note = 2 -O-methyl-adenosine
  modified_base 14: mod_base = OTHER; note = 2'-Fluoro-uridine
  modified_base 15: mod_base = gm
  modified_base 16: mod_base = um
  modified_base 17: mod_base = OTHER; note = 2 -O-methyl-adenosine
  modified_base 18: mod_base = cm
  modified_base 19..21: mod_base = OTHER; note = linked via phosphorothioate
  modified_base 19: mod_base = OTHER; note = 2 -O-methyl-adenosine
  modified_base 20: mod_base = OTHER; note = 2 -O-methyl-adenosine
  modified_base 21: mod_base = OTHER; note = 2 -O-methyl-adenosine
  source 1..21: mol_type = other RNA
taatttagtgtaatgtacaaa

SEQ ID NO: 322 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = OTHER; note = 2 -O-methyl-adenosine
  modified_base 4: mod_base = OTHER; note = 2 -O-methyl-adenosine
  modified_base 5: mod_base = gm
  modified_base 6: mod_base = OTHER; note = 2 -O-methyl-adenosine
  modified_base 7: mod_base = OTHER; note = 2 -O-methyl-adenosine
  modified_base 8: mod_base = gm
  modified_base 9: mod_base = um
  modified_base 10: mod_base = OTHER; note = 2 -O-methyl-adenosine
  modified_base 11: mod_base = OTHER; note = 2 -O-methyl-adenosine
  modified_base 12: mod_base = um
  modified_base 13: mod_base = cm
  modified_base 14: mod_base = OTHER; note = 2'-Fluoro-adenosine
  modified_base 15: mod_base = OTHER; note = 2 -O-methyl-adenosine
  modified_base 16: mod_base = cm
  modified_base 17: mod_base = um
  modified_base 18: mod_base = gm
  modified_base 19..21: mod_base = OTHER; note = linked via phosphorothioate
  modified_base 19: mod_base = cm
  modified_base 20: mod_base = OTHER; note = 2 -O-methyl-adenosine
  modified_base 21: mod_base = um
  source 1..21: mol_type = other RNA
ttaagaagtaatcaactgcat

SEQ ID NO: 323 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = cm
  modified_base 4: mod_base = OTHER; note = 2 -O-methyl-adenosine
  modified_base 5: mod_base = gm
  modified_base 6: mod_base = OTHER; note = 2 -O-methyl-adenosine
  modified_base 7: mod_base = OTHER; note = 2 -O-methyl-adenosine
  modified_base 8: mod_base = um
  modified_base 9: mod_base = OTHER; note = 2 -O-methyl-adenosine
  modified_base 10: mod_base = gm
  modified_base 11: mod_base = gm
  modified_base 12: mod_base = gm
  modified_base 13: mod_base = OTHER; note = 2 -O-methyl-adenosine
  modified_base 14: mod_base = OTHER; note = 2'-Fluoro-uridine
  modified_base 15: mod_base = gm
  modified_base 16: mod_base = OTHER; note = 2 -O-methyl-adenosine
  modified_base 17: mod_base = um
  modified_base 18: mod_base = um
  modified_base 19..21: mod_base = OTHER; note = linked via phosphorothioate
  modified_base 19: mod_base = cm
  modified_base 20: mod_base = OTHER; note = 2 -O-methyl-adenosine
  modified_base 21: mod_base = OTHER; note = 2 -O-methyl-adenosine
  source 1..21: mol_type = other RNA
tacagaatagggatgattcaa

SEQ ID NO: 324 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = OTHER; note = 2 -O-methyl-adenosine
  modified_base 4: mod_base = cm
  modified_base 5: mod_base = um
  modified_base 6: mod_base = OTHER; note = 2 -O-methyl-adenosine
  modified_base 7: mod_base = um
  modified_base 8: mod_base = OTHER; note = 2 -O-methyl-adenosine
  modified_base 9: mod_base = gm
  modified_base 10: mod_base = gm
  modified_base 11: mod_base = OTHER; note = 2 -O-methyl-adenosine
  modified_base 12: mod_base = cm
  modified_base 13: mod_base = OTHER; note = 2 -O-methyl-adenosine
  modified_base 14: mod_base = OTHER; note = 2'-Fluoro-uridine
  modified_base 15: mod_base = gm
  modified_base 16: mod_base = OTHER; note = 2 -O-methyl-adenosine
  modified_base 17: mod_base = um
  modified_base 18: mod_base = gm
  modified_base 19..21: mod_base = OTHER; note = linked via phosphorothioate
  modified_base 19: mod_base = cm
  modified_base 20: mod_base = cm
  modified_base 21: mod_base = um
  source 1..21: mol_type = other RNA
taactataggacatgatgcct

SEQ ID NO: 325 - RNA, 21 nt, synthetic construct
  modified_base 1..3: mod_base = OTHER; note = linked via phosphorothioate
  modified_base 1: mod_base = um
  modified_base 2: mod_base = OTHER; note = 2 -Fluoro-cytidine
  modified_base 3: mod_base = cm
  modified_base 4: mod_base = OTHER; note = 2 -O-methyl-adenosine
  modified_base 5: mod_base = um
  modified_base 6: mod_base = gm
  modified_base 7: mod_base = OTHER; note = 2 -O-methyl-adenosine
  modified_base 8: mod_base = cm
  modified_base 9: mod_base = um
  modified_base 10: mod_base = OTHER; note = 2 -O-methyl-adenosine
  modified_base 11: mod_base = OTHER; note = 2 -O-methyl-adenosine
  modified_base 12: mod_base = um
  modified_base 13: mod_base = OTHER; note = 2 -O-methyl-adenosine
  modified_base 14: mod_base = OTHER; note = 2'-Fluoro-guanosine
  modified_base 15: mod_base = cm
  modified_base 16: mod_base = OTHER; note = 2 -O-methyl-adenosine
  modified_base 17: mod_base = gm
  modified_base 18: mod_base = um
  modified_base 19..21: mod_base = OTHER; note = linked via phosphorothioate
  modified_base 19: mod_base = gm
  modified_base 20: mod_base = gm
  modified_base 21: mod_base = OTHER; note = 2 -O-methyl-adenosine
  source 1..21: mol_type = other RNA
tccatgactaatagcagtgga

SEQ ID NO: 326 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = gm
  modified_base 4: mod_base = OTHER; note = 2 -O-methyl-adenosine
  modified_base 5: mod_base = cm
  modified_base 6: mod_base = cm
  modified_base 7: mod_base = OTHER; note = 2 -O-methyl-adenosine
  modified_base 8: mod_base = um
  modified_base 9: mod_base = gm
  modified_base 10: mod_base = OTHER; note = 2 -O-methyl-adenosine
  modified_base 11: mod_base = cm
  modified_base 12: mod_base = um
  modified_base 13: mod_base = OTHER; note = 2 -O-methyl-adenosine
  modified_base 14: mod_base = OTHER; note = 2'-Fluoro-adenosine
  modified_base 15: mod_base = um
  modified_base 16: mod_base = OTHER; note = 2 -O-methyl-adenosine
  modified_base 17: mod_base = gm
  modified_base 18: mod_base = cm
  modified_base 19..21: mod_base = OTHER; note = linked via phosphorothioate
  modified_base 19: mod_base = OTHER; note = 2 -O-methyl-adenosine
  modified_base 20: mod_base = gm
  modified_base 21: mod_base = um
  source 1..21: mol_type = other RNA
ttgaccatgactaatagcagt

SEQ ID NO: 327 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = OTHER; note = 2 -O-methyl-adenosine
  modified_base 4: mod_base = gm
  modified_base 5: mod_base = um
  modified_base 6: mod_base = OTHER; note = 2 -O-methyl-adenosine
  modified_base 7: mod_base = OTHER; note = 2 -O-methyl-adenosine
  modified_base 8: mod_base = um
  modified_base 9: mod_base = um
  modified_base 10: mod_base = um
  modified_base 11: mod_base = OTHER; note = 2 -O-methyl-adenosine
  modified_base 12: mod_base = um
  modified_base 13: mod_base = cm
  modified_base 14: mod_base = OTHER; note = 2'-Fluoro-uridine
  modified_base 15: mod_base = OTHER; note = 2 -O-methyl-adenosine
  modified_base 16: mod_base = OTHER; note = 2 -O-methyl-adenosine
  modified_base 17: mod_base = um
  modified_base 18: mod_base = gm
  modified_base 19..21: mod_base = OTHER; note = linked via phosphorothioate
  modified_base 19: mod_base = um
  modified_base 20: mod_base = gm
  modified_base 21: mod_base = OTHER; note = 2 -O-methyl-adenosine
  source 1..21: mol_type = other RNA
ttagtaatttatctaatgtga

SEQ ID NO: 328 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = OTHER; note = 2 -O-methyl-adenosine
  modified_base 4: mod_base = OTHER; note = 2 -O-methyl-adenosine
  modified_base 5: mod_base = um
  modified_base 6: mod_base = cm
  modified_base 7: mod_base = cm
  modified_base 8: mod_base = cm
  modified_base 9: mod_base = cm
  modified_base 10: mod_base = OTHER; note = 2 -O-methyl-adenosine
  modified_base 11: mod_base = um
  modified_base 12: mod_base = um
  modified_base 13: mod_base = um
  modified_base 14: mod_base = OTHER; note = 2 -Fluoro-cytidine
  modified_base 15: mod_base = OTHER; note = 2 -O-methyl-adenosine
  modified_base 16: mod_base = um
  modified_base 17: mod_base = OTHER; note = 2 -O-methyl-adenosine
  modified_base 18: mod_base = cm
  modified_base 19..21: mod_base = OTHER; note = linked via phosphorothioate
  modified_base 19: mod_base = um
  modified_base 20: mod_base = gm
  modified_base 21: mod_base = gm
  source 1..21: mol_type = other RNA
ttaatccccatttcatactgg

SEQ ID NO: 329 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = um
  modified_base 4: mod_base = OTHER; note = 2 -O-methyl-adenosine
  modified_base 5: mod_base = um
  modified_base 6: mod_base = OTHER; note = 2 -O-methyl-adenosine
  modified_base 7: mod_base = gm
  modified_base 8: mod_base = cm
  modified_base 9: mod_base = cm
  modified_base 10: mod_base = cm
  modified_base 11: mod_base = cm
  modified_base 12: mod_base = OTHER; note = 2 -O-methyl-adenosine
  modified_base 13: mod_base = OTHER; note = 2 -O-methyl-adenosine
  modified_base 14: mod_base = OTHER; note = 2'-Fluoro-adenosine
  modified_base 15: mod_base = OTHER; note = 2 -O-methyl-adenosine
  modified_base 16: mod_base = um
  modified_base 17: mod_base = gm
  modified_base 18: mod_base = gm
  modified_base 19..21: mod_base = OTHER; note = linked via phosphorothioate
  modified_base 19: mod_base = um
  modified_base 20: mod_base = um
  modified_base 21: mod_base = gm
  source 1..21: mol_type = other RNA
tatatagccccaaaatggttg

SEQ ID NO: 330 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = gm
  modified_base 4: mod_base = cm
  modified_base 5: mod_base = um
  modified_base 6: mod_base = OTHER; note = 2 -O-methyl-adenosine
  modified_base 7: mod_base = um
  modified_base 8: mod_base = OTHER; note = 2 -O-methyl-adenosine
  modified_base 9: mod_base = OTHER; note = 2 -O-methyl-adenosine
  modified_base 10: mod_base = cm
  modified_base 11: mod_base = um
  modified_base 12: mod_base = gm
  modified_base 13: mod_base = gm
  modified_base 14: mod_base = OTHER; note = 2 -Fluoro-cytidine
  modified_base 15: mod_base = cm
  modified_base 16: mod_base = cm
  modified_base 17: mod_base = OTHER; note = 2 -O-methyl-adenosine
  modified_base 18: mod_base = OTHER; note = 2 -O-methyl-adenosine
  modified_base 19..21: mod_base = OTHER; note = linked via phosphorothioate
  modified_base 19: mod_base = OTHER; note = 2 -O-methyl-adenosine
  modified_base 20: mod_base = um
  modified_base 21: mod_base = OTHER; note = 2 -O-methyl-adenosine
  source 1..21: mol_type = other RNA
tagctataactggcccaaata

SEQ ID NO: 331 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = gm
  modified_base 4: mod_base = OTHER; note = 2 -O-methyl-adenosine
  modified_base 5: mod_base = um
  modified_base 6: mod_base = um
  modified_base 7: mod_base = um
  modified_base 8: mod_base = gm
  modified_base 9: mod_base = um
  modified_base 10: mod_base = cm
  modified_base 11: mod_base = OTHER; note = 2 -O-methyl-adenosine
  modified_base 12: mod_base = gm
  modified_base 13: mod_base = cm
  modified_base 14: mod_base = OTHER; note = 2'-Fluoro-adenosine
  modified_base 15: mod_base = gm
  modified_base 16: mod_base = gm
  modified_base 17: mod_base = OTHER; note = 2 -O-methyl-adenosine
  modified_base 18: mod_base = cm
  modified_base 19..21: mod_base = OTHER; note = linked via phosphorothioate
  modified_base 19: mod_base = cm
  modified_base 20: mod_base = OTHER; note = 2 -O-methyl-adenosine
  modified_base 21: mod_base = cm
  source 1..21: mol_type = other RNA
ttgatttgtcagcaggaccac

SEQ ID NO: 332 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = cm
  modified_base 4: mod_base = cm
  modified_base 5: mod_base = OTHER; note = 2 -O-methyl-adenosine
  modified_base 6: mod_base = OTHER; note = 2 -O-methyl-adenosine
  modified_base 7: mod_base = OTHER; note = 2 -O-methyl-adenosine
  modified_base 8: mod_base = cm
  modified_base 9: mod_base = cm
  modified_base 10: mod_base = um
  modified_base 11: mod_base = OTHER; note = 2 -O-methyl-adenosine
  modified_base 12: mod_base = gm
  modified_base 13: mod_base = OTHER; note = 2 -O-methyl-adenosine
  modified_base 14: mod_base = OTHER; note = 2'-Fluoro-guanosine
  modified_base 15: mod_base = OTHER; note = 2 -O-methyl-adenosine
  modified_base 16: mod_base = um
  modified_base 17: mod_base = um
  modified_base 18: mod_base = gm
  modified_base 19..21: mod_base = OTHER; note = linked via phosphorothioate
  modified_base 19: mod_base = um
  modified_base 20: mod_base = OTHER; note = 2 -O-methyl-adenosine
  modified_base 21: mod_base = OTHER; note = 2 -O-methyl-adenosine
  source 1..21: mol_type = other RNA
tgccaaacctagagattgtaa

SEQ ID NO: 333 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = OTHER; note = 2 -O-methyl-adenosine
  modified_base 4: mod_base = OTHER; note = 2 -O-methyl-adenosine
  modified_base 5: mod_base = cm
  modified_base 6: mod_base = um
  modified_base 7: mod_base = OTHER; note = 2 -O-methyl-adenosine
  modified_base 8: mod_base = gm
  modified_base 9: mod_base = cm
  modified_base 10: mod_base = cm
  modified_base 11: mod_base = OTHER; note = 2 -O-methyl-adenosine
  modified_base 12: mod_base = OTHER; note = 2 -O-methyl-adenosine
  modified_base 13: mod_base = OTHER; note = 2 -O-methyl-adenosine
  modified_base 14: mod_base = OTHER; note = 2 -Fluoro-cytidine
  modified_base 15: mod_base = cm
  modified_base 16: mod_base = um
  modified_base 17: mod_base = OTHER; note = 2 -O-methyl-adenosine
  modified_base 18: mod_base = gm
  modified_base 19..21: mod_base = OTHER; note = linked via phosphorothioate
  modified_base 19: mod_base = OTHER; note = 2 -O-methyl-adenosine
  modified_base 20: mod_base = gm
  modified_base 21: mod_base = OTHER; note = 2 -O-methyl-adenosine
  source 1..21: mol_type = other RNA
tgaactagccaaacctagaga

SEQ ID NO: 334 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = OTHER; note = 2 -O-methyl-adenosine
  modified_base 4: mod_base = OTHER; note = 2 -O-methyl-adenosine
  modified_base 5: mod_base = gm
  modified_base 6: mod_base = OTHER; note = 2 -O-methyl-adenosine
  modified_base 7: mod_base = gm
  modified_base 8: mod_base = OTHER; note = 2 -O-methyl-adenosine
  modified_base 9: mod_base = OTHER; note = 2 -O-methyl-adenosine
  modified_base 10: mod_base = cm
  modified_base 11: mod_base = um
  modified_base 12: mod_base = OTHER; note = 2 -O-methyl-adenosine
  modified_base 13: mod_base = gm
  modified_base 14: mod_base = OTHER; note = 2 -Fluoro-cytidine
  modified_base 15: mod_base = cm
  modified_base 16: mod_base = OTHER; note = 2 -O-methyl-adenosine
  modified_base 17: mod_base = OTHER; note = 2 -O-methyl-adenosine
  modified_base 18: mod_base = OTHER; note = 2 -O-methyl-adenosine
  modified_base 19..21: mod_base = OTHER; note = linked via phosphorothioate
  modified_base 19: mod_base = cm
  modified_base 20: mod_base = cm
  modified_base 21: mod_base = um
  source 1..21: mol_type = other RNA
ttaagagaactagccaaacct

SEQ ID NO: 335 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = um
  modified_base 4: mod_base = um
  modified_base 5: mod_base = OTHER; note = 2 -O-methyl-adenosine
  modified_base 6: mod_base = OTHER; note = 2 -O-methyl-adenosine
  modified_base 7: mod_base = gm
  modified_base 8: mod_base = OTHER; note = 2 -O-methyl-adenosine
  modified_base 9: mod_base = gm
  modified_base 10: mod_base = OTHER; note = 2 -O-methyl-adenosine
  modified_base 11: mod_base = OTHER; note = 2 -O-methyl-adenosine
  modified_base 12: mod_base = cm
  modified_base 13: mod_base = um
  modified_base 14: mod_base = OTHER; note = 2'-Fluoro-adenosine
  modified_base 15: mod_base = gm
  modified_base 16: mod_base = cm
  modified_base 17: mod_base = cm
  modified_base 18: mod_base = OTHER; note = 2 -O-methyl-adenosine
  modified_base 19..21: mod_base = OTHER; note = linked via phosphorothioate
  modified_base 19: mod_base = OTHER; note = 2 -O-methyl-adenosine
  modified_base 20: mod_base = OTHER; note = 2 -O-methyl-adenosine
  modified_base 21: mod_base = cm
  source 1..21: mol_type = other RNA
tgttaagagaactagccaaac

SEQ ID NO: 336 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = cm
  modified_base 4: mod_base = um
  modified_base 5: mod_base = um
  modified_base 6: mod_base = um
  modified_base 7: mod_base = cm
  modified_base 8: mod_base = OTHER; note = 2 -O-methyl-adenosine
  modified_base 9: mod_base = cm
  modified_base 10: mod_base = cm
  modified_base 11: mod_base = um
  modified_base 12: mod_base = cm
  modified_base 13: mod_base = OTHER; note = 2 -O-methyl-adenosine
  modified_base 14: mod_base = OTHER; note = 2 -Fluoro-cytidine
  modified_base 15: mod_base = cm
  modified_base 16: mod_base = OTHER; note = 2 -O-methyl-adenosine
  modified_base 17: mod_base = um
  modified_base 18: mod_base = gm
  modified_base 19..21: mod_base = OTHER; note = linked via phosphorothioate
  modified_base 19: mod_base = cm
  modified_base 20: mod_base = cm
  modified_base 21: mod_base = OTHER; note = 2 -O-methyl-adenosine
  source 1..21: mol_type = other RNA
tactttcacctcaccatgcca

SEQ ID NO: 337 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = OTHER; note = 2 -O-methyl-adenosine
  modified_base 4: mod_base = cm
  modified_base 5: mod_base = um
  modified_base 6: mod_base = um
  modified_base 7: mod_base = um
  modified_base 8: mod_base = cm
  modified_base 9: mod_base = OTHER; note = 2 -O-methyl-adenosine
  modified_base 10: mod_base = cm
  modified_base 11: mod_base = cm
  modified_base 12: mod_base = um
  modified_base 13: mod_base = cm
  modified_base 14: mod_base = OTHER; note = 2'-Fluoro-adenosine
  modified_base 15: mod_base = cm
  modified_base 16: mod_base = cm
  modified_base 17: mod_base = OTHER; note = 2 -O-methyl-adenosine
  modified_base 18: mod_base = um
  modified_base 19..21: mod_base = OTHER; note = linked via phosphorothioate
  modified_base 19: mod_base = gm
  modified_base 20: mod_base = cm
  modified_base 21: mod_base = cm
  source 1..21: mol_type = other RNA
ttactttcacctcaccatgcc

SEQ ID NO: 338 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = um
  modified_base 4: mod_base = cm
  modified_base 5: mod_base = um
  modified_base 6: mod_base = OTHER; note = 2 -O-methyl-adenosine
  modified_base 7: mod_base = gm
  modified_base 8: mod_base = OTHER; note = 2 -O-methyl-adenosine
  modified_base 9: mod_base = OTHER; note = 2 -O-methyl-adenosine
  modified_base 10: mod_base = cm
  modified_base 11: mod_base = cm
  modified_base 12: mod_base = um
  modified_base 13: mod_base = OTHER; note = 2 -O-methyl-adenosine
  modified_base 14: mod_base = OTHER; note = 2'-Fluoro-adenosine
  modified_base 15: mod_base = gm
  modified_base 16: mod_base = um
  modified_base 17: mod_base = cm
  modified_base 18: mod_base = OTHER; note = 2 -O-methyl-adenosine
  modified_base 19..21: mod_base = OTHER; note = linked via phosphorothioate
  modified_base 19: mod_base = cm
  modified_base 20: mod_base = cm
  modified_base 21: mod_base = um
  source 1..21: mol_type = other RNA
tatctagaacctaagtcacct

SEQ ID NO: 339 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = OTHER; note = 2 -O-methyl-adenosine
  modified_base 4: mod_base = um
  modified_base 5: mod_base = cm
  modified_base 6: mod_base = um
  modified_base 7: mod_base = OTHER; note = 2 -O-methyl-adenosine
  modified_base 8: mod_base = gm
  modified_base 9: mod_base = OTHER; note = 2 -O-methyl-adenosine
  modified_base 10: mod_base = OTHER; note = 2 -O-methyl-adenosine
  modified_base 11: mod_base = cm
  modified_base 12: mod_base = cm
  modified_base 13: mod_base = um
  modified_base 14: mod_base = OTHER; note = 2'-Fluoro-adenosine
  modified_base 15: mod_base = OTHER; note = 2 -O-methyl-adenosine
  modified_base 16: mod_base = gm
  modified_base 17: mod_base = um
  modified_base 18: mod_base = cm
  modified_base 19..21: mod_base = OTHER; note = linked via phosphorothioate
  modified_base 19: mod_base = OTHER; note = 2 -O-methyl-adenosine
  modified_base 20: mod_base = cm
  modified_base 21: mod_base = cm
  source 1..21: mol_type = other RNA
ttatctagaacctaagtcacc

SEQ ID NO: 340 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = OTHER; note = 2 -O-methyl-adenosine
  modified_base 4: mod_base = um
  modified_base 5: mod_base = OTHER; note = 2 -O-methyl-adenosine
  modified_base 6: mod_base = gm
  modified_base 7: mod_base = um
  modified_base 8: mod_base = OTHER; note = 2 -O-methyl-adenosine
  modified_base 9: mod_base = OTHER; note = 2 -O-methyl-adenosine
  modified_base 10: mod_base = gm
  modified_base 11: mod_base = um
  modified_base 12: mod_base = gm
  modified_base 13: mod_base = OTHER; note = 2 -O-methyl-adenosine
  modified_base 14: mod_base = OTHER; note = 2'-Fluoro-uridine
  modified_base 15: mod_base = gm
  modified_base 16: mod_base = um
  modified_base 17: mod_base = cm
  modified_base 18: mod_base = cm
  modified_base 19..21: mod_base = OTHER; note = linked via phosphorothioate
  modified_base 19: mod_base = um
  modified_base 20: mod_base = cm
  modified_base 21: mod_base = OTHER; note = 2 -O-methyl-adenosine
  source 1..21: mol_type = other RNA
tgatagtaagtgatgtcctca

SEQ ID NO: 341 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = gm
  modified_base 4: mod_base = OTHER; note = 2 -O-methyl-adenosine
  modified_base 5: mod_base = cm
  modified_base 6: mod_base = OTHER; note = 2 -O-methyl-adenosine
  modified_base 7: mod_base = OTHER; note = 2 -O-methyl-adenosine
  modified_base 8: mod_base = OTHER; note = 2 -O-methyl-adenosine
  modified_base 9: mod_base = um
  modified_base 10: mod_base = OTHER; note = 2 -O-methyl-adenosine
  modified_base 11: mod_base = OTHER; note = 2 -O-methyl-adenosine
  modified_base 12: mod_base = gm
  modified_base 13: mod_base = um
  modified_base 14: mod_base = OTHER; note = 2'-Fluoro-guanosine
  modified_base 15: mod_base = um
  modified_base 16: mod_base = OTHER; note = 2 -O-methyl-adenosine
  modified_base 17: mod_base = um
  modified_base 18: mod_base = cm
  modified_base 19..21: mod_base = OTHER; note = linked via phosphorothioate
  modified_base 19: mod_base = cm
  modified_base 20: mod_base = um
  modified_base 21: mod_base = um
  source 1..21: mol_type = other RNA
ttgacaaataagtgtatcctt

SEQ ID NO: 342 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = gm
  modified_base 4: mod_base = um
  modified_base 5: mod_base = gm
  modified_base 6: mod_base = um
  modified_base 7: mod_base = OTHER; note = 2 -O-methyl-adenosine
  modified_base 8: mod_base = OTHER; note = 2 -O-methyl-adenosine
  modified_base 9: mod_base = cm
  modified_base 10: mod_base = OTHER; note = 2 -O-methyl-adenosine
  modified_base 11: mod_base = um
  modified_base 12: mod_base = OTHER; note = 2 -O-methyl-adenosine
  modified_base 13: mod_base = gm
  modified_base 14: mod_base = OTHER; note = 2'-Fluoro-guanosine
  modified_base 15: mod_base = um
  modified_base 16: mod_base = um
  modified_base 17: mod_base = OTHER; note = 2 -O-methyl-adenosine
  modified_base 18: mod_base = OTHER; note = 2 -O-methyl-adenosine
  modified_base 19..21: mod_base = OTHER; note = linked via phosphorothioate
  modified_base 19: mod_base = OTHER; note = 2 -O-methyl-adenosine
  modified_base 20: mod_base = OTHER; note = 2 -O-methyl-adenosine
  modified_base 21: mod_base = OTHER; note = 2 -O-methyl-adenosine
  source 1..21: mol_type = other RNA
tggtgtaacataggttaaaaa

SEQ ID NO: 343 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = gm
  modified_base 4: mod_base = gm
  modified_base 5: mod_base = um
  modified_base 6: mod_base = gm
  modified_base 7: mod_base = um
  modified_base 8: mod_base = OTHER; note = 2 -O-methyl-adenosine
  modified_base 9: mod_base = OTHER; note = 2 -O-methyl-adenosine
  modified_base 10: mod_base = cm
  modified_base 11: mod_base = OTHER; note = 2 -O-methyl-adenosine
  modified_base 12: mod_base = um
  modified_base 13: mod_base = OTHER; note = 2 -O-methyl-adenosine
  modified_base 14: mod_base = OTHER; note = 2'-Fluoro-guanosine
  modified_base 15: mod_base = gm
  modified_base 16: mod_base = um
  modified_base 17: mod_base = um
  modified_base 18: mod_base = OTHER; note = 2 -O-methyl-adenosine
  modified_base 19..21: mod_base = OTHER; note = linked via phosphorothioate
  modified_base 19: mod_base = OTHER; note = 2 -O-methyl-adenosine
  modified_base 20: mod_base = OTHER; note = 2 -O-methyl-adenosine
  modified_base 21: mod_base = OTHER; note = 2 -O-methyl-adenosine
  source 1..21: mol_type = other RNA
ttggtgtaacataggttaaaa

SEQ ID NO: 344 - RNA, 21 nt, synthetic construct
  modified_base 1..3: mod_base = OTHER; note = linked via phosphorothioate
  modified_base 1: mod_base = um
  modified_base 2: mod_base = OTHER; note = 2 -Fluoro-cytidine
  modified_base 3: mod_base = um
  modified_base 4: mod_base = um
  modified_base 5: mod_base = gm
  modified_base 6: mod_base = cm
  modified_base 7: mod_base = OTHER; note = 2 -O-methyl-adenosine
  modified_base 8: mod_base = cm
  modified_base 9: mod_base = OTHER; note = 2 -O-methyl-adenosine
  modified_base 10: mod_base = OTHER; note = 2 -O-methyl-adenosine
  modified_base 11: mod_base = um
  modified_base 12: mod_base = um
  modified_base 13: mod_base = um
  modified_base 14: mod_base = OTHER; note = 2'-Fluoro-uridine
  modified_base 15: mod_base = gm
  modified_base 16: mod_base = cm
  modified_base 17: mod_base = cm
  modified_base 18: mod_base = cm
  modified_base 19..21: mod_base = OTHER; note = linked via phosphorothioate
  modified_base 19: mod_base = OTHER; note = 2 -O-methyl-adenosine
  modified_base 20: mod_base = OTHER; note = 2 -O-methyl-adenosine
  modified_base 21: mod_base = gm
  source 1..21: mol_type = other RNA
tcttgcacaattttgcccaag

SEQ ID NO: 345 - RNA, 21 nt, synthetic construct
  modified_base 1..3: mod_base = OTHER; note = linked via phosphorothioate
  modified_base 1: mod_base = um
  modified_base 2: mod_base = OTHER; note = 2 -Fluoro-cytidine
  modified_base 3: mod_base = gm
  modified_base 4: mod_base = OTHER; note = 2 -O-methyl-adenosine
  modified_base 5: mod_base = gm
  modified_base 6: mod_base = OTHER; note = 2 -O-methyl-adenosine
  modified_base 7: mod_base = OTHER; note = 2 -O-methyl-adenosine
  modified_base 8: mod_base = um
  modified_base 9: mod_base = gm
  modified_base 10: mod_base = gm
  modified_base 11: mod_base = OTHER; note = 2 -O-methyl-adenosine
  modified_base 12: mod_base = um
  modified_base 13: mod_base = OTHER; note = 2 -O-methyl-adenosine
  modified_base 14: mod_base = OTHER; note = 2'-Fluoro-uridine
  modified_base 15: mod_base = um
  modified_base 16: mod_base = cm
  modified_base 17: mod_base = OTHER; note = 2 -O-methyl-adenosine
  modified_base 18: mod_base = OTHER; note = 2 -O-methyl-adenosine
  modified_base 19..21: mod_base = OTHER; note = linked via phosphorothioate
  modified_base 19: mod_base = OTHER; note = 2 -O-methyl-adenosine
  modified_base 20: mod_base = um
  modified_base 21: mod_base = OTHER; note = 2 -O-methyl-adenosine
  source 1..21: mol_type = other RNA
tcgagaatggatattcaaata

SEQ ID NO: 346 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = OTHER; note = 2 -O-methyl-adenosine
  modified_base 4: mod_base = um
  modified_base 5: mod_base = cm
  modified_base 6: mod_base = um
  modified_base 7: mod_base = um
  modified_base 8: mod_base = OTHER; note = 2 -O-methyl-adenosine
  modified_base 9: mod_base = um
  modified_base 10: mod_base = gm
  modified_base 11: mod_base = gm
  modified_base 12: mod_base = um
  modified_base 13: mod_base = um
  modified_base 14: mod_base = OTHER; note = 2'-Fluoro-adenosine
  modified_base 15: mod_base = gm
  modified_base 16: mod_base = gm
  modified_base 17: mod_base = gm
  modified_base 18: mod_base = gm
  modified_base 19..21: mod_base = OTHER; note = linked via phosphorothioate
  modified_base 19: mod_base = OTHER; note = 2 -O-methyl-adenosine
  modified_base 20: mod_base = OTHER; note = 2 -O-methyl-adenosine
  modified_base 21: mod_base = um
  source 1..21: mol_type = other RNA
taatcttatggttaggggaat

SEQ ID NO: 347 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = OTHER; note = 2 -O-methyl-adenosine
  modified_base 4: mod_base = OTHER; note = 2 -O-methyl-adenosine
  modified_base 5: mod_base = um
  modified_base 6: mod_base = cm
  modified_base 7: mod_base = um
  modified_base 8: mod_base = um
  modified_base 9: mod_base = OTHER; note = 2 -O-methyl-adenosine
  modified_base 10: mod_base = um
  modified_base 11: mod_base = gm
  modified_base 12: mod_base = gm
  modified_base 13: mod_base = um
  modified_base 14: mod_base = OTHER; note = 2'-Fluoro-uridine
  modified_base 15: mod_base = OTHER; note = 2 -O-methyl-adenosine
  modified_base 16: mod_base = gm
  modified_base 17: mod_base = gm
  modified_base 18: mod_base = gm
  modified_base 19..21: mod_base = OTHER; note = linked via phosphorothioate
  modified_base 19: mod_base = gm
  modified_base 20: mod_base = OTHER; note = 2 -O-methyl-adenosine
  modified_base 21: mod_base = OTHER; note = 2 -O-methyl-adenosine
  source 1..21: mol_type = other RNA
taaatcttatggttaggggaa

SEQ ID NO: 348 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = OTHER; note = 2 -O-methyl-adenosine
  modified_base 4: mod_base = OTHER; note = 2 -O-methyl-adenosine
  modified_base 5: mod_base = OTHER; note = 2 -O-methyl-adenosine
  modified_base 6: mod_base = um
  modified_base 7: mod_base = cm
  modified_base 8: mod_base = um
  modified_base 9: mod_base = um
  modified_base 10: mod_base = OTHER; note = 2 -O-methyl-adenosine
  modified_base 11: mod_base = um
  modified_base 12: mod_base = gm
  modified_base 13: mod_base = gm
  modified_base 14: mod_base = OTHER; note = 2'-Fluoro-uridine
  modified_base 15: mod_base = um
  modified_base 16: mod_base = OTHER; note = 2 -O-methyl-adenosine
  modified_base 17: mod_base = gm
  modified_base 18: mod_base = gm
  modified_base 19..21: mod_base = OTHER; note = linked via phosphorothioate
  modified_base 19: mod_base = gm
  modified_base 20: mod_base = gm
  modified_base 21: mod_base = OTHER; note = 2 -O-methyl-adenosine
  source 1..21: mol_type = other RNA
ttaaatcttatggttagggga

SEQ ID NO: 349 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = um
  modified_base 4: mod_base = OTHER; note = 2 -O-methyl-adenosine
  modified_base 5: mod_base = OTHER; note = 2 -O-methyl-adenosine
  modified_base 6: mod_base = OTHER; note = 2 -O-methyl-adenosine
  modified_base 7: mod_base = um
  modified_base 8: mod_base = cm
  modified_base 9: mod_base = um
  modified_base 10: mod_base = um
  modified_base 11: mod_base = OTHER; note = 2 -O-methyl-adenosine
  modified_base 12: mod_base = um
  modified_base 13: mod_base = gm
  modified_base 14: mod_base = OTHER; note = 2'-Fluoro-guanosine
  modified_base 15: mod_base = um
  modified_base 16: mod_base = um
  modified_base 17: mod_base = OTHER; note = 2 -O-methyl-adenosine
  modified_base 18: mod_base = gm
  modified_base 19..21: mod_base = OTHER; note = linked via phosphorothioate
  modified_base 19: mod_base = gm
  modified_base 20: mod_base = gm
  modified_base 21: mod_base = gm
  source 1..21: mol_type = other RNA
tgtaaatcttatggttagggg

SEQ ID NO: 350 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = gm
  modified_base 4: mod_base = OTHER; note = 2 -O-methyl-adenosine
  modified_base 5: mod_base = um
  modified_base 6: mod_base = um
  modified_base 7: mod_base = OTHER; note = 2 -O-methyl-adenosine
  modified_base 8: mod_base = gm
  modified_base 9: mod_base = gm
  modified_base 10: mod_base = um
  modified_base 11: mod_base = cm
  modified_base 12: mod_base = OTHER; note = 2 -O-methyl-adenosine
  modified_base 13: mod_base = OTHER; note = 2 -O-methyl-adenosine
  modified_base 14: mod_base = OTHER; note = 2'-Fluoro-adenosine
  modified_base 15: mod_base = um
  modified_base 16: mod_base = cm
  modified_base 17: mod_base = cm
  modified_base 18: mod_base = cm
  modified_base 19..21: mod_base = OTHER; note = linked via phosphorothioate
  modified_base 19: mod_base = um
  modified_base 20: mod_base = um
  modified_base 21: mod_base = um
  source 1..21: mol_type = other RNA
ttgattaggtcaaatcccttt

SEQ ID NO: 351 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = OTHER; note = 2 -O-methyl-adenosine
  modified_base 4: mod_base = OTHER; note = 2 -O-methyl-adenosine
  modified_base 5: mod_base = um
  modified_base 6: mod_base = um
  modified_base 7: mod_base = OTHER; note = 2 -O-methyl-adenosine
  modified_base 8: mod_base = gm
  modified_base 9: mod_base = um
  modified_base 10: mod_base = gm
  modified_base 11: mod_base = OTHER; note = 2 -O-methyl-adenosine
  modified_base 12: mod_base = um
  modified_base 13: mod_base = um
  modified_base 14: mod_base = OTHER; note = 2'-Fluoro-adenosine
  modified_base 15: mod_base = gm
  modified_base 16: mod_base = gm
  modified_base 17: mod_base = um
  modified_base 18: mod_base = cm
  modified_base 19..21: mod_base = OTHER; note = linked via phosphorothioate
  modified_base 19: mod_base = OTHER; note = 2 -O-methyl-adenosine
  modified_base 20: mod_base = OTHER; note = 2 -O-methyl-adenosine
  modified_base 21: mod_base = OTHER; note = 2 -O-methyl-adenosine
  source 1..21: mol_type = other RNA
taaattagtgattaggtcaaa

SEQ ID NO: 352 - RNA, 21 nt, synthetic construct
  modified_base 1..3: mod_base = OTHER; note = linked via phosphorothioate
  modified_base 1: mod_base = um
  modified_base 2: mod_base = OTHER; note = 2 -Fluoro-cytidine
  modified_base 3: mod_base = gm
  modified_base 4: mod_base = cm
  modified_base 5: mod_base = um
  modified_base 6: mod_base = OTHER; note = 2 -O-methyl-adenosine
  modified_base 7: mod_base = OTHER; note = 2 -O-methyl-adenosine
  modified_base 8: mod_base = um
  modified_base 9: mod_base = gm
  modified_base 10: mod_base = gm
  modified_base 11: mod_base = OTHER; note = 2 -O-methyl-adenosine
  modified_base 12: mod_base = um
  modified_base 13: mod_base = um
  modified_base 14: mod_base = OTHER; note = 2'-Fluoro-guanosine
  modified_base 15: mod_base = gm
  modified_base 16: mod_base = gm
  modified_base 17: mod_base = cm
  modified_base 18: mod_base = OTHER; note = 2 -O-methyl-adenosine
  modified_base 19..21: mod_base = OTHER; note = linked via phosphorothioate
  modified_base 19: mod_base = gm
  modified_base 20: mod_base = cm
  modified_base 21: mod_base = OTHER; note = 2 -O-methyl-adenosine
  source 1..21: mol_type = other RNA
tcgctaatggattgggcagca

SEQ ID NO: 353 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = cm
  modified_base 4: mod_base = gm
  modified_base 5: mod_base = cm
  modified_base 6: mod_base = um
  modified_base 7: mod_base = OTHER; note = 2 -O-methyl-adenosine
  modified_base 8: mod_base = OTHER; note = 2 -O-methyl-adenosine
  modified_base 9: mod_base = um
  modified_base 10: mod_base = gm
  modified_base 11: mod_base = gm
  modified_base 12: mod_base = OTHER; note = 2 -O-methyl-adenosine
  modified_base 13: mod_base = um
  modified_base 14: mod_base = OTHER; note = 2'-Fluoro-uridine
  modified_base 15: mod_base = gm
  modified_base 16: mod_base = gm
  modified_base 17: mod_base = gm
  modified_base 18: mod_base = cm
  modified_base 19..21: mod_base = OTHER; note = linked via phosphorothioate
  modified_base 19: mod_base = OTHER; note = 2 -O-methyl-adenosine
  modified_base 20: mod_base = gm
  modified_base 21: mod_base = cm
  source 1..21: mol_type = other RNA
ttcgctaatggattgggcagc

SEQ ID NO: 354 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = um
  modified_base 4: mod_base = cm
  modified_base 5: mod_base = gm
  modified_base 6: mod_base = cm
  modified_base 7: mod_base = um
  modified_base 8: mod_base = OTHER; note = 2 -O-methyl-adenosine
  modified_base 9: mod_base = OTHER; note = 2 -O-methyl-adenosine
  modified_base 10: mod_base = um
  modified_base 11: mod_base = gm
  modified_base 12: mod_base = gm
  modified_base 13: mod_base = OTHER; note = 2 -O-methyl-adenosine
  modified_base 14: mod_base = OTHER; note = 2'-Fluoro-uridine
  modified_base 15: mod_base = um
  modified_base 16: mod_base = gm
  modified_base 17: mod_base = gm
  modified_base 18: mod_base = gm
  modified_base 19..21: mod_base = OTHER; note = linked via phosphorothioate
  modified_base 19: mod_base = cm
  modified_base 20: mod_base = OTHER; note = 2 -O-methyl-adenosine
  modified_base 21: mod_base = gm
  source 1..21: mol_type = other RNA
tgtcgctaatggattgggcag

SEQ ID NO: 355 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = gm
  modified_base 4: mod_base = um
  modified_base 5: mod_base = cm
  modified_base 6: mod_base = gm
  modified_base 7: mod_base = cm
  modified_base 8: mod_base = um
  modified_base 9: mod_base = OTHER; note = 2 -O-methyl-adenosine
  modified_base 10: mod_base = OTHER; note = 2 -O-methyl-adenosine
  modified_base 11: mod_base = um
  modified_base 12: mod_base = gm
  modified_base 13: mod_base = gm
  modified_base 14: mod_base = OTHER; note = 2'-Fluoro-adenosine
  modified_base 15: mod_base = um
  modified_base 16: mod_base = um
  modified_base 17: mod_base = gm
  modified_base 18: mod_base = gm
  modified_base 19..21: mod_base = OTHER; note = linked via phosphorothioate
  modified_base 19: mod_base = gm
  modified_base 20: mod_base = cm
  modified_base 21: mod_base = OTHER; note = 2 -O-methyl-adenosine
  source 1..21: mol_type = other RNA
ttgtcgctaatggattgggca

SEQ ID NO: 356 - RNA, 21 nt, synthetic construct
  modified_base 1..3: mod_base = OTHER; note = linked via phosphorothioate
  modified_base 1: mod_base = um
  modified_base 2: mod_base = OTHER; note = 2 -Fluoro-cytidine
  modified_base 3: mod_base = um
  modified_base 4: mod_base = gm
  modified_base 5: mod_base = um
  modified_base 6: mod_base = cm
  modified_base 7: mod_base = gm
  modified_base 8: mod_base = cm
  modified_base 9: mod_base = um
  modified_base 10: mod_base = OTHER; note = 2 -O-methyl-adenosine
  modified_base 11: mod_base = OTHER; note = 2 -O-methyl-adenosine
  modified_base 12: mod_base = um
  modified_base 13: mod_base = gm
  modified_base 14: mod_base = OTHER; note = 2'-Fluoro-guanosine
  modified_base 15: mod_base = OTHER; note = 2 -O-methyl-adenosine
  modified_base 16: mod_base = um
  modified_base 17: mod_base = um
  modified_base 18: mod_base = gm
  modified_base 19..21: mod_base = OTHER; note = linked via phosphorothioate
  modified_base 19: mod_base = gm
  modified_base 20: mod_base = gm
  modified_base 21: mod_base = cm
  source 1..21: mol_type = other RNA
tctgtcgctaatggattgggc

SEQ ID NO: 357 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = cm
  modified_base 4: mod_base = um
  modified_base 5: mod_base = gm
  modified_base 6: mod_base = um
  modified_base 7: mod_base = cm
  modified_base 8: mod_base = gm
  modified_base 9: mod_base = cm
  modified_base 10: mod_base = um
  modified_base 11: mod_base = OTHER; note = 2 -O-methyl-adenosine
  modified_base 12: mod_base = OTHER; note = 2 -O-methyl-adenosine
  modified_base 13: mod_base = um
  modified_base 14: mod_base = OTHER; note = 2'-Fluoro-guanosine
  modified_base 15: mod_base = gm
  modified_base 16: mod_base = OTHER; note = 2 -O-methyl-adenosine
  modified_base 17: mod_base = um
  modified_base 18: mod_base = um
  modified_base 19..21: mod_base = OTHER; note = linked via phosphorothioate
  modified_base 19: mod_base = gm
  modified_base 20: mod_base = gm
  modified_base 21: mod_base = gm
  source 1..21: mol_type = other RNA
tactgtcgctaatggattggg

SEQ ID NO: 358 - RNA, 21 nt, synthetic construct
  modified_base 1..3: mod_base = OTHER; note = linked via phosphorothioate
  modified_base 1: mod_base = um
  modified_base 2: mod_base = OTHER; note = 2 -Fluoro-cytidine
  modified_base 3: mod_base = um
  modified_base 4: mod_base = OTHER; note = 2 -O-methyl-adenosine
  modified_base 5: mod_base = cm
  modified_base 6: mod_base = um
  modified_base 7: mod_base = gm
  modified_base 8: mod_base = um
  modified_base 9: mod_base = cm
  modified_base 10: mod_base = gm
  modified_base 11: mod_base = cm
  modified_base 12: mod_base = um
  modified_base 13: mod_base = OTHER; note = 2 -O-methyl-adenosine
  modified_base 14: mod_base = OTHER; note = 2'-Fluoro-adenosine
  modified_base 15: mod_base = um
  modified_base 16: mod_base = gm
  modified_base 17: mod_base = gm
  modified_base 18: mod_base = OTHER; note = 2 -O-methyl-adenosine
  modified_base 19..21: mod_base = OTHER; note = linked via phosphorothioate
  modified_base 19: mod_base = um
  modified_base 20: mod_base = um
  modified_base 21: mod_base = gm
  source 1..21: mol_type = other RNA
tctactgtcgctaatggattg

SEQ ID NO: 359 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = um
  modified_base 4: mod_base = cm
  modified_base 5: mod_base = cm
  modified_base 6: mod_base = um
  modified_base 7: mod_base = OTHER; note = 2 -O-methyl-adenosine
  modified_base 8: mod_base = cm
  modified_base 9: mod_base = um
  modified_base 10: mod_base = gm
  modified_base 11: mod_base = um
  modified_base 12: mod_base = cm
  modified_base 13: mod_base = gm
  modified_base 14: mod_base = OTHER; note = 2 -Fluoro-cytidine
  modified_base 15: mod_base = um
  modified_base 16: mod_base = OTHER; note = 2 -O-methyl-adenosine
  modified_base 17: mod_base = OTHER; note = 2 -O-methyl-adenosine
  modified_base 18: mod_base = um
  modified_base 19..21: mod_base = OTHER; note = linked via phosphorothioate
  modified_base 19: mod_base = gm
  modified_base 20: mod_base = gm
  modified_base 21: mod_base = OTHER; note = 2 -O-methyl-adenosine
  source 1..21: mol_type = other RNA
tatcctactgtcgctaatgga

SEQ ID NO: 360 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = um
  modified_base 4: mod_base = um
  modified_base 5: mod_base = cm
  modified_base 6: mod_base = um
  modified_base 7: mod_base = gm
  modified_base 8: mod_base = um
  modified_base 9: mod_base = cm
  modified_base 10: mod_base = um
  modified_base 11: mod_base = OTHER; note = 2 -O-methyl-adenosine
  modified_base 12: mod_base = um
  modified_base 13: mod_base = um
  modified_base 14: mod_base = OTHER; note = 2 -Fluoro-cytidine
  modified_base 15: mod_base = OTHER; note = 2 -O-methyl-adenosine
  modified_base 16: mod_base = um
  modified_base 17: mod_base = OTHER; note = 2 -O-methyl-adenosine
  modified_base 18: mod_base = cm
  modified_base 19..21: mod_base = OTHER; note = linked via phosphorothioate
  modified_base 19: mod_base = cm
  modified_base 20: mod_base = OTHER; note = 2 -O-methyl-adenosine
  modified_base 21: mod_base = gm
  source 1..21: mol_type = other RNA
tgttctgtctattcataccag

SEQ ID NO: 361 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = OTHER; note = 2 -O-methyl-adenosine
  modified_base 4: mod_base = um
  modified_base 5: mod_base = OTHER; note = 2 -O-methyl-adenosine
  modified_base 6: mod_base = gm
  modified_base 7: mod_base = gm
  modified_base 8: mod_base = gm
  modified_base 9: mod_base = um
  modified_base 10: mod_base = um
  modified_base 11: mod_base = cm
  modified_base 12: mod_base = um
  modified_base 13: mod_base = gm
  modified_base 14: mod_base = OTHER; note = 2'-Fluoro-uridine
  modified_base 15: mod_base = cm
  modified_base 16: mod_base = um
  modified_base 17: mod_base = OTHER; note = 2 -O-methyl-adenosine
  modified_base 18: mod_base = um
  modified_base 19..21: mod_base = OTHER; note = linked via phosphorothioate
  modified_base 19: mod_base = um
  modified_base 20: mod_base = cm
  modified_base 21: mod_base = OTHER; note = 2 -O-methyl-adenosine
  source 1..21: mol_type = other RNA
tgatagggttctgtctattca

SEQ ID NO: 362 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = um
  modified_base 4: mod_base = OTHER; note = 2 -O-methyl-adenosine
  modified_base 5: mod_base = um
  modified_base 6: mod_base = um
  modified_base 7: mod_base = OTHER; note = 2 -O-methyl-adenosine
  modified_base 8: mod_base = cm
  modified_base 9: mod_base = um
  modified_base 10: mod_base = gm
  modified_base 11: mod_base = um
  modified_base 12: mod_base = um
  modified_base 13: mod_base = OTHER; note = 2 -O-methyl-adenosine
  modified_base 14: mod_base = OTHER; note = 2 -Fluoro-cytidine
  modified_base 15: mod_base = cm
  modified_base 16: mod_base = OTHER; note = 2 -O-methyl-adenosine
  modified_base 17: mod_base = gm
  modified_base 18: mod_base = gm
  modified_base 19..21: mod_base = OTHER; note = linked via phosphorothioate
  modified_base 19: mod_base = OTHER; note = 2 -O-methyl-adenosine
  modified_base 20: mod_base = gm
  modified_base 21: mod_base = um
  source 1..21: mol_type = other RNA
tgtattactgttaccaggagt

SEQ ID NO: 363 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = um
  modified_base 4: mod_base = gm
  modified_base 5: mod_base = um
  modified_base 6: mod_base = OTHER; note = 2 -O-methyl-adenosine
  modified_base 7: mod_base = cm
  modified_base 8: mod_base = cm
  modified_base 9: mod_base = cm
  modified_base 10: mod_base = OTHER; note = 2 -O-methyl-adenosine
  modified_base 11: mod_base = gm
  modified_base 12: mod_base = OTHER; note = 2 -O-methyl-adenosine
  modified_base 13: mod_base = um
  modified_base 14: mod_base = OTHER; note = 2'-Fluoro-adenosine
  modified_base 15: mod_base = OTHER; note = 2 -O-methyl-adenosine
  modified_base 16: mod_base = OTHER; note = 2 -O-methyl-adenosine
  modified_base 17: mod_base = OTHER; note = 2 -O-methyl-adenosine
  modified_base 18: mod_base = cm
  modified_base 19..21: mod_base = OTHER; note = linked via phosphorothioate
  modified_base 19: mod_base = um
  modified_base 20: mod_base = OTHER; note = 2 -O-methyl-adenosine
  modified_base 21: mod_base = um
  source 1..21: mol_type = other RNA
tttgtacccagataaaactat

SEQ ID NO: 364 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = OTHER; note = 2 -O-methyl-adenosine
  modified_base 4: mod_base = OTHER; note = 2 -O-methyl-adenosine
  modified_base 5: mod_base = gm
  modified_base 6: mod_base = um
  modified_base 7: mod_base = cm
  modified_base 8: mod_base = um
  modified_base 9: mod_base = um
  modified_base 10: mod_base = OTHER; note = 2 -O-methyl-adenosine
  modified_base 11: mod_base = OTHER; note = 2 -O-methyl-adenosine
  modified_base 12: mod_base = cm
  modified_base 13: mod_base = OTHER; note = 2 -O-methyl-adenosine
  modified_base 14: mod_base = OTHER; note = 2 -Fluoro-cytidine
  modified_base 15: mod_base = cm
  modified_base 16: mod_base = cm
  modified_base 17: mod_base = um
  modified_base 18: mod_base = OTHER; note = 2 -O-methyl-adenosine
  modified_base 19..21: mod_base = OTHER; note = linked via phosphorothioate
  modified_base 19: mod_base = cm
  modified_base 20: mod_base = cm
  modified_base 21: mod_base = um
  source 1..21: mol_type = other RNA
ttaagtcttaacaccctacct

SEQ ID NO: 365 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = gm
  modified_base 4: mod_base = OTHER; note = 2 -O-methyl-adenosine
  modified_base 5: mod_base = gm
  modified_base 6: mod_base = gm
  modified_base 7: mod_base = cm
  modified_base 8: mod_base = cm
  modified_base 9: mod_base = um
  modified_base 10: mod_base = OTHER; note = 2 -O-methyl-adenosine
  modified_base 11: mod_base = OTHER; note = 2 -O-methyl-adenosine
  modified_base 12: mod_base = OTHER; note = 2 -O-methyl-adenosine
  modified_base 13: mod_base = um
  modified_base 14: mod_base = OTHER; note = 2'-Fluoro-adenosine
  modified_base 15: mod_base = um
  modified_base 16: mod_base = cm
  modified_base 17: mod_base = cm
  modified_base 18: mod_base = cm
  modified_base 19..21: mod_base = OTHER; note = linked via phosphorothioate
  modified_base 19: mod_base = cm
  modified_base 20: mod_base = um
  modified_base 21: mod_base = cm
  source 1..21: mol_type = other RNA
tagaggcctaaatatcccctc

SEQ ID NO: 366 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = OTHER; note = 2 -O-methyl-adenosine
  modified_base 4: mod_base = cm
  modified_base 5: mod_base = OTHER; note = 2 -O-methyl-adenosine
  modified_base 6: mod_base = um
  modified_base 7: mod_base = gm
  modified_base 8: mod_base = um
  modified_base 9: mod_base = um
  modified_base 10: mod_base = OTHER; note = 2 -O-methyl-adenosine
  modified_base 11: mod_base = OTHER; note = 2 -O-methyl-adenosine
  modified_base 12: mod_base = um
  modified_base 13: mod_base = gm
  modified_base 14: mod_base = OTHER; note = 2 -Fluoro-cytidine
  modified_base 15: mod_base = cm
  modified_base 16: mod_base = um
  modified_base 17: mod_base = OTHER; note = 2 -O-methyl-adenosine
  modified_base 18: mod_base = OTHER; note = 2 -O-methyl-adenosine
  modified_base 19..21: mod_base = OTHER; note = linked via phosphorothioate
  modified_base 19: mod_base = gm
  modified_base 20: mod_base = um
  modified_base 21: mod_base = cm
  source 1..21: mol_type = other RNA
taacatgttaatgcctaagtc

SEQ ID NO: 367 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = OTHER; note = 2 -O-methyl-adenosine
  modified_base 4: mod_base = OTHER; note = 2 -O-methyl-adenosine
  modified_base 5: mod_base = cm
  modified_base 6: mod_base = cm
  modified_base 7: mod_base = um
  modified_base 8: mod_base = OTHER; note = 2 -O-methyl-adenosine
  modified_base 9: mod_base = um
  modified_base 10: mod_base = OTHER; note = 2 -O-methyl-adenosine
  modified_base 11: mod_base = OTHER; note = 2 -O-methyl-adenosine
  modified_base 12: mod_base = OTHER; note = 2 -O-methyl-adenosine
  modified_base 13: mod_base = OTHER; note = 2 -O-methyl-adenosine
  modified_base 14: mod_base = OTHER; note = 2'-Fluoro-guanosine
  modified_base 15: mod_base = um
  modified_base 16: mod_base = um
  modified_base 17: mod_base = OTHER; note = 2 -O-methyl-adenosine
  modified_base 18: mod_base = gm
  modified_base 19..21: mod_base = OTHER; note = linked via phosphorothioate
  modified_base 19: mod_base = gm
  modified_base 20: mod_base = um
  modified_base 21: mod_base = um
  source 1..21: mol_type = other RNA
ttaacctataaaagttaggtt

SEQ ID NO: 368 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = um
  modified_base 4: mod_base = cm
  modified_base 5: mod_base = um
  modified_base 6: mod_base = OTHER; note = 2 -O-methyl-adenosine
  modified_base 7: mod_base = gm
  modified_base 8: mod_base = um
  modified_base 9: mod_base = um
  modified_base 10: mod_base = OTHER; note = 2 -O-methyl-adenosine
  modified_base 11: mod_base = um
  modified_base 12: mod_base = gm
  modified_base 13: mod_base = OTHER; note = 2 -O-methyl-adenosine
  modified_base 14: mod_base = OTHER; note = 2 -Fluoro-cytidine
  modified_base 15: mod_base = um
  modified_base 16: mod_base = OTHER; note = 2 -O-methyl-adenosine
  modified_base 17: mod_base = um
  modified_base 18: mod_base = um
  modified_base 19..21: mod_base = OTHER; note = linked via phosphorothioate
  modified_base 19: mod_base = cm
  modified_base 20: mod_base = um
  modified_base 21: mod_base = um
  source 1..21: mol_type = other RNA
tatctagttatgactattctt

SEQ ID NO: 369 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = OTHER; note = 2 -O-methyl-adenosine
  modified_base 4: mod_base = um
  modified_base 5: mod_base = cm
  modified_base 6: mod_base = um
  modified_base 7: mod_base = um
  modified_base 8: mod_base = OTHER; note = 2 -O-methyl-adenosine
  modified_base 9: mod_base = OTHER; note = 2 -O-methyl-adenosine
  modified_base 10: mod_base = um
  modified_base 11: mod_base = cm
  modified_base 12: mod_base = um
  modified_base 13: mod_base = OTHER; note = 2 -O-methyl-adenosine
  modified_base 14: mod_base = OTHER; note = 2'-Fluoro-guanosine
  modified_base 15: mod_base = um
  modified_base 16: mod_base = um
  modified_base 17: mod_base = OTHER; note = 2 -O-methyl-adenosine
  modified_base 18: mod_base = um
  modified_base 19..21: mod_base = OTHER; note = linked via phosphorothioate
  modified_base 19: mod_base = gm
  modified_base 20: mod_base = OTHER; note = 2 -O-methyl-adenosine
  modified_base 21: mod_base = cm
  source 1..21: mol_type = other RNA
tgatcttaatctagttatgac

SEQ ID NO: 370 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = um
  modified_base 4: mod_base = um
  modified_base 5: mod_base = OTHER; note = 2 -O-methyl-adenosine
  modified_base 6: mod_base = cm
  modified_base 7: mod_base = cm
  modified_base 8: mod_base = um
  modified_base 9: mod_base = OTHER; note = 2 -O-methyl-adenosine
  modified_base 10: mod_base = um
  modified_base 11: mod_base = cm
  modified_base 12: mod_base = OTHER; note = 2 -O-methyl-adenosine
  modified_base 13: mod_base = um
  modified_base 14: mod_base = OTHER; note = 2'-Fluoro-uridine
  modified_base 15: mod_base = OTHER; note = 2 -O-methyl-adenosine
  modified_base 16: mod_base = um
  modified_base 17: mod_base = cm
  modified_base 18: mod_base = cm
  modified_base 19..21: mod_base = OTHER; note = linked via phosphorothioate
  modified_base 19: mod_base = cm
  modified_base 20: mod_base = OTHER; note = 2 -O-methyl-adenosine
  modified_base 21: mod_base = OTHER; note = 2 -O-methyl-adenosine
  source 1..21: mol_type = other RNA
tattacctatcattatcccaa

SEQ ID NO: 371 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = OTHER; note = 2 -O-methyl-adenosine
  modified_base 4: mod_base = um
  modified_base 5: mod_base = um
  modified_base 6: mod_base = OTHER; note = 2 -O-methyl-adenosine
  modified_base 7: mod_base = cm
  modified_base 8: mod_base = cm
  modified_base 9: mod_base = um
  modified_base 10: mod_base = OTHER; note = 2 -O-methyl-adenosine
  modified_base 11: mod_base = um
  modified_base 12: mod_base = cm
  modified_base 13: mod_base = OTHER; note = 2 -O-methyl-adenosine
  modified_base 14: mod_base = OTHER; note = 2'-Fluoro-uridine
  modified_base 15: mod_base = um
  modified_base 16: mod_base = OTHER; note = 2 -O-methyl-adenosine
  modified_base 17: mod_base = um
  modified_base 18: mod_base = cm
  modified_base 19..21: mod_base = OTHER; note = linked via phosphorothioate
  modified_base 19: mod_base = cm
  modified_base 20: mod_base = cm
  modified_base 21: mod_base = OTHER; note = 2 -O-methyl-adenosine
  source 1..21: mol_type = other RNA
taattacctatcattatccca

SEQ ID NO: 372 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = OTHER; note = 2 -O-methyl-adenosine
  modified_base 4: mod_base = cm
  modified_base 5: mod_base = um
  modified_base 6: mod_base = um
  modified_base 7: mod_base = um
  modified_base 8: mod_base = cm
  modified_base 9: mod_base = gm
  modified_base 10: mod_base = gm
  modified_base 11: mod_base = OTHER; note = 2 -O-methyl-adenosine
  modified_base 12: mod_base = um
  modified_base 13: mod_base = OTHER; note = 2 -O-methyl-adenosine
  modified_base 14: mod_base = OTHER; note = 2'-Fluoro-adenosine
  modified_base 15: mod_base = OTHER; note = 2 -O-methyl-adenosine
  modified_base 16: mod_base = OTHER; note = 2 -O-methyl-adenosine
  modified_base 17: mod_base = cm
  modified_base 18: mod_base = OTHER; note = 2 -O-methyl-adenosine
  modified_base 19..21: mod_base = OTHER; note = linked via phosphorothioate
  modified_base 19: mod_base = cm
  modified_base 20: mod_base = um
  modified_base 21: mod_base = gm
  source 1..21: mol_type = other RNA
taactttcggataaaacactg

SEQ ID NO: 373 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = OTHER; note = 2 -O-methyl-adenosine
  modified_base 4: mod_base = OTHER; note = 2 -O-methyl-adenosine
  modified_base 5: mod_base = cm
  modified_base 6: mod_base = um
  modified_base 7: mod_base = um
  modified_base 8: mod_base = um
  modified_base 9: mod_base = cm
  modified_base 10: mod_base = gm
  modified_base 11: mod_base = gm
  modified_base 12: mod_base = OTHER; note = 2 -O-methyl-adenosine
  modified_base 13: mod_base = um
  modified_base 14: mod_base = OTHER; note = 2'-Fluoro-adenosine
  modified_base 15: mod_base = OTHER; note = 2 -O-methyl-adenosine
  modified_base 16: mod_base = OTHER; note = 2 -O-methyl-adenosine
  modified_base 17: mod_base = OTHER; note = 2 -O-methyl-adenosine
  modified_base 18: mod_base = cm
  modified_base 19..21: mod_base = OTHER; note = linked via phosphorothioate
  modified_base 19: mod_base = OTHER; note = 2 -O-methyl-adenosine
  modified_base 20: mod_base = cm
  modified_base 21: mod_base = um
  source 1..21: mol_type = other RNA
taaactttcggataaaacact

SEQ ID NO: 374 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = um
  modified_base 4: mod_base = um
  modified_base 5: mod_base = gm
  modified_base 6: mod_base = gm
  modified_base 7: mod_base = OTHER; note = 2 -O-methyl-adenosine
  modified_base 8: mod_base = OTHER; note = 2 -O-methyl-adenosine
  modified_base 9: mod_base = OTHER; note = 2 -O-methyl-adenosine
  modified_base 10: mod_base = cm
  modified_base 11: mod_base = um
  modified_base 12: mod_base = um
  modified_base 13: mod_base = um
  modified_base 14: mod_base = OTHER; note = 2 -Fluoro-cytidine
  modified_base 15: mod_base = gm
  modified_base 16: mod_base = gm
  modified_base 17: mod_base = OTHER; note = 2 -O-methyl-adenosine
  modified_base 18: mod_base = um
  modified_base 19..21: mod_base = OTHER; note = linked via phosphorothioate
  modified_base 19: mod_base = OTHER; note = 2 -O-methyl-adenosine
  modified_base 20: mod_base = OTHER; note = 2 -O-methyl-adenosine
  modified_base 21: mod_base = OTHER; note = 2 -O-methyl-adenosine
  source 1..21: mol_type = other RNA
tattggaaactttcggataaa

SEQ ID NO: 375 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = gm
  modified_base 4: mod_base = OTHER; note = 2 -O-methyl-adenosine
  modified_base 5: mod_base = OTHER; note = 2 -O-methyl-adenosine
  modified_base 6: mod_base = um
  modified_base 7: mod_base = um
  modified_base 8: mod_base = gm
  modified_base 9: mod_base = gm
  modified_base 10: mod_base = OTHER; note = 2 -O-methyl-adenosine
  modified_base 11: mod_base = OTHER; note = 2 -O-methyl-adenosine
  modified_base 12: mod_base = OTHER; note = 2 -O-methyl-adenosine
  modified_base 13: mod_base = cm
  modified_base 14: mod_base = OTHER; note = 2'-Fluoro-uridine
  modified_base 15: mod_base = um
  modified_base 16: mod_base = um
  modified_base 17: mod_base = cm
  modified_base 18: mod_base = gm
  modified_base 19..21: mod_base = OTHER; note = linked via phosphorothioate
  modified_base 19: mod_base = gm
  modified_base 20: mod_base = OTHER; note = 2 -O-methyl-adenosine
  modified_base 21: mod_base = um
  source 1..21: mol_type = other RNA
tggaattggaaactttcggat

SEQ ID NO: 376 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = OTHER; note = 2 -O-methyl-adenosine
  modified_base 4: mod_base = cm
  modified_base 5: mod_base = um
  modified_base 6: mod_base = OTHER; note = 2 -O-methyl-adenosine
  modified_base 7: mod_base = gm
  modified_base 8: mod_base = um
  modified_base 9: mod_base = OTHER; note = 2 -O-methyl-adenosine
  modified_base 10: mod_base = OTHER; note = 2 -O-methyl-adenosine
  modified_base 11: mod_base = gm
  modified_base 12: mod_base = OTHER; note = 2 -O-methyl-adenosine
  modified_base 13: mod_base = OTHER; note = 2 -O-methyl-adenosine
  modified_base 14: mod_base = OTHER; note = 2'-Fluoro-adenosine
  modified_base 15: mod_base = um
  modified_base 16: mod_base = um
  modified_base 17: mod_base = gm
  modified_base 18: mod_base = gm
  modified_base 19..21: mod_base = OTHER; note = linked via phosphorothioate
  modified_base 19: mod_base = cm
  modified_base 20: mod_base = OTHER; note = 2 -O-methyl-adenosine
  modified_base 21: mod_base = cm
  source 1..21: mol_type = other RNA
ttactagtaagaaattggcac

SEQ ID NO: 377 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = um
  modified_base 4: mod_base = OTHER; note = 2 -O-methyl-adenosine
  modified_base 5: mod_base = cm
  modified_base 6: mod_base = um
  modified_base 7: mod_base = OTHER; note = 2 -O-methyl-adenosine
  modified_base 8: mod_base = gm
  modified_base 9: mod_base = um
  modified_base 10: mod_base = OTHER; note = 2 -O-methyl-adenosine
  modified_base 11: mod_base = OTHER; note = 2 -O-methyl-adenosine
  modified_base 12: mod_base = gm
  modified_base 13: mod_base = OTHER; note = 2 -O-methyl-adenosine
  modified_base 14: mod_base = OTHER; note = 2'-Fluoro-adenosine
  modified_base 15: mod_base = OTHER; note = 2 -O-methyl-adenosine
  modified_base 16: mod_base = um
  modified_base 17: mod_base = um
  modified_base 18: mod_base = gm
  modified_base 19..21: mod_base = OTHER; note = linked via phosphorothioate
  modified_base 19: mod_base = gm
  modified_base 20: mod_base = cm
  modified_base 21: mod_base = OTHER; note = 2 -O-methyl-adenosine
  source 1..21: mol_type = other RNA
tgtactagtaagaaattggca

SEQ ID NO: 378 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = gm
  modified_base 4: mod_base = cm
  modified_base 5: mod_base = gm
  modified_base 6: mod_base = cm
  modified_base 7: mod_base = OTHER; note = 2 -O-methyl-adenosine
  modified_base 8: mod_base = OTHER; note = 2 -O-methyl-adenosine
  modified_base 9: mod_base = cm
  modified_base 10: mod_base = cm
  modified_base 11: mod_base = OTHER; note = 2 -O-methyl-adenosine
  modified_base 12: mod_base = OTHER; note = 2 -O-methyl-adenosine
  modified_base 13: mod_base = OTHER; note = 2 -O-methyl-adenosine
  modified_base 14: mod_base = OTHER; note = 2'-Fluoro-uridine
  modified_base 15: mod_base = gm
  modified_base 16: mod_base = OTHER; note = 2 -O-methyl-adenosine
  modified_base 17: mod_base = um
  modified_base 18: mod_base = gm
  modified_base 19..21: mod_base = OTHER; note = linked via phosphorothioate
  modified_base 19: mod_base = gm
  modified_base 20: mod_base = OTHER; note = 2 -O-methyl-adenosine
  modified_base 21: mod_base = OTHER; note = 2 -O-methyl-adenosine
  source 1..21: mol_type = other RNA
tagcgcaaccaaatgatggaa

SEQ ID NO: 379 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-guanosine
  modified_base 3: mod_base = um
  modified_base 4: mod_base = cm
  modified_base 5: mod_base = OTHER; note = 2 -O-methyl-adenosine
  modified_base 6: mod_base = gm
  modified_base 7: mod_base = cm
  modified_base 8: mod_base = gm
  modified_base 9: mod_base = cm
  modified_base 10: mod_base = OTHER; note = 2 -O-methyl-adenosine
  modified_base 11: mod_base = OTHER; note = 2 -O-methyl-adenosine
  modified_base 12: mod_base = cm
  modified_base 13: mod_base = cm
  modified_base 14: mod_base = OTHER; note = 2'-Fluoro-adenosine
  modified_base 15: mod_base = OTHER; note = 2 -O-methyl-adenosine
  modified_base 16: mod_base = OTHER; note = 2 -O-methyl-adenosine
  modified_base 17: mod_base = um
  modified_base 18: mod_base = gm
  modified_base 19..21: mod_base = OTHER; note = linked via phosphorothioate
  modified_base 19: mod_base = OTHER; note = 2 -O-methyl-adenosine
  modified_base 20: mod_base = um
  modified_base 21: mod_base = gm
  source 1..21: mol_type = other RNA
tgtcagcgcaaccaaatgatg

SEQ ID NO: 380 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = gm
  modified_base 4: mod_base = OTHER; note = 2 -O-methyl-adenosine
  modified_base 5: mod_base = cm
  modified_base 6: mod_base = cm
  modified_base 7: mod_base = OTHER; note = 2 -O-methyl-adenosine
  modified_base 8: mod_base = OTHER; note = 2 -O-methyl-adenosine
  modified_base 9: mod_base = cm
  modified_base 10: mod_base = OTHER; note = 2 -O-methyl-adenosine
  modified_base 11: mod_base = um
  modified_base 12: mod_base = um
  modified_base 13: mod_base = cm
  modified_base 14: mod_base = OTHER; note = 2 -Fluoro-cytidine
  modified_base 15: mod_base = um
  modified_base 16: mod_base = OTHER; note = 2 -O-methyl-adenosine
  modified_base 17: mod_base = gm
  modified_base 18: mod_base = gm
  modified_base 19..21: mod_base = OTHER; note = linked via phosphorothioate
  modified_base 19: mod_base = um
  modified_base 20: mod_base = cm
  modified_base 21: mod_base = OTHER; note = 2 -O-methyl-adenosine
  source 1..21: mol_type = other RNA
ttgaccaacattcctaggtca

SEQ ID NO: 381 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = um
  modified_base 4: mod_base = gm
  modified_base 5: mod_base = OTHER; note = 2 -O-methyl-adenosine
  modified_base 6: mod_base = cm
  modified_base 7: mod_base = cm
  modified_base 8: mod_base = OTHER; note = 2 -O-methyl-adenosine
  modified_base 9: mod_base = OTHER; note = 2 -O-methyl-adenosine
  modified_base 10: mod_base = cm
  modified_base 11: mod_base = OTHER; note = 2 -O-methyl-adenosine
  modified_base 12: mod_base = um
  modified_base 13: mod_base = um
  modified_base 14: mod_base = OTHER; note = 2 -Fluoro-cytidine
  modified_base 15: mod_base = cm
  modified_base 16: mod_base = um
  modified_base 17: mod_base = OTHER; note = 2 -O-methyl-adenosine
  modified_base 18: mod_base = gm
  modified_base 19..21: mod_base = OTHER; note = linked via phosphorothioate
  modified_base 19: mod_base = gm
  modified_base 20: mod_base = um
  modified_base 21: mod_base = cm
  source 1..21: mol_type = other RNA
tatgaccaacattcctaggtc

SEQ ID NO: 382 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = OTHER; note = 2 -O-methyl-adenosine
  modified_base 4: mod_base = um
  modified_base 5: mod_base = gm
  modified_base 6: mod_base = OTHER; note = 2 -O-methyl-adenosine
  modified_base 7: mod_base = cm
  modified_base 8: mod_base = cm
  modified_base 9: mod_base = OTHER; note = 2 -O-methyl-adenosine
  modified_base 10: mod_base = OTHER; note = 2 -O-methyl-adenosine
  modified_base 11: mod_base = cm
  modified_base 12: mod_base = OTHER; note = 2 -O-methyl-adenosine
  modified_base 13: mod_base = um
  modified_base 14: mod_base = OTHER; note = 2'-Fluoro-uridine
  modified_base 15: mod_base = cm
  modified_base 16: mod_base = cm
  modified_base 17: mod_base = um
  modified_base 18: mod_base = OTHER; note = 2 -O-methyl-adenosine
  modified_base 19..21: mod_base = OTHER; note = linked via phosphorothioate
  modified_base 19: mod_base = gm
  modified_base 20: mod_base = gm
  modified_base 21: mod_base = um
  source 1..21: mol_type = other RNA
ttatgaccaacattcctaggt

SEQ ID NO: 383 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = um
  modified_base 4: mod_base = OTHER; note = 2 -O-methyl-adenosine
  modified_base 5: mod_base = um
  modified_base 6: mod_base = gm
  modified_base 7: mod_base = OTHER; note = 2 -O-methyl-adenosine
  modified_base 8: mod_base = cm
  modified_base 9: mod_base = cm
  modified_base 10: mod_base = OTHER; note = 2 -O-methyl-adenosine
  modified_base 11: mod_base = OTHER; note = 2 -O-methyl-adenosine
  modified_base 12: mod_base = cm
  modified_base 13: mod_base = OTHER; note = 2 -O-methyl-adenosine
  modified_base 14: mod_base = OTHER; note = 2'-Fluoro-uridine
  modified_base 15: mod_base = um
  modified_base 16: mod_base = cm
  modified_base 17: mod_base = cm
  modified_base 18: mod_base = um
  modified_base 19..21: mod_base = OTHER; note = linked via phosphorothioate
  modified_base 19: mod_base = OTHER; note = 2 -O-methyl-adenosine
  modified_base 20: mod_base = gm
  modified_base 21: mod_base = gm
  source 1..21: mol_type = other RNA
tatatgaccaacattcctagg

SEQ ID NO: 384 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = gm
  modified_base 4: mod_base = OTHER; note = 2 -O-methyl-adenosine
  modified_base 5: mod_base = um
  modified_base 6: mod_base = OTHER; note = 2 -O-methyl-adenosine
  modified_base 7: mod_base = um
  modified_base 8: mod_base = gm
  modified_base 9: mod_base = OTHER; note = 2 -O-methyl-adenosine
  modified_base 10: mod_base = cm
  modified_base 11: mod_base = cm
  modified_base 12: mod_base = OTHER; note = 2 -O-methyl-adenosine
  modified_base 13: mod_base = OTHER; note = 2 -O-methyl-adenosine
  modified_base 14: mod_base = OTHER; note = 2 -Fluoro-cytidine
  modified_base 15: mod_base = OTHER; note = 2 -O-methyl-adenosine
  modified_base 16: mod_base = um
  modified_base 17: mod_base = um
  modified_base 18: mod_base = cm
  modified_base 19..21: mod_base = OTHER; note = linked via phosphorothioate
  modified_base 19: mod_base = cm
  modified_base 20: mod_base = um
  modified_base 21: mod_base = OTHER; note = 2 -O-methyl-adenosine
  source 1..21: mol_type = other RNA
ttgatatgaccaacattccta

SEQ ID NO: 385 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = OTHER; note = 2 -O-methyl-adenosine
  modified_base 4: mod_base = um
  modified_base 5: mod_base = um
  modified_base 6: mod_base = OTHER; note = 2 -O-methyl-adenosine
  modified_base 7: mod_base = gm
  modified_base 8: mod_base = gm
  modified_base 9: mod_base = OTHER; note = 2 -O-methyl-adenosine
  modified_base 10: mod_base = gm
  modified_base 11: mod_base = um
  modified_base 12: mod_base = OTHER; note = 2 -O-methyl-adenosine
  modified_base 13: mod_base = gm
  modified_base 14: mod_base = OTHER; note = 2'-Fluoro-uridine
  modified_base 15: mod_base = OTHER; note = 2 -O-methyl-adenosine
  modified_base 16: mod_base = cm
  modified_base 17: mod_base = OTHER; note = 2 -O-methyl-adenosine
  modified_base 18: mod_base = gm
  modified_base 19..21: mod_base = OTHER; note = linked via phosphorothioate
  modified_base 19: mod_base = um
  modified_base 20: mod_base = um
  modified_base 21: mod_base = cm
  source 1..21: mol_type = other RNA
taattaggagtagtacagttc

SEQ ID NO: 386 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-uridine
  modified_base 3: mod_base = OTHER; note = 2 -O-methyl-adenosine
  modified_base 4: mod_base = OTHER; note = 2 -O-methyl-adenosine
  modified_base 5: mod_base = um
  modified_base 6: mod_base = um
  modified_base 7: mod_base = OTHER; note = 2 -O-methyl-adenosine
  modified_base 8: mod_base = gm
  modified_base 9: mod_base = gm
  modified_base 10: mod_base = OTHER; note = 2 -O-methyl-adenosine
  modified_base 11: mod_base = gm
  modified_base 12: mod_base = um
  modified_base 13: mod_base = OTHER; note = 2 -O-methyl-adenosine
  modified_base 14: mod_base = OTHER; note = 2'-Fluoro-guanosine
  modified_base 15: mod_base = um
  modified_base 16: mod_base = OTHER; note = 2 -O-methyl-adenosine
  modified_base 17: mod_base = cm
  modified_base 18: mod_base = OTHER; note = 2 -O-methyl-adenosine
  modified_base 19..21: mod_base = OTHER; note = linked via phosphorothioate
  modified_base 19: mod_base = gm
  modified_base 20: mod_base = um
  modified_base 21: mod_base = um
  source 1..21: mol_type = other RNA
ttaattaggagtagtacagtt

SEQ ID NO: 387 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = um
  modified_base 4: mod_base = OTHER; note = 2 -O-methyl-adenosine
  modified_base 5: mod_base = OTHER; note = 2 -O-methyl-adenosine
  modified_base 6: mod_base = um
  modified_base 7: mod_base = um
  modified_base 8: mod_base = OTHER; note = 2 -O-methyl-adenosine
  modified_base 9: mod_base = gm
  modified_base 10: mod_base = gm
  modified_base 11: mod_base = OTHER; note = 2 -O-methyl-adenosine
  modified_base 12: mod_base = gm
  modified_base 13: mod_base = um
  modified_base 14: mod_base = OTHER; note = 2'-Fluoro-adenosine
  modified_base 15: mod_base = gm
  modified_base 16: mod_base = um
  modified_base 17: mod_base = OTHER; note = 2 -O-methyl-adenosine
  modified_base 18: mod_base = cm
  modified_base 19..21: mod_base = OTHER; note = linked via phosphorothioate
  modified_base 19: mod_base = OTHER; note = 2 -O-methyl-adenosine
  modified_base 20: mod_base = gm
  modified_base 21: mod_base = um
  source 1..21: mol_type = other RNA
tataattaggagtagtacagt

SEQ ID NO: 388 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = OTHER; note = 2 -O-methyl-adenosine
  modified_base 4: mod_base = um
  modified_base 5: mod_base = OTHER; note = 2 -O-methyl-adenosine
  modified_base 6: mod_base = OTHER; note = 2 -O-methyl-adenosine
  modified_base 7: mod_base = um
  modified_base 8: mod_base = um
  modified_base 9: mod_base = OTHER; note = 2 -O-methyl-adenosine
  modified_base 10: mod_base = gm
  modified_base 11: mod_base = gm
  modified_base 12: mod_base = OTHER; note = 2 -O-methyl-adenosine
  modified_base 13: mod_base = gm
  modified_base 14: mod_base = OTHER; note = 2'-Fluoro-uridine
  modified_base 15: mod_base = OTHER; note = 2 -O-methyl-adenosine
  modified_base 16: mod_base = gm
  modified_base 17: mod_base = um
  modified_base 18: mod_base = OTHER; note = 2 -O-methyl-adenosine
  modified_base 19..21: mod_base = OTHER; note = linked via phosphorothioate
  modified_base 19: mod_base = cm
  modified_base 20: mod_base = OTHER; note = 2 -O-methyl-adenosine
  modified_base 21: mod_base = gm
  source 1..21: mol_type = other RNA
taataattaggagtagtacag

(19) United States
(12) Patent Application Publication
(10) Pub. No.: US 2026/0199515 A1
(43) Pub. Date: Jul. 16, 2026

(54) COMPOSITIONS AND METHODS FOR KRAS INHIBITION FOR THE TREATMENT OF DISEASE

(71) Applicant: ALTAMIRA THERAPEUTICS AG, Basel (CH)

(72) Inventors: Samuel A. WICKLINE, Basel (CH); Covadonga Pañeda VAZQUEZ-PRADA, Basel (CH); Reda JUSKEVICIENE, Basel (CH)

(21) Appl. No.: 19/158,137

(22) PCT Filed: Feb. 21, 2024
(86) PCT No.: PCT/IB2024/051690

§ 371 Date: Aug. 20, 2025

Related U.S. Application Data
(60) Provisional application No. 63/486,339, filed on Feb. 22, 2023.
(60) Provisional application No. 63/624,088, filed on Jan. 23, 2024.

(51) Int. Cl. A61K 47/69; A61K 31/712; A61K 31/7125; A61K 31/713; A61K 47/64; A61P 35/00; B82Y 5/00; C12N 15/113

(52) U.S. Cl. CPC A61K 47/6911; A61K 31/712; A61K 31/7125; A61K 31/713; A61K 47/64; A61P 35/00; C12N 15/1137; B82Y 5/00; C12N 2310/11; C12N 2310/14; C12N 2310/315; C12N 2310/321; C12N 2310/322; C12Y 306/05002

(57) ABSTRACT

The present disclosure provides pharmaceutical compositions comprising a peptide-polynucleotide complex, wherein the peptide comprises an amino acid sequence with at least 80%, at least 85%, at least 90%, at least 95%, at least 98%, at least 99%, or 100% identity to the amino acid sequence of SEQ ID NO: 1, SEQ ID NO: 2, or SEQ ID NO: 3; and wherein the polynucleotide is a small interfering RNA (siRNA) targeting human KRAS mRNA, wherein the target sequence of human KRAS mRNA does not encode G12, G13, or Q61 with reference to SEQ ID NO: 4 or a mutant amino acid at position 12, 13, or 61 with reference to SEQ ID NO: 4.

### 1. CROSS-REFERENCE TO RELATED APPLICATIONS

This application claims priority to U.S. Provisional Application No. 63/486,339, filed Feb. 22, 2023 and U.S. Provisional Application No. 63/624,088, filed Jan. 23, 2024; each of which is hereby incorporated by reference in its entirety.

### 2. DESCRIPTION OF THE TEXT FILE SUBMITTED ELECTRONICALLY

The contents of the electronic sequence listing (AURS_010_02WO_SeqList_ST26.xml; Size: 2,214,874 bytes; and Date of Creation: Feb. 10, 2024) are herein incorporated by reference in its entirety.

### 3. FIELD

The present disclosure generally relates to pharmaceutical compositions for knocking down KRAS. The present disclosure also relates to treating a disease or disorder in a subject using the pharmaceutical compositions disclosed herein.

### 4. BACKGROUND

The KRAS gene provides instructions for making a protein called KRAS, which plays important roles in cell division, cell differentiation, and the self-destruction of cells (apoptosis). Mutational activation of KRAS is a common oncogenic event.

### 5. SUMMARY

In one aspect, disclosed herein is a pharmaceutical composition comprising a peptide-polynucleotide complex, wherein the peptide comprises an amino acid sequence with at least 80%, at least 85%, at least 90%, at least 95%, at least 98%, at least 99%, or 100% idesomentity to the amino acid sequence of SEQ ID NO: 1, SEQ ID NO: 2, or SEQ ID NO: 3; and wherein the polynucleotide is a small interfering RNA (siRNA) targeting human KRAS mRNA, wherein the target sequence of human KRAS mRNA does not encode G12, G13, or Q61 with reference to SEQ ID NO: 4 or a mutant amino acid at position 12, 13, or 61 with reference to SEQ ID NO: 4. In some embodiments, the peptide is non-lytic, non-cytotoxic, and capable of affecting the release of the polynucleotide from an endosome of a cell. In some embodiments, the peptide comprises two or more contiguous, basic amino acids (a cationic region) and one or more histidine residues located adjacent to the cationic region. In some embodiments, the peptide comprises an amino acid sequence of SEQ ID NO: 1, SEQ ID NO: 2, or SEQ ID NO: 3. In some embodiments, the siRNA comprises a sense strand and an antisense strand. In some embodiments, the sense strand and the antisense strand are each 16-24 bases in length. In some embodiments, the sense strand is 19 bases in length. In some embodiments, the antisense strand is 21 bases in length. In some embodiments, the sense strand and the antisense strand are modified. In some embodiments, the modifications are selected from the group consisting of 2′-methoxy (2′-OMe), 2′-fluoro (2′-F), 2′-O-methoxyethyl(2′-O-MOE), 5′-vinylphosphonate, phosphorothioate (PTO), locked nucleic acid (LNA), locked nucleic acid (UNA), glycol nucleic acid (GNA), and DNA. In some embodiments, the modifications of the sense strand comprise: PTO at positions 1 and 2; 2′-F at positions 3, 7-9, 12, and 17; and 2′-OMe at positions 1, 2, 4-6, 10, 11, 13-16, 18, and 19. In some embodiments, the modifications of the antisense strand comprise: PTO at positions 1, 2, 19, and 20; 2′-F at positions 2 and 14; and 2′-OMe at positions 1, 3-13, and 15-21. In some embodiments, the last nucleotide of the sense strand is adenine (A). In some embodiments, the first nucleotide of the antisense strand is uracil (U). In some embodiments, the sense strand comprises a nucleotide sequence with at least at least 80%, at least 85%, at least 90%, at least 95%, at least 98%, at least 99%, or 100% identity to the nucleotide sequence of any one of the sense strands listed in Table 1 and Table 2. In some embodiments, the antisense strand comprises a nucleotide sequence with at least at least 80%, at least 85%, at least 90%, at least 95%, at least 98%, at least 99%, or 100% identity to the nucleotide sequence of any one of the antisense strands listed in Table 1 and Table 2. In some embodiments, the ratio of peptide to polynucleotide is about 6:1 to about 18:1, wherein the ratio is the ratio of positively-chargeable polymer amine groups to negatively-charged nucleic acid phosphate groups. In some embodiments, the charge ratio of peptide to polynucleotide is about 12:1. In some embodiments, the ratio of peptide to polynucleotide is about 2:1 to about 3500:1, wherein the ratio is the molar ratio. In some embodiments, the molar ratio of peptide to polynucleotide is about 4:1 to about 1000:1. In some embodiments, the molar ratio of peptide to polynucleotide is about 5:1 to about 200:1. In some embodiments, the molar ratio of peptide to polynucleotide is about 50:1 to about 200:1. In some embodiments, the molar ratio of peptide to polynucleotide is about 5:1. In some embodiments, the molar ratio of peptide to polynucleotide is about 100:1. In some embodiments, the peptide-polynucleotide complex is a nanoparticle with a diameter of about 10 nm to about 300 nm. In some embodiments, the peptide-polynucleotide complex is coated with albumin and/or hyaluronic acid. In some embodiments, the pharmaceutical composition further comprises a pharmaceutical acceptable carrier.

In another aspect, provided herein is a method of treating a disease or disorder in a subject, comprising administering to the subject a therapeutically effective amount of the pharmaceutical composition disclosed herein. In some embodiments, the disease or disorder is cancer. In some embodiments, the cancer is blood cancer or solid tumor cancer.

### 6. BRIEF DESCRIPTION OF THE FIGURES

FIG. 1 shows an exemplary modification pattern of the siRNA disclosed herein. 2′-methoxy is referred to as 2′-OMe, 2′-fluoro is referred to as 2′-F, and phosphorothioate is referred to as PTO.

FIGS. 2A-2F show the knowdown of KRAS in NCI-H23 cells carrying a KRAS G12C mutation by two exemplary siRNAs: XD-39946 and XD-39966. FIG. 2A shows the dose-response curve of XD-39946; FIG. 2B shows the dose-response curve of XD-39966; FIG. 2C shows the relative mRNA expression level of KRAS at different concentrations of XD-39946 and XD-39966; FIG. 2D shows the relative mRNA expression level of GAPDH at different concentrations of XD-39946 and XD-39966; FIG. 2E shows the original data regarding the mRNA expression levels of KRAS and GAPDH at different concentrations of XD-39946 and XD-39966. FIG. 2F shows the bar charts of the original data in FIG. 2E.

FIGS. 3A-3C show the knowdown of KRAS in cell lines harboring either wildtype or mutant KRAS by two exemplary siRNAs: XD-39951 and XD-39947. FIG. 3A shows the knowdown of KRAS in SW480 cells (G12V mutation). FIG. 3B shows the knowdown of KRAS in HT-29 cells (wildtype). FIG. 3C shows the knowdown of KRAS in LS174T cells (G12D mutation).

FIGS. 4A-4B show the knowdown of KRAS in more cell lines harboring additional mutations of KRAS by exemplary siRNA XD-39951 and its effect on cell viability. FIG. 4A shows the knowdown of KRAS in PDAC, NSCLC, and CRC cells harboring different mutations of KRAS. FIG. 4B shows the cell viability after the knowdown of KRAS in PDAC, NSCLC, and CRC cells.

### 7. DETAILED DESCRIPTION

Disclosed herein are pharmaceutical compositions comprising a peptide-polynucleotide complex for KRAS inhibition for the treatment of disease.

### 7.1. Definitions

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The terms “homologous,” “identical,” or percent “identity” in relation to two or more peptides, refers to two or more sequences or subsequences that have a specified percentage of amino acid residues that are the same (i.e., about 60% identity, about 70%, about 75%, about 80%, about 85%, about 90%, about 91%, about 92%, about 93%, about 94%, about 95%, about 96%, about 97%, about 98%, about 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/or the like). The definition also includes sequences that have deletions and/or additions, as well as those that have substitutions, as well as naturally occurring, e.g., polymorphic or allelic variants, and man-made variants. As described below, the algorithms can account for gaps and the like.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. The term “purified” in some embodiments denotes that a nucleic acid or protein gives rise to essentially one band in an electrophoretic gel., it means that the nucleic acid or protein is at least 85% pure, at least 95% pure, and most at least 99% pure. “Purify” or “purification” in other embodiments means removing at least one contaminant from the composition to be purified. In this sense, purification does not require that the purified compound be homogenous, e.g., 100% pure.

The term “target sequence” refers to a sequence of nucleotides found within the mRNA of a target gene (e.g., the KRAS gene). Such a sequence of nucleotides is complementary to the anti-sense strand of an siRNA disclosed herein.

### 7.2. Peptide-Polynucleotide Complex

One aspect of the present invention encompasses a peptide-polynucleotide complex. A peptide-polynucleotide complex of the invention is capable of efficient transfection of a polynucleotide associated with the peptide into the cytoplasm of a cell. The peptide, the polynucleotide, the peptide-polynucleotide complex, and the cell are described below.

### 7.2.1. Peptide

In an aspect, a peptide-polynucleotide complex of the invention comprises a peptide. In general, and as described in the examples, a peptide of the invention is derived from melittin and modified to attenuate its cytotoxicity while maintaining its propensity for interacting with membrane bilayers. Furthermore, the peptide is substantially non-lytic and non-cytotoxic to cells., a peptide-polynucleotide complex of the invention comprises a peptide that (1) has a function substantially similar to a peptide with an amino acid sequence of SEQ ID NO: 1 (VLTTGLPALISWIRRRHRRHC), SEQ ID NO: 2 (VLTTGLPALISWIRRRHRRHG), or SEQ ID NO: 3 (VLTTGLPALISWIKRKRQHRWRRRR), and (2) has an amino acid sequence with similarity or identity to the amino acid sequence of SEQ ID NO: 1, SEQ ID NO: 2, or SEQ ID NO: 3.

As used herein, the phrase “functions substantially similar to a peptide comprising SEQ ID NO: 1, 2, or 3” refers to a substantially non-lytic and/or non-cytotoxic peptide that is capable of affecting the release of a polynucleotide from an endosome. In some embodiments a peptide of the invention is non-lytic. The term “non-lytic” means that the lipid bilayer of a cell typically is not compromised upon contact with the peptide. The integrity of the lipid bilayer may be assessed by the improper entry or exit of cellular or extracellular components into a cell. For example, cellular proteins and/or organelles may leak out of a cell with a compromised lipid bilayer. Alternatively, extracellular components (i.e., those that normally do not enter via gap junctions, for example) may enter a cell with a compromised lipid bilayer. It should be noted, however, that the peptide may penetrate the lipid bilayer of a cell and enter the interior of the cell, but in doing so the integrity of the lipid bilayer is not affected. In other embodiments, a peptide of the invention is substantially non-cytotoxic. The term “non-cytotoxic” indicates that the cell typically is not killed upon contact with the peptide. Typically, a peptide of the invention decreases cell viability by no more than about 10%, no more than about 7%, no more than about 5%, or no more than about 3%. In certain embodiments, a peptide of the invention is non-lytic and non-cytotoxic.

A peptide of the invention is capable of associating with a polynucleotide. Thus, in one aspect, a peptide of the invention comprises at least one cationic region that interacts with a polynucleotide. Typically, a cationic region has 2 or more contiguous, basic amino acids. Importantly, a peptide of the invention also possesses an endosomolytic capacity, which allows it to affect the release of a polynucleotide from an endosome and into the cytoplasm of a cell. The term “endosomolytic” can be used to describe substances that initiate or facilitate the lysis of endosomes. As described in the Examples, protonation of histidine residues of a peptide of the invention promotes disassembly of the peptide-polynucleotide complex, which releases the peptide to permeabilize the endosomal membrane for polynucleotide release. Thus, in another aspect, a peptide of the invention comprises one or more histidine residues located adjacent to or within at least one cationic region of the peptide. By way of non-limiting example, if a peptide of the invention comprises three cationic regions, the peptide may have at least one histidine adjacent to or within the first cationic region of the peptide, at least one histidine adjacent to or within the second cationic region of the peptide, at least one histidine adjacent to or within the third cationic region of the peptide, at least one histidine adjacent to or within each of the first and second cationic region of the peptide, at least one histidine adjacent to or within each of the first and third cationic region of the peptide, at least one histidine adjacent to or within each of the second and third cationic region of the peptide, or at least one histidine adjacent to or within each of the first, second and third cationic region of the peptide. A histidine residue adjacent to a cationic region may be positioned before or after the cationic region. In some embodiments, a histidine residue adjacent to a cationic region is immediately adjacent to the region. In other embodiments, a histidine residue adjacent to a cationic region is not immediately adjacent to the region. For example, the histidine residue may be within about 2, 3, 4 or 5 positions from the cationic region. In other embodiments, a histidine residue is within a cationic region. The endosomolytic capacity of a peptide of the invention obviates the need for additional endosomolytic agents, such as chloroquine, fusogenic peptides, inactivated adenoviruses and polyethyleneimine, for releasing transfected polynucleotides from endosomes for delivery into the cytoplasm of a cell. Such known endosomolytic agents have negative effects on cells, and may increase cytotoxicity during transfection.

In some embodiments, a peptide of the invention comprises SEQ ID NO: 1. In other embodiments, a peptide of the inventions consists of SEQ ID NO: 1. In certain embodiments, a peptide of the invention is a variant of SEQ ID NO: 1, wherein the variant comprises at least 10 contiguous amino acids of SEQ ID NO: 1 and functions substantially similar to a peptide comprising SEQ ID NO: 1. For instance, a peptide of the invention may encompass at least 10, 11, 12, 13, 14, 15, 16, 17, 18, 19, or 20 contiguous amino acids of SEQ ID NO: 1.

In some embodiments, a peptide of the invention comprises SEQ ID NO: 2. In other embodiments, a peptide of the inventions consists of SEQ ID NO: 2. In certain embodiments, a peptide of the invention is a variant of SEQ ID NO: 2, wherein the variant comprises at least 10 contiguous amino acids of SEQ ID NO: 2 and functions substantially similar to a peptide comprising SEQ ID NO: 2. For instance, a peptide of the invention may encompass at least 10, 11, 12, 13, 14, 15, 16, 17, 18, 19, or 20 contiguous amino acids of SEQ ID NO: 2.

In some embodiments, a peptide of the invention comprises SEQ ID NO: 3. In other embodiments, a peptide of the inventions consists of SEQ ID NO: 3. In certain embodiments, a peptide of the invention is a variant of SEQ ID NO: 3, wherein the variant comprises at least 10 contiguous amino acids of SEQ ID NO: 3 and functions substantially similar to a peptide comprising SEQ ID NO: 3. For instance, a peptide of the invention may encompass at least 10, 11, 12, 13, 14, 15, 16, 17, 18, 19, or 20 contiguous amino acids of SEQ ID NO: 3.

In some embodiments, a peptide of the invention comprises an amino acid sequence that has at least 80% identity to SEQ ID NO: 1, wherein the peptide is non-lytic and is capable of affecting the release of a polynucleotide from an endosome of a cell. The peptide comprising an amino acid sequence that has at least 80% identity to SEQ ID NO: 1, can have about 80%, about 85%, about 90%, about 95% identity to the amino acid sequence of SEQ ID NO: 1. A peptide of the invention comprising an amino acid sequence that has at least 80% identity to SEQ ID NO: 1 may comprise one or more amino acids that have been conservatively substituted. For instance, one, two, three, four, five, six, seven, eight, nine, or more than nine amino acids may be conservatively substituted as long as the resulting peptide functions substantially similar to a peptide comprising SEQ ID NO: 1.

In some embodiments, a peptide of the invention comprises an amino acid sequence that has at least 80% identity to SEQ ID NO: 2, wherein the peptide is non-lytic and is capable of affecting the release of a polynucleotide from an endosome of a cell. The peptide comprising an amino acid sequence that has at least 80% identity to SEQ ID NO: 2, can have about 80%, about 85%, about 90%, about 95% identity to the amino acid sequence of SEQ ID NO: 2. A peptide of the invention comprising an amino acid sequence that has at least 80% identity to SEQ ID NO: 2 may comprise one or more amino acids that have been conservatively substituted. For instance, one, two, three, four, five, six, seven, eight, nine, or more than nine amino acids may be conservatively substituted as long as the resulting peptide functions substantially similar to a peptide comprising SEQ ID NO: 2.

In some embodiments, a peptide of the invention comprises an amino acid sequence that has at least 80% identity to SEQ ID NO: 3, wherein the peptide is non-lytic and is capable of affecting the release of a polynucleotide from an endosome of a cell. The peptide comprising an amino acid sequence that has at least 80% identity to SEQ ID NO: 3, can have about 80%, about 85%, about 90%, about 95% identity to the amino acid sequence of SEQ ID NO: 3. A peptide of the invention comprising an amino acid sequence that has at least 80% identity to SEQ ID NO: 3 may comprise one or more amino acids that have been conservatively substituted. For instance, one, two, three, four, five, six, seven, eight, nine, or more than nine amino acids may be conservatively substituted as long as the resulting peptide functions substantially similar to a peptide comprising SEQ ID NO: 3.

A peptide of the invention may be produced using a variety of techniques known in the art. The peptides may be isolated using standard techniques, may be synthesized using standard techniques, or may be purchased or obtained from a depository.

When a peptide of the invention contains a C-terminal thiol in the form of a cysteine residue, a peptide of the invention may be able to form a disulfide bond with another free thiol group, for example, with a free thiol group from the same or different peptide. A skilled artisan can readily determine whether dimer formation does or does not improve the delivery of plasmid DNA. Without wishing to be bound by theory, dimer formation may improve the delivery of plasmid DNA for certain peptides of the invention due to improved DNA condensation. Dimerization may be induced by incubation of free peptide in 20% DMSO for 24-72 hours, or by other methods known in other art. As a non-limiting example, free thiols may be quantified by colorimetric assays using Ellman's Reagent.

A peptide of the invention may be labeled. Non-limiting examples of suitable labels include fluorescent labels, chemiluminescent labels, radioactive labels, colorimetric labels, and resonance labels. Methods of labeling peptides are well known in the art.

A peptide may be bound to a cargo complex. As used herein, the term “cargo complex” may refer to any molecule or agent that may be carried by or bound to the peptide other than a polynucleotide of the invention. Stated another way, a peptide of the invention may be bound to a cargo complex in addition to a polynucleotide of the invention. For instance, a cargo complex may be an imaging cargo, a therapeutic cargo, a cytotoxic cargo, or a targeting cargo.

Non-limiting examples of imaging cargo molecules and agents may include any molecule, agent, or material having a detectable physical or chemical property. Such imaging cargos have been well-developed in the field of fluorescent imaging, magnetic resonance imaging, positron emission tomography, Raman imaging, optical coherence tomography, photoacoustic imaging, Fourier transform infrared imaging, or immunoassays and, in general, most any label useful in such methods may be applied to the present invention. For a review of various labeling or signal producing systems that may be used, see U.S. Pat. No. 4,391,904, incorporated herein by reference in its entirety.

Non-limiting examples of therapeutic cargo may include any substance that has a biological activity, such as pharmacological agents. Such therapeutic cargo may include analgesics, antipyretics, antiasthmatics, antibiotics, antidepressants, antidiabetics, antifungal agents, antihypertensive agents, anti-inflammatories including non-steroidal and steroidal, antineoplastics, antianxiety agents, immunosuppressive agents, anti migraine agents, sedatives, hypnotics, antianginal agents, antipsychotic agents, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines, anti-restenosis agents, antipruritics, agents useful for calcium regulation, antibacterial agents, antiviral agents, antimicrobials, anti-infectives, bronchodilators, steroidal compounds and hormones, and combinations thereof. Alternatively, a cargo complex may be in the form of components of molecular complexes or pharmacologically acceptable salts.

Cytotoxic cargo refers to a molecule or agent that is detrimental to (e.g., kills or damages) a cell. Examples may include anti-microtubule drugs such as the taxols (paclitaxel, docetaxel) and vinca alkaloids (vincristine, vinblastine). For instance, examples may include taxol, cytochalasin B, gramicidin D, ethidium bromide, emetine, mitomycin, etoposide, tenoposide, vincristine, vinblastine, colchicin, doxorubicin, daunorubicin, dihydroxy anthracin didne, mitoxantrone, mithramycin, actinomycin D, 1-dehydrotestosterone, glucocorticoids, procaine, tetracaine, lidocaine, propranolol, and puromycin and analogs or homologs thereof.

A targeting cargo may be any molecule or agent that directs a peptide-polynucleotide complex of the invention to a cell. A targeting cargo may be directed to a eukaryotic target cell or a prokaryotic target cell. Non-limiting examples of targeting agents may include an antibody or an antibody fragment, a receptor ligand, a small molecule, a peptide, a polypeptide, a lipid, a carbohydrate, a nucleic acid, a siRNA, a shRNA, an antisense RNA, a dendrimer, a microbubble, or an aptamer.

The means by which a cargo complex is bound to a peptide of the invention can and will vary depending on the embodiment. A cargo complex may be bound to a peptide of the invention by any means known in the art, including covalently or non-covalently.

### 7.2.2. Polynucleotide

In another aspect, a peptide-polynucleotide complex of the invention comprises a polynucleotide. A polynucleotide may be single stranded, double stranded, or a combination thereof. In some embodiments, a polynucleotide is double stranded. In other embodiments, a polynucleotide is single stranded. In yet other embodiments, a polynucleotide is a combination of single stranded and double stranded.

A polynucleotide of the invention may comprise a ribonucleic acid (RNA), a deoxyribonucleic acid (DNA), or a combination of RNA and DNA. Additionally, a polynucleotide may comprise modified nucleic acid bases, such as modified DNA bases or modified RNA bases. Modifications may occur at, but are not restricted to, the sugar 2′ position, the C-5 position of pyrimidines, and the 8-position of purines. Examples of suitable modified DNA or RNA bases include 2′-fluoro nucleotides, 2′-amino nucleotides, 5′-aminoallyl-2′-fluoro nucleotides and phosphorothioate nucleotides (monothiophosphate and dithiophosphate). Alternatively, a polynucleotide may be a nucleotide mimic. Examples of nucleotide mimics include locked nucleic acids (LNA), peptide nucleic acids (PNA), and phosphorodiamidate morpholino oligomers (PMO).

In some embodiments, a polynucleotide of the invention is a combination of RNA and DNA. In other embodiments, a polynucleotide comprises DNA. When a polynucleotide is DNA, the polynucleotide may comprise an expression cassette. As used herein, an “expression cassette” is a nucleic acid construct comprising a nucleic acid sequence encoding a protein or peptide operably linked to a promoter. In certain embodiments, a nucleic acid construct further comprises additional regulatory sequences. A non-limiting example of an additional regulatory sequence includes a transcription termination sequence. Other additional regulatory sequences are known in the art. As used herein, the term promoter may mean a synthetic or naturally-derived molecule capable of conferring or activating expression of a target nucleic acid sequence in a cell. A promoter may be the promoter normally associated with a DNA polynucleotide of the invention, or may be a heterologous promoter. A heterologous promoter may be derived from such sources as viruses, bacteria, fungi, plants, insects, and animals. A promoter may regulate the expression of a DNA sequence constitutively or differentially with respect to the cell, the tissue or organ in which expression occurs. Or, a promoter may regulate expression with respect to developmental stage, or in response to external stimuli such as physiological stresses, pathogens, metal ions, or inducing agents or activators (i.e. an inducible promoter). Non-limiting representative examples of promoters may include the bacteriophage T7 promoter, bacteriophage T3 promoter, SP6 promoter, HSP70 basal promoter, lac operator-promoter, tac promoter, SV40 late promoter, SV40 early promoter, RSV-LTR promoter, CMV IE promoter, a promoter comprising the tetracycline response element (TRE) nucleic acid sequence, and the CMV IE promoter. In some alternatives of these embodiments, a DNA polynucleotide of the invention is incorporated into a vector. One of skill in the art would be able to construct a vector through standard recombinant techniques (see, for example, Sambrook et al., 2001 and Ausubel et al., 1996, both incorporated herein by reference). Vectors include but are not limited to plasmids, cosmids, transposable elements, viruses (bacteriophage, animal viruses, and plant viruses), and artificial chromosomes (e.g., YACs), such as retroviral vectors (e.g., derived from Moloney murine leukemia virus vectors (MoMLV), MSCV, SFFV, MPSV, SNV etc.), lentiviral vectors (e.g., derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus, herpes virus vectors, vaccinia virus vectors, Harvey murine sarcoma virus vectors, murine mammary tumor virus vectors, and Rous sarcoma virus vectors.

In yet other embodiments, a polynucleotide comprises RNA. Non-limiting examples of RNA sequences may include mRNA capable of encoding a protein, and non-coding RNA such as tRNA, rRNA, snoRNAs, microRNAs, siRNAs, saRNA, piRNAs and the long noncoding RNA (lncRNA). For instance, a nucleic acid may comprise mRNA. In some embodiments, when a nucleic acid comprises mRNA, the mRNA molecule may be 5′ capped, polyadenylated, or capped and polyadenylated. Alternatively, a mRNA molecule may comprise an internal ribosomal entry sites (IRES) for translation of an internal open reading frame of the mRNA.

In certain embodiments, a polynucleotide comprises non-coding RNA capable of regulating or inhibiting the expression of a nucleic acid sequence expressed in a cell. Non-limiting examples of non-coding RNA capable of regulating or inhibiting the expression of a nucleic acid sequence expressed in a cell include microRNAs (also known as miRNAs), siRNAs, piRNAs and lncRNAs. In general, transfection of a cell with a non-coding RNA capable of regulating or inhibiting the expression of a nucleic acid sequence may lead to cleavage of the nucleic acid sequence, may enhance, prevent, or disrupt translation of the nucleic acid sequence into a protein, or may regulate the transcription of a nucleic acid sequence.

In some embodiments, a polynucleotide of the invention comprises a non-coding RNA capable of disrupting expression of a nucleic acid sequence expressed in a cell. As used herein, “disrupting expression of a nucleic acid sequence” may be used to describe any decrease in the expression level of a nucleic acid sequence, or a protein translated from the nucleic acid sequence, when compared to a level of expression of the nucleic acid sequence in a cell that was not treated with a peptide-polynucleotide complex of the invention. In some alternatives of the embodiments, a polynucleotide comprises a short interfering RNA (siRNA).

In general, a siRNA comprises a double-stranded RNA molecule that ranges from about 15 to about 29 nucleotides in length. In some embodiments, the siRNA may be 16, 17, 18, 19, 20, 21, 22, 23, 24, 25, 26, 27, 28 or 29 nucleotides in length. In other embodiments, the siRNA may be about 16 to about 18, about 17 to about 19, about 21 to about 23, about 24 to about 27, or about 27 to about 29 nucleotides in length. In some embodiments, the siRNA may be about 21 nucleotides in length. A siRNA may optionally further comprise one or two single-stranded overhangs, e.g., a 5′ overhang on one or both ends, a 3′ overhang on one or both ends, or a combination thereof. The siRNA may be formed from two RNA molecules that hybridize together or, alternatively, may be generated from a short hairpin RNA (shRNA) (see below). In some embodiments, the two strands of the siRNA may be completely complementary, such that no mismatches or bulges exist in the duplex formed between the two sequences. In other embodiments, the two strands of the siRNA may be substantially complementary, such that one or more mismatches and/or bulges may exist in the duplex formed between the two sequences. In certain embodiments, one or both of the 5′ ends of the siRNA may have a phosphate group, while in other embodiments one or both of the 5′ ends lack a phosphate group. In other embodiments, one or both of the 3′ ends of the siRNA may have a hydroxyl group, while in other embodiments one or both of the 5′ ends lack a hydroxyl group.

One strand of the siRNA, which is referred to as the “antisense strand” or “guide strand,” includes a portion that hybridizes with a target transcript. A target transcript refers to a nucleic acid sequence expressed by a cell for which it is desired expression be disrupted. In the context of a therapeutic composition of the invention, disrupting expression of a target transcript may produce a beneficial effect. In some embodiments, the antisense strand of the siRNA may be completely complementary with a region of the target transcript, i.e., it hybridizes to the target transcript without a single mismatch or bulge over a target region between about 15 and about 29 nucleotides in length, at least 16 nucleotides in length, and about 18-20 nucleotides in length. In other embodiments, the antisense strand may be substantially complementary to the target region, i.e., one or more mismatches and/or bulges may exist in the duplex formed by the antisense strand and the target transcript. Typically, siRNAs are targeted to exonic sequences of the target transcript. Those of skill in the art are familiar with programs, algorithms, and/or commercial services that design siRNAs for target transcripts. An exemplary example is the Rosetta siRNA Design Algorithm (Rosetta Inpharmatics, North Seattle, Wash.), MISSION® siRNA (Sigma-Aldrich, St. Louis, Mo.) and siGENOME siRNA (Thermo Scientific). The siRNA may be enzymatically synthesized in vitro using methods well known to those of skill in the art. Alternatively, the siRNA may be chemically synthesized using oligonucleotide synthesis techniques that are well known in the art.

In some embodiments, a polynucleotide of the invention comprises a non-coding RNA capable of disrupting the expression of a nucleic acid sequence encoding KRAS. In some embodiments, the non-coding RNA is siRNA. In some embodiments, the target sequence of human KRAS mRNA does not encode G12, G13, or Q61 with reference to the wildtype human KRAS protein or a mutant amino acid at position 12, 13, or 61 with reference to the wildtype human KRAS protein. In some embodiments, the amino acid sequence of wildtype human KRAS protein is:

(SEQ ID NO: 4)
MTEYKLVVVGAGGVGKSALTIQLIQNHFVDEYDPTIEDSYRKQVV
IDGETCLLDILDTAGQEEYSAMRDQYMRTGEGFLCVFAINNTKSF
EDIHHYREQIKRVKDSEDVPMVLVGNKCDLPSRTVDTKQAQDLAR
SYGIPFIETSAKTRQRVEDAFYTLVREIRQYRLKKISKEEKTPGC
VKIKKCIIM

Exemplary siRNAs compatible with the polypeptide-polynucleotide complex disclosed herein are shown in Table 1 below.

TABLE 1
Selected siRNAs

| | | SEQ | | SEQ |
|---|---|---|---|---|
| | sense strand | ID | antisense strand | ID |
| name | sequence (5'-3') | NO | sequence (5'-3') | NO |
| 66 | CGGCUCGGCCAGUACUCCC | 5 | GGGAGUACUGGCCGAGCCG | 101 |
| 245 | UUGACGAUACAGCUAAUUC | 6 | GAAUUAGCUGUAUCGUCAA | 102 |
| 246 | UGACGAUACAGCUAAUUCA | 7 | UGAAUUAGCUGUAUCGUCA | 103 |
| 247 | GACGAUACAGCUAAUUCAG | 8 | CUGAAUUAGCUGUAUCGUC | 104 |
| 248 | ACGAUACAGCUAAUUCAGA | 9 | UCUGAAUUAGCUGUAUCGU | 105 |
| 250 | GAUACAGCUAAUUCAGAAU | 10 | AUUCUGAAUUAGCUGUAUC | 106 |
| 277 | GGACGAAUAUGAUCCAACA | 11 | UGUUGGAUCAUAUUCGUCC | 107 |
| 278 | GACGAAUAUGAUCCAACAA | 12 | UUGUUGGAUCAUAUUCGUC | 108 |
| 279 | ACGAAUAUGAUCCAACAAU | 13 | AUUGUUGGAUCAUAUUCGU | 109 |
| 280 | CGAAUAUGAUCCAACAAUA | 14 | UAUUGUUGGAUCAUAUUCG | 110 |
| 342 | GUCUCUUGGAUAUUCUCGA | 15 | UCGAGAAUAUCCAAGAGAC | 111 |
| 349 | GGAUAUUCUCGACACAGCA | 16 | UGCUGUGUCGAGAAUAUCC | 112 |
| 386 | GCAAUGAGGGACCAGUACA | 17 | UGUACUGGUCCCUCAUUGC | 113 |
| 387 | CAAUGAGGGACCAGUACAU | 18 | AUGUACUGGUCCCUCAUUG | 114 |
| 463 | AGAUAUUCACCAUUAUAGA | 19 | UCUAUAAUGGUGAAUAUCU | 115 |
| 464 | GAUAUUCACCAUUAUAGAG | 20 | CUCUAUAAUGGUGAAUAUC | 116 |
| 465 | AUAUUCACCAUUAUAGAGA | 21 | UCUCUAUAAUGGUGAAUAU | 117 |
| 466 | UAUUCACCAUUAUAGAGAA | 22 | UUCUCUAUAAUGGUGAAUA | 118 |
| 517 | ACCUAUGGUCCUAGUAGGA | 23 | UCCUACUAGGACCAUAGGU | 119 |
| 583 | GGACUUAGCAAGAAGUUAU | 24 | AUAACUUCUUGCUAAGUCC | 120 |
| 656 | UUCUAUACAUUAGUUCGAG | 25 | CUCGAACUAAUGUAUAGAA | 121 |
| 659 | UAUACAUUAGUUCGAGAAA | 26 | UUUCUCGAACUAAUGUAUA | 122 |
| 663 | CAUUAGUUCGAGAAAUUCG | 27 | CGAAUUUCUCGAACUAAUG | 123 |
| 664 | AUUAGUUCGAGAAAUUCGA | 28 | UCGAAUUUCUCGAACUAAU | 124 |
| 665 | UUAGUUCGAGAAAUUCGAA | 29 | UUCGAAUUUCUCGAACUAA | 125 |
| 667 | AGUUCGAGAAAUUCGAAAA | 30 | UUUUCGAAUUUCUCGAACU | 126 |
| 673 | AGAAAUUCGAAAACAUAAA | 31 | UUUAUGUUUUCGAAUUUCU | 127 |
| 676 | AAUUCGAAAACAUAAAGAA | 32 | UUCUUUAUGUUUUCGAAUU | 128 |
| 808 | UGUACAUUACACUAAAUUA | 33 | UAAUUUAGUGUAAUGUACA | 129 |
| 1033 | GCAGUUGAUUACUUCUUAU | 34 | AUAAGAAGUAAUCAACUGC | 130 |
| 1102 | GAAUCAUCCCUAUUCUGUG | 35 | CACAGAAUAGGGAUGAUUC | 131 |
| 1239 | GCAUCAUGUCCUAUAGUUU | 36 | AAACUAUAGGACAUGAUGC | 132 |
| 1312 | CACUGCUAUUAGUCAUGGU | 37 | ACCAUGACUAAUAGCAGUG | 133 |
| 1315 | UGCUAUUAGUCAUGGUCAC | 38 | GUGACCAUGACUAAUAGCA | 134 |
| 1426 | ACAUUAGAUAAAUUACUAU | 39 | AUAGUAAUUUAUCUAAUGU | 135 |
| 1482 | AGUAUGAAAUGGGGAUUAU | 40 | AUAAUCCCCAUUUCAUACU | 136 |
| 1508 | ACCAUUUUGGGGCUAUAUU | 41 | AAUAUAGCCCCAAAAUGGU | 137 |
| 1706 | UUUGGGCCAGUUAUAGCUU | 42 | AAGCUAUAACUGGCCCAAA | 138 |
| 1912 | GGUCCUGCUGACAAAUCAA | 43 | UUGAUUUGUCAGCAGGACC | 139 |
| 2070 | ACAAUCUCUAGGUUUGGCU | 44 | AGCCAAACCUAGAGAUUGU | 140 |
| 2076 | UCUAGGUUUGGCUAGUUCU | 45 | AGAACUAGCCAAACCUAGA | 141 |
| 2081 | GUUUGGCUAGUUCUCUUAA | 46 | UUAAGAGAACUAGCCAAAC | 142 |
| 2083 | UUGGCUAGUUCUCUUAACA | 47 | UGUUAAGAGAACUAGCCAA | 143 |
| 2234 | GCAUGGUGAGGUGAAAGUA | 48 | UACUUUCACCUCACCAUGC | 144 |
| 2235 | CAUGGUGAGGUGAAAGUAU | 49 | AUACUUUCACCUCACCAUG | 145 |
| 2272 | GUGACUUAGGUUCUAGAUA | 50 | UAUCUAGAACCUAAGUCAC | 146 |
| 2273 | UGACUUAGGUUCUAGAUAG | 51 | CUAUCUAGAACCUAAGUCA | 147 |
| 2316 | AGGACAUCACUUACUAUCC | 52 | GGAUAGUAAGUGAUGUCCU | 148 |
| 2417 | GGAUACACUUAUUUGUCAA | 53 | UUGACAAAUAAGUGUAUCC | 149 |
| 2457 | UUUAACCUAUGUUACACCA | 54 | UGGUGUAACAUAGGUUAAA | 150 |
| 2458 | UUAACCUAUGUUACACCAU | 55 | AUGGUGUAACAUAGGUUAA | 151 |
| 2492 | UGGGCAAAAUUGUGCAAGA | 56 | UCUUGCACAAUUUUGCCCA | 152 |
| 2524 | UUUGAAUAUCCAUUCUCGU | 57 | ACGAGAAUGGAUAUUCAAA | 153 |
| 2630 | UCCCCUAACCAUAAGAUUU | 58 | AAAUCUUAUGGUUAGGGGA | 154 |
| 2631 | CCCCUAACCAUAAGAUUUA | 59 | UAAAUCUUAUGGUUAGGGG | 155 |
| 2632 | CCCUAACCAUAAGAUUUAC | 60 | GUAAAUCUUAUGGUUAGGG | 156 |
| 2633 | CCUAACCAUAAGAUUUACU | 61 | AGUAAAUCUUAUGGUUAGG | 157 |
| 2753 | AGGGAUUUGACCUAAUCAC | 62 | GUGAUUAGGUCAAAUCCCU | 158 |
| 2760 | UGACCUAAUCACUAAUUUU | 63 | AAAAUUAGUGAUUAGGUCA | 159 |
| 2812 | CUGCCCAAUCCAUUAGCGA | 64 | UCGCUAAUGGAUUGGGCAG | 160 |
| 2813 | UGCCCAAUCCAUUAGCGAC | 65 | GUCGCUAAUGGAUUGGGCA | 161 |
| 2814 | GCCCAAUCCAUUAGCGACA | 66 | UGUCGCUAAUGGAUUGGGC | 162 |
| 2815 | CCCAAUCCAUUAGCGACAG | 67 | CUGUCGCUAAUGGAUUGGG | 163 |
| 2816 | CCAAUCCAUUAGCGACAGU | 68 | ACUGUCGCUAAUGGAUUGG | 164 |
| 2817 | CAAUCCAUUAGCGACAGUA | 69 | UACUGUCGCUAAUGGAUUG | 165 |
| 2819 | AUCCAUUAGCGACAGUAGG | 70 | CCUACUGUCGCUAAUGGAU | 166 |
| 2822 | CAUUAGCGACAGUAGGAUU | 71 | AAUCCUACUGUCGCUAAUG | 167 |
| 2851 | GGUAUGAAUAGACAGAACC | 72 | GGUUCUGUCUAUUCAUACC | 168 |
| 2857 | AAUAGACAGAACCCUAUCC | 73 | GGAUAGGGUUCUGUCUAUU | 169 |
| 2943 | UCCUGGUAACAGUAAUACA | 74 | UGUAUUACUGUUACCAGGA | 170 |
| 3755 | AGUUUUAUCUGGGUACAAA | 75 | UUUGUACCCAGAUAAAACU | 171 |
| 3880 | GUAGGGUGUUAAGACUUAC | 76 | GUAAGUCUUAACACCCUAC | 172 |
| 3962 | GGGGAUAUUUAGGCCUCUU | 77 | AAGAGGCCUAAAUAUCCCC | 173 |
| 4181 | CUUAGGCAUUAACAUGUUU | 78 | AAACAUGUUAAUGCCUAAG | 174 |
| 4303 | CCUAACUUUUAUAGGUUAU | 79 | AUAACCUAUAAAAGUUAGG | 175 |
| 4722 | GAAUAGUCAUAACUAGAUU | 80 | AAUCUAGUUAUGACUAUUC | 176 |
| 4729 | CAUAACUAGAUUAAGAUCU | 81 | AGAUCUUAAUCUAGUUAUG | 177 |
| 4781 | GGGAUAAUGAUAGGUAAUU | 82 | AAUUACCUAUCAUUAUCCC | 178 |
| 4782 | GGAUAAUGAUAGGUAAUUU | 83 | AAAUUACCUAUCAUUAUCC | 179 |
| 4890 | GUGUUUUAUCCGAAAGUUU | 84 | AAACUUUCGGAUAAAACAC | 180 |
| 4891 | UGUUUUAUCCGAAAGUUUC | 85 | GAAACUUUCGGAUAAAACA | 181 |
| 4896 | UAUCCGAAAGUUUCCAAUU | 86 | AAUUGGAAACUUUCGGAUA | 182 |
| 4899 | CCGAAAGUUUCCAAUUCCA | 87 | UGGAAUUGGAAACUUUCGG | 183 |
| 4967 | GCCAAUUUCUUACUAGUAC | 88 | GUACUAGUAAGAAAUUGGC | 184 |
| 4968 | CCAAUUUCUUACUAGUACU | 89 | AGUACUAGUAAGAAAUUGG | 185 |
| 5115 | CCAUCAUUUGGUUGCGCUG | 90 | CAGCGCAACCAAAUGAUGG | 186 |
| 5118 | UCAUUUGGUUGCGCUGACC | 91 | GGUCAGCGCAACCAAAUGA | 187 |
| 5134 | ACCUAGGAAUGUUGGUCAU | 92 | AUGACCAACAUUCCUAGGU | 188 |
| 5135 | CCUAGGAAUGUUGGUCAUA | 93 | UAUGACCAACAUUCCUAGG | 189 |
| 5136 | CUAGGAAUGUUGGUCAUAU | 94 | AUAUGACCAACAUUCCUAG | 190 |
| 5137 | UAGGAAUGUUGGUCAUAUC | 95 | GAUAUGACCAACAUUCCUA | 191 |
| 5139 | GGAAUGUUGGUCAUAUCAA | 96 | UUGAUAUGACCAACAUUCC | 192 |
| 5258 | ACUGUACUACUCCUAAUUA | 97 | UAAUUAGGAGUAGUACAGU | 193 |
| 5259 | CUGUACUACUCCUAAUUAU | 98 | AUAAUUAGGAGUAGUACAG | 194 |
| 5260 | UGUACUACUCCUAAUUAUU | 99 | AAUAAUUAGGAGUAGUACA | 195 |
| 5261 | GUACUACUCCUAAUUAUUG | 100 | CAAUAAUUAGGAGUAGUAC | 196 |

In some embodiments, the siRNAs disclosed herein are modified. In some embodiments, the modifications are selected from the group consisting of 2′-methoxy (2′-OMe), 2′-fluoro (2′-F), 2′-O-methoxyethyl(2′-O-MOE), 5′-vinylphosphonate, phosphorothioate (PTO), locked nucleic acid (LNA), locked nucleic acid (UNA), glycol nucleic acid (GNA), and DNA.

In some embodiments, the modifications of the sense strand comprise PTO at positions 1 and/or 2. In some embodiments, the modifications of the sense strand comprise 2′-F at one or more positions of 3, 7-9, 12, and 17. In some embodiments, the modifications of the sense strand comprise 2′-OMe at one or more positions of 1, 2, 4-6, 10, 11, 13-16, 18, and 19. In some embodiments, the modifications of the antisense strand comprise PTO at one or more positions of 1, 2, 19, and 20. In some embodiments, the modifications of the antisense strand comprise 2′-F at positions 2 and/or 14. In some embodiments, the modifications of the antisense strand comprise 2′-OMe at any position of 1, 3-13, and 15-21.

In some embodiments, the last nucleotide of the sense strand is adenine (A). In some embodiments, the first nucleotide of the antisense strand is uracil (U). In some embodiments, the last nucleotide of the sense strand is uracil (U). In some embodiments, the first nucleotide of the antisense strand is adenine (A). In some embodiments, the last nucleotide of the sense strand is cytosine (C). In some embodiments, the first nucleotide of the antisense strand is guanine (G). In some embodiments, the last nucleotide of the sense strand is guanine (G). In some embodiments, the first nucleotide of the antisense strand is cytosine (C).

Exemplary modified siRNAs compatible with the polypeptide-polynucleotide complex disclosed herein are shown in Table 2 below. n=2′O-methyl RNA, Nf=2′-fluoro RNA, s=phosphorothioate.

TABLE 2
Modification of Selected siRNAs

| | sense strand sequence | SEQ ID | antisense strand sequence | SEQ ID |
|---|---|---|---|---|
| name | (5'-3') | NO | (5'-3') | NO |
| 66 | csgsGfcucGfGfCfcaGfuacuCfca | 197 | usGfsgaguacuggccGfagccgscsc | 293 |
| 245 | ususGfacgAfUfAfcaGfcuaaUfua | 198 | usAfsauuagcuguauCfgucaasgsg | 294 |
| 246 | usgsAfcgaUfAfCfagCfuaauUfca | 199 | usGfsaauuagcuguaUfcgucasasg | 295 |
| 247 | gsasCfgauAfCfAfgcUfaauuCfaa | 200 | usUfsgaauuagcuguAfucgucsasa | 296 |
| 248 | ascsGfauaCfAfGfcuAfauucAfga | 201 | usCfsugaauuagcugUfaucguscsa | 297 |
| 250 | gsasUfacaGfCfUfaaUfucagAfaa | 202 | usUfsucugaauuagcUfguaucsgsu | 298 |
| 277 | gsgsAfcgaAfUfAfugAfuccaAfca | 203 | usGfsuuggaucauauUfcguccsasc | 299 |
| 278 | gsasCfgaaUfAfUfgaUfccaaCfaa | 204 | usUfsguuggaucauaUfucgucscsa | 300 |
| 279 | ascsGfaauAfUfGfauCfcaacAfaa | 205 | usUfsuguuggaucauAfuucguscsc | 301 |
| 280 | csgsAfauaUfGfAfucCfaacaAfua | 206 | usAfsuuguuggaucaUfauucgsusc | 302 |
| 342 | gsusCfucuUfGfGfauAfuucuCfga | 207 | usCfsgagaauauccaAfgagacsasg | 303 |
| 349 | gsgsAfuauUfCfUfcgAfcacaGfca | 208 | usGfscugugucgagaAfuauccsasa | 304 |
| 386 | gscsAfaugAfGfGfgaCfcaguAfca | 209 | usGfsuacuggucccuCfauugcsasc | 305 |
| 387 | csasAfugaGfGfGfacCfaguaCfaa | 210 | usUfsguacuggucccUfcauugscsa | 306 |
| 463 | asgsAfuauUfCfAfccAfuuauAfga | 211 | usCfsuauaauggugaAfuaucususc | 307 |
| 464 | gsasUfauuCfAfCfcaUfuauaGfaa | 212 | usUfscuauaauggugAfauaucsusu | 308 |
| 465 | asusAfuucAfCfCfauUfauagAfga | 213 | usCfsucuauaaugguGfaauauscsu | 309 |
| 466 | usasUfucaCfCfAfuuAfuagaGfaa | 214 | usUfscucuauaauggUfgaauasusc | 310 |
| 517 | ascsCfuauGfGfUfccUfaguaGfga | 215 | usCfscuacuaggaccAfuaggusasc | 311 |
| 583 | gsgsAfcuuAfGfCfaaGfaaguUfaa | 216 | usUfsaacuucuugcuAfaguccsusg | 312 |
| 656 | ususCfuauAfCfAfuuAfguucGfaa | 217 | usUfscgaacuaauguAfuagaasgsg | 313 |
| 659 | usasUfacaUfUfAfguUfcgagAfaa | 218 | usUfsucucgaacuaaUfguauasgsa | 314 |
| 663 | csasUfuagUfUfCfgaGfaaauUfca | 219 | usGfsaauuucucgaaCfuaaugsusa | 315 |
| 664 | asusUfaguUfCfGfagAfaauuCfga | 220 | usCfsgaauuucucgaAfcuaausgsu | 316 |
| 665 | ususAfguuCfGfAfgaAfauucGfaa | 221 | usUfscgaauuucucgAfacuaasusg | 317 |
| 667 | asgsUfucgAfGfAfaaUfucgaAfaa | 222 | usUfsuucgaauuucuCfgaacusasa | 318 |
| 673 | asgsAfaauUfCfGfaaAfacauAfaa | 223 | usUfsuauguuuucgaAfuuucuscsg | 319 |
| 676 | asasUfucgAfAfAfacAfuaaaGfaa | 224 | usUfscuuuauguuuuCfgaauususc | 320 |
| 808 | usgsUfacaUfUfAfcaCfuaaaUfua | 225 | usAfsauuuaguguaaUfguacasasa | 321 |
| 1033 | gscsAfguuGfAfUfuaCfuucuUfaa | 226 | usUfsaagaaguaaucAfacugcsasu | 322 |
| 1102 | gsasAfucaUfCfCfcuAfuucuGfua | 227 | usAfscagaauagggaUfgauucsasa | 323 |
| 1239 | gscsAfucaUfGfUfccUfauagUfua | 228 | usAfsacuauaggacaUfgaugcscsu | 324 |
| 1312 | csasCfugcUfAfUfuaGfucauGfga | 229 | usCfscaugacuaauaGfcagugsgsa | 325 |
| 1315 | usgsCfuauUfAfGfucAfugguCfaa | 230 | usUfsgaccaugacuaAfuagcasgsu | 326 |
| 1426 | ascsAfuuaGfAfUfaaAfuuacUfaa | 231 | usUfsaguaauuuaucUfaaugusgsa | 327 |
| 1482 | asgsUfaugAfAfAfugGfggauUfaa | 232 | usUfsaauccccauuuCfauacusgsg | 328 |
| 1508 | ascsCfauuUfUfGfggGfcuauAfua | 233 | usAfsuauagccccaaAfauggususg | 329 |
| 1706 | ususUfgggCfCfAfguUfauagCfua | 234 | usAfsgcuauaacuggCfccaaasusa | 330 |
| 1912 | gsgsUfccuGfCfUfgaCfaaauCfaa | 235 | usUfsgauuugucagcAfggaccsasc | 331 |
| 2070 | ascsAfaucUfCfUfagGfuuugGfca | 236 | usGfsccaaaccuagaGfauugusasa | 332 |
| 2076 | uscsUfaggUfUfUfggCfuaguUfca | 237 | usGfsaacuagccaaaCfcuagasgsa | 333 |
| 2081 | gsusUfuggCfUfAfguUfcucuUfaa | 238 | usUfsaagagaacuagCfcaaacscsu | 334 |
| 2083 | ususGfgcuAfGfUfucUfcuuaAfca | 239 | usGfsuuaagagaacuAfgccaasasc | 335 |
| 2234 | gscsAfuggUfGfAfggUfgaaaGfua | 240 | usAfscuuucaccucaCfcaugcscsa | 336 |
| 2235 | csasUfgguGfAfGfguGfaaagUfaa | 241 | usUfsacuuucaccucAfccaugscsc | 337 |
| 2272 | gsusGfacuUfAfGfguUfcuagAfua | 242 | usAfsucuagaaccuaAfgucacscsu | 338 |
| 2273 | usgsAfcuuAfGfGfuuCfuagaUfaa | 243 | usUfsaucuagaaccuAfagucascsc | 339 |
| 2316 | asgsGfacaUfCfAfcuUfacuaUfca | 244 | usGfsauaguaagugaUfguccuscsa | 340 |
| 2417 | gsgsAfuacAfCfUfuaUfuuguCfaa | 245 | usUfsgacaaauaaguGfuauccsusu | 341 |
| 2457 | ususUfaacCfUfAfugUfuacaCfca | 246 | usGfsguguaacauagGfuuaaasasa | 342 |
| 2458 | ususAfaccUfAfUfguUfacacCfaa | 247 | usUfsgguguaacauaGfguuaasasa | 343 |
| 2492 | usgsGfgcaAfAfAfuuGfugcaAfga | 248 | usCfsuugcacaauuuUfgcccasasg | 344 |
| 2524 | ususUfgaaUfAfUfccAfuucuCfga | 249 | usCfsgagaauggauaUfucaaasusa | 345 |
| 2630 | uscsCfccuAfAfCfcaUfaagaUfua | 250 | usAfsaucuuaugguuAfggggasasu | 346 |
| 2631 | cscsCfcuaAfCfCfauAfagauUfua | 251 | usAfsaaucuuaugguUfaggggsasa | 347 |
| 2632 | cscsCfuaaCfCfAfuaAfgauuUfaa | 252 | usUfsaaaucuuauggUfuagggsgsa | 348 |
| 2633 | cscsUfaacCfAfUfaaGfauuuAfca | 253 | usGfsuaaaucuuaugGfuuaggsgsg | 349 |
| 2753 | asgsGfgauUfUfGfacCfuaauCfaa | 254 | usUfsgauuaggucaaAfucccususu | 350 |
| 2760 | usgsAfccuAfAfUfcaCfuaauUfua | 255 | usAfsaauuagugauuAfggucasasa | 351 |
| 2812 | csusGfcccAfAfUfccAfuuagCfga | 256 | usCfsgcuaauggauuGfggcagscsa | 352 |
| 2813 | usgsCfccaAfUfCfcaUfuagcGfaa | 257 | usUfscgcuaauggauUfgggcasgsc | 353 |
| 2814 | gscsCfcaaUfCfCfauUfagcgAfca | 258 | usGfsucgcuaauggaUfugggcsasg | 354 |
| 2815 | cscsCfaauCfCfAfuuAfgcgaCfaa | 259 | usUfsgucgcuaauggAfuugggscsa | 355 |
| 2816 | cscsAfaucCfAfUfuaGfcgacAfga | 260 | usCfsugucgcuaaugGfauuggsgsc | 356 |
| 2817 | csasAfuccAfUfUfagCfgacaGfua | 261 | usAfscugucgcuaauGfgauugsgsg | 357 |
| 2819 | asusCfcauUfAfGfcgAfcaguAfga | 262 | usCfsuacugucgcuaAfuggaususg | 358 |
| 2822 | csasUfuagCfGfAfcaGfuaggAfua | 263 | usAfsuccuacugucgCfuaaugsgsa | 359 |
| 2851 | gsgsUfaugAfAfUfagAfcagaAfca | 264 | usGfsuucugucuauuCfauaccsasg | 360 |
| 2857 | asasUfagaCfAfGfaaCfccuaUfca | 265 | usGfsauaggguucugUfcuauuscsa | 361 |
| 2943 | uscsCfuggUfAfAfcaGfuaauAfca | 266 | usGfsuauuacuguuaCfcaggasgsu | 362 |
| 3755 | asgsUfuuuAfUfCfugGfguacAfaa | 267 | usUfsuguacccagauAfaaacusasu | 363 |
| 3880 | gsusAfgggUfGfUfuaAfgacuUfaa | 268 | usUfsaagucuuaacaCfccuacscsu | 364 |
| 3962 | gsgsGfgauAfUfUfuaGfgccuCfua | 269 | usAfsgaggccuaaauAfuccccsusc | 365 |
| 4181 | csusUfaggCfAfUfuaAfcaugUfua | 270 | usAfsacauguuaaugCfcuaagsusc | 366 |
| 4303 | cscsUfaacUfUfUfuaUfagguUfaa | 271 | usUfsaaccuauaaaaGfuuaggsusu | 367 |
| 4722 | gsasAfuagUfCfAfuaAfcuagAfua | 272 | usAfsucuaguuaugaCfuauucsusu | 368 |
| 4729 | csasUfaacUfAfGfauUfaagaUfca | 273 | usGfsaucuuaaucuaGfuuaugsasc | 369 |
| 4781 | gsgsGfauaAfUfGfauAfgguaAfua | 274 | usAfsuuaccuaucauUfaucccsasa | 370 |
| 4782 | gsgsAfuaaUfGfAfuaGfguaaUfua | 275 | usAfsauuaccuaucaUfuauccscsa | 371 |
| 4890 | gsusGfuuuUfAfUfccGfaaagUfua | 276 | usAfsacuuucggauaAfaacacsusg | 372 |
| 4891 | usgsUfuuuAfUfCfcgAfaaguUfua | 277 | usAfsaacuuucggauAfaaacascsu | 373 |
| 4896 | usasUfccgAfAfAfguUfuccaAfua | 278 | usAfsuuggaaacuuuCfggauasasa | 374 |
| 4899 | cscsGfaaaGfUfUfucCfaauuCfca | 279 | usGfsgaauuggaaacUfuucggsasu | 375 |
| 4967 | gscsCfaauUfUfCfuuAfcuagUfaa | 280 | usUfsacuaguaagaaAfuuggcsasc | 376 |
| 4968 | cscsAfauuUfCfUfuaCfuaguAfca | 281 | usGfsuacuaguaagaAfauuggscsa | 377 |
| 5115 | cscsAfucaUfUfUfggUfugcgCfua | 282 | usAfsgcgcaaccaaaUfgauggsasa | 378 |
| 5118 | uscsAfuuuGfGfUfugCfgcugAfca | 283 | usGfsucagcgcaaccAfaaugasusg | 379 |
| 5134 | ascsCfuagGfAfAfugUfugguCfaa | 284 | usUfsgaccaacauucCfuagguscsa | 380 |
| 5135 | cscsUfaggAfAfUfguUfggucAfua | 285 | usAfsugaccaacauuCfcuaggsusc | 381 |
| 5136 | csusAfggaAfUfGfuuGfgucaUfaa | 286 | usUfsaugaccaacauUfccuagsgsu | 382 |
| 5137 | usasGfgaaUfGfUfugGfucauAfua | 287 | usAfsuaugaccaacaUfuccuasgsg | 383 |
| 5139 | gsgsAfaugUfUfGfguCfauauCfaa | 288 | usUfsgauaugaccaaCfauuccsusa | 384 |
| 5258 | ascsUfguaCfUfAfcuCfcuaaUfua | 289 | usAfsauuaggaguagUfacagususc | 385 |
| 5259 | csusGfuacUfAfCfucCfuaauUfaa | 290 | usUfsaauuaggaguaGfuacagsusu | 386 |
| 5260 | usgsUfacuAfCfUfccUfaauuAfua | 291 | usAfsuaauuaggaguAfguacasgsu | 387 |
| 5261 | gsusAfcuaCfUfCfcuAfauuaUfua | 292 | usAfsauaauuaggagUfaguacsasg | 388 |

In some embodiments, the sense strand comprises a nucleotide sequence with at least at least 80% identity to the nucleotide sequence of any one of the sensen strands listed in Table 1 and Table 2. In some embodiments, the sense strand comprises a nucleotide sequence with at least at least 85% identity to the nucleotide sequence of any one of the sensen strands listed in Table 1 and Table 2. In some embodiments, the sense strand comprises a nucleotide sequence with at least at least 90% identity to the nucleotide sequence of any one of the sensen strands listed in Table 1 and Table 2. In some embodiments, the sense strand comprises a nucleotide sequence with at least at least 95% identity to the nucleotide sequence of any one of the sensen strands listed in Table 1 and Table 2. In some embodiments, the sense strand comprises a nucleotide sequence with at least at least 98% identity to the nucleotide sequence of any one of the sensen strands listed in Table 1 and Table 2. In some embodiments, the sense strand comprises a nucleotide sequence with at least at least 99% identity to the nucleotide sequence of any one of the sensen strands listed in Table 1 and Table 2. In some embodiments, the sense strand comprises a nucleotide sequence with 100% identity to the nucleotide sequence of any one of the sensen strands listed in Table 1 and Table 2.

In some embodiments, the antisense strand comprises a nucleotide sequence with at least at least 80% identity to the nucleotide sequence of any one of the antisensen strands listed in Table 1 and Table 2. In some embodiments, the antisense strand comprises a nucleotide sequence with at least at least 85% identity to the nucleotide sequence of any one of the antisensen strands listed in Table 1 and Table 2. In some embodiments, the antisense strand comprises a nucleotide sequence with at least at least 90% identity to the nucleotide sequence of any one of the antisensen strands listed in Table 1 and Table 2. In some embodiments, the antisense strand comprises a nucleotide sequence with at least at least 95% identity to the nucleotide sequence of any one of the antisensen strands listed in Table 1 and Table 2. In some embodiments, the antisense strand comprises a nucleotide sequence with at least at least 98% identity to the nucleotide sequence of any one of the antisensen strands listed in Table 1 and Table 2. In some embodiments, the antisense strand comprises a nucleotide sequence with at least at least 99% identity to the nucleotide sequence of any one of the antisensen strands listed in Table 1 and Table 2. In some embodiments, the antisense strand comprises a nucleotide sequence with 100% identity to the nucleotide sequence of any one of the antisensen strands listed in Table 1 and Table 2.

Generally speaking, the promoters utilized to direct in vivo expression of the one or more siRNA or shRNA transcription units may be promoters for RNA polymerase III (Pol III). Certain Pol III promoters, such as U6 or H1 promoters, do not require cis-acting regulatory elements within the transcribed region, and thus, are in certain embodiments. In other embodiments, promoters for Pol II may be used to drive expression of the one or more siRNA or shRNA transcription units. In some embodiments, tissue-specific, cell-specific, or inducible Pol II promoters may be used.

A construct that provides a template for the synthesis of siRNA or shRNA may be produced using standard recombinant DNA methods and inserted into any of a wide variety of different vectors suitable for expression in eukaryotic cells. Guidance may be found in Current Protocols in Molecular Biology (Ausubel et al., John Wiley & Sons, New York, 2003) or Molecular Cloning: A Laboratory Manual (Sambrook & Russell, Cold Spring Harbor Press, Cold Spring Harbor, N.Y., 3rd edition, 2001). Those of skill in the art also appreciate that vectors may comprise additional regulatory sequences (e.g., termination sequence, translational control sequence, etc.), as well as selectable marker sequences. DNA plasmids are known in the art, including those based on pBR322, PUC, and so forth. Since many expression vectors already contain a suitable promoter or promoters, it may only be necessary to insert the nucleic acid sequence that encodes the RNAi agent of interest at an appropriate location with respect to the promoter(s). Viral vectors may also be used to provide intracellular expression of RNAi agents. Suitable viral vectors include retroviral vectors, lentiviral vectors, adenoviral vectors, adeno-associated virus vectors, herpes virus vectors, and so forth. In some embodiments, the RNAi expression vector is a shRNA lentiviral-based vector or lentiviral particle, such as that provided in MISSION® TRC shRNA products (Sigma-Aldrich).

Nucleic acid sequences of the invention may be obtained using a variety of different techniques known in the art. The nucleotide sequences, as well as homologous sequences, may be isolated using standard techniques, may be synthesized using standard techniques, or may be purchased or obtained from a depository. Once the nucleotide sequence is obtained, it may be amplified for use in a variety of applications, using methods known in the art.

### 7.2.3. Polypeptide-Polynucleotide Complex

In another aspect of the invention, a polypeptide and a polynucleotide of the invention associate to form a complex. As used herein, the term “associate” may refer to the interaction of a peptide and a polynucleotide through non-covalent bonds, or to the covalent bonding of a peptide and a polynucleotide. In some embodiments, a polypeptide and a polynucleotide of the invention associate through non-covalent bonds such as a hydrogen bond, an ionic bond, a bond based on Van der Waals, a hydrophobic bond, or electrostatic interactions. For instance, a peptide of the invention may have an overall net positive charge, which may allow the peptide to associate with a polynucleotide of the invention through electrostatic interactions to form a complex of the invention. Methods for forming a polypeptide-polynucleotide complex of the invention are known in the art and further described herein.

The ratio of peptide to polynucleotide at which a peptide of the invention associates with a polynucleotide of the invention can and will vary depending on the peptide, the polynucleotide composition, or the size of the polynucleotide, and may be determined experimentally. In essence, a suitable molar ratio of a peptide of the invention to a polynucleotide of the invention may be a molar ratio wherein the peptide completely complexes the polynucleotide, while minimizing exposure of a subject to the peptide.

In some embodiments, the ratio is the molar ratio. In some embodiments, the molar ratio is about 2:1 to about 3500:1. In some embodiments, the molar ratio of peptide to polynucleotide is about 4:1 to about 1000:1. In some embodiments, the molar ratio of peptide to polynucleotide is about 10:1 to about 500:1. In some embodiments, the molar ratio of peptide to polynucleotide is about 5:1 to about 200:1. In some embodiments, the molar ratio of peptide to polynucleotide is about 50:1 to about 200:1. In some embodiments, the molar ratio of peptide to polynucleotide is about 100:1. In some embodiments, the molar ratio of peptide to polynucleotide is about 5:1.

In some embodiments, the ratio is the ratio of positively-chargeable polymer amine groups to negatively-charged nucleic acid phosphate groups. In some embodiments, the charge ratio of peptide to polynucleotide is about 6:1 to about 18:1. In some embodiments, the charge ratio of peptide to polynucleotide is about 6:1. In some embodiments, the charge ratio of peptide to polynucleotide is about 7:1. In some embodiments, the charge ratio of peptide to polynucleotide is about 8:1. In some embodiments, the charge ratio of peptide to polynucleotide is about 9:1. In some embodiments, the charge ratio of peptide to polynucleotide is about 10:1. In some embodiments, the charge ratio of peptide to polynucleotide is about 11:1. In some embodiments, the charge ratio of peptide to polynucleotide is about 12:1. In some embodiments, the charge ratio of peptide to polynucleotide is about 13:1. In some embodiments, the charge ratio of peptide to polynucleotide is about 14:1. In some embodiments, the charge ratio of peptide to polynucleotide is about 15:1. In some embodiments, the charge ratio of peptide to polynucleotide is about 16:1. In some embodiments, the charge ratio of peptide to polynucleotide is about 17:1. In some embodiments, the charge ratio of peptide to polynucleotide is about 18:1.

Methods of determining the ratio wherein the peptide is capable of completely complexing the polynucleotide are known in the art, and may include gel retardation assays as described in the examples. Methods of determining a molar ratio wherein exposure of a subject to the peptide is minimized are known in the art, and may include cytotoxicity measurements using increasing doses of the polypeptide.

A peptide-polynucleotide complex of the invention may be about 10 nm to about 500 nm in diameter. In some embodiments, the diameter of the peptide-polynucleotide complex is about 10 nm to about 300 nm. In some embodiments, the diameter of the peptide-polynucleotide complex is at least about 10 nm. In some embodiments, the diameter of the peptide-polynucleotide complex is at most about 300 nm. In some embodiments, the diameter of the peptide-polynucleotide complex is about 10 nm to about 50 nm, about 10 nm to about 100 nm, about 10 nm to about 150 nm, about 10 nm to about 200 nm, about 10 nm to about 250 nm, about 10 nm to about 300 nm, about 50 nm to about 100 nm, about 50 nm to about 150 nm, about 50 nm to about 200 nm, about 50 nm to about 250 nm, about 50 nm to about 300 nm, about 100 nm to about 150 nm, about 100 nm to about 200 nm, about 100 nm to about 250 nm, about 100 nm to about 300 nm, about 150 nm to about 200 nm, about 150 nm to about 250 nm, about 150 nm to about 300 nm, about 200 nm to about 250 nm, about 200 nm to about 300 nm, or about 250 nm to about 300 nm. In some embodiments, the diameter of the peptide-polynucleotide complex is about 10 nm, about 50 nm, about 100 nm, about 150 nm, about 200 nm, about 250 nm, or about 300 nm.

A nanoparticle of the invention may be further modified to enhance stability of the nanoparticle. For instance, a nanoparticle of the invention may be coated with albumin and/or hyaluronic acid to enhance stability. A nanoparticle of the invention coated with albumin may be about 5 to about 90 nm or more in diameter.

Particle size and/or charge may be assessed using methods known in the art. Non-limiting examples of methods of measuring the size of a particle may include dynamic light scattering, light scattering, multi-angle light scattering, field-flow fractionation systems, laser diffraction, electrozone (electric sensing zone), light obscuration—also referred to as photozone and single particle optical sensing (SPOS), sieve analysis, aerodynamic measurements, air permeability diameter, sedimentation, nanoparticle tracking analysis, electron microspcopy, atomic force microscopy, small-angle X ray scattering, flow cytometry, measuring the zeta potential of the particle, analytical ultracentrifugation or combinations thereof. In some embodiments, particle size is assessed by dynamic light scattering. In some embodiments, particle charge is assessed by measuring the zeta potential of the particle. In yet some embodiments, particle size and/or charge is assessed by dynamic light scattering or by measuring the zeta potential of the particle.

A nanoparticle of the invention may have a zeta potential of about-15 to about 20 mV, about 0 mV or more. For instance, a nanoparticle may have a zeta potential of about 1, about 2, about 3, about 4, about 5, about 6, about 7, about 8, about 9, about 10, about 11, about 12, about 13, about 14, about 15, about 16, about 17, about 18, about 19 or about 20 mV or more. In some embodiments, a nanoparticle has a zeta potential of about 1, about 2, about 3, about 4, or about 5 mV. In other embodiments, a nanoparticle has a zeta potential of about 10, 11, 12, 13, or about 14 mV. In yet other embodiments, a nanoparticle has a zeta potential of about 11, about 12, about 13, about 14, or about 15 mV. In an exemplary embodiment, a nanoparticle has a zeta potential of about 1, about 2, about 3, about 4, or about 5 mV. In other embodiments, a nanoparticle has a zeta potential of about 10, about 11, 12, about 13, or about 14 mV. In an exemplary embodiment, a nanoparticle has a zeta potential of about 3.72 mV. In another exemplary embodiment, a nanoparticle has a zeta potential of about 12 mV. In yet another exemplary embodiment, a nanoparticle has a zeta potential of about 13.1 mV.

A peptide-polynucleotide complex is capable of efficient release of the polynucleotide into the cytoplasm of a cell. A peptide-polynucleotide complex may also be capable of protecting the polynucleotide from degradation upon administration in a subject. As such, a peptide-polynucleotide nanoparticle of the invention may remain stable in the presence of serum. A nanoparticle may remain stable in the presence of serum for about 10, 20, 30, 40, 50, 60 minutes, about 1, 2, 3, 4, 5, 6, 7, 8, 9, 10, 11, 12, 13, 14, 15, 16, 17, 18, 19 hours, about 1, 2, 3, 4, 5, 6, 7 days or longer. A nanoparticle may remain stable in the presence of about 5, 10, 15, 25, 50, 100, 150, 200, or about 300 μg/ml or more human serum albumin. Stability of a nanoparticle may be determined by measuring the ability of a nanoparticle to maintain the activity of a polynucleotide of the peptide-polynucleotide complex of the nanoparticle, or by measuring changes in the size of a nanoparticle over time. Methods of measuring the size of a nanoparticle may be as described in this Section.

Methods of preparing a peptide-polynucleotide complex of the invention generally comprise contacting a peptide of the invention with a polynucleotide of the invention to form a peptide-polynucleotide complex. Typically, a peptide and a polynucleotide are contacted by incubating under conditions suitable for a peptide-polynucleotide complex to form. Conditions suitable for a peptide-polynucleotide complex to form may be as described in the examples. Typically, such conditions may comprise a temperature of about 30° C. to about 40° C., and incubation times of between about 20 sec to about 60 min or more. Suitable temperatures may also be lower than about 30° C. For example, incubation may occur on ice. One skilled in the art will appreciate that the length and temperature of incubation can and will vary depending on the peptide and the polynucleotide, and may be determined experimentally.

A nanoparticle comprising a peptide-polynucleotide complex of the invention may be further modified to enhance stability of the nanoparticle. For instance, a peptide-polynucleotide complex of the invention may be crosslinked to enhance the stability of nanoparticles. One of ordinary skill in the art would recognize that a suitable cross-linker can and will vary depending on the composition of the nanoparticle and the antibody or antibody fragment. In some aspects, a peptide-polynucleotide complex of the invention may be chemically crosslinked using chemical crosslinkers such as glutaraldehyde, bis-carboxylic acid spacers, bis-carboxylic acid-active esters, using a bis-linker amine/acid by carbodiimide coupling protocol, or using a click chemistry protocol, carbodimide-coupling chemistry, acylation, active ester coupling, or alkylation.

Alternatively, a peptide-polynucleotide complex of the invention may be coated with a compound capable of enhancing the stability of nanoparticles. Methods of modifying a nanoparticle to enhance stability are known in the art, and may be as described in Nicolas et al., 2013 Acta Biomater. 9:4754-4762, the disclosure of which is incorporated herein by reference in its entirety.

As used herein, the term “coating” may refer to the interaction of a peptide-polynucleotide complex with a compound through non-covalent bonds, or to the covalent bonding of a peptide-polynucleotide complex and a compound. In some embodiments, a peptide-polynucleotide complex of the invention and a coating compound associate through non-covalent bonds such as a hydrogen bond, an ionic bond, a bond based on Van der Waals, a hydrophobic bond, or electrostatic interactions. For instance, a peptide-polynucleotide complex of the invention may have an overall net positive charge, and a coating compound may have an overall negative charge which may allow the peptide-polynucleotide complex and compound to associate through electrostatic interactions to form a complex of the invention.

Non-limiting examples of compounds that may be used to coat a nanoparticle to enhance stability of the nanoparticle include albumin, fatty acids such as oleic acid, polyethylene glycol, polysaccharides such as chitosan, heparin or heparans and other glycosaminoglycans, or other published coating materials known to those skilled in the art. In some embodiments, stability of a peptide-polynucleotide complex of the invention may be enhanced by coating nanoparticles with a fatty acid. In other embodiments, stability of a peptide-polynucleotide complex of the invention may be enhanced by coating nanoparticles with a polysaccharide.

In some embodiments, stability of a nanoparticle comprising a peptide-polynucleotide complex of the invention may be enhanced by coating nanoparticles with albumin. Albumins are negatively charged globular proteins commonly found in blood serum. While not wishing to be bound by theory, it is believed that coating nanoparticles of the invention with albumin may enhance stability of nanoparticles by preventing flocculation, albumins that may be used to coat a nanoparticle comprising a peptide-polynucleotide complex of the invention are serum albumins, and may include bovine serum albumin and human serum albumin. In exemplary embodiments, stability of a nanoparticle comprising a peptide-polynucleotide complex of the invention may be enhanced by coating nanoparticles with human serum albumin.

In essence, a nanoparticle is coated with albumin by incubating the nanoparticle with a solution comprising albumin. Nanoparticles may be incubated in a solution comprising about 0.1, 0.2, 0.3, 0.4, 0.5, 0.6, 0.7, 0.8, 0.9, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0 mg/ml or more albumin. In some embodiments, nanoparticles comprising a peptide-polynucleotide complex of the invention may be incubated in a solution comprising about 0.1, 0.3, 0.5, 0.7, or 0.9 mg/ml albumin. In other embodiments, nanoparticles comprising a peptide-polynucleotide complex of the invention may be incubated in a solution comprising about 1.0, 1.2, 1.4, 1.6, or 1.8 mg/ml albumin. In yet other embodiments, nanoparticles comprising a peptide-polynucleotide complex of the invention may be incubated in a solution comprising about 2.0, 2.2, 2.4, 2.6, or 2.8 mg/ml albumin. In other embodiments, nanoparticles comprising a peptide-polynucleotide complex of the invention may be incubated in a solution comprising about 3.0, 3.2, 3.4, 3.6, or 3.8 mg/ml albumin. In additional embodiments, nanoparticles comprising a peptide-polynucleotide complex of the invention may be incubated in a solution comprising about 4.0, 4.2, 4.4, 4.6, 4.8, or 5.0 mg/ml albumin. In some embodiments, nanoparticles comprising a peptide-polynucleotide complex of the invention may be incubated in a solution comprising about 4.0 mg/ml albumin.

A peptide-polynucleotide complex may be incubated with albumin for about 1, 2, 5, 10, 15, 20, 25, 30, 35, 40, 45, 50, 55, or about 60 minutes or more to coat the peptide-polynucleotide complex. In some embodiments, a particle comprising a peptide-polynucleotide complex of the invention is incubated with albumin for about 5, 10, 15, or about 20 minutes. In other embodiments, a particle comprising a peptide-polynucleotide complex of the invention is incubated with albumin for about 20, 25, 30, or about 35 minutes. In yet other embodiments, a particle comprising a peptide-polynucleotide complex of the invention is incubated with albumin for about 35, 40, 45, or about 50 minutes. In other embodiments, a particle comprising a peptide-polynucleotide complex of the invention is incubated with albumin for about 50, 55, or about 60 minutes or more. In some embodiments, a particle comprising a peptide-polynucleotide complex of the invention is incubated with albumin for about 25, 30, or about 35 minutes.

A peptide-polynucleotide complex may be incubated with hyaluronic acid for about 1, 2, 5, 10, 15, 20, 25, 30, 35, 40, 45, 50, 55, or about 60 minutes or more to allow the hyaluronic acid coat the peptide-polynucleotide complex or integrate into the peptide-polynucleotide complex. A peptide-polynucleotide complex may be incubated with hyaluronic acid for about 1, 2, 3, 4, 5, 10, 12, 18, or 24 hours or more, to allow the hyaluronic acid coat the peptide-polynucleotide complex or integrate into the peptide-polynucleotide complex. In some embodiments, a peptide-polynucleotide complex may be incubated with hyaluronic acid for about 45 minutes. Shorter times could be used in some embodiments, for example, when using flow processes or microfluidic devices.

### 7.2.4. Cell

In another aspect of the invention, a peptide-polynucleotide complex of the invention is capable transfecting the polynucleotide into the cytoplasm of a cell. In some embodiments, a cell is a prokaryotic cell. In some embodiments, a cell is a eukaryotic cell. A cell may be in vitro, in vivo, in situ, or ex vivo. A cell may be a single cell, or may comprise a tissue or an organ. The term “cell” also refers to a cell in a subject.

A peptide-polynucleotide complex of the invention may be administered to a cell in vitro by incubating a cell in the presence of a peptide-polynucleotide complex of the invention under conditions suitable for transfection of a polynucleotide of a peptide-polynucleotide complex. Conditions suitable for transfection of a polynucleotide in a peptide-polynucleotide complex may be as described in the examples. One skilled in the art will appreciate that the length of incubation can and will vary depending on the peptide-polynucleotide complex, and the cells. Typically, such conditions may comprise incubation times of between about ten minutes and 24 hours, transfection conditions may comprise incubation times of between about 15 minutes and 3 hours.

A peptide-polynucleotide complex of the invention may be administered to a cell in vivo (i.e. in a subject) by administering to a subject a composition comprising a peptide-polynucleotide complex of the invention.

### 7.3. Pharmaceutical Composition

In another aspect of the invention, a peptide-polynucleotide complex of the invention may be incorporated into pharmaceutical compositions suitable for administration. A pharmaceutical composition of the invention may be used to disrupt the expression of one or more than one nucleic acid sequence normally expressed in a cell. For instance, a pharmaceutical composition of the invention may be used to disrupt the expression of 1, 2, 3, 4, 5, 6, 7, 8, 9, 10 or more nucleic acid sequences normally expressed in a cell. A skilled artisan will appreciate that pharmaceutical compositions may be administered to treat a disease, to prevent a disease, or to promote good health. As such, a pharmaceutical composition of the invention may be used to disrupt expression of any nucleic acid sequence normally expressed in a cell, such that disrupted expression leads to measurable and beneficial effects for the subject administered the composition (i.e. significant efficacy)

In some embodiments, a pharmaceutical composition of the invention is used to disrupt the expression of one nucleic acid sequence normally expressed in a cell. In some embodiments, a pharmaceutical composition of the invention is used to disrupt the expression of a nucleic acid sequence encoding KRAS. In some embodiments, a pharmaceutical composition of the invention is used to disrupt the expression of a nucleic acid sequence encoding STAT3. In some embodiments, a pharmaceutical composition of the invention is used to disrupt the expression of a nucleic acid sequence encoding JNK2. In yet some embodiments, a pharmaceutical composition of the invention is used to disrupt the expression of a nucleic acid sequence encoding the p65 subunit of the canonical NFκB signaling pathway. In some embodiments, a pharmaceutical composition of the invention is used to disrupt the expression of a nucleic acid sequence encoding the p100/p52 subunit of the canonical NFκB signaling pathway.

In other embodiments, a pharmaceutical composition of the invention is used to disrupt the expression of two nucleic acid sequences normally expressed in a cell. In some embodiments, a pharmaceutical composition of the invention is used to disrupt the expression of a nucleic acid sequence encoding the p65 subunit of the canonical NFκB signaling pathway, and a nucleic acid sequence encoding the p100/p52 subunit of the canonical NFκB signaling pathway.

When a pharmaceutical composition of the invention is used to disrupt the expression of more than one nucleic acid sequence normally expressed in a cell, a pharmaceutical composition may be formulated using a mixture of more than one peptide-polynucleotide complex, wherein each complex comprises a polynucleotide capable of disrupting the expression of a different nucleic acid sequence normally expressed in a cell. Alternatively, more than one polynucleotide may be used for generating a mixture of peptide-polynucleotide complexes, wherein each polynucleotide is capable of disrupting the expression of a different nucleic acid sequence normally expressed in a cell.

A pharmaceutical composition of the invention may also comprise one or more nontoxic pharmaceutically acceptable carriers, adjuvants, excipients, and vehicles as desired. As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with nanoparticles of the invention, use thereof in the compositions is contemplated. Supplementary active compounds may also be incorporated into the compositions.

A pharmaceutical composition of the invention may be formulated to be compatible with its intended route of administration. Suitable routes of administration include parenteral, oral, pulmonary, transdermal, transmucosal, and rectal administration. The term parenteral, as used herein, includes subcutaneous, intravenous, intramuscular, intrathecal, or intrasternal injection, or infusion techniques.

Solutions or suspensions used for parenteral, intradermal, or subcutaneous application can include the following components: a sterile diluent such as water for injection, saline solution, fixed oils, polyethylene glycols, glycerine, propylene glycol, polysorbates, poloxamers or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride, glucose or dextrose. The pH may be adjusted with acids or bases, such as hydrochloric acid or sodium hydroxide. The parenteral preparation may be enclosed in ampoules, disposable syringes or multiple dose vials made of glass or plastic.

Oral compositions generally may include an inert diluent or an edible carrier. Oral compositions may be enclosed in gelatin capsules or compressed into tablets. For the purpose of oral therapeutic administration, the active compound may be incorporated with excipients and used in the form of tablets, troches, or capsules. Oral compositions may also be prepared using a fluid carrier for use as a mouthwash, wherein the compound in the fluid carrier is applied orally and swished and expectorated or swallowed. Pharmaceutically compatible binding agents and/or adjuvant materials may be included as part of the composition. The tablets, pills, capsules, troches, and the like, may contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from a pressured container or dispenser which contains a suitable propellant, e.g., a gas such as carbon dioxide, or a nebulizer.

In some embodiments, a pharmaceutical composition of the invention is formulated to be compatible with parenteral administration. For instance, pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include physiological saline, balanced salt solution, bacteriostatic water, Cremophor EL (BASF; Parsippany, N.J.), or phosphate buffered saline (PBS). In exemplary embodiments, a pharmaceutical composition of the invention is formulated with phosphate buffered saline (PBS).

In all cases, a composition may be sterile and may be fluid to the extent that easy syringeability exists. A composition may be stable under the conditions of manufacture and storage, and may be preserved against the contaminating action of microorganisms such as bacteria and fungi. The carrier may be a solvent or dispersion medium containing, for example, water, ethanol, polyol (for example, glycerol, propylene glycol, and liquid polyetheylene glycol, and the like), and suitable mixtures thereof. The proper fluidity may be maintained, for example, by the use of a coating such as lecithin, by the maintenance of the required particle size in the case of dispersion, and by the use of surfactants. Prevention of the action of microorganisms may be achieved by various antibacterial and antifungal agents, for example, parabens, chlorobutanol, phenol, ascorbic acid, thimerosal, and the like. In many cases, it may include isotonic agents, for example, sugars, polyalcohols such as mannitol, sorbitol, or sodium chloride, in the composition. Prolonged absorption of the injectable compositions may be brought about by including in the composition an agent which delays absorption, for example, aluminum monostearate and gelatin.

Sterile injectable solutions may be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation are vacuum drying and freeze-drying, which yields a powder of the active ingredient plus any additional desired ingredient from a previously sterile-filtered solution thereof.

Systemic administration may also be by transmucosal or transdermal means. For transmucosal or transdermal administration, penetrants appropriate to the barrier to be permeated are used in the formulation. Such penetrants are generally known in the art, and may include, for example, for transmucosal administration, detergents, bile salts, and fusidic acid derivatives. Transmucosal administration may be accomplished through the use of nasal sprays or suppositories. For transdermal administration, the active compounds are formulated into ointments, salves, gels, or creams as generally known in the art. The compounds may also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, chitosans, and polylactic acid. Methods for preparation of such formulations will be apparent to those skilled in the art. These may be prepared according to methods known to those skilled in the art, for example, as described in U.S. Pat. No. 4,522,811.

Additional formulations of pharmaceutical compositions may be in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners.

One of skill in the art will recognize that the concentration of a peptide-polynucleotide complex of the invention in a pharmaceutical composition can and will vary depending in part on the route of administration, the subject, and the reason for the administration, and may be determined experimentally. Methods of experimentally determining the concentration of an active agent such as nanoparticles of the invention in a pharmaceutical composition are known in the art. In general, a pharmaceutical composition may be formulated to comprise about 0.1 nM to about 50 μM of a polynucleotide in a peptide-polynucleotide complex of the invention. For example, a pharmaceutical composition may be formulated to comprise about 0.1 nm, 0.2 nm, 0.3 nm, 0.4 nm, 0.5 nm, 0.6 nm, 0.7 nm, 0.8 nm, 0.9 nm, 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, 220 nm, 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, 241 nm, 242 nm, 243 nm, 244 nm, 245 nm, 246 nm, 247 nm, 248 nm, 249 nm, 251 nm, 252 nm, 253 nm, 254 nm, 255 nm, 256 nm, 257 nm, 258 nm, 259 nm, 261 nm, 262 nm, 263 nm, 264 nm, 265 nm, 266 nm, 267 nm, 268 nm, 269 nm, 271 nm, 272 nm, 273 nm, 274 nm, 275 nm, 276 nm, 277 nm, 278 nm, 279 nm, 281 nm, 282 nm, 283 nm, 284 nm, 285 nm, 286 nm, 287 nm, 288 nm, 289 nm, 291 nm, 292 nm, 293 nm, 294 nm, 295 nm, 296 nm, 297 nm, 298 nm, 299 nm, 300 nm, 301 nm, 302 nm, 303 nm, 304 nm, 305 nm, 306 nm, 307 nm, 308 nm, 309 nm, 310 nm, 311 nm, 312 nm, 313 nm, 314 nm, 315 nm, 316 nm, 317 nm, 318 nm, 319 nm, 320 nm, 321 nm, 322 nm, 323 nm, 324 nm, 325 nm, 326 nm, 327 nm, 328 nm, 329 nm, 330 nm, 331 nm, 332 nm, 333 nm, 334 nm, 335 nm, 336 nm, 337 nm, 338 nm, 339 nm, 340 nm, 341 nm, 342 nm, 343 nm, 344 nm, 345 nm, 346 nm, 347 nm, 348 nm, 349 nm, 350 nm, 351 nm, 352 nm, 353 nm, 354 nm, 355 nm, 356 nm, 357 nm, 358 nm, 359 nm, 360 nm, 361 nm, 362 nm, 363 nm, 364 nm, 365 nm, 366 nm, 367 nm, 368 nm, 369 nm, 370 nm, 371 nm, 372 nm, 373 nm, 374 nm, 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, 425 nm, 426 nm, 427 nm, 428 nm, 429 nm, 430 nm, 431 nm, 432 nm, 433 nm, 434 nm, 435 nm, 436 nm, 437 nm, 438 nm, 439 nm, 440 nm, 441 nm, 442 nm, 443 nm, 444 nm, 445 nm, 446 nm, 447 nm, 448 nm, 449 nm, 450 nm, 451 nm, 452 nm, 453 nm, 454 nm, 455 nm, 456 nm, 457 nm, 458 nm, 459 nm, 460 nm, 461 nm, 462 nm, 463 nm, 464 nm, 465 nm, 466 nm, 467 nm, 468 nm, 469 nm, 470 nm, 471 nm, 472 nm, 473 nm, 474 nm, 475 nm, 476 nm, 477 nm, 478 nm, 479 nm, 480 nm, 481 nm, 482 nm, 483 nm, 484 nm, 485 nm, 486 nm, 487 nm, 488 nm, 489 nm, 490 nm, 491 nm, 492 nm, 493 nm, 494 nm, 495 nm, 496 nm, 497 nm, 498 nm, 499 nm, 500 nm, 501 nm, 502 nm, 503 nm, 504 nm, 505 nm, 506 nm, 507 nm, 508 nm, 509 nm, 510 nm, 511 nm, 512 nm, 513 nm, 514 nm, 515 nm, 516 nm, 517 nm, 518 nm, 519 nm, 520 nm, 521 nm, 522 nm, 523 nm, 524 nm, 525 nm, 526 nm, 527 nm, 528 nm, 529 nm, 530 nm, 531 nm, 532 nm, 533 nm, 534 nm, 535 nm, 536 nm, 537 nm, 538 nm, 539 nm, 540 nm, 541 nm, 542 nm, 543 nm, 544 nm, 545 nm, 546 nm, 547 nm, 548 nm, 549 nm, 550 nm, 551 nm, 552 nm, 553 nm, 554 nm, 555 nm, 556 nm, 557 nm, 558 nm, 559 nm, 560 nm, 561 nm, 562 nm, 563 nm, 564 nm, 565 nm, 566 nm, 567 nm, 568 nm, 569 nm, 570 nm, 571 nm, 572 nm, 573 nm, 574 nm, 575 nm, 576 nm, 577 nm, 578 nm, 579 nm, 580 nm, 581 nm, 582 nm, 583 nm, 584 nm, 585 nm, 586 nm, 587 nm, 588 nm, 589 nm, 590 nm, 591 nm, 592 nm, 593 nm, 594 nm, 595 nm, 596 nm, 597 nm, 598 nm, 599 nm, 600 nm, 601 nm, 602 nm, 603 nm, 604 nm, 605 nm, 606 nm, 607 nm, 608 nm, 609 nm, 610 nm, 611 nm, 612 nm, 613 nm, 614 nm, 615 nm, 616 nm, 617 nm, 618 nm, 619 nm, 620 nm, 621 nm, 622 nm, 623 nm, 624 nm, 625 nm, 626 nm, 627 nm, 628 nm, 629 nm, 630 nm, 631 nm, 632 nm, 633 nm, 634 nm, 635 nm, 636 nm, 637 nm, 638 nm, 639 nm, 640 nm, 641 nm, 642 nm, 643 nm, 644 nm, 645 nm, 646 nm, 647 nm, 648 nm, 649 nm, 650 nm, 651 nm, 652 nm, 653 nm, 654 nm, 655 nm, 656 nm, 657 nm, 658 nm, 659 nm, 660 nm, 661 nm, 662 nm, 663 nm, 664 nm, 665 nm, 666 nm, 667 nm, 668 nm, 669 nm, 670 nm, 671 nm, 672 nm, 673 nm, 674 nm, 675 nm, 676 nm, 677 nm, 678 nm, 679 nm, 680 nm, 681 nm, 682 nm, 683 nm, 684 nm, 685 nm, 686 nm, 687 nm, 688 nm, 689 nm, 690 nm, 691 nm, 692 nm, 693 nm, 694 nm, 695 nm, 696 nm, 697 nm, 698 nm, 699 nm, 700 nm, 701 nm, 702 nm, 703 nm, 704 nm, 705 nm, 706 nm, 707 nm, 708 nm, 709 nm, 710 nm, 711 nm, 712 nm, 713 nm, 714 nm, 715 nm, 716 nm, 717 nm, 718 nm, 719 nm, 720 nm, 721 nm, 722 nm, 723 nm, 724 nm, 725 nm, 726 nm, 727 nm, 728 nm, 729 nm, 730 nm, 731 nm, 732 nm, 733 nm, 734 nm, 735 nm, 736 nm, 737 nm, 738 nm, 739 nm, 740 nm, 741 nm, 742 nm, 743 nm, 744 nm, 745 nm, 746 nm, 747 nm, 748 nm, 749 nm, 750 nm, 751 nm, 752 nm, 753 nm, 754 nm, 755 nm, 756 nm, 757 nm, 758 nm, 759 nm, 760 nm, 761 nm, 762 nm, 763 nm, 764 nm, 765 nm, 766 nm, 767 nm, 768 nm, 769 nm, 770 nm, 771 nm, 772 nm, 773 nm, 774 nm, 775 nm, 776 nm, 777 nm, 778 nm, 779 nm, 780 nm, 781 nm, 782 nm, 783 nm, 784 nm, 785 nm, 786 nm, 787 nm, 788 nm, 789 nm, 790 nm, 791 nm, 792 nm, 793 nm, 794 nm, 795 nm, 796 nm, 797 nm, 798 nm, 799 nm, 800 nm, 801 nm, 802 nm, 803 nm, 804 nm, 805 nm, 806 nm, 807 nm, 808 nm, 809 nm, 810 nm, 811 nm, 812 nm, 813 nm, 814 nm, 815 nm, 816 nm, 817 nm, 818 nm, 819 nm, 820 nm, 821 nm, 822 nm, 823 nm, 824 nm, 825 nm, 826 nm, 827 nm, 828 nm, 829 nm, 830 nm, 831 nm, 832 nm, 833 nm, 834 nm, 835 nm, 836 nm, 837 nm, 838 nm, 839 nm, 840 nm, 841 nm, 842 nm, 843 nm, 844 nm, 845 nm, 846 nm, 847 nm, 848 nm, 849 nm, 850 nm, 851 nm, 852 nm, 853 nm, 854 nm, 855 nm, 856 nm, 857 nm, 858 nm, 859 nm, 860 nm, 861 nm, 862 nm, 863 nm, 864 nm, 865 nm, 866 nm, 867 nm, 868 nm, 869 nm, 870 nm, 871 nm, 872 nm, 873 nm, 874 nm, 875 nm, 876 nm, 877 nm, 878 nm, 879 nm, 880 nm, 881 nm, 882 nm, 883 nm, 884 nm, 885 nm, 886 nm, 887 nm, 888 nm, 889 nm, 890 nm, 891 nm, 892 nm, 893 nm, 894 nm, 895 nm, 896 nm, 897 nm, 898 nm, 899 nm, 900 nm, 901 nm, 902 nm, 903 nm, 904 nm, 905 nm, 906 nm, 907 nm, 908 nm, 909 nm, 910 nm, 911 nm, 912 nm, 913 nm, 914 nm, 915 nm, 916 nm, 917 nm, 918 nm, 919 nm, 920 nm, 921 nm, 922 nm, 923 nm, 924 nm, 925 nm, 926 nm, 927 nm, 928 nm, 929 nm, 930 nm, 931 nm, 932 nm, 933 nm, 934 nm, 935 nm, 936 nm, 937 nm, 938 nm, 939 nm, 940 nm, 941 nm, 942 nm, 943 nm, 944 nm, 945 nm, 946 nm, 947 nm, 948 nm, 949 nm, 950 nm, 951 nm, 952 nm, 953 nm, 954 nm, 955 nm, 956 nm, 957 nm, 958 nm, 959 nm, 960 nm, 961 nm, 962 nm, 963 nm, 964 nm, 965 nm, 966 nm, 967 nm, 968 nm, 969 nm, 970 nm, 971 nm, 972 nm, 973 nm, 974 nm, 975 nm, 976 nm, 977 nm, 978 nm, 979 nm, 980 nm, 981 nm, 982 nm, 983 nm, 984 nm, 985 nm, 986 nm, 987 nm, 988 nm, 989 nm, 990 nm, 991 nm, 992 nm, 993 nm, 994 nm, 995 nm, 996 nm, 997 nm, 998 nm, 999 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, or about 50 μm of a polynucleotide in a peptide-polynucleotide complex of the invention. In some embodiments, a pharmaceutical composition may be formulated to comprise about 0.1 nM to about 1.0 nM of a polynucleotide in a peptide-polynucleotide complex of the invention. In other embodiments, a pharmaceutical composition may be formulated to comprise about 1 nM to about 10 nM of a polynucleotide in a peptide-polynucleotide complex of the invention. In other embodiments, a pharmaceutical composition may be formulated to comprise about 1 nM to about 100 nM of a polynucleotide in a peptide-polynucleotide complex of the invention. In other embodiments, a pharmaceutical composition may be formulated to comprise about 1 nM to about 200 nM of a polynucleotide in a peptide-polynucleotide complex of the invention. In other embodiments, a pharmaceutical composition may be formulated to comprise about 1 nM to about 50 nM of a polynucleotide in a peptide-polynucleotide complex of the invention. In other embodiments, a pharmaceutical composition may be formulated to comprise about 10 nM to about 100 nM of a polynucleotide in a peptide-polynucleotide complex of the invention. In other embodiments, a pharmaceutical composition may be formulated to comprise about 10 nM to about 200 nM of a polynucleotide in a peptide-polynucleotide complex of the invention. In other embodiments, a pharmaceutical composition may be formulated to comprise about 50 nM to about 100 nM of a polynucleotide in a peptide-polynucleotide complex of the invention. In other embodiments, a pharmaceutical composition may be formulated to comprise about 50 nM to about 200 nM of a polynucleotide in a peptide-polynucleotide complex of the invention. In other embodiments, a pharmaceutical composition may be formulated to comprise about 100 nM to about 200 nM of a polynucleotide in a peptide-polynucleotide complex of the invention. In other embodiments, a pharmaceutical composition may be formulated to comprise about 150 nM to about 200 nM of a polynucleotide in a peptide-polynucleotide complex of the invention. In other embodiments, a pharmaceutical composition may be formulated to comprise about 200 nM to about 100 nM of a polynucleotide in a peptide-polynucleotide complex of the invention. In other embodiments, a pharmaceutical composition may be formulated to comprise about 500 nM to about 1000 nM of a polynucleotide in a peptide-polynucleotide complex of the invention. In other embodiments, a pharmaceutical composition may be formulated to comprise about 1 μM to about 50 μM of a polynucleotide in a peptide-polynucleotide complex of the invention. A concentration of peptide in a peptide-polynucleotide complex of the invention may be calculated based on the desired concentration of polynucleotide and the ratio of peptide to polynucleotide in the peptide-polynucleotide complex of the invention.

A pharmaceutical composition may also be formulated to comprise about 30, 40, 50, 60, 70, 80, 90, 100, 150, 200, 250, 300, 350, 400, 450, 500, 550, 600, 650, or about 700 μg/ml or more of a peptide-polynucleotide complex of the invention. In some embodiments, a pharmaceutical composition is formulated to comprise 30, 35, 40, 45, 50, 55, 60, 65, 70, 75, 80, 85, 90, 95, or about 100 μg/ml of a peptide-polynucleotide complex of the invention. In other embodiments, a pharmaceutical composition is formulated to comprise 100, 110, 120, 130, 140, 150, 160, 170, 180, 190, 200, 210, 220, 230, 240, 250, 260, 270, 280, 290, or about 300 μg/ml of a peptide-polynucleotide complex of the invention. In yet other embodiments, a pharmaceutical composition is formulated to comprise 300, 310, 320, 330, 340, 350, 360, 370, 380, 390, 400, 410, 420, 430, 440, 450, 460, 470, 480, 490, or about 500 μg/ml of a peptide-polynucleotide complex of the invention. In yet other embodiments, a pharmaceutical composition is formulated to comprise 500, 510, 520, 530, 540, 550, 560, 570, 580, 590, 600, 610, 620, 630, 640, 650, 660, 670, 680, 690, or about 700 μg/ml or more of a peptide-polynucleotide complex of the invention.

### 7.4. Method of Use

In another aspect, the invention encompasses a method for using a peptide-polynucleotide complex of the invention to transfect the polynucleotide into the cytoplasm of a cell. In some embodiments, the cell is in vitro. In other embodiments, the cell is in vivo. Thus, the present invention also provides a method for using a peptide-polynucleotide complex of the invention to transfect the polynucleotide into the cytoplasm of a cell in a subject in need thereof. Generally speaking, a method of the invention comprises contacting a cell with a peptide-polynucleotide complex of the invention under conditions suitable for transfection of a polynucleotide. Suitable cells and conditions are described above. In embodiments where the cell is in vivo, a method of the invention typically comprises administering a pharmaceutical composition comprising a peptide-polynucleotide complex of the invention to a subject in need thereof. Suitable pharmaceutical compositions are described herein.

In another aspect, the invention encompasses a method for treating a condition in a subject. The method comprises administering to a subject in need thereof a therapeutically effective amount of a pharmaceutical composition comprising a peptide-polynucleotide complex. A peptide-polynucleotide complex of the invention is capable of efficiently transfecting, or delivering, the polynucleotide of the peptide-polynucleotide complex into a cell of the subject.

In some embodiments, a polynucleotide of the invention comprises non-coding RNA capable of regulating or inhibiting expression of a nucleic acid sequence expressed in a cell. By efficiently transfecting a polynucleotide capable of regulating or inhibiting expression of a nucleic acid sequence expressed in a cell, a method of the invention may be used to treat any condition that can be treated by regulating or inhibiting the expression of a nucleic acid sequence normally expressed in a cell. In some embodiments, the invention encompasses a method of administering a peptide-polynucleotide complex of the invention to a subject to treat an NFκB-mediated condition in the subject. In some embodiments, the invention encompasses a method of administering to a subject a peptide-polynucleotide complex of the invention to treat a condition associated with overexpression or aberrant expression of KRAS in the subject. In some embodiments, the invention encompasses a method of administering to a subject a peptide-polynucleotide complex of the invention to treat a condition associated with STAT3 dysregulation in the subject. In some embodiments, the invention encompasses a method of administering to a subject a peptide-polynucleotide complex of the invention to treat a condition associated with JNK2 dysregulation in the subject.

The peptide, the polynucleotide and peptide-polynucleotide complex may be as described herein. Pharmaceutical compositions comprising a peptide-polynucleotide complex of the invention may be as described herein. Methods of administering a peptide-polynucleotide complex of the invention, and methods of treating a condition are described below.

### 7.4.1. Administration to a Subject in Need Thereof

In an aspect, the present invention encompasses administering a therapeutically effective amount of a pharmaceutical composition to a subject in need thereof. As used herein, the phrase “a subject in need thereof” refers to a subject in need of preventative or therapeutic treatment. A subject may be a rodent, a human, a livestock animal, a companion animal, or a zoological animal. In one embodiment, a subject may be a rodent, e.g., a mouse, a rat, a guinea pig, etc. In another embodiment, a subject may be a livestock animal. Non-limiting examples of suitable livestock animals may include pigs, cows, horses, goats, sheep, llamas and alpacas. In still another embodiment, a subject may be a companion animal. Non-limiting examples of companion animals may include pets such as dogs, cats, rabbits, and birds. In yet another embodiment, a subject may be a zoological animal. As used herein, a “zoological animal” refers to an animal that may be found in a zoo. Such animals may include non-human primates, large cats, wolves, and bears. In some embodiments, a subject is a mouse. In some embodiments, a subject is a human.

As described herein, a pharmaceutical composition of the invention is formulated to be compatible with its intended route of administration. Suitable routes of administration include parenteral, oral, pulmonary, transdermal, transmucosal, and rectal administration. In some embodiments, a pharmaceutical composition of the invention is administered by injection.

One of skill in the art will recognize that the amount and concentration of the composition administered to a subject will depend in part on the subject and the reason for the administration. Methods for determining optimal amounts are known in the art. In general, the concentration of a peptide-polynucleotide complex of the invention in a pharmaceutical composition may be as described herein.

Compositions of the invention are typically administered to a subject in need thereof in an amount sufficient to provide a benefit to the subject. This amount is defined as a “therapeutically effective amount.” A therapeutically effective amount may be determined by the efficacy or potency of the particular composition, the disorder being treated, the duration or frequency of administration, the method of administration, and the size and condition of the subject, including that subject's particular treatment response. A therapeutically effective amount may be determined using methods known in the art, and may be determined experimentally, derived from therapeutically effective amounts determined in model animals such as the mouse, or a combination thereof. Additionally, the route of administration may be considered when determining the therapeutically effective amount. In determining therapeutically effective amounts, one skilled in the art may also consider the existence, nature, and extent of any adverse effects that accompany the administration of a particular compound in a particular subject.

When a pharmaceutical composition of the invention is administered to a subject by injection, a composition may be administered to the subject in a bolus in an amount of about 0.1 mg/kg to about 100 mg/kg or more. In some embodiments, a pharmaceutical composition of the invention is administered to a subject in an amount of about 0.1 mg/kg to about 5 mg/kg. In other embodiments, a pharmaceutical composition of the invention is administered to a subject in an amount of about 5 mg/kg to about 15 mg/kg. In yet other embodiments, a pharmaceutical composition of the invention is administered to a subject in an amount of about 15 mg/kg to about 30 mg/kg. In other embodiments, a pharmaceutical composition of the invention is administered to a subject in an amount of about 30 mg/kg to about 45 mg/kg. In additional embodiments, a pharmaceutical composition of the invention is administered to a subject in an amount of about 45 mg/kg to about 100 mg/kg or more. In some embodiments, a composition is administered to the subject in a bolus in an amount of about 0.5 to about 1.5 mg/kg.

A composition may also be administered by injecting more than one bolus into the subject over a period of time. For instance, a composition may be administered by injecting 1, 2, 3, 4, 5, 6, 7, 8, 9, 10 or more boluses into the subject. In some embodiments, a composition is administered by injecting 1, 2, 3, 4, or 5 boluses into the subject. In other embodiments, a composition is administered by injecting 5, 6, 7, 8, 9, 10 or more boluses into the subject. In some embodiments, a composition is administered by injecting 2, 3, or 4 boluses into the subject. The boluses may be injected about every 1, 2, 3, 4, 5, 6, 7, 8, 9, 10, 11, or about every 12 hours, or they may be injected about every 1, 2, 3, 4, 5, 6, or about every 7 days. In some embodiments, boluses may be injected about every day.

### 7.4.2. Treating Cancer

In some embodiments, a method of the invention is used to treat a neoplasm or cancer. The neoplasm may be malignant or benign, the cancer may be primary or metastatic; the neoplasm or cancer may be early stage or late stage. The cancer may be a blood cancer or a solid tumor cancer. A cancer or a neoplasm may be treated by delivering a nucleic acid sequence to a cancer tumor in a subject. The cancer or neoplasm may be treated by slowing cancer cell growth, killing cancer cells or reducing the spreading of cancer cells to generate metastases. In some embodiments, the cancer cell expresses KRAS or a mutated version of KRAS. The present invention is particularly suited to treating patients exhibiting one or more of a wide variety of KRAS mutations since the nucleic acid sequences of the present invention have been carefully selected as targeting locations of KRAS outside regions of known mutation hotspots, for example, mutations at amino acid G12, G13 and Q61. The complexes of the present invention instead selectively target cancer cells by nature of the entry of the complex into cancer tissues, and as a result have been designed to target cancer cells in particular despite the identity of any KRAS mutation.

In some embodiments, a polynucleotide of a peptide-polynucleotide complex of the invention may treat a cancer or a neoplasm by delivering a polynucleotide of the nanoparticle to a cancer cell in a subject in vivo. In some embodiments, a polynucleotide of a peptide-polynucleotide complex of the invention may treat a cancer or a neoplasm by delivering a polynucleotide of the nanoparticle to cells of the tumor microenvironment or to other cells in the surroundings of a tumor. Non-limiting examples of neoplasms or cancers that may be treated with a method of the invention may include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sezary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-cell lymphoma (cutaneous), T-cell leukemia and lymphoma, testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood). In some embodiments, a method of the invention is used to treat T-cell leukemia and lymphoma. In an exemplary embodiment, a method of the invention is used to treat Human T-Lymphotropic Virus-1 (HTLV-1) induced adult T-cell leukemia/lymphoma (ATLL).

In other embodiments, a polynucleotide of a peptide-polynucleotide complex of the invention may be delivered to a cancer cell in vitro. For instance, a polynucleotide of a peptide-polynucleotide complex of the invention may be delivered to a cancer cell line in vitro. A cancer cell may be a cancer cell line cultured in vitro. In some alternatives of the embodiments, a cancer cell line may be a primary cell line that is not yet described. Methods of preparing a primary cancer cell line utilize standard techniques known to individuals skilled in the art. In other alternatives, a cancer cell line may be an established cancer cell line. A cancer cell line may be adherent or non-adherent, or a cell line may be grown under conditions that encourage adherent, non-adherent or organotypic growth using standard techniques known to individuals skilled in the art. A cancer cell line may be contact inhibited or non-contact inhibited.

In some embodiments, the cancer cell line may be an established human cell line derived from a tumor. Non-limiting examples of cancer cell lines derived from a tumor may include the osteosarcoma cell lines 143B, CAL-72, G-292, HOS, KHOS, MG-63, Saos-2, and U-2 OS; the prostate cancer cell lines DU145, PC3 and Lncap; the breast cancer cell lines MCF-7, MDA-MB-438 and T47D; the myeloid leukemia cell line THP-1, the glioblastoma cell line U87; the neuroblastoma cell line SHSY5Y; the bone cancer cell line Saos-2; the colon cancer cell lines WiDr, COLO 320DM, HT29, DLD-1, COLO 205, COLO 201, HCT-15, SW620, LoVo, SW403, SW403, SW1116, SW1463, SW837, SW948, SW1417, GPC-16, HCT-8, HCT 116, NCI-H716, NCI-H747, NCI-HSO8, NCI-H498, COLO 320HSR, SNU-C2A, LS 180, LS 174T, MOLT-4, LS513, LS1034, LS411N, Hs 675.T, CO 88BV59-1, Co88BV59H21-2, Co88BV59H21-2V67-66, 1116-NS-19-9, TA 99, AS 33, TS 106, Caco-2, HT-29, SK-CO-1, SNU-C2B and SW480; the non-small cell lung cancer (NSCLC) cell lines H358, H2122, H441, H727, SK-Lu-1, H2009, the melanoma cell line B16-F10, the macrophage cell line RAW264.7, the F8 cell line, and the pancreatic carcinoma cell lines Panc1, PANC 10.05, CAPAN-1, CAPAN-2, PSN1, MIA-PaCa2. In an exemplary embodiment, a peptide-polynucleotide complex of the invention may be administered to a F8 cell line. In another exemplary embodiment, a peptide-polynucleotide complex of the invention may be administered to a B16-F10 cell line.

### 7.5. Kit

Another aspect of the invention encompasses a kit. The kit comprises a first composition comprising a peptide of the invention, and optionally a second composition comprising a polynucleotide. Alternatively, a polynucleotide of interest may be provided by a user of the kit. By following directions provided by the kit, a user of the kit may mix the composition comprising a peptide of the invention and a composition comprising a polynucleotide to form a peptide-polynucleotide complex. The directions of the kit may include instructions to mix the peptide and polynucleotide at a suitable ratio. The kit may also include suitable buffers, water, cross-linking reagents or albumin.

### 8. EXAMPLES

The following is a description of various methods and materials used in the studies. They are put forth so as to provide those of ordinary skill in the art with a complete disclosure and description of how to make and use the present invention, and are not intended to limit the scope of what the inventors regard as their invention, nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, percentages, etc.), but some experimental errors and deviations should be accounted for.

### Example 1—siRNA Targeting KRAS

siRNA avoiding sites that harbor mutations were developed with the aim of using the same compound to knock down KRAS regardless of the mutation. The mutation sites that were avoided are G12, G13 and Q61.

We started out with an in silico evaluation. The bioinformatical approach assumed a canonical siRNA structure. Positions 2-18 (5′-3′) of the sense and antisense strand were used for the specificity calculations. Positions 1-19 (5′-3′) of the antisense strand were used for cross reactivity and human SNP analysis. The following parameters were assessed.
Species cross-reactivity for human, cynomolgus monkey, rhesus monkey and mouse: Analysis based on a canonical siRNA design using 19 bases and 17 bases (without considering positions 1 and 19) for cross-reactivity. Full match as well as single mismatch analysis were included.
Predicted specificity in human, rhesus monkey, cynomolgus monkey and mouse. Sense and antisense strand were analyzed separately.
Identicalness of siRNA seed region and seed region of known miRNAs
Analysis of human SNP database (NCBI-DB-SNP) to identify siRNAs targeting regions with known SNPs. Information included positions of SNPs within the target sequence as well as minor allele frequency (MAF) in case data were available.
siRNA activity prediction based on canonical siRNA design.

96 sequences were selected for the first in vitro analysis. The sequences are shown in Table 1 above.

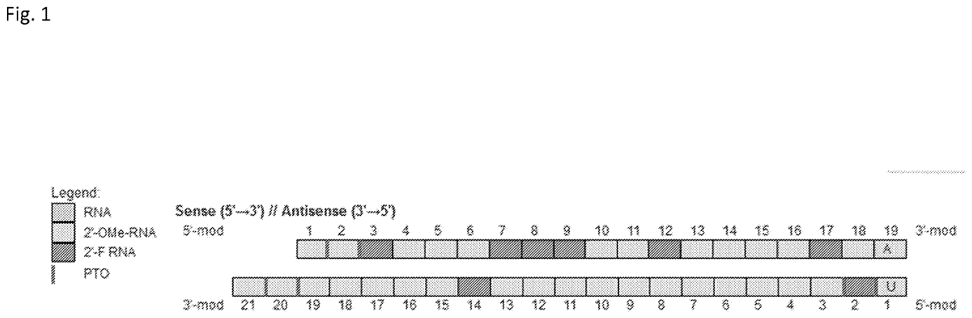

The 96 selected sequences were further modified. The modification pattern is shown in FIG. 1. The modified sequences are shown in Table 2 above.

These 96 sequences were synthetized and tested in NCI-H23 cells carrying the KRAS G12C mutation at two different doses (0.1 and 10 nM), the results of this analysis are shown in Table 3 below.

TABLE 3
Two Dose Analysis in NCI-H23

| | | KRAS | | | | KRAS | | | | |
|---|---|---|---|---|---|---|---|---|---|---|
| | | MV % | | GAPDH | | MV % | | GAPDH | | |
| sense | antisense | residual | SD | MV | SD | residual | SD | MV | SD | selected |
| gsgsAfcuuAf | usUfsaacuucu | 12.6 | 2.3 | 92.0 | 2.9 | 30.6 | 1.6 | 101.9 | 6.4 | X |
| GfCfaaGfaagu | ugcuAfaguccs | | | | | | | | | |
| Ufaa | usg | | | | | | | | | |
| gsusCfucuUf | usCfsgagaaua | 12.9 | 3.4 | 100.0 | 4.6 | 62.1 | 3.7 | 99.9 | 4.6 | X |
| GfGfauAfuuc | uccaAfgagacs | | | | | | | | | |
| uCfga | asg | | | | | | | | | |
| gscsAfguuGf | usUfsaagaagu | 14.7 | 1.5 | 89.3 | 3.5 | 22.0 | 0.9 | 93.6 | 3.9 | X |
| AfUfuaCfuuc | aaucAfacugcs | | | | | | | | | |
| uUfaa | asu | | | | | | | | | |
| asgsAfaauUfC | usUfsuauguuu | 15.0 | 0.9 | 84.8 | 3.9 | 65.9 | 4.6 | 86.4 | 4.7 | X |
| fGfaaAfacauA | ucgaAfuuucus | | | | | | | | | |
| faa | csg | | | | | | | | | |
| asgsAfuauUfC | usCfsuauaaug | 15.1 | 1.9 | 90.5 | 6.5 | 41.0 | 2.7 | 90.8 | 5.1 | X |
| fAfccAfuuau | gugaAfuaucus | | | | | | | | | |
| Afga | usc | | | | | | | | | |
| gsasAfucaUfC | usAfscagaaua | 15.9 | 1.1 | 95.3 | 1.4 | 47.4 | 2.6 | 96.2 | 2.4 | X |
| fCfcuAfuucu | gggaUfgauucs | | | | | | | | | |
| Gfua | asa | | | | | | | | | |
| ususCfuauAfC | usUfscgaacua | 16.1 | 1.6 | 98.9 | 3.8 | 62.9 | 3.0 | 95.9 | 5.2 | X |
| fAfuuAfguuc | auguAfuagaas | | | | | | | | | |
| Gfaa | gsg | | | | | | | | | |
| gsasUfauuCfA | usUfscuauaau | 16.4 | 0.9 | 98.2 | 7.5 | 42.6 | 3.9 | 91.9 | 9.9 | X |
| fCfcaUfuauaG | ggugAfauaucs | | | | | | | | | |
| faa | usu | | | | | | | | | |
| usasUfucaCfC | usUfscucuaua | 17.4 | 2.2 | 89.8 | 5.0 | 49.9 | 5.1 | 94.8 | 8.2 | X |
| fAfuuAfuaga | auggUfgaauas | | | | | | | | | |
| Gfaa | usc | | | | | | | | | |
| ususAfguuCf | usUfscgaauuu | 17.6 | 1.3 | 90.8 | 6.6 | 54.7 | 7.5 | 90.9 | 9.7 | X |
| GfAfgaAfauu | cucgAfacuaas | | | | | | | | | |
| cGfaa | usg | | | | | | | | | |
| csgsAfauaUfG | usAfsuuguugg | 17.6 | 1.0 | 74.1 | 6.5 | 30.1 | 1.9 | 94.3 | 3.8 | X |
| fAfucCfaacaA | aucaUfauucgs | | | | | | | | | |
| fua | usc | | | | | | | | | |
| usgsAfcgaUf | usGfsaauuagc | 17.9 | 1.1 | 89.7 | 4.4 | 57.5 | 4.5 | 95.9 | 8.7 | X |
| AfCfagCfuaau | uguaUfcgucas | | | | | | | | | |
| Ufca | asg | | | | | | | | | |
| ascsGfauaCfA | usCfsugaauua | 18.6 | 0.9 | 82.0 | 11.6 | 38.7 | 2.5 | 88.1 | 5.4 | X |
| fGfcuAfauuc | gcugUfaucgus | | | | | | | | | |
| Afga | csa | | | | | | | | | |
| asusUfaguUfC | usCfsgaauuuc | 18.8 | 2.7 | 95.4 | 6.1 | 69.8 | 6.4 | 90.8 | 7.0 | X |
| fGfagAfaauuC | ucgaAfcuaaus | | | | | | | | | |
| fga | gsu | | | | | | | | | |
| gsasUfacaGfC | usUfsucugaau | 19.8 | 0.5 | 86.5 | 6.2 | 62.9 | 3.0 | 91.7 | 7.9 | X |
| fUfaaUfucagA | uagcUfguaucs | | | | | | | | | |
| faa | gsu | | | | | | | | | |
| gsgsAfuauUf | usGfscuguguc | 21.8 | 7.0 | 88.8 | 15.8 | 77.7 | 4.2 | 97.0 | 6.8 | X |
| CfUfcgAfcaca | gagaAfuauccs | | | | | | | | | |
| Gfca | asa | | | | | | | | | |
| asgsGfacaUfC | usGfsauaguaa | 21.9 | 0.6 | 70.8 | 18.7 | 47.8 | 9.2 | 98.2 | 8.5 | X |
| fAfcuUfacuaU | gugaUfguccus | | | | | | | | | |
| fca | csa | | | | | | | | | |
| cscsGfaaaGfU | usGfsgaauugg | 24.3 | 1.7 | 108.8 | 10.8 | 56.6 | 3.7 | 91.9 | 4.1 | X |
| fUfucCfaauuC | aaacUfuucggs | | | | | | | | | |
| fca | asu | | | | | | | | | |
| cscsAfauuUfC | usGfsuacuagu | 24.5 | 3.4 | 95.5 | 6.4 | 33.2 | 4.4 | 87.3 | 3.1 | X |
| fUfuaCfuagu | aagaAfauuggs | | | | | | | | | |
| Afca | csa | | | | | | | | | |
| ascsAfuuaGfA | usUfsaguaauu | 25.3 | 2.7 | 90.1 | 7.5 | 48.2 | 2.4 | 86.7 | 1.7 | X |
| fUfaaAfuuacU | uaucUfaaugus | | | | | | | | | |
| faa | gsa | | | | | | | | | |
| usgsUfacaUfU | usAfsauuuagu | 25.8 | 4.4 | 81.4 | 7.8 | 46.0 | 1.9 | 91.0 | 4.5 | X |
| fAfcaCfuaaaU | guaaUfguacas | | | | | | | | | |
| fua | asa | | | | | | | | | |
| cscsCfuaaCfC | usUfsaaaucuu | 26.1 | 2.0 | 90.2 | 8.5 | 43.1 | 2.6 | 101.1 | 5.7 | X |
| fAfuaAfgauu | auggUfuagggs | | | | | | | | | |
| Ufaa | gsa | | | | | | | | | |
| asgsUfaugAf | usUfsaaucccca | 27.3 | 4.7 | 92.0 | 10.7 | 54.8 | 4.7 | 75.1 | 28.7 | X |
| AfAfugGfgga | uuuCfauacusg | | | | | | | | | |
| uUfaa | sg | | | | | | | | | |
| gscsCfaauUfU | usUfsacuagua | 29.3 | 5.8 | 79.6 | 19.3 | 39.6 | 4.8 | 86.7 | 5.9 | X |
| fCfuuAfcuag | agaaAfuuggcs | | | | | | | | | |
| Ufaa | asc | | | | | | | | | |
| csasAfugaGfG | usUfsguacugg | 23.3 | 1.3 | 101.1 | 5.3 | 79.1 | 1.6 | 96.1 | 5.8 | |
| fGfacCfaguaC | ucccUfcauugs | | | | | | | | | |
| faa | csa | | | | | | | | | |
| gsgsAfcgaAf | usGfsuuggauc | 24.0 | 0.5 | 81.3 | 20.3 | 101.0 | 6.1 | 94.8 | 5.1 | |
| UfAfugAfucc | auauUfcguccs | | | | | | | | | |
| aAfca | asc | | | | | | | | | |
| ususGfgcuAf | usGfsuuaagag | 26.0 | 2.2 | 98.9 | 1.5 | 81.8 | 5.9 | 99.9 | 0.2 | |
| GfUfucUfcuu | aacuAfgccaas | | | | | | | | | |
| aAfca | asc | | | | | | | | | |
| ususUfgaaUf | usCfsgagaaug | 26.3 | 1.4 | 90.2 | 1.0 | 68.9 | 4.6 | 100.1 | 2.2 | |
| AfUfccAfuuc | gauaUfucaaas | | | | | | | | | |
| uCfga | usa | | | | | | | | | |
| gsgsAfuacAfC | usUfsgacaaau | 28.1 | 4.1 | 87.2 | 6.7 | 72.9 | 9.7 | 96.6 | 4.9 | |
| fUfuaUfuugu | aaguGfuauccs | | | | | | | | | |
| Cfaa | usu | | | | | | | | | |
| uscsUfaggUf | usGfsaacuagc | 28.2 | 1.4 | 90.7 | 0.7 | 71.2 | 3.2 | 104.3 | 3.8 | |
| UfUfggCfuag | caaaCfcuagas | | | | | | | | | |
| uUfca | gsa | | | | | | | | | |
| asasUfucgAfA | usUfscuuuaug | 28.4 | 5.1 | 82.3 | 7.9 | 91.3 | 5.8 | 91.1 | 6.3 | |
| fAfacAfuaaaG | uuuuCfgaauus | | | | | | | | | |
| faa | usc | | | | | | | | | |
| usgsGfgcaAf | usCfsuugcaca | 29.1 | 5.6 | 92.6 | 2.3 | 71.8 | 2.8 | 104.0 | 1.9 | |
| AfAfuuGfugc | auuuUfgcccas | | | | | | | | | |
| aAfga | asg | | | | | | | | | |
| usgsAfcuuAf | usUfsaucuaga | 29.2 | 2.1 | 98.4 | 4.4 | 81.9 | 16.2 | 98.3 | 6.9 | |
| GfGfuuCfuag | accuAfagucas | | | | | | | | | |
| aUfaa | CSC | | | | | | | | | |
| gsusAfgggUf | usUfsaagucuu | 30.6 | 5.3 | 88.0 | 8.1 | 49.3 | 8.1 | 95.6 | 3.4 | |
| GfUfuaAfgac | aacaCfccuacsc | | | | | | | | | |
| uUfaa | su | | | | | | | | | |
| gsusUfuggCf | usUfsaagagaa | 30.7 | 0.7 | 88.1 | 22.1 | 64.5 | 5.4 | 102.0 | 3.6 | |
| UfAfguUfcuc | cuagCfcaaacsc | | | | | | | | | |
| uUfaa | su | | | | | | | | | |
| gsasCfgauAfC | usUfsgaauuag | 30.8 | 2.4 | 84.5 | 3.1 | 86.9 | 5.4 | 92.8 | 4.9 | |
| fAfgcUfaauuC | cuguAfucgucs | | | | | | | | | |
| faa | asa | | | | | | | | | |
| gsgsAfuaaUf | usAfsauuaccu | 31.6 | 5.6 | 81.9 | 7.3 | 50.6 | 2.3 | 96.5 | 4.3 | |
| GfAfuaGfgua | aucaUfuauccs | | | | | | | | | |
| aUfua | csa | | | | | | | | | |
| uscsCfuggUf | usGfsuauuacu | 32.8 | 9.9 | 92.4 | 5.0 | 72.2 | 3.5 | 102.5 | 1.6 | |
| AfAfcaGfuaau | guuaCfcaggas | | | | | | | | | |
| Afca | gsu | | | | | | | | | |
| csusUfaggCfA | usAfsacauguu | 33.2 | 4.8 | 97.5 | 8.5 | 50.0 | 3.8 | 98.4 | 4.5 | |
| fUfuaAfcaug | aaugCfcuaags | | | | | | | | | |
| Ufua | usc | | | | | | | | | |
| gsasAfuagUfC | usAfsucuaguu | 33.7 | 4.8 | 87.0 | 7.7 | 77.1 | 3.2 | 97.1 | 5.2 | |
| fAfuaAfcuag | augaCfuauucs | | | | | | | | | |
| Afua | usu | | | | | | | | | |
| usasGfgaaUfG | usAfsuaugacc | 33.8 | 2.0 | 69.4 | 23.2 | 39.1 | 1.6 | 89.4 | 2.9 | |
| fUfugGfucau | aacaUfuccuas | | | | | | | | | |
| Afua | gsg | | | | | | | | | |
| ascsUfguaCfU | usAfsauuagga | 34.0 | 5.6 | 91.1 | 5.2 | 73.3 | 9.0 | 96.0 | 5.2 | |
| fAfcuCfcuaaU | guagUfacagus | | | | | | | | | |
| fua | usc | | | | | | | | | |
| uscsAfuuuGf | usGfsucagcgc | 35.2 | 4.4 | 94.2 | 13.1 | 56.3 | 6.2 | 92.3 | 3.6 | |
| GfUfugCfgcu | aaccAfaaugas | | | | | | | | | |
| gAfca | usg | | | | | | | | | |
| gscsAfucaUfG | usAfsacuauag | 35.3 | 3.9 | 89.8 | 3.5 | 82.8 | 5.5 | 80.2 | 23.3 | |
| fUfccUfauagU | gacaUfgaugcs | | | | | | | | | |
| fua | csu | | | | | | | | | |
| usgsCfuauUf | usUfsgaccaug | 35.5 | 12.1 | 79.4 | 20.2 | 69.6 | 1.3 | 94.2 | 4.1 | |
| AfGfucAfugg | acuaAfuagcas | | | | | | | | | |
| uCfaa | gsu | | | | | | | | | |
| usgsUfuuuAf | usAfsaacuuuc | 35.6 | 3.1 | 99.4 | 4.1 | 45.5 | 5.4 | 92.5 | 3.0 | |
| UfCfcgAfaagu | ggauAfaaacas | | | | | | | | | |
| Ufua | csu | | | | | | | | | |
| asgsUfuuuAf | usUfsuguaccc | 35.6 | 5.9 | 102.8 | 12.0 | 69.7 | 4.4 | 102.7 | 1.4 | |
| UfCfugGfgua | agauAfaaacus | | | | | | | | | |
| cAfaa | asu | | | | | | | | | |
| gsusGfuuuUf | usAfsacuuucg | 36.1 | 5.9 | 87.1 | 4.0 | $1.5 | 3.4 | 99.1 | 4.5 | |
| AfUfccGfaaag | gauaAfaacacs | | | | | | | | | |
| Ufua | usg | | | | | | | | | |
| usasUfacaUfU | usUfsucucgaa | 36.2 | 2.8 | 91.6 | 2.5 | 71.1 | 4.0 | 93.2 | 5.6 | |
| fAfguUfcgag | cuaaUfguauas | | | | | | | | | |
| Afaa | gsa | | | | | | | | | |
| asusAfuucAfC | usCfsucuauaa | 36.3 | 2.2 | 102.2 | 4.9 | 111.3 | 5.5 | 98.6 | 8.1 | |
| fCfauUfauagA | ugguGfaauaus | | | | | | | | | |
| fga | csu | | | | | | | | | |
| cscsUfaacCfA | usGfsuaaaucu | 36.9 | 2.6 | 98.2 | 2.5 | 68.6 | 1.0 | 97.2 | 4.2 | |
| fUfaaGfauuu | uaugGfuuaggs | | | | | | | | | |
| Afca | gsg | | | | | | | | | |
| asasUfagaCfA | usGfsauagggu | 38.5 | 12.9 | 83.2 | 7.8 | 77.6 | 4.4 | 100.2 | 5.0 | |
| fGfaaCfccuaU | ucugUfcuauus | | | | | | | | | |
| fca | csa | | | | | | | | | |
| csusGfuacUfA | usUfsaauuagg | 38.9 | 6.2 | 94.7 | 4.4 | 68.1 | 2.9 | 95.1 | 4.9 | |
| fCfucCfuaauU | aguaGfuacags | | | | | | | | | |
| faa | usu | | | | | | | | | |
| gsgsAfaugUf | usUfsgauauga | 39.3 | 4.5 | 86.2 | 3.1 | 57.7 | 2.9 | 91.3 | 3.0 | |
| UfGfguCfaua | ccaaCfauuccs | | | | | | | | | |
| uCfaa | usa | | | | | | | | | |
| cscsAfucaUfU | usAfsgcgcaac | 39.3 | 3.0 | 101.7 | 3.3 | 62.2 | 11.6 | 90.8 | 5.9 | |
| fUfggUfugcg | caaaUfgauggs | | | | | | | | | |
| Cfua | asa | | | | | | | | | |
| asgsGfgauUf | usUfsgauuagg | 39.4 | 5.1 | 83.5 | 5.6 | 74.3 | 6.8 | 96.6 | 5.3 | |
| UfGfacCfuaau | ucaaAfucccus | | | | | | | | | |
| Cfaa | usu | | | | | | | | | |
| ususUfgggCf | usAfsgcuauaa | 39.6 | 16.8 | 82.7 | 5.8 | 95.0 | 14.1 | 92.6 | 4.6 | |
| CfAfguUfaua | cuggCfccaaas | | | | | | | | | |
| gCfua | usa | | | | | | | | | |
| csusAfggaAf | usUfsaugacca | 40.7 | 1.9 | 100.2 | 6.6 | 45.7 | 0.9 | 84.3 | 2.2 | |
| UfGfuuGfguc | acauUfccuags | | | | | | | | | |
| aUfaa | gsu | | | | | | | | | |
| usasUfccgAfA | usAfsuuggaaa | 40.9 | 2.4 | 98.6 | 3.1 | 50.0 | 3.6 | 90.0 | 3.2 | |
| fAfguUfucca | cuuuCfggauas | | | | | | | | | |
| Afua | asa | | | | | | | | | |
| cscsCfaauCfC | usUfsgucgcua | 41.6 | 7.1 | 76.3 | 6.7 | 74.0 | 2.1 | 94.1 | 6.6 | |
| fAfuuAfgcga | auggAfuugggs | | | | | | | | | |
| Cfaa | csa | | | | | | | | | |
| gsusGfacuUf | usAfsucuagaa | 43.2 | 4.2 | 85.3 | 10.8 | 92.0 | 13.3 | 101.1 | 8.1 | |
| AfGfguUfcua | ccuaAfgucacs | | | | | | | | | |
| gAfua | csu | | | | | | | | | |
| uscsCfccuAfA | usAfsaucuuau | 43.6 | 3.8 | 96.5 | 2.3 | 80.9 | 3.3 | 103.4 | 3.0 | |
| fCfcaUfaagaU | gguuAfggggas | | | | | | | | | |
| fua | asu | | | | | | | | | |
| ascsCfuagGfA | usUfsgaccaaca | 45.3 | 8.9 | 90.0 | 4.4 | 71.3 | 7.7 | 85.4 | 4.5 | |
| fAfugUfuggu | uucCfuaggusc | | | | | | | | | |
| Cfaa | sa | | | | | | | | | |
| cscsCfcuaAfC | usAfsaaucuua | 46.0 | 7.5 | 71.9 | 21.5 | 67.1 | 4.0 | 99.4 | 4.1 | |
| fCfauAfagauU | ugguUfaggggs | | | | | | | | | |
| fua | asa | | | | | | | | | |
| gsgsUfccuGfC | usUfsgauuugu | 46.3 | 8.3 | 84.2 | 9.4 | 69.4 | 4.5 | 97.2 | 4.9 | |
| fUfgaCfaaauC | cagcAfggaccs | | | | | | | | | |
| faa | asc | | | | | | | | | |
| cscsUfaggAfA | usAfsugaccaa | 46.4 | 9.6 | 97.5 | 11.0 | 55.3 | 8.4 | 90.2 | 2.4 | |
| fUfguUfgguc | cauuCfcuaggs | | | | | | | | | |
| Afua | usc | | | | | | | | | |
| gsgsGfgauAf | usAfsgaggccu | 48.2 | 8.2 | 93.5 | 10.5 | 92.9 | 4.0 | 96.5 | 4.2 | |
| UfUfuaGfgcc | aaauAfuccccs | | | | | | | | | |
| uCfua | usc | | | | | | | | | |
| usgsCfccaAfU | usUfscgcuaau | 49.5 | 2.6 | 89.9 | 2.1 | 92.4 | 3.8 | 100.4 | 2.2 | |
| fCfcaUfuagcG | ggauUfgggcas | | | | | | | | | |
| faa | gsc | | | | | | | | | |
| csasUfuagUfU | usGfsaauuucu | 49.7 | 4.7 | 87.7 | 6.7 | 92.3 | 5.0 | 92.2 | 7.6 | |
| fCfgaGfaaauU | cgaaCfuaaugs | | | | | | | | | |
| fca | usa | | | | | | | | | |
| gsgsUfaugAf | usGfsuucuguc | 49.7 | 5.5 | 76.6 | 4.4 | 88.2 | 4.2 | 96.6 | 5.9 | |
| AfUfagAfcaga | uauuCfauaccs | | | | | | | | | |
| Afca | asg | | | | | | | | | |
| gscsCfcaaUfC | usGfsucgcuaa | 51.6 | 8.0 | 77.9 | 6.2 | 99.8 | 12.8 | 86.9 | 19.1 | |
| fCfauUfagcgA | uggaUfugggcs | | | | | | | | | |
| fca | asg | | | | | | | | | |
| csasUfuagCfG | usAfsuccuacu | 51.6 | 15.2 | 72.9 | 26.7 | 85.6 | 4.8 | 96.6 | 5.8 | |
| fAfcaGfuagg | gucgCfuaaugs | | | | | | | | | |
| Afua | gsa | | | | | | | | | |
| csasUfaacUfA | usGfsaucuuaa | 52.7 | 6.5 | 89.4 | 6.6 | 104.0 | 9.1 | 96.6 | 6.7 | |
| fGfauUfaagaU | ucuaGfuuaugs | | | | | | | | | |
| fca | asc | | | | | | | | | |
| gsasCfgaaUfA | usUfsguuggau | 53.9 | 2.5 | 78.1 | 8.3 | 112.4 | 8.7 | 91.8 | 8.9 | |
| fUfgaUfccaaC | cauaUfucgucs | | | | | | | | | |
| faa | csa | | | | | | | | | |
| gsgsGfauaAf | usAfsuuaccua | 54.9 | 9.7 | 81.3 | 16.4 | 82.6 | 7.5 | 95.4 | 4.7 | |
| UfGfauAfggu | ucauUfaucccs | | | | | | | | | |
| aAfua | asa | | | | | | | | | |
| ascsAfaucUfC | usGfsccaaaccu | 55.6 | 15.1 | 77.0 | 15.1 | 88.9 | 3.0 | 99.8 | 2.0 | |
| fUfagGfuuug | agaGfauugusa | | | | | | | | | |
| Gfca | sa | | | | | | | | | |
| ususGfacgAf | usAfsauuagcu | 56.7 | 1.9 | 87.1 | 12.0 | 93.5 | 3.2 | 97.7 | 6.8 | |
| UfAfcaGfcuaa | guauCfgucaas | | | | | | | | | |
| Ufua | gsg | | | | | | | | | |
| gsusAfcuaCfU | usAfsauaauua | 57.5 | 7.7 | 91.2 | 9.7 | 91.7 | 11.6 | 95.7 | 5.3 | |
| fCfcuAfauuaU | ggagUfaguacs | | | | | | | | | |
| fua | asg | | | | | | | | | |
| gscsAfuggUf | usAfscuuucac | 60.6 | 5.0 | 101.0 | 4.0 | 94.5 | 12.2 | 101.3 | 3.8 | |
| GfAfggUfgaa | cucaCfcaugcs | | | | | | | | | |
| aGfua | csa | | | | | | | | | |
| csasUfgguGf | usUfsacuuuca | 61.1 | 11.3 | 86.0 | 2.0 | 86.6 | 11.0 | 93.6 | 7.6 | |
| AfGfguGfaaa | ccucAfccaugs | | | | | | | | | |
| gUfaa | csc | | | | | | | | | |
| cscsAfaucCfA | usCfsugucgcu | 61.8 | 14.6 | 82.2 | 16.1 | 95.1 | 5.6 | 97.0 | 3.5 | |
| fUfuaGfcgacA | aaugGfauuggs | | | | | | | | | |
| fga | gsc | | | | | | | | | |
| gscsAfaugAf | usGfsuacuggu | 62.7 | 3.3 | 104.8 | 7.7 | 114.1 | 1.6 | 95.4 | 5.1 | |
| GfGfgaCfcagu | cccuCfauugcs | | | | | | | | | |
| Afca | asc | | | | | | | | | |
| ascsCfauuUfU | usAfsuauagcc | 62.8 | 8.0 | 88.2 | 6.6 | 98.0 | 5.7 | 91.6 | 7.9 | |
| fGfggGfcuau | ccaaAfauggus | | | | | | | | | |
| Afua | usg | | | | | | | | | |
| csasCfugcUfA | usCfscaugacu | 67.8 | 4.8 | 92.6 | 6.9 | 91.8 | 11.7 | 90.8 | 3.7 | |
| fUfuaGfucau | aauaGfcagugs | | | | | | | | | |
| Gfga | gsa | | | | | | | | | |
| ascsCfuauGfG | usCfscuacuag | 69.2 | 8.9 | 102.9 | 4.2 | 105.8 | 4.0 | 101.8 | 8.1 | |
| fUfccUfaguaG | gaccAfuaggus | | | | | | | | | |
| fga | asc | | | | | | | | | |
| usgsUfacuAfC | usAfsuaauuag | 69.3 | 13.3 | 83.1 | 9.0 | 94.8 | 12.9 | 89.7 | 7.7 | |
| fUfccUfaauuA | gaguAfguacas | | | | | | | | | |
| fua | gsu | | | | | | | | | |
| usgsAfccuAf | usAfsaauuagu | 72.5 | 5.6 | 90.9 | 4.8 | 87.1 | 6.3 | 100.2 | 4.1 | |
| AfUfcaCfuaau | gauuAfggucas | | | | | | | | | |
| Ufua | asa | | | | | | | | | |
| ususUfaacCfU | usGfsguguaac | 78.9 | 6.9 | 92.0 | 1.4 | 99.1 | 10.6 | 98.8 | 4.5 | |
| fAfugUfuacaC | auagGfuuaaas | | | | | | | | | |
| fca | asa | | | | | | | | | |
| cscsUfaacUfU | usUfsaaccuau | 84.0 | 5.3 | 79.5 | 8.8 | 105.4 | 3.9 | 95.1 | 7.8 | |
| fUfuaUfaggu | aaaaGfuuaggs | | | | | | | | | |
| Ufaa | usu | | | | | | | | | |
| asgsUfucgAf | usUfsuucgaau | 88.3 | 9.5 | 92.3 | 4.4 | 105.8 | 5.7 | 90.9 | 6.9 | |
| GfAfaaUfucga | uucuCfgaacus | | | | | | | | | |
| Afaa | asa | | | | | | | | | |
| ascsGfaauAfU | usUfsuguugga | 89.5 | 4.6 | 78.9 | 7.4 | 113.7 | 7.8 | 95.9 | 3.2 | |
| fGfauCfcaacA | ucauAfuucgus | | | | | | | | | |
| faa | CSC | | | | | | | | | |
| asusCfcauUfA | usCfsuacuguc | 92.0 | 14.8 | 84.4 | 5.6 | 112.3 | 6.0 | 93.4 | 7.1 | |
| fGfcgAfcagu | gcuaAfuggaus | | | | | | | | | |
| Afga | usg | | | | | | | | | |
| csusGfcccAfA | usCfsgcuaaug | 99.2 | 3.0 | 95.6 | 2.2 | 88.9 | 3.0 | 102.2 | 2.6 | |
| fUfccAfuuagC | gauuGfggcags | | | | | | | | | |
| fga | csa | | | | | | | | | |
| csgsGfcucGfG | usGfsgaguacu | 99.5 | 4.9 | 98.0 | 7.0 | 111.8 | 8.2 | 97.4 | 2.1 | |
| fCfcaGfuacuC | ggccGfagccgs | | | | | | | | | |
| fca | csc | | | | | | | | | |
| ususAfaccUfA | usUfsgguguaa | 103.4 | 5.3 | 91.9 | 3.9 | 93.1 | 4.3 | 107.5 | 3.4 | |
| fUfguUfacacC | cauaGfguuaas | | | | | | | | | |
| faa | asa | | | | | | | | | |
| csasAfuccAfU | usAfscugucgc | 134.5 | 8.9 | 85.6 | 10.6 | 111.4 | 6.8 | 96.5 | 4.2 | |
| fUfagCfgacaG | uaauGfgauugs | | | | | | | | | |
| fua | gsg | | | | | | | | | |

The best 24 were selected and a dose response to assess Kras silencing was performed. The results of the dose response (IC50s and percentage inhibition) are included in Table 4 below. The screen of these siRNAs showed dose response curves of quite diverse shapes: some well reaching a plateau of 100% target expression, whereas some surprisingly looked as if a saturation/the max knock down reached over all doses tested.

TABLE 4
Dose Response

| | | IC20 | IC50 | IC80 | max. inhib. |
|---|---|---|---|---|---|
| sense | antisense | [nM] | [nM] | [nM] | [%] |
| usgsAfcgaUfAfCfagCfua | usGfsaauuagcuguaUfcg | 0.00 | 0.00 | 0.2 | 88.2 |
| auUfca | ucasasg | | | | |
| ascsGfauaCfAfGfcuAfau | usCfsugaauuagcugUfau | 0.00 | 0.10 | 30.2 | 82.9 |
| ucAfga | cguscsa | | | | |
| gsasUfacaGfCfUfaaUfuc | usUfsucugaauuagcUfgu | 0.13 | 0.54 | #N/A | 76.0 |
| agAfaa | aucsgsu | | | | |
| csgsAfauaUfGfAfucCfaa | usAfsuuguuggaucaUfau | 0.00 | 0.00 | 0.2 | 82.0 |
| caAfua | ucgsusc | | | | |
| gsusCfucuUfGfGfauAfu | usCfsgagaauauccaAfgag | 0.05 | 0.33 | 7.6 | 84.6 |
| ucuCfga | acsasg | | | | |
| gsgsAfuauUfCfUfcgAfca | usGfscugugucgagaAfua | 0.30 | 1.19 | 128.0 | 79.7 |
| caGfca | uccsasa | | | | |
| asgsAfuauUfCfAfccAfuu | usCfsuauaauggugaAfua | 0.00 | 0.00 | 0.7 | 85.8 |
| auAfga | ucususc | | | | |
| gsasUfauuCfAfCfcaUfua | usUfscuauaauggugAfau | 0.00 | 0.01 | 1.7 | 86.2 |
| uaGfaa | aucsusu | | | | |
| usasUfucaCfCfAfuuAfua | usUfscucuauaauggUfga | 0.00 | 0.02 | 4.2 | 82.3 |
| gaGfaa | auasusc | | | | |
| gsgsAfcuuAfGfCfaaGfaa | usUfsaacuucuugcuAfag | 0.00 | 0.05 | 1.8 | 88.1 |
| guUfaa | uccsusg | | | | |
| ususCfuauAfCfAfuuAfg | usUfscgaacuaauguAfua | 0.00 | 0.02 | 5.0 | 86.0 |
| uucGfaa | gaasgsg | | | | |
| asusUfaguUfCfGfagAfaa | usCfsgaauuucucgaAfcua | 0.02 | 0.26 | 9.4 | 87.5 |
| uuCfga | ausgsu | | | | |
| ususAfguuCfGfAfgaAfa | usUfscgaauuucucgAfac | 0.00 | 0.02 | 0.5 | 86.4 |
| uucGfaa | uaasusg | | | | |
| asgsAfaauUfCfGfaaAfac | usUfsuauguuuucgaAfuu | 0.00 | 0.02 | 0.9 | 88.7 |
| auAfaa | ucuscsg | | | | |
| usgsUfacaUfUfAfcaCfua | usAfsauuuaguguaaUfgu | 0.00 | 0.06 | #N/A | 78.2 |
| aaUfua | acasasa | | | | |
| gscsAfguuGfAfUfuaCfu | usUfsaagaaguaaucAfacu | 0.00 | 0.00 | 0.0 | 89.5 |
| ucuUfaa | gcsasu | | | | |
| gsasAfucaUfCfCfcuAfuu | usAfscagaauagggaUfga | 0.00 | 0.04 | 4.2 | 87.1 |
| cuGfua | uucsasa | | | | |
| ascsAfuuaGfAfUfaaAfuu | usUfsaguaauuuaucUfaa | 0.01 | 0.12 | #N/A | 78.7 |
| acUfaa | ugusgsa | | | | |
| asgsUfaugAfAfAfugGfg | usUfsaauccccauuuCfaua | 0.00 | 0.12 | #N/A | 71.0 |
| gauUfaa | cusgsg | | | | |
| asgsGfacaUfCfAfcuUfac | usGfsauaguaagugaUfgu | 0.02 | 0.23 | 9.6 | 84.6 |
| uaUfca | ccuscsa | | | | |
| cscsCfuaaCfCfAfuaAfga | usUfsaaaucuuauggUfua | 0.00 | 0.06 | #N/A | 75.6 |
| uuUfaa | gggsgsa | | | | |
| cscsGfaaaGfUfUfucCfaa | usGfsgaauuggaaacUfuu | 0.07 | 0.51 | 23.6 | 82.3 |
| uuCfca | cggsasu | | | | |
| gscsCfaauUfUfCfuuAfcu | usUfsacuaguaagaaAfuu | 0.00 | 0.01 | #N/A | 76.6 |
| agUfaa | ggcsasc | | | | |
| cscsAfauuUfCfUfuaCfua | usGfsuacuaguaagaAfau | 0.00 | 0.01 | 28222.7 | 79.2 |
| guAfca | uggscsa | | | | |

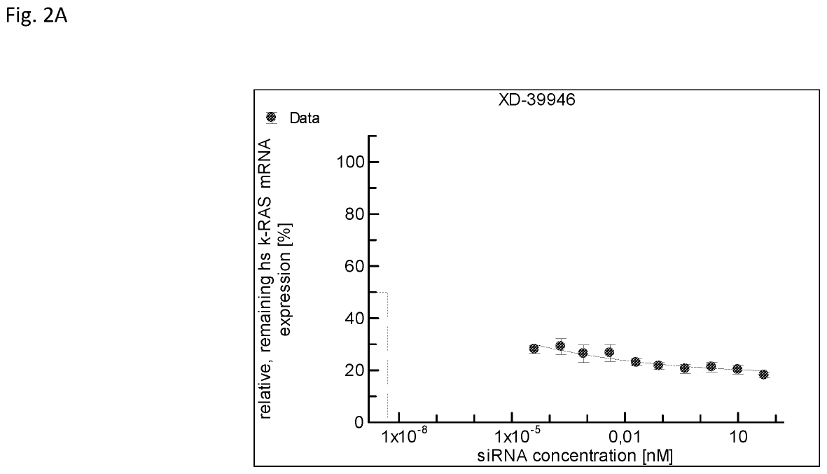

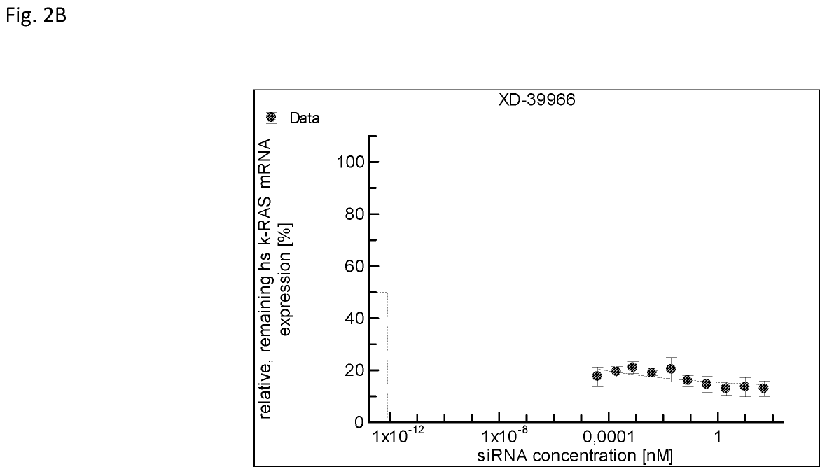

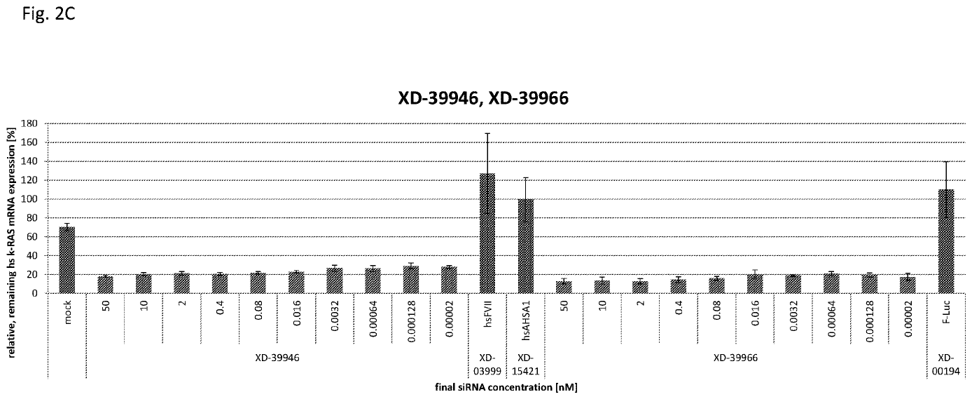

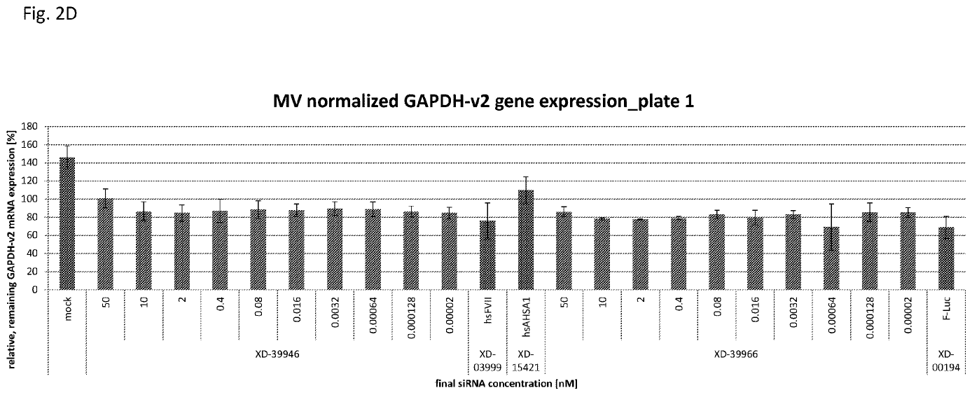

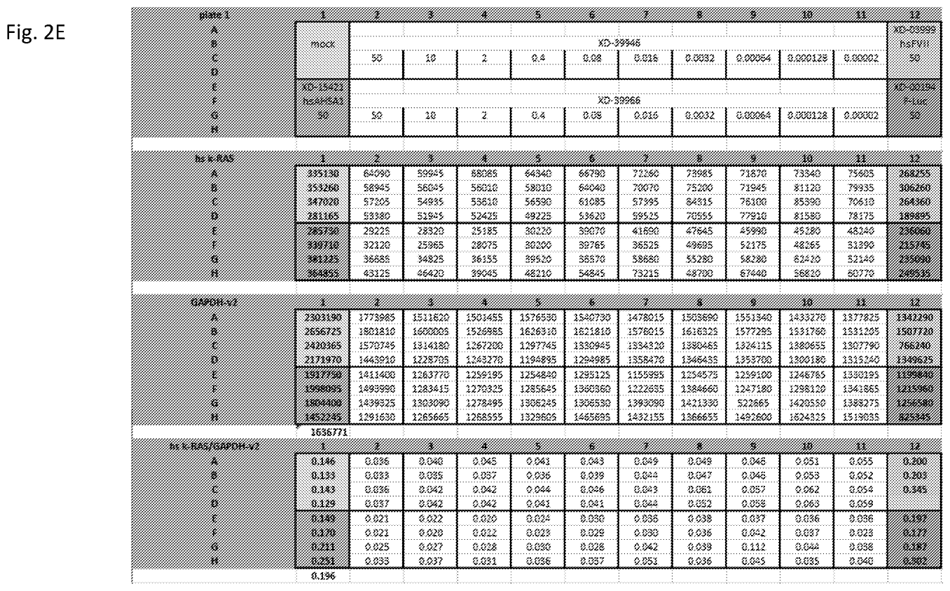

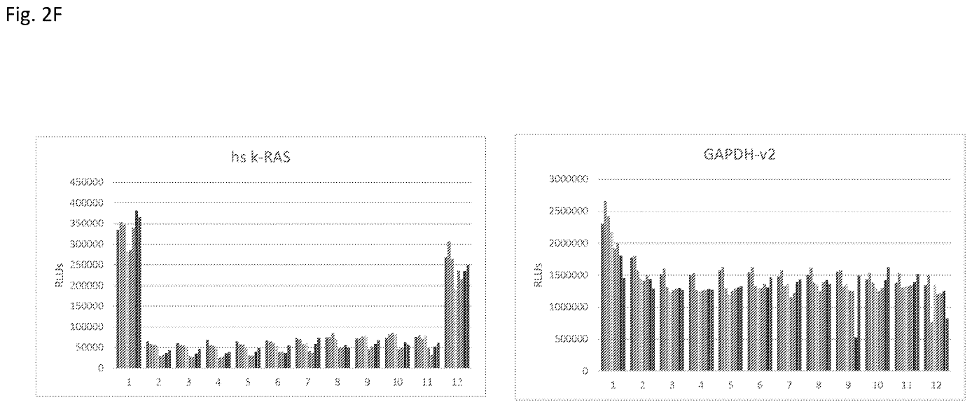

Two of the siRNAs (shown in Table 5 below) had an unexpectedly high activity and were tested at lower doses to reach the concentration of 0.00002 nM (20fM). This dose-response curve also showed maximal knock down at all doses. The results of this analysis are shown in FIGS. 2A-2F.

TABLE 5

| sense | antisense |
|---|---|
| csgsAfauaUfGfA | usAfsuuguuggau |
| fucCfaacaAfua | caUfauucgsusc |
| gscsAfguuGfAfU | usUfsaagaaguaa |
| fuaCfuucuUfaa | ucAfacugcsasu |

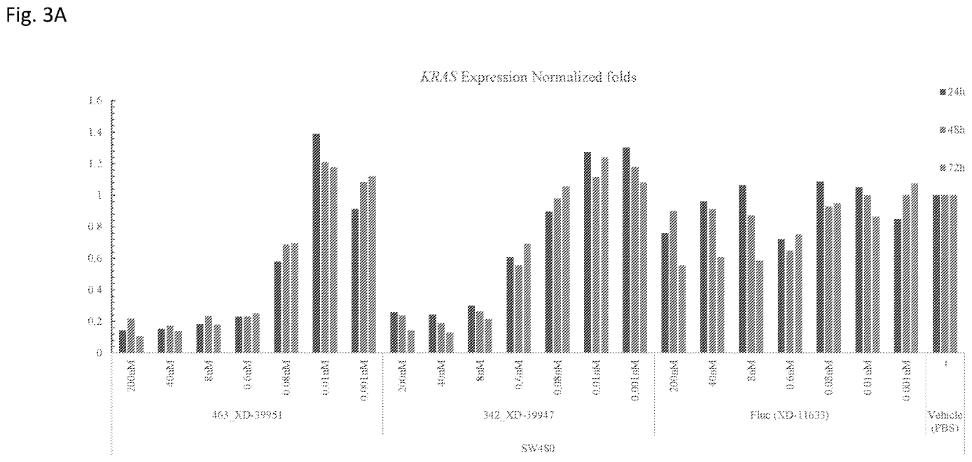

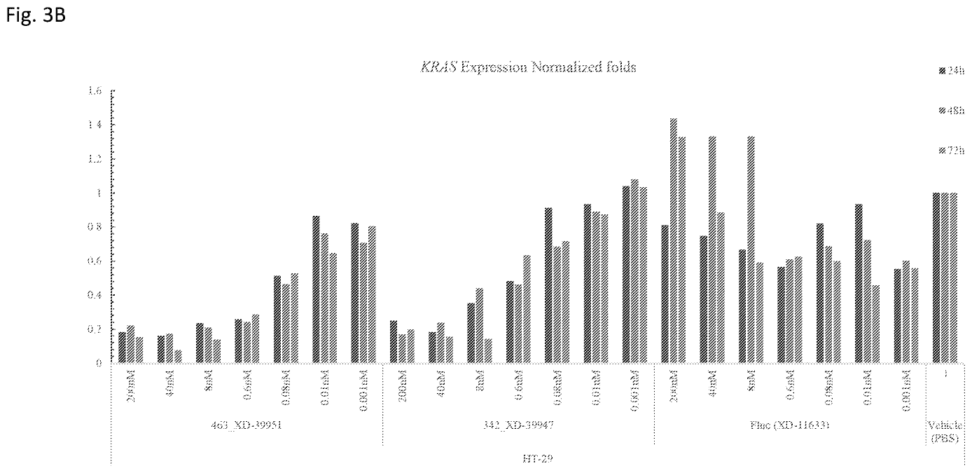

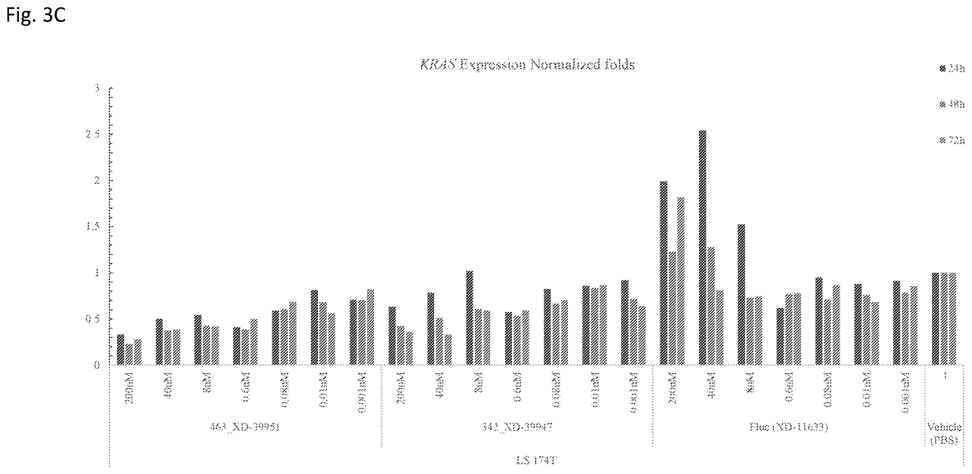

Finally, two candidates (XD-39951 and XD-39947) have been tested in cell lines harboring different mutations of KRAS. These assessments have been performed by transfecting the siRNAs into cells carrying either wt KRAS or KRAS mutations (HT-29 cells: wt; SW480: G12V mutation; LS174T: G12D mutation). As shown in FIGS. 3A-3C, both sequences were able to knock down the KRAS, regardless of the mutation.

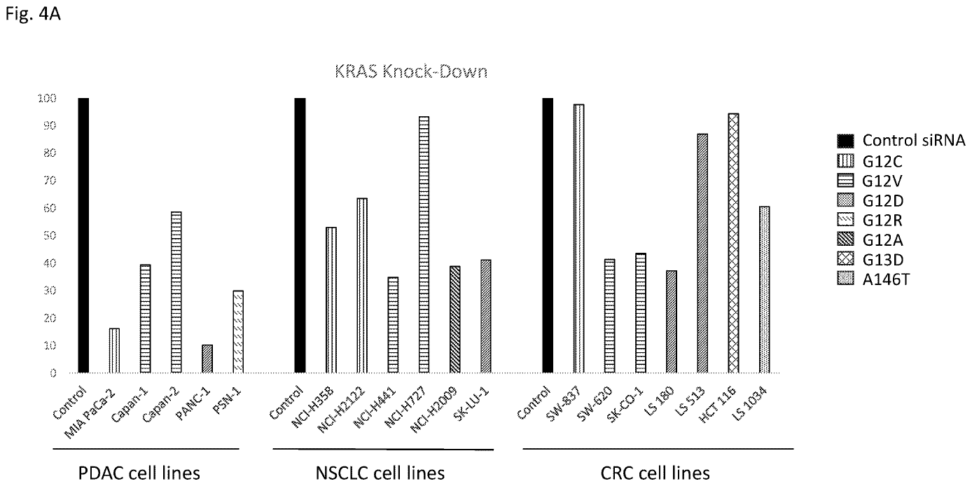

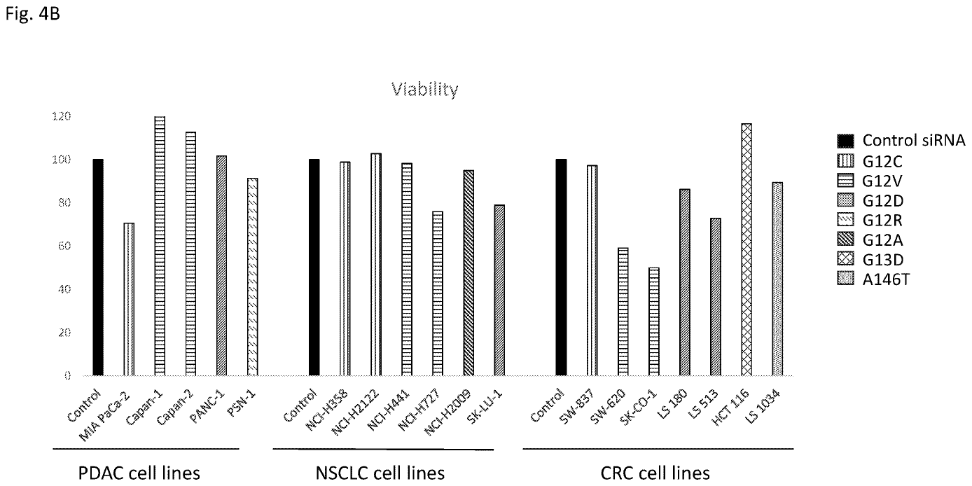

The candidate of XD-39951 was further tested in more cell lines harboring additional mutations of KRAS. These assessments have been performed by transfecting the siRNAs into cells carrying the additional KRAS mutations. Ash shown in FIG. 4A, in addition to G12V and G12D, XD-39951 is able to knock down KRAS mutations G12C, G12R, G12A and A146T. As shown in FIG. 4B, knocking down of KRAS leads to reduced cell viability in some cases.

The formulation allows for specific delivery to the tumors. This is because tumors usually have leaky vasculature allowing for extravasation of the nanoparticle disclosed herein due to its physicochemical characteristics.

The coating of the nanoparticles with albumin enriches the local concentration of the nanoparticles through binding to the receptors pg60 and/or SPARC. These receptors are upregulated in certain tumors.

Coating the nanoparticle with hyaluronic acid can have the same effect on other tumor types through the CD44 receptor.

### Example 2—Material and Methods

1.1 Knock-Down Analysis of Kras in NCI-H23 Cells Following Transfection of Different siRNAs

### Materials

| Materials | Vendor |
|---|---|
| NCI-H23 cells | ATCC |
| RNAiMax | Invitrogene |
| bDNA | ThermoFisher |

### Methods

NCI-H23 cells (ATCC) at a density of 20.000 cells per well were transfected with increasing concentrations of Kras siRNAs (0.00002 nM-50 nM) using RNAiMax transfection agent (Invitrogene) following the manufacturer's instructions. 24 h after transfection Kras knock down was analysed using a Quantigene® branched DNA assay.

1.2 Knock-Down Analysis of Kras in HT-29, SW480 and LS174T Cells Following Transfection of Different siRNAs
Cell Line Used in this Study

| | Cell | | | Tissue | |
|---|---|---|---|---|---|
| No. | Line | Species | Category | Origin | Medium |
| 1 | SW480 | Human | Tumor | Colon | L-15 + 10% FBS |
| 2 | HT-29 | Human | Tumor | Colon | McCoy's 5a + 10% FBS |
| 3 | LS174T | Human | Tumor | Colon | MEM + 0.01 mM |
| | | | | | NEAA + 10% FBS |

Note:
Cells were cultured in 37° C. incubator with 5% CO₂ and 95% air (except SW480 with 100% air).

### Reagents for Cell Culture and Transfection

| Item | Supplier | Cat# |
|---|---|---|
| L-15 Medium | Gibco | 11415-064 |
| McCoy's 5a Medium | Gibco | 12330-031 |
| MEM | Hyclone | SH30024.01 |
| FBS | Gibco | 10099-141 |
| Trypsin 0.25% EDTA | Gibco | 25200-072 |
| NEAA | Gibco | 11140-050 |
| siRNA | Axolabs | N/A |
| Lipofectamine RNAiMAX | Invitrogen | 13778150 |

### Reagents for RT-PCR

| Reagent | Vendor | Cat No. |
|---|---|---|
| RNeasy Mini Kit | Qiagen | 74106 |
| RNase-Free DNase Set | TIANGEN | RT411 |
| High Capacity cDNA Reverse Transcriptase | ABI | 4374966 |
| Kit | | |
| TaqMan Universal PCR Master Mix | ABI | 4304437 |

### Probe Information

| | | | Catalog |
|---|---|---|---|
| Gene | Assay ID | Vendor | number |
| KRAS | Hs00364282_m1 | Thermo | 4331182 |
| | | Fisher | |
| GAPDH | Hs02758991_g1 | Thermo | 4331182 |
| | | Fisher | |

### Equipment

1) Applied Biosystems Inc. (ABI), fast PCR system 7900H, 384 well format

### Equipment ID: BEPCR0030

2) Data analysis software: ABI SDS2.4
3) Nanodrop™ 2000 spectrophotometer

### Equipment ID: BENOP0020

4) Qiagen Tissue lyserII

### Equipment ID: BETIS0010

### Methods

Resuspension of siRNA (Aim 1-3)
a) Briefly centrifuge the screw cap vial at low speed (maximum 4000×g) to ensure that all material is collected at the bottom of the vial or well before opening.
b) Carefully remove the screw cap.
c) Add nuclease-free water to achieve the stocking concentration 100 μM.
d) Let the vial or plate stand for a few minutes at ambient temperature.
e) Gently pipette up and down 5 times to resuspend.
f) Repeat steps d and e.
g) Aliquot the resuspended siRNA into multiple tubes or plates to limit the number of freeze-thaw cycles. Store at −80° C.
h) Note: siRNA solution was on ice when preparing transfection reaction.

Cell seeding and TO plate reading (Aim 1-3)
a) Plate cells in 96-well plates at a pre-determined density in 90 μL culture medium 24 hours before transfection (Day 0). Cells should reach 30-50% confluency on the next day.
b) Take plate TO group (Day 1), and add 10 μL culture medium to each well for TO reading.
c) Add 100 μL CellTiter-Glo Reagent to each well.
d) Mix contents for 20 mins on an orbital shaker to facilitate cell lysis.
e) Allow the plate to incubate at room temperature for 10 mins to stabilize luminescent signal. (Note: Uneven luminescent signal within standard plates can be caused by temperature gradients, uneven seeding of cells, and edge effects in multiwall plates.)
f) Place Backseal black sticker to the bottom of each plate.
g) Record luminescence using En Vision Multilabel Reader.
   siRNA Transfection (Aim 1-3)
a) On the day of transfection (Day 1), refresh the culture medium with 90 μL culture medium.
b) Transfect cells with siRNA at a range of final concentration in triplicate (see Appendix). Prepare siRNA-lipid complex as shown below:

| | | |
|---|---|---|
| Diluted siRNA | Opti-MEM Medium (μL) | 5 |
| | siRNA (μL) | 0.1 |
| Diluted | Opti-MEM Medium (μL) | 5 |
| Lipofectamine | Lipofectamine RNAiMAX Reagent | 0.3 |
| | (μL) | |

c) Add 5 μL diluted siRNA to 5 μL diluted Lipofectamine and incubate the mix for 5 mins at RT. Add the siRNA-lipid complexes dropwise to each well and mix gently by rocking the plate back and forth. Incubate the cells for a specific time before cell viability assay (see Appendix). Refresh the culture medium after 24 hrs of incubation if required.
d) To evaluate cell viability, add 100 μL CellTiter-Glo Reagent to each well. Mix the contents for 20 mins on an orbital shaker to facilitate cell lysis. Incubate the plate at RT for 10 mins to stabilize luminescent signals. Place a Backseal black sticker to the bottom of each plate and measure the luminescence data using an En Vision Multi Label Reader.
   qPCR Sample Collection (Aim 1-3)
a) Plate cells in 6-well plates at a pre-determined density in 2.25 mL culture medium 24 hours before transfection (Day 0). Cells should reach 30-50% confluency on the next day.
b) On the day of transfection (Day 1), refresh the culture medium with 2.25 mL growth medium.
c) Transfect cells with siRNA at a range of final concentration (see Appendix). Prepare siRNA-lipid complex as shown below:

| | | |
|---|---|---|
| Diluted siRNA | Opti-MEM Medium (μL) | 125 |
| | siRNA (μL) | 2.5 |
| Diluted | Opti-MEM Medium (μL) | 125 |
| Lipofectamine | Lipofectamine RNAiMAX Reagent | 7.5 |
| | (μL) | |

d) Add 125 μL diluted siRNA to 125 μL diluted Lipofectamine and incubate the mix for 5 mins at RT. Add the siRNA-lipid complexes dropwise to each well and mix gently by rocking the plate back and forth. Incubate the cells for a specific time before harvested. Refresh the culture medium after 24 hrs of incubation if required.
e) Remove the culture medium and freeze the transfected cells in liquid nitrogen and store the cells at −80° C.

### Sample Information

366 samples from in-vitro efficacy study were used for gene expression detection. The detailed information samples are displayed in Table 6 below.

TABLE 6

| | | | | | Treatment |
|---|---|---|---|---|---|
| No. | N | Cell Lines | Treatment | Doses | time |
| Aim1 | 90 | SW480 | 463_XD-39951 | 200, 40, 8 | 24, 48 and |
| | | HT-29 | 342_XD-39947 | nM | 72 h |
| | | LS174T | Fluc (XD-11633) | | |
| | | | Vehicle | — | |
| Aim2 | 75 | SW480 | 2 siRNAs + Fluc | TBD by | 24, 48 or |
| | | HT-29 | (XD-11633) | client | 72 h |
| | | LS174T | (TBD) | (8 dose) | (TBD) |
| | | | Vehicle | — | |
| Aim3 | 201 | SW480 | 10 siRNAs + Fluc | TBD by | 24, 48 or |
| | | HT-29 | (XD-11633) | client | 72 h |
| | | LS174T | (TBD) | (6 dose) | (TBD) |
| | | | Vehicle | — | |

### Total RNA Extraction

1) Place a stainless-steel bead (5 mm mean diameter) and 350 μL Buffer RLT into a 2 mL microcentrifuge tube containing cell. Place the tubes in the TissueLyser adapter set and operate the TissueLyser for 5 min at 20 Hz. Proceed with RNA extraction.
2) Add 1 volume of 70% ethanol to the lysate and mix well by pipetting. Do not centrifuge. Proceed immediately to step 3.
3) Transfer up to 700 μL of the sample, including any precipitate, to a RNeasy Mini spin column placed in a 2 mL collection tube (supplied). Close the lid, and centrifuge for 15 s at ≥8000×g. Discard the flow-through. Transfer the remaining lysate to the same tube and repeat the step 3.
4) Add 350 μL Buffer RW1 to the RNeasy column. Close lid, centrifuge for 15 s at 8000×g. Discard the flow-through.
5) Add 10 μL DNase I stock solution to 70 μL Buffer RDD. Mix by gently inverting the tube, and centrifuge briefly.
6) Add the DNase I incubation mix (80 μL) directly to the RNeasy column membrane, and place the tubes on the bench top (20-30° C.) for 15 min.
7) Add 350 μL Buffer RW1 to the RNeasy column. Close the lid, centrifuge for 15s at 8000×g. Discard the flow-through.
8) Add 500 μL Buffer RPE to the spin column. Close the lid gently, and centrifuge for 2 min at 8000×g to wash the spin column membrane. Discard the flow-through.
9) Add 500 μL Buffer RPE to the spin column. Close the lid gently, and centrifuge for 2 min at 8000×g to wash the spin column membrane.
10) Place the RNeasy spin column in a new 1.5 mL collection tube (supplied). Add 30-50 μL RNase-free water directly to the center of the spin column membrane. Close the lid gently, and centrifuge for 1 min at full speed to elute the RNA.

### RNA Quantification

Total RNA quantification by Nanodrop™ 2000 spectrophotometer.

cDNA Synthesis (Reverse Transcription, RT)
1) Set up the RT reaction as follows:

| Reagent | Volume | |
|---|---|---|
| 10 × RT Buffer Mix | 2 | μL |
| 25 × dNTP Mix | 0.8 | μL |
| 10 × RT Random Primers | 2 | μL |
| RNase inhibitor | 1 | μL |
| 50 U/uL MultiScribe Reverse Transcriptase | 1 | μL |
| RNA | 2000 | ng |

| | |
|---|---|
| Nuclease/RNase free Water | To 20 μL |

2) RT reaction conditions:

| | |
|---|---|
| Temperature | Time |

| | | | |
|---|---|---|---|
| 25° | C. | 10 | min |
| 37° | C. | 120 | min |
| 85° | C. | 5 | min |

| | | |
|---|---|---|
| 4° | C. | Forever |

3) To achieve highest yield of cDNA, 20 μL RT reactions were performed with up to 2 μg total RNA. cDNA samples were stored at −20° C. or used for Real-Time PCR immediately.

### Real-Time PCR Reaction (TaqMan Method)

1) Prepare the Real-Time PCR as follows:

| Reagent | Volume | |
|---|---|---|
| TaqMan 2 × Universal PCR | 5 | μL |
| MasterMix | | |
| Probe | 0.5 | μL |
| RNase-free Water | 2.5 | μL |
| cDNA | 2 | μL |
| Total | 10 | μL |

2) Procedure of Real-Time PCR

| | | |
|---|---|---|
| Temperature | Time | Cycle |

| | | | | |
|---|---|---|---|---|
| 50° C. | 5 | min | 1 | cycle |
| 95° C. | 10 | min | 1 | cycle |
| 95° C. | 15 | sec | 45 | cycles |

| | | |
|---|---|---|
| 60° C. | 1 | min |

3) ddH2O was used as a no template control (NTC). RNA sample was used as a no reverse transcription control (No RT). Each sample had three technical replicates.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

Various publications, articles and patents are cited or described in the background and throughout the specification; each of these references is herein incorporated by reference in its entirety. The incorporated patents include, but are not limited to, U.S. Pat. Nos. 9,987,371, 10,758,627, and 11,529,388. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

1. A pharmaceutical composition comprising a peptide-polynucleotide complex,
wherein the peptide comprises an amino acid sequence with at least 80% identity to the amino acid sequence of SEQ ID NO: 1, SEQ ID NO: 2, or SEQ ID NO: 3; and
wherein the polynucleotide is a small interfering RNA (siRNA) targeting human KRAS mRNA, wherein the target sequence of human KRAS mRNA does not encode G12, G13, or Q61 with reference to SEQ ID NO: 4 or a mutant amino acid at position 12, 13, or 61 with reference to SEQ ID NO: 4.

2. The pharmaceutical composition of claim 1, wherein the peptide is non-lytic, non-cytotoxic, and capable of affecting the release of the polynucleotide from an endosome of a cell.

3. The pharmaceutical composition of claim 1, wherein the peptide comprises two or more contiguous, basic amino acids (a cationic region) and one or more histidine residues located adjacent to the cationic region.

4. The pharmaceutical composition of claim 1, wherein the peptide comprises an amino acid sequence of SEQ ID NO: 1, SEQ ID NO: 2, or SEQ ID NO: 3.

5. The pharmaceutical composition of claim 1, wherein the siRNA comprises a sense strand and an antisense strand.

6. The pharmaceutical composition of claim 5, wherein the sense strand and the antisense strand are each 16-24 bases in length.

7. The pharmaceutical composition of claim 5, wherein the sense strand is 19 bases in length.

8. The pharmaceutical composition of claim 5, wherein the antisense strand is 21 bases in length.

9. The pharmaceutical composition of claim 5, wherein the sense strand and the antisense strand are modified.

10. The pharmaceutical composition of claim 9, wherein the modifications are selected from the group consisting of 2′-methoxy (2′-OMe), 2′-fluoro (2′-F), 2′-O-methoxyethyl (2′-O-MOE), 5′-vinylphosphonate, phosphorothioate (PTO), locked nucleic acid (LNA), locked nucleic acid (UNA), glycol nucleic acid (GNA), and DNA.

11. The pharmaceutical composition of claim 10, wherein the modifications of the sense strand comprise:
1) PTO at positions 1 and 2;
2) 2′-F at positions 3, 7-9, 12, and 17; and
3) 2′-OMe at positions 1, 2, 4-6, 10, 11, 13-16, 18, and 19.

12. The pharmaceutical composition of claim 10, wherein the modifications of the antisense strand comprise:
1) PTO at positions 1, 2, 19, and 20;
2) 2′-F at positions 2 and 14; and
3) 2′-OMe at positions 1, 3-13, and 15-21.

13. The pharmaceutical composition of claim 5, wherein the last nucleotide of the sense strand is adenine (A).

14. The pharmaceutical composition of claim 5, wherein the first nucleotide of the antisense strand is uracil (U).

15. The pharmaceutical composition of claim 5, wherein the sense strand comprises a nucleotide sequence with at least at least 80% identity to the nucleotide sequence of any one of the sensen strands listed in Error! Reference source not found, and Error! Reference source not found.

16. The pharmaceutical composition of claim 5, wherein the antisense strand comprises a nucleotide sequence with at least at least 80% identity to the nucleotide sequence of any one of the antisensen strands listed in Error! Reference source not found. and Error! Reference source not found.

17. The pharmaceutical composition of claim 1, wherein the ratio of peptide to polynucleotide is about 2:1 to about 3500:1, and wherein the ratio is the molar ratio.

18. The pharmaceutical composition of claim 17, wherein the molar ratio of peptide to polynucleotide is about 5:1 to about 200:1.

19. The pharmaceutical composition of claim 17, wherein the molar ratio of peptide to polynucleotide is about 100:1.

20. The pharmaceutical composition of claim 17, wherein the molar ratio of peptide to polynucleotide is about 5:1.

21. The pharmaceutical composition of claim 1, wherein the ratio of peptide to polynucleotide is about 6:1 to about 18:1, and wherein the ratio is the ratio of positively-chargeable polymer amine groups to negatively-charged nucleic acid phosphate groups.

22. The pharmaceutical composition of claim 21, wherein the charge ratio of peptide to polynucleotide is about 12:1.

23. The pharmaceutical composition of claim 1, wherein the peptide-polynucleotide complex is a nanoparticle with a diameter of about 10 nm to about 300 nm.

24. The pharmaceutical composition of claim 1, wherein the peptide-polynucleotide complex is coated with albumin and/or hyaluronic acid.

25. The pharmaceutical composition of claim 1, wherein the pharmaceutical composition further comprises a pharmaceutical acceptable carrier.

26. A method of treating a disease or disorder in a subject, the method comprising administering to the subject a therapeutically effective amount of the pharmaceutical composition of claim 1.

27. The method of claim 26, wherein the disease or disorder is cancer.

28. The method of claim 27, wherein the cancer is blood cancer or solid tumor cancer.
